SEQUENCE LISTING (1006 sequences)

SEQ ID NO: 1 - RNA, 19 nt, Artificial Sequence
  Description of Artificial Sequence: Synthetic oligonucleotide
caaagaaugucgacccuaa

SEQ ID NO: 2 - RNA, 19 nt, Artificial Sequence
  Description of Artificial Sequence: Synthetic oligonucleotide
gcucuuugcugcacuauuu

SEQ ID NO: 3 - RNA, 19 nt, Artificial Sequence
  Description of Artificial Sequence: Synthetic oligonucleotide
gcacgccgcucuucacugu

SEQ ID NO: 4 - RNA, 19 nt, Artificial Sequence
  Description of Artificial Sequence: Synthetic oligonucleotide
gaucaugggugucaucuac

SEQ ID NO: 5 - RNA, 19 nt, Artificial Sequence
  Description of Artificial Sequence: Synthetic oligonucleotide
gacaaggacgggcacauua

SEQ ID NO: 6 - RNA, 19 nt, Artificial Sequence
  Description of Artificial Sequence: Synthetic oligonucleotide
gcuacuuccuccucaagaa

SEQ ID NO: 7 - RNA, 19 nt, Artificial Sequence
  Description of Artificial Sequence: Synthetic oligonucleotide
gaccgccucugcuuuguca

SEQ ID NO: 8 - RNA, 19 nt, Artificial Sequence
  Description of Artificial Sequence: Synthetic oligonucleotide
ggcagcacguguacgagaa

SEQ ID NO: 9 - RNA, 19 nt, Artificial Sequence
  Description of Artificial Sequence: Synthetic oligonucleotide
caacuacuccaacgacuug

SEQ ID NO: 10 - RNA, 19 nt, Artificial Sequence
  Description of Artificial Sequence: Synthetic oligonucleotide
ccaacaagaagaugaguaa

SEQ ID NO: 11 - RNA, 19 nt, Artificial Sequence
  Description of Artificial Sequence: Synthetic oligonucleotide
gcccgcagccuguuucuuu

SEQ ID NO: 12 - RNA, 19 nt, Artificial Sequence
  Description of Artificial Sequence: Synthetic oligonucleotide
gcaggagcuucucgacuuc

SEQ ID NO: 13 - RNA, 19 nt, Artificial Sequence
  Description of Artificial Sequence: Synthetic oligonucleotide
gggagauagugaugaagua

SEQ ID NO: 14 - RNA, 19 nt, Artificial Sequence
  Description of Artificial Sequence: Synthetic oligonucleotide
gaaguacauccauuauaag

SEQ ID NO: 15 - RNA, 19 nt, Artificial Sequence
  Description of Artificial Sequence: Synthetic oligonucleotide
ggaggauuguggccuucuu

SEQ ID NO: 16 - RNA, 19 nt, Artificial Sequence
  Description of Artificial Sequence: Synthetic oligonucleotide
ucauguguguggagagcgu

SEQ ID NO: 17 - RNA, 19 nt, Artificial Sequence
  Description of Artificial Sequence: Synthetic oligonucleotide
ccuacaagcuuucccagaa

SEQ ID NO: 18 - RNA, 19 nt, Artificial Sequence
  Description of Artificial Sequence: Synthetic oligonucleotide
ggaggcaggcgacgaguuu

SEQ ID NO: 19 - RNA, 19 nt, Artificial Sequence
  Description of Artificial Sequence: Synthetic oligonucleotide
gauucaggcugcuugggau

SEQ ID NO: 20 - RNA, 19 nt, Artificial Sequence
  Description of Artificial Sequence: Synthetic oligonucleotide
gaaagggccaggaacgcuu

SEQ ID NO: 21 - RNA, 19 nt, Artificial Sequence
  Description of Artificial Sequence: Synthetic oligonucleotide
caaaggaaaccaacaauaa

SEQ ID NO: 22 - RNA, 19 nt, Artificial Sequence
  Description of Artificial Sequence: Synthetic oligonucleotide
gcaaaggaaaccaacaaua

SEQ ID NO: 23 - RNA, 19 nt, Artificial Sequence
  Description of Artificial Sequence: Synthetic oligonucleotide
caccgcaucucuacauuca

SEQ ID NO: 24 - RNA, 19 nt, Artificial Sequence
  Description of Artificial Sequence: Synthetic oligonucleotide
ccacugagaacgagccaga

SEQ ID NO: 25 - RNA, 19 nt, Artificial Sequence
  Description of Artificial Sequence: Synthetic oligonucleotide
gaacagauuggaucggaaa

SEQ ID NO: 26 - RNA, 19 nt, Artificial Sequence
  Description of Artificial Sequence: Synthetic oligonucleotide
gagauggacugacaaaugc

SEQ ID NO: 27 - RNA, 19 nt, Artificial Sequence
  Description of Artificial Sequence: Synthetic oligonucleotide
ggauauugccuacauuuau

SEQ ID NO: 28 - RNA, 19 nt, Artificial Sequence
  Description of Artificial Sequence: Synthetic oligonucleotide
gaacaacgagaaucaagau

SEQ ID NO: 29 - RNA, 19 nt, Artificial Sequence
  Description of Artificial Sequence: Synthetic oligonucleotide
acagagaccucaagaguaa

SEQ ID NO: 30 - RNA, 19 nt, Artificial Sequence
  Description of Artificial Sequence: Synthetic oligonucleotide
gaaucgggcugguuuccaa

SEQ ID NO: 31 - RNA, 19 nt, Artificial Sequence
  Description of Artificial Sequence: Synthetic oligonucleotide
caacaacagggaccagaua

SEQ ID NO: 32 - RNA, 19 nt, Artificial Sequence
  Description of Artificial Sequence: Synthetic oligonucleotide
gagaugaucaaacuuauag

SEQ ID NO: 33 - RNA, 19 nt, Artificial Sequence
  Description of Artificial Sequence: Synthetic oligonucleotide
ugaaaggacucacuuggua

SEQ ID NO: 34 - RNA, 19 nt, Artificial Sequence
  Description of Artificial Sequence: Synthetic oligonucleotide
cauaguagcuacagacaga

SEQ ID NO: 35 - RNA, 19 nt, Artificial Sequence
  Description of Artificial Sequence: Synthetic oligonucleotide
agaccuggcugcacuaauu

SEQ ID NO: 36 - RNA, 19 nt, Artificial Sequence
  Description of Artificial Sequence: Synthetic oligonucleotide
ggaccuauaugugguagag

SEQ ID NO: 37 - RNA, 19 nt, Artificial Sequence
  Description of Artificial Sequence: Synthetic oligonucleotide
gcaaaccgcucaucuauaa

SEQ ID NO: 38 - RNA, 19 nt, Artificial Sequence
  Description of Artificial Sequence: Synthetic oligonucleotide
caucaagucuggagaaaga

SEQ ID NO: 39 - RNA, 19 nt, Artificial Sequence
  Description of Artificial Sequence: Synthetic oligonucleotide
caacauuacaguucucaua

SEQ ID NO: 40 - RNA, 19 nt, Artificial Sequence
  Description of Artificial Sequence: Synthetic oligonucleotide
gagaaucacuuugucauag

SEQ ID NO: 41 - RNA, 19 nt, Artificial Sequence
  Description of Artificial Sequence: Synthetic oligonucleotide
caaagaagcugacaauacu

SEQ ID NO: 42 - RNA, 19 nt, Artificial Sequence
  Description of Artificial Sequence: Synthetic oligonucleotide
ccagauacuuucuuuacua

SEQ ID NO: 43 - RNA, 19 nt, Artificial Sequence
  Description of Artificial Sequence: Synthetic oligonucleotide
gaaguucgguuuaguagcc

SEQ ID NO: 44 - RNA, 19 nt, Artificial Sequence
  Description of Artificial Sequence: Synthetic oligonucleotide
aaauauggcaucuccuuaa

SEQ ID NO: 45 - RNA, 19 nt, Artificial Sequence
  Description of Artificial Sequence: Synthetic oligonucleotide
gaacccagauuuauguaau

SEQ ID NO: 46 - RNA, 19 nt, Artificial Sequence
  Description of Artificial Sequence: Synthetic oligonucleotide
gaaccucacuauccaagga

SEQ ID NO: 47 - RNA, 19 nt, Artificial Sequence
  Description of Artificial Sequence: Synthetic oligonucleotide
auauaaaguuggaugcgga

SEQ ID NO: 48 - RNA, 19 nt, Artificial Sequence
  Description of Artificial Sequence: Synthetic oligonucleotide
cuaggaagcuccaguucga

SEQ ID NO: 49 - RNA, 19 nt, Artificial Sequence
  Description of Artificial Sequence: Synthetic oligonucleotide
gcugaaacaugcaguugua

SEQ ID NO: 50 - RNA, 19 nt, Artificial Sequence
  Description of Artificial Sequence: Synthetic oligonucleotide
gauaaaggcuacuguugga

SEQ ID NO: 51 - RNA, 19 nt, Artificial Sequence
  Description of Artificial Sequence: Synthetic oligonucleotide
ccacuaauguccagcguuu

SEQ ID NO: 52 - RNA, 19 nt, Artificial Sequence
  Description of Artificial Sequence: Synthetic oligonucleotide
acaaguagcugauauugau

SEQ ID NO: 53 - RNA, 19 nt, Artificial Sequence
  Description of Artificial Sequence: Synthetic oligonucleotide
gcauaagguucagagccua

SEQ ID NO: 54 - RNA, 19 nt, Artificial Sequence
  Description of Artificial Sequence: Synthetic oligonucleotide
gagauugaugcacugagua

SEQ ID NO: 55 - RNA, 19 nt, Artificial Sequence
  Description of Artificial Sequence: Synthetic oligonucleotide
ccgacgagauugaaaugau

SEQ ID NO: 56 - RNA, 19 nt, Artificial Sequence
  Description of Artificial Sequence: Synthetic oligonucleotide
ccgcaacgguauuggcgaa

SEQ ID NO: 57 - RNA, 19 nt, Artificial Sequence
  Description of Artificial Sequence: Synthetic oligonucleotide
gaagcaugacggacaagua

SEQ ID NO: 58 - RNA, 19 nt, Artificial Sequence
  Description of Artificial Sequence: Synthetic oligonucleotide
uaacuacaccgaggaaaug

SEQ ID NO: 59 - RNA, 19 nt, Artificial Sequence
  Description of Artificial Sequence: Synthetic oligonucleotide
gagucugagucuucaaguu

SEQ ID NO: 60 - RNA, 19 nt, Artificial Sequence
  Description of Artificial Sequence: Synthetic oligonucleotide
caagcaagggugugaguuu

SEQ ID NO: 61 - RNA, 19 nt, Artificial Sequence
  Description of Artificial Sequence: Synthetic oligonucleotide
ggacggacuuuauaagaug

SEQ ID NO: 62 - RNA, 19 nt, Artificial Sequence
  Description of Artificial Sequence: Synthetic oligonucleotide
ggaccaagcugucauuaac

SEQ ID NO: 63 - RNA, 19 nt, Artificial Sequence
  Description of Artificial Sequence: Synthetic oligonucleotide
ggccauaaauuccgaaauc

SEQ ID NO: 64 - RNA, 19 nt, Artificial Sequence
  Description of Artificial Sequence: Synthetic oligonucleotide
aggaauugcugcaggauuu

SEQ ID NO: 65 - RNA, 19 nt, Artificial Sequence
  Description of Artificial Sequence: Synthetic oligonucleotide
ccgcaaauuccgagacgaa

SEQ ID NO: 66 - RNA, 19 nt, Artificial Sequence
  Description of Artificial Sequence: Synthetic oligonucleotide
caaaguguguaacggaaua

SEQ ID NO: 67 - RNA, 19 nt, Artificial Sequence
  Description of Artificial Sequence: Synthetic oligonucleotide
guaacaagcucacgcaguu

SEQ ID NO: 68 - RNA, 19 nt, Artificial Sequence
  Description of Artificial Sequence: Synthetic oligonucleotide
gaggaaauauguacuacga

SEQ ID NO: 69 - RNA, 19 nt, Artificial Sequence
  Description of Artificial Sequence: Synthetic oligonucleotide
caaacagcguggucaagua

SEQ ID NO: 70 - RNA, 19 nt, Artificial Sequence
  Description of Artificial Sequence: Synthetic oligonucleotide
caagaaaggccacuacgaa

SEQ ID NO: 71 - RNA, 19 nt, Artificial Sequence
  Description of Artificial Sequence: Synthetic oligonucleotide
ggaauucugucuucacaag

SEQ ID NO: 72 - RNA, 19 nt, Artificial Sequence
  Description of Artificial Sequence: Synthetic oligonucleotide
caaagcagcuguacuucuc

SEQ ID NO: 73 - RNA, 19 nt, Artificial Sequence
  Description of Artificial Sequence: Synthetic oligonucleotide
gccagaaguucgucuacaa

SEQ ID NO: 74 - RNA, 19 nt, Artificial Sequence
  Description of Artificial Sequence: Synthetic oligonucleotide
gcaagaacaagaccaacau

SEQ ID NO: 75 - RNA, 19 nt, Artificial Sequence
  Description of Artificial Sequence: Synthetic oligonucleotide
acgggaugguggugaauuc

SEQ ID NO: 76 - RNA, 19 nt, Artificial Sequence
  Description of Artificial Sequence: Synthetic oligonucleotide
uuuaauggguugggagucu

SEQ ID NO: 77 - RNA, 19 nt, Artificial Sequence
  Description of Artificial Sequence: Synthetic oligonucleotide
ggacgaauucugcacaaug

SEQ ID NO: 78 - RNA, 19 nt, Artificial Sequence
  Description of Artificial Sequence: Synthetic oligonucleotide
gacgaauucugcacaaugg

SEQ ID NO: 79 - RNA, 19 nt, Artificial Sequence
  Description of Artificial Sequence: Synthetic oligonucleotide
cuacaacacagacacguuu

SEQ ID NO: 80 - RNA, 19 nt, Artificial Sequence
  Description of Artificial Sequence: Synthetic oligonucleotide
agacgaagcauacgugaug

SEQ ID NO: 81 - RNA, 19 nt, Artificial Sequence
  Description of Artificial Sequence: Synthetic oligonucleotide
caaagaaucuagcaucaua

SEQ ID NO: 82 - RNA, 19 nt, Artificial Sequence
  Description of Artificial Sequence: Synthetic oligonucleotide
gaggacggcuucccaauaa

SEQ ID NO: 83 - RNA, 19 nt, Artificial Sequence
  Description of Artificial Sequence: Synthetic oligonucleotide
gcugaagccucaauguuua

SEQ ID NO: 84 - RNA, 19 nt, Artificial Sequence
  Description of Artificial Sequence: Synthetic oligonucleotide
gaauggaaacagcgaagga

SEQ ID NO: 85 - RNA, 19 nt, Artificial Sequence
  Description of Artificial Sequence: Synthetic oligonucleotide
auucaaaccugaccacaga

SEQ ID NO: 86 - RNA, 19 nt, Artificial Sequence
  Description of Artificial Sequence: Synthetic oligonucleotide
ccacagaauuggaggcuac

SEQ ID NO: 87 - RNA, 19 nt, Artificial Sequence
  Description of Artificial Sequence: Synthetic oligonucleotide
ccgcacauccaguggcuaa

SEQ ID NO: 88 - RNA, 19 nt, Artificial Sequence
  Description of Artificial Sequence: Synthetic oligonucleotide
acguggaguucauguguaa

SEQ ID NO: 89 - RNA, 19 nt, Artificial Sequence
  Description of Artificial Sequence: Synthetic oligonucleotide
ccaaaucucucaaccagaa

SEQ ID NO: 90 - RNA, 19 nt, Artificial Sequence
  Description of Artificial Sequence: Synthetic oligonucleotide
gaacaguauucaccuaguu

SEQ ID NO: 91 - RNA, 19 nt, Artificial Sequence
  Description of Artificial Sequence: Synthetic oligonucleotide
ggccaacacugucaaguuu

SEQ ID NO: 92 - RNA, 19 nt, Artificial Sequence
  Description of Artificial Sequence: Synthetic oligonucleotide
gugaagauguugaaagaug

SEQ ID NO: 93 - RNA, 19 nt, Artificial Sequence
  Description of Artificial Sequence: Synthetic oligonucleotide
ugucggaccuggugucuga

SEQ ID NO: 94 - RNA, 19 nt, Artificial Sequence
  Description of Artificial Sequence: Synthetic oligonucleotide
gcaucaagcugcggcauca

SEQ ID NO: 95 - RNA, 19 nt, Artificial Sequence
  Description of Artificial Sequence: Synthetic oligonucleotide
ggacggcacacccuacguu

SEQ ID NO: 96 - RNA, 19 nt, Artificial Sequence
  Description of Artificial Sequence: Synthetic oligonucleotide
ugcacaaccucgacuacua

SEQ ID NO: 97 - RNA, 19 nt, Artificial Sequence
  Description of Artificial Sequence: Synthetic oligonucleotide
gcacuggagucucgugaug

SEQ ID NO: 98 - RNA, 19 nt, Artificial Sequence
  Description of Artificial Sequence: Synthetic oligonucleotide
ccucgaauaggcacaguua

SEQ ID NO: 99 - RNA, 19 nt, Artificial Sequence
  Description of Artificial Sequence: Synthetic oligonucleotide
auaacuaccugcuagaugu

SEQ ID NO: 100 - RNA, 19 nt, Artificial Sequence
  Description of Artificial Sequence: Synthetic oligonucleotide
gcauucggcugcgccauca

SEQ ID NO: 101 - RNA, 19 nt, Artificial Sequence
  Description of Artificial Sequence: Synthetic oligonucleotide
gcggugaaggcuaugcuaa

SEQ ID NO: 102 - RNA, 19 nt, Artificial Sequence
  Description of Artificial Sequence: Synthetic oligonucleotide
gcucuaugccaauauguuu

SEQ ID NO: 103 - RNA, 19 nt, Artificial Sequence
  Description of Artificial Sequence: Synthetic oligonucleotide
gagaacauuccaucgugua

SEQ ID NO: 104 - RNA, 19 nt, Artificial Sequence
  Description of Artificial Sequence: Synthetic oligonucleotide
agggagaagaucugacgga

SEQ ID NO: 105 - RNA, 19 nt, Artificial Sequence
  Description of Artificial Sequence: Synthetic oligonucleotide
gaaggcaucuacaccauua

SEQ ID NO: 106 - RNA, 19 nt, Artificial Sequence
  Description of Artificial Sequence: Synthetic oligonucleotide
gaaggagucuggaauagaa

SEQ ID NO: 107 - RNA, 19 nt, Artificial Sequence
  Description of Artificial Sequence: Synthetic oligonucleotide
gaauuuaagucguguguuc

SEQ ID NO: 108 - RNA, 19 nt, Artificial Sequence
  Description of Artificial Sequence: Synthetic oligonucleotide
ggaauucauuucacucuga

SEQ ID NO: 109 - RNA, 19 nt, Artificial Sequence
  Description of Artificial Sequence: Synthetic oligonucleotide
caacaggagucuaaucaag

SEQ ID NO: 110 - RNA, 19 nt, Artificial Sequence
  Description of Artificial Sequence: Synthetic oligonucleotide
ggaccacuuucccuacuuu

SEQ ID NO: 111 - RNA, 19 nt, Artificial Sequence
  Description of Artificial Sequence: Synthetic oligonucleotide
guagugggcccaacaaauu

SEQ ID NO: 112 - RNA, 19 nt, Artificial Sequence
  Description of Artificial Sequence: Synthetic oligonucleotide
gcugggaucaagauuauua

SEQ ID NO: 113 - RNA, 19 nt, Artificial Sequence
  Description of Artificial Sequence: Synthetic oligonucleotide
ggaaaugacuggcaaugca

SEQ ID NO: 114 - RNA, 19 nt, Artificial Sequence
  Description of Artificial Sequence: Synthetic oligonucleotide
gcacuggucuguccacucu

SEQ ID NO: 115 - RNA, 19 nt, Artificial Sequence
  Description of Artificial Sequence: Synthetic oligonucleotide
guccucgacuugaacauua

SEQ ID NO: 116 - RNA, 19 nt, Artificial Sequence
  Description of Artificial Sequence: Synthetic oligonucleotide
aggcucagcuuguguguaa

SEQ ID NO: 117 - RNA, 19 nt, Artificial Sequence
  Description of Artificial Sequence: Synthetic oligonucleotide
agaagugccucagauaaua

SEQ ID NO: 118 - RNA, 19 nt, Artificial Sequence
  Description of Artificial Sequence: Synthetic oligonucleotide
uaguaaagguguacaguga

SEQ ID NO: 119 - RNA, 19 nt, Artificial Sequence
  Description of Artificial Sequence: Synthetic oligonucleotide
ugcagaaagugaagcauua

SEQ ID NO: 120 - RNA, 19 nt, Artificial Sequence
  Description of Artificial Sequence: Synthetic oligonucleotide
ggagauagccgcuucgucu

SEQ ID NO: 121 - RNA, 19 nt, Artificial Sequence
  Description of Artificial Sequence: Synthetic oligonucleotide
agaaagaguugagaaguua

SEQ ID NO: 122 - RNA, 19 nt, Artificial Sequence
  Description of Artificial Sequence: Synthetic oligonucleotide
gaaauauugcuguuccuua

SEQ ID NO: 123 - RNA, 19 nt, Artificial Sequence
  Description of Artificial Sequence: Synthetic oligonucleotide
gaacgggacacuuugcuaa

SEQ ID NO: 124 - RNA, 19 nt, Artificial Sequence
  Description of Artificial Sequence: Synthetic oligonucleotide
gggcaaaggucauggagau

SEQ ID NO: 125 - RNA, 19 nt, Artificial Sequence
  Description of Artificial Sequence: Synthetic oligonucleotide
ggccagaaauggagaauaa

SEQ ID NO: 126 - RNA, 19 nt, Artificial Sequence
  Description of Artificial Sequence: Synthetic oligonucleotide
gcagacaccuacaacauca

SEQ ID NO: 127 - RNA, 19 nt, Artificial Sequence
  Description of Artificial Sequence: Synthetic oligonucleotide
ggacucaguacgccguuua

SEQ ID NO: 128 - RNA, 19 nt, Artificial Sequence
  Description of Artificial Sequence: Synthetic oligonucleotide
gugggaggguuggugauua

SEQ ID NO: 129 - RNA, 19 nt, Artificial Sequence
  Description of Artificial Sequence: Synthetic oligonucleotide
gaauugaccuguacaaaga

SEQ ID NO: 130 - RNA, 19 nt, Artificial Sequence
  Description of Artificial Sequence: Synthetic oligonucleotide
gaaauugaguggauuacug

SEQ ID NO: 131 - RNA, 19 nt, Artificial Sequence
  Description of Artificial Sequence: Synthetic oligonucleotide
gaagugguuuccaucagua

SEQ ID NO: 132 - RNA, 19 nt, Artificial Sequence
  Description of Artificial Sequence: Synthetic oligonucleotide
guggagagauggcauaauu

SEQ ID NO: 133 - RNA, 19 nt, Artificial Sequence
  Description of Artificial Sequence: Synthetic oligonucleotide
ggaaguaccugaaccaguu

SEQ ID NO: 134 - RNA, 19 nt, Artificial Sequence
  Description of Artificial Sequence: Synthetic oligonucleotide
ccaauaaucuuaacagugu

SEQ ID NO: 135 - RNA, 19 nt, Artificial Sequence
  Description of Artificial Sequence: Synthetic oligonucleotide
ggauucagcuucuccuaaa

SEQ ID NO: 136 - RNA, 19 nt, Artificial Sequence
  Description of Artificial Sequence: Synthetic oligonucleotide
guggugagcuuaaugaaug

SEQ ID NO: 137 - RNA, 19 nt, Artificial Sequence
  Description of Artificial Sequence: Synthetic oligonucleotide
cgaauaugauccaacaaua

SEQ ID NO: 138 - RNA, 19 nt, Artificial Sequence
  Description of Artificial Sequence: Synthetic oligonucleotide
uaaggacucugaagaugua

SEQ ID NO: 139 - RNA, 19 nt, Artificial Sequence
  Description of Artificial Sequence: Synthetic oligonucleotide
gacaaaguguguaauuaug

SEQ ID NO: 140 - RNA, 19 nt, Artificial Sequence
  Description of Artificial Sequence: Synthetic oligonucleotide
gcucaggacuuagcaagaa

SEQ ID NO: 141 - RNA, 19 nt, Artificial Sequence
  Description of Artificial Sequence: Synthetic oligonucleotide
gcacauggauggagguucu

SEQ ID NO: 142 - RNA, 19 nt, Artificial Sequence
  Description of Artificial Sequence: Synthetic oligonucleotide
gcagagagagcagauuuga

SEQ ID NO: 143 - RNA, 19 nt, Artificial Sequence
  Description of Artificial Sequence: Synthetic oligonucleotide
gagcagauuugaagcaacu

SEQ ID NO: 144 - RNA, 19 nt, Artificial Sequence
  Description of Artificial Sequence: Synthetic oligonucleotide
ccagaaagcuaauucaucu

SEQ ID NO: 145 - RNA, 19 nt, Artificial Sequence
  Description of Artificial Sequence: Synthetic oligonucleotide
caaagacgaugacuucgaa

SEQ ID NO: 146 - RNA, 19 nt, Artificial Sequence
  Description of Artificial Sequence: Synthetic oligonucleotide
gaucagcauuugcauggaa

SEQ ID NO: 147 - RNA, 19 nt, Artificial Sequence
  Description of Artificial Sequence: Synthetic oligonucleotide
ggaagcugauccaccuuga

SEQ ID NO: 148 - RNA, 19 nt, Artificial Sequence
  Description of Artificial Sequence: Synthetic oligonucleotide
gaaagucagcaucgcgguu

SEQ ID NO: 149 - RNA, 19 nt, Artificial Sequence
  Description of Artificial Sequence: Synthetic oligonucleotide
gcugaaaggucaacagaua

SEQ ID NO: 150 - RNA, 19 nt, Artificial Sequence
  Description of Artificial Sequence: Synthetic oligonucleotide
agaagaagcuuuagcaauu

SEQ ID NO: 151 - RNA, 19 nt, Artificial Sequence
  Description of Artificial Sequence: Synthetic oligonucleotide
gagcucaacuccaguauug

SEQ ID NO: 152 - RNA, 19 nt, Artificial Sequence
  Description of Artificial Sequence: Synthetic oligonucleotide
gaacagauguguccuauuu

SEQ ID NO: 153 - RNA, 19 nt, Artificial Sequence
  Description of Artificial Sequence: Synthetic oligonucleotide
ccaaagcucuggacuuauu

SEQ ID NO: 154 - RNA, 19 nt, Artificial Sequence
  Description of Artificial Sequence: Synthetic oligonucleotide
aaacagaucuuuacaagcu

SEQ ID NO: 155 - RNA, 19 nt, Artificial Sequence
  Description of Artificial Sequence: Synthetic oligonucleotide
caagaggauugaaguagaa

SEQ ID NO: 156 - RNA, 19 nt, Artificial Sequence
  Description of Artificial Sequence: Synthetic oligonucleotide
guacagggcuccagaaauu

SEQ ID NO: 157 - RNA, 19 nt, Artificial Sequence
  Description of Artificial Sequence: Synthetic oligonucleotide
caaggucucuggaggaauu

SEQ ID NO: 158 - RNA, 19 nt, Artificial Sequence
  Description of Artificial Sequence: Synthetic oligonucleotide
gucagaagcuuacagauga

SEQ ID NO: 159 - RNA, 19 nt, Artificial Sequence
  Description of Artificial Sequence: Synthetic oligonucleotide
guccaucauucaugcgaaa

SEQ ID NO: 160 - RNA, 19 nt, Artificial Sequence
  Description of Artificial Sequence: Synthetic oligonucleotide
cuacagagaacugcgguua

SEQ ID NO: 161 - RNA, 19 nt, Artificial Sequence
  Description of Artificial Sequence: Synthetic oligonucleotide
gaccggauguuaaccuuua

SEQ ID NO: 162 - RNA, 19 nt, Artificial Sequence
  Description of Artificial Sequence: Synthetic oligonucleotide
agacugaccuguacaaguu

SEQ ID NO: 163 - RNA, 19 nt, Artificial Sequence
  Description of Artificial Sequence: Synthetic oligonucleotide
gcuacacgcaguugcagua

SEQ ID NO: 164 - RNA, 19 nt, Artificial Sequence
  Description of Artificial Sequence: Synthetic oligonucleotide
ccggcuuccugacggagua

SEQ ID NO: 165 - RNA, 19 nt, Artificial Sequence
  Description of Artificial Sequence: Synthetic oligonucleotide
cgaaggaaguaucgaauuu

SEQ ID NO: 166 - RNA, 19 nt, Artificial Sequence
  Description of Artificial Sequence: Synthetic oligonucleotide
agaacgaauugauguguaa

SEQ ID NO: 167 - RNA, 19 nt, Artificial Sequence
  Description of Artificial Sequence: Synthetic oligonucleotide
ggaccaacuacaaauuaau

SEQ ID NO: 168 - RNA, 19 nt, Artificial Sequence
  Description of Artificial Sequence: Synthetic oligonucleotide
gcuacguaguucgggcaaa

SEQ ID NO: 169 - RNA, 19 nt, Artificial Sequence
  Description of Artificial Sequence: Synthetic oligonucleotide
gccaguauauuaugacuaa

SEQ ID NO: 170 - RNA, 19 nt, Artificial Sequence
  Description of Artificial Sequence: Synthetic oligonucleotide
gaugagaagcaacaacaua

SEQ ID NO: 171 - RNA, 19 nt, Artificial Sequence
  Description of Artificial Sequence: Synthetic oligonucleotide
cccuaggaauuuagacaac

SEQ ID NO: 172 - RNA, 19 nt, Artificial Sequence
  Description of Artificial Sequence: Synthetic oligonucleotide
aaagucuguuggugcacaa

SEQ ID NO: 173 - RNA, 19 nt, Artificial Sequence
  Description of Artificial Sequence: Synthetic oligonucleotide
gcaguuagguguuggaaua

SEQ ID NO: 174 - RNA, 19 nt, Artificial Sequence
  Description of Artificial Sequence: Synthetic oligonucleotide
ugauaccgauguagagguu

SEQ ID NO: 175 - RNA, 19 nt, Artificial Sequence
  Description of Artificial Sequence: Synthetic oligonucleotide
gcauaaugguaguacgaac

SEQ ID NO: 176 - RNA, 19 nt, Artificial Sequence
  Description of Artificial Sequence: Synthetic oligonucleotide
ccacgagacgggaacauua

SEQ ID NO: 177 - RNA, 19 nt, Artificial Sequence
  Description of Artificial Sequence: Synthetic oligonucleotide
gaagaagcauauacacuua

SEQ ID NO: 178 - RNA, 19 nt, Artificial Sequence
  Description of Artificial Sequence: Synthetic oligonucleotide
ugaaaggccuguaauauuu

SEQ ID NO: 179 - RNA, 19 nt, Artificial Sequence
  Description of Artificial Sequence: Synthetic oligonucleotide
gaacauggcggagugaaua

SEQ ID NO: 180 - RNA, 19 nt, Artificial Sequence
  Description of Artificial Sequence: Synthetic oligonucleotide
auuaguuaagggacggaaa

SEQ ID NO: 181 - RNA, 19 nt, Artificial Sequence
  Description of Artificial Sequence: Synthetic oligonucleotide
caacaaugcugaauucuug

SEQ ID NO: 182 - RNA, 19 nt, Artificial Sequence
  Description of Artificial Sequence: Synthetic oligonucleotide
acaaauaccguaacaagua

SEQ ID NO: 183 - RNA, 19 nt, Artificial Sequence
  Description of Artificial Sequence: Synthetic oligonucleotide
ggagccaccugaaccauga

SEQ ID NO: 184 - RNA, 19 nt, Artificial Sequence
  Description of Artificial Sequence: Synthetic oligonucleotide
cuaaagggcucauugucaa

SEQ ID NO: 185 - RNA, 19 nt, Artificial Sequence
  Description of Artificial Sequence: Synthetic oligonucleotide
ggacucaguuggcagacua

SEQ ID NO: 186 - RNA, 19 nt, Artificial Sequence
  Description of Artificial Sequence: Synthetic oligonucleotide
aggaagggcugcgcgaaua

SEQ ID NO: 187 - RNA, 19 nt, Artificial Sequence
  Description of Artificial Sequence: Synthetic oligonucleotide
auaaagugcuggcgcaauc

SEQ ID NO: 188 - RNA, 19 nt, Artificial Sequence
  Description of Artificial Sequence: Synthetic oligonucleotide
gagucaugcccuacggaau

SEQ ID NO: 189 - RNA, 19 nt, Artificial Sequence
  Description of Artificial Sequence: Synthetic oligonucleotide
caaugcugauguuugauaa

SEQ ID NO: 190 - RNA, 19 nt, Artificial Sequence
  Description of Artificial Sequence: Synthetic oligonucleotide
ccagauagccuuagagacu

SEQ ID NO: 191 - RNA, 19 nt, Artificial Sequence
  Description of Artificial Sequence: Synthetic oligonucleotide
gaguuagauuccaagacga

SEQ ID NO: 192 - RNA, 19 nt, Artificial Sequence
  Description of Artificial Sequence: Synthetic oligonucleotide
ccaacuucguggcgaccua

SEQ ID NO: 193 - RNA, 19 nt, Artificial Sequence
  Description of Artificial Sequence: Synthetic oligonucleotide
gagaagaaauggaagaaau

SEQ ID NO: 194 - RNA, 19 nt, Artificial Sequence
  Description of Artificial Sequence: Synthetic oligonucleotide
ccaaagugcugcaguacua

SEQ ID NO: 195 - RNA, 19 nt, Artificial Sequence
  Description of Artificial Sequence: Synthetic oligonucleotide
ggucugaacugaaugaaga

SEQ ID NO: 196 - RNA, 19 nt, Artificial Sequence
  Description of Artificial Sequence: Synthetic oligonucleotide
auaaaguucuggugcgaca

SEQ ID NO: 197 - RNA, 19 nt, Artificial Sequence
  Description of Artificial Sequence: Synthetic oligonucleotide
aacguuagcuucaccaaca

SEQ ID NO: 198 - RNA, 19 nt, Artificial Sequence
  Description of Artificial Sequence: Synthetic oligonucleotide
ggaacuaugaccucgacua

SEQ ID NO: 199 - RNA, 19 nt, Artificial Sequence
  Description of Artificial Sequence: Synthetic oligonucleotide
gaacacacaacgucuugga

SEQ ID NO: 200 - RNA, 19 nt, Artificial Sequence
  Description of Artificial Sequence: Synthetic oligonucleotide
cuaccaggcugcgcgcaaa

SEQ ID NO: 201 - RNA, 19 nt, Artificial Sequence
  Description of Artificial Sequence: Synthetic oligonucleotide
gaacccagaccucgaguuu

SEQ ID NO: 202 - RNA, 19 nt, Artificial Sequence
  Description of Artificial Sequence: Synthetic oligonucleotide
gaagaaaucgacgugguca

SEQ ID NO: 203 - RNA, 19 nt, Artificial Sequence
  Description of Artificial Sequence: Synthetic oligonucleotide
ggacagugagcgucgcaga

SEQ ID NO: 204 - RNA, 19 nt, Artificial Sequence
  Description of Artificial Sequence: Synthetic oligonucleotide
gagaggacacccugagcga

SEQ ID NO: 205 - RNA, 19 nt, Artificial Sequence
  Description of Artificial Sequence: Synthetic oligonucleotide
gcaccuucauggacggcua

SEQ ID NO: 206 - RNA, 19 nt, Artificial Sequence
  Description of Artificial Sequence: Synthetic oligonucleotide
gcauuaugguguccgcaca

SEQ ID NO: 207 - RNA, 19 nt, Artificial Sequence
  Description of Artificial Sequence: Synthetic oligonucleotide
ugaaggaaguuguccgaac

SEQ ID NO: 208 - RNA, 19 nt, Artificial Sequence
  Description of Artificial Sequence: Synthetic oligonucleotide
cugcagaaccgcaucgcca

SEQ ID NO: 209 - RNA, 19 nt, Artificial Sequence
  Description of Artificial Sequence: Synthetic oligonucleotide
ccacgugacuuucaacaau

SEQ ID NO: 210 - RNA, 19 nt, Artificial Sequence
  Description of Artificial Sequence: Synthetic oligonucleotide
acauucagaugcacaacua

SEQ ID NO: 211 - RNA, 19 nt, Artificial Sequence
  Description of Artificial Sequence: Synthetic oligonucleotide
ggacaagcgucgccggaau

SEQ ID NO: 212 - RNA, 19 nt, Artificial Sequence
  Description of Artificial Sequence: Synthetic oligonucleotide
ccaccuugcuuguaccaaa

SEQ ID NO: 213 - RNA, 19 nt, Artificial Sequence
  Description of Artificial Sequence: Synthetic oligonucleotide
gcaggacucuuaucuuaaa

SEQ ID NO: 214 - RNA, 19 nt, Artificial Sequence
  Description of Artificial Sequence: Synthetic oligonucleotide
aaacuugacugcacauuaa

SEQ ID NO: 215 - RNA, 19 nt, Artificial Sequence
  Description of Artificial Sequence: Synthetic oligonucleotide
ugaauaagguguugaaaga

SEQ ID NO: 216 - RNA, 19 nt, Artificial Sequence
  Description of Artificial Sequence: Synthetic oligonucleotide
gagauucacugucagcuuu

SEQ ID NO: 217 - RNA, 19 nt, Artificial Sequence
  Description of Artificial Sequence: Synthetic oligonucleotide
gaacaaacgcccgucaaag

SEQ ID NO: 218 - RNA, 19 nt, Artificial Sequence
  Description of Artificial Sequence: Synthetic oligonucleotide
gaacacucuucgccaucuu

SEQ ID NO: 219 - RNA, 19 nt, Artificial Sequence
  Description of Artificial Sequence: Synthetic oligonucleotide
acaaagacaggaacaauuc

SEQ ID NO: 220 - RNA, 19 nt, Artificial Sequence
  Description of Artificial Sequence: Synthetic oligonucleotide
gguguucacugaccuccaa

SEQ ID NO: 221 - RNA, 19 nt, Artificial Sequence
  Description of Artificial Sequence: Synthetic oligonucleotide
caucaaauaucacuaaguc

SEQ ID NO: 222 - RNA, 19 nt, Artificial Sequence
  Description of Artificial Sequence: Synthetic oligonucleotide
caacaaagaacaaucacua

SEQ ID NO: 223 - RNA, 19 nt, Artificial Sequence
  Description of Artificial Sequence: Synthetic oligonucleotide
agaaauaccagcacuauga

SEQ ID NO: 224 - RNA, 19 nt, Artificial Sequence
  Description of Artificial Sequence: Synthetic oligonucleotide
gugaaaugcucucggcuau

SEQ ID NO: 225 - RNA, 19 nt, Artificial Sequence
  Description of Artificial Sequence: Synthetic oligonucleotide
ggacaguaccaaugcauaa

SEQ ID NO: 226 - RNA, 19 nt, Artificial Sequence
  Description of Artificial Sequence: Synthetic oligonucleotide
gaacuuacuuuggccagca

SEQ ID NO: 227 - RNA, 19 nt, Artificial Sequence
  Description of Artificial Sequence: Synthetic oligonucleotide
ccuaaggaacuguacauaa

SEQ ID NO: 228 - RNA, 19 nt, Artificial Sequence
  Description of Artificial Sequence: Synthetic oligonucleotide
ugugaaccuuggagcaaua

SEQ ID NO: 229 - RNA, 19 nt, Artificial Sequence
  Description of Artificial Sequence: Synthetic oligonucleotide
caucagagcuggaucuaga

SEQ ID NO: 230 - RNA, 19 nt, Artificial Sequence
  Description of Artificial Sequence: Synthetic oligonucleotide
ggccuuacuuuauuggauu

SEQ ID NO: 231 - RNA, 19 nt, Artificial Sequence
  Description of Artificial Sequence: Synthetic oligonucleotide
gagcuucaccuaucaaguu

SEQ ID NO: 232 - RNA, 19 nt, Artificial Sequence
  Description of Artificial Sequence: Synthetic oligonucleotide
ccucuaggaaugacggauu

SEQ ID NO: 233 - RNA, 19 nt, Artificial Sequence
  Description of Artificial Sequence: Synthetic oligonucleotide
gaaaggagacgucaaauau

SEQ ID NO: 234 - RNA, 19 nt, Artificial Sequence
  Description of Artificial Sequence: Synthetic oligonucleotide
ggaaugagguggucaacuu

SEQ ID NO: 235 - RNA, 19 nt, Artificial Sequence
  Description of Artificial Sequence: Synthetic oligonucleotide
caacgagucuccagugcua

SEQ ID NO: 236 - RNA, 19 nt, Artificial Sequence
  Description of Artificial Sequence: Synthetic oligonucleotide
ugacaacgacuauaucauc

SEQ ID NO: 237 - RNA, 19 nt, Artificial Sequence
  Description of Artificial Sequence: Synthetic oligonucleotide
ggaccuaacccgcucauga

SEQ ID NO: 238 - RNA, 19 nt, Artificial Sequence
  Description of Artificial Sequence: Synthetic oligonucleotide
gaggaucaguugcuaugaa

SEQ ID NO: 239 - RNA, 19 nt, Artificial Sequence
  Description of Artificial Sequence: Synthetic oligonucleotide
aaagcuaccuggcgaaaga

SEQ ID NO: 240 - RNA, 19 nt, Artificial Sequence
  Description of Artificial Sequence: Synthetic oligonucleotide
acaaguacuauuaccgcua

SEQ ID NO: 241 - RNA, 19 nt, Artificial Sequence
  Description of Artificial Sequence: Synthetic oligonucleotide
gagcgaccaucuuuaauuu

SEQ ID NO: 242 - RNA, 19 nt, Artificial Sequence
  Description of Artificial Sequence: Synthetic oligonucleotide
gaaaugaccuacgaggaaa

SEQ ID NO: 243 - RNA, 19 nt, Artificial Sequence
  Description of Artificial Sequence: Synthetic oligonucleotide
caucguauauuaccucaug

SEQ ID NO: 244 - RNA, 19 nt, Artificial Sequence
  Description of Artificial Sequence: Synthetic oligonucleotide
gggacaggccucagaacgu

SEQ ID NO: 245 - RNA, 19 nt, Artificial Sequence
  Description of Artificial Sequence: Synthetic oligonucleotide
ggacaacuguuucauauag

SEQ ID NO: 246 - RNA, 19 nt, Artificial Sequence
  Description of Artificial Sequence: Synthetic oligonucleotide
gccaguaccucauggauua

SEQ ID NO: 247 - RNA, 19 nt, Artificial Sequence
  Description of Artificial Sequence: Synthetic oligonucleotide
cuugaagagugucgaauua

SEQ ID NO: 248 - RNA, 19 nt, Artificial Sequence
  Description of Artificial Sequence: Synthetic oligonucleotide
gcuugaagagugucgaauu

SEQ ID NO: 249 - RNA, 19 nt, Artificial Sequence
  Description of Artificial Sequence: Synthetic oligonucleotide
gaauggaguuucaggauca

SEQ ID NO: 250 - RNA, 19 nt, Artificial Sequence
  Description of Artificial Sequence: Synthetic oligonucleotide
ggaaaucuccugcucgaaa

SEQ ID NO: 251 - RNA, 19 nt, Artificial Sequence
  Description of Artificial Sequence: Synthetic oligonucleotide
ugaagaaggugacgauaau

SEQ ID NO: 252 - RNA, 19 nt, Artificial Sequence
  Description of Artificial Sequence: Synthetic oligonucleotide
ggacucugcuaaugauuug

SEQ ID NO: 253 - RNA, 19 nt, Artificial Sequence
  Description of Artificial Sequence: Synthetic oligonucleotide
gaacaucacgggcaauuaa

SEQ ID NO: 254 - RNA, 19 nt, Artificial Sequence
  Description of Artificial Sequence: Synthetic oligonucleotide
gaacacuggugauuacuau

SEQ ID NO: 255 - RNA, 19 nt, Artificial Sequence
  Description of Artificial Sequence: Synthetic oligonucleotide
gaaaccaagugcaacaauu

SEQ ID NO: 256 - RNA, 19 nt, Artificial Sequence
  Description of Artificial Sequence: Synthetic oligonucleotide
gaagcacaguaccgauuua

SEQ ID NO: 257 - RNA, 19 nt, Artificial Sequence
  Description of Artificial Sequence: Synthetic oligonucleotide
caacacaacuuuaaucugc

SEQ ID NO: 258 - RNA, 19 nt, Artificial Sequence
  Description of Artificial Sequence: Synthetic oligonucleotide
ggaucgggauuuauuucua

SEQ ID NO: 259 - RNA, 19 nt, Artificial Sequence
  Description of Artificial Sequence: Synthetic oligonucleotide
ccaaacggcuggaauucgu

SEQ ID NO: 260 - RNA, 19 nt, Artificial Sequence
  Description of Artificial Sequence: Synthetic oligonucleotide
uaaugggcaugucuccuau

SEQ ID NO: 261 - RNA, 19 nt, Artificial Sequence
  Description of Artificial Sequence: Synthetic oligonucleotide
uagaacagcugcaguacaa

SEQ ID NO: 262 - RNA, 19 nt, Artificial Sequence
  Description of Artificial Sequence: Synthetic oligonucleotide
ucaccgaggccauucagua

SEQ ID NO: 263 - RNA, 19 nt, Artificial Sequence
  Description of Artificial Sequence: Synthetic oligonucleotide
gaacagcugcaguacaauc

SEQ ID NO: 264 - RNA, 19 nt, Artificial Sequence
  Description of Artificial Sequence: Synthetic oligonucleotide
ccucaugccaccuugaaua

SEQ ID NO: 265 - RNA, 19 nt, Artificial Sequence
  Description of Artificial Sequence: Synthetic oligonucleotide
caucuagacuuaagugaua

SEQ ID NO: 266 - RNA, 19 nt, Artificial Sequence
  Description of Artificial Sequence: Synthetic oligonucleotide
cuaaaggucucugguguuu

SEQ ID NO: 267 - RNA, 19 nt, Artificial Sequence
  Description of Artificial Sequence: Synthetic oligonucleotide
gguaucgccuagcgucuga

SEQ ID NO: 268 - RNA, 19 nt, Artificial Sequence
  Description of Artificial Sequence: Synthetic oligonucleotide
ugucaauacucucgcaaaa

SEQ ID NO: 269 - RNA, 19 nt, Artificial Sequence
  Description of Artificial Sequence: Synthetic oligonucleotide
gaauaugccuuggcuguuu

SEQ ID NO: 270 - RNA, 19 nt, Artificial Sequence
  Description of Artificial Sequence: Synthetic oligonucleotide
gaacgugccucggguacua

SEQ ID NO: 271 - RNA, 19 nt, Artificial Sequence
  Description of Artificial Sequence: Synthetic oligonucleotide
gcacacuuguaguagauua

SEQ ID NO: 272 - RNA, 19 nt, Artificial Sequence
  Description of Artificial Sequence: Synthetic oligonucleotide
gauuggaguucuagguuuu

SEQ ID NO: 273 - RNA, 19 nt, Artificial Sequence
  Description of Artificial Sequence: Synthetic oligonucleotide
gagcggagcuggucuguga

SEQ ID NO: 274 - RNA, 19 nt, Artificial Sequence
  Description of Artificial Sequence: Synthetic oligonucleotide
gaagagagcugugaauucu

SEQ ID NO: 275 - RNA, 19 nt, Artificial Sequence
  Description of Artificial Sequence: Synthetic oligonucleotide
gcacaagucuggcguucug

SEQ ID NO: 276 - RNA, 19 nt, Artificial Sequence
  Description of Artificial Sequence: Synthetic oligonucleotide
aaacucagcugcaacaaca

SEQ ID NO: 277 - RNA, 19 nt, Artificial Sequence
  Description of Artificial Sequence: Synthetic oligonucleotide
ggagaagcaucgugaguga

SEQ ID NO: 278 - RNA, 19 nt, Artificial Sequence
  Description of Artificial Sequence: Synthetic oligonucleotide
ccacuuugguguuucauaa

SEQ ID NO: 279 - RNA, 19 nt, Artificial Sequence
  Description of Artificial Sequence: Synthetic oligonucleotide
ucagguugcuggucaaauu

SEQ ID NO: 280 - RNA, 19 nt, Artificial Sequence
  Description of Artificial Sequence: Synthetic oligonucleotide
cguuauauaggaaccguaa

SEQ ID NO: 281 - RNA, 19 nt, Artificial Sequence
  Description of Artificial Sequence: Synthetic oligonucleotide
ccaacaccauguccaacua

SEQ ID NO: 282 - RNA, 19 nt, Artificial Sequence
  Description of Artificial Sequence: Synthetic oligonucleotide
gggcaaagacaacuuccua

SEQ ID NO: 283 - RNA, 19 nt, Artificial Sequence
  Description of Artificial Sequence: Synthetic oligonucleotide
gggacuuccucaaaggcga

SEQ ID NO: 284 - RNA, 19 nt, Artificial Sequence
  Description of Artificial Sequence: Synthetic oligonucleotide
cauaauaaacacgucgauu

SEQ ID NO: 285 - RNA, 19 nt, Artificial Sequence
  Description of Artificial Sequence: Synthetic oligonucleotide
cgaaggaaaucaagggaaa

SEQ ID NO: 286 - RNA, 19 nt, Artificial Sequence
  Description of Artificial Sequence: Synthetic oligonucleotide
gcaguugauucuuaccaga

SEQ ID NO: 287 - RNA, 19 nt, Artificial Sequence
  Description of Artificial Sequence: Synthetic oligonucleotide
ggaaugaacugaucgcccg

SEQ ID NO: 288 - RNA, 19 nt, Artificial Sequence
  Description of Artificial Sequence: Synthetic oligonucleotide
ggaagacccgaacucgaaa

SEQ ID NO: 289 - RNA, 19 nt, Artificial Sequence
  Description of Artificial Sequence: Synthetic oligonucleotide
gguaugccgugguccagaa

SEQ ID NO: 290 - RNA, 19 nt, Artificial Sequence
  Description of Artificial Sequence: Synthetic oligonucleotide
ccacgucucuaccuugaca

SEQ ID NO: 291 - RNA, 19 nt, Artificial Sequence
  Description of Artificial Sequence: Synthetic oligonucleotide
ucacggagaccacguuuca

SEQ ID NO: 292 - RNA, 19 nt, Artificial Sequence
  Description of Artificial Sequence: Synthetic oligonucleotide
gcguggugaacuugcggaa

SEQ ID NO: 293 - RNA, 19 nt, Artificial Sequence
  Description of Artificial Sequence: Synthetic oligonucleotide
gcaaugucgauaucuauaa

SEQ ID NO: 294 - RNA, 19 nt, Artificial Sequence
  Description of Artificial Sequence: Synthetic oligonucleotide
gcuauuggaccuuacuaug

SEQ ID NO: 295 - RNA, 19 nt, Artificial Sequence
  Description of Artificial Sequence: Synthetic oligonucleotide
acgggaaugugaugguauu

SEQ ID NO: 296 - RNA, 19 nt, Artificial Sequence
  Description of Artificial Sequence: Synthetic oligonucleotide
cggacuggauuuaucgaca

SEQ ID NO: 297 - RNA, 19 nt, Artificial Sequence
  Description of Artificial Sequence: Synthetic oligonucleotide
agagacagcugcauuguug

SEQ ID NO: 298 - RNA, 19 nt, Artificial Sequence
  Description of Artificial Sequence: Synthetic oligonucleotide
guagagauccugcuuacga

SEQ ID NO: 299 - RNA, 19 nt, Artificial Sequence
  Description of Artificial Sequence: Synthetic oligonucleotide
ccuaagauuuuaaguacga

SEQ ID NO: 300 - RNA, 19 nt, Artificial Sequence
  Description of Artificial Sequence: Synthetic oligonucleotide
uguaggaucauauggcgua

SEQ ID NO: 301 - RNA, 19 nt, Artificial Sequence
  Description of Artificial Sequence: Synthetic oligonucleotide
gccguacucuuucugacua

SEQ ID NO: 302 - RNA, 19 nt, Artificial Sequence
  Description of Artificial Sequence: Synthetic oligonucleotide
guaugcagaucuucgugaa

SEQ ID NO: 303 - RNA, 19 nt, Artificial Sequence
  Description of Artificial Sequence: Synthetic oligonucleotide
gaccaucacucuggaggug

SEQ ID NO: 304 - RNA, 19 nt, Artificial Sequence
  Description of Artificial Sequence: Synthetic oligonucleotide
cccagugacaccaucgaaa

SEQ ID NO: 305 - RNA, 19 nt, Artificial Sequence
  Description of Artificial Sequence: Synthetic oligonucleotide
gugaagacccugacuggua

SEQ ID NO: 306 - RNA, 19 nt, Artificial Sequence
  Description of Artificial Sequence: Synthetic oligonucleotide
aagcaaagauccaggacaa

SEQ ID NO: 307 - RNA, 19 nt, Artificial Sequence
  Description of Artificial Sequence: Synthetic oligonucleotide
gaagauggacgcacccugu

SEQ ID NO: 308 - RNA, 19 nt, Artificial Sequence
  Description of Artificial Sequence: Synthetic oligonucleotide
guaagaccaucacucucga

SEQ ID NO: 309 - RNA, 19 nt, Artificial Sequence
  Description of Artificial Sequence: Synthetic oligonucleotide
ccuaaaacgugucuuauuu

SEQ ID NO: 310 - RNA, 19 nt, Artificial Sequence
  Description of Artificial Sequence: Synthetic oligonucleotide
acuuguagguguuuaagaa

SEQ ID NO: 311 - RNA, 19 nt, Artificial Sequence
  Description of Artificial Sequence: Synthetic oligonucleotide
gguuagcccugccugaauc

SEQ ID NO: 312 - RNA, 19 nt, Artificial Sequence
  Description of Artificial Sequence: Synthetic oligonucleotide
uguagguguuuaagaauug

SEQ ID NO: 313 - RNA, 19 nt, Artificial Sequence
  Description of Artificial Sequence: Synthetic oligonucleotide
ggucagagauacuucuuaa

SEQ ID NO: 314 - RNA, 19 nt, Artificial Sequence
  Description of Artificial Sequence: Synthetic oligonucleotide
ccaccaagcuagauaaaga

SEQ ID NO: 315 - RNA, 19 nt, Artificial Sequence
  Description of Artificial Sequence: Synthetic oligonucleotide
gaacauagaaggagaggag

SEQ ID NO: 316 - RNA, 19 nt, Artificial Sequence
  Description of Artificial Sequence: Synthetic oligonucleotide
gcaccuaucacucucgaga

SEQ ID NO: 317 - RNA, 19 nt, Artificial Sequence
  Description of Artificial Sequence: Synthetic oligonucleotide
ggauaacucggccaugaga

SEQ ID NO: 318 - RNA, 19 nt, Artificial Sequence
  Description of Artificial Sequence: Synthetic oligonucleotide
caagaagaguuaccucagc

SEQ ID NO: 319 - RNA, 19 nt, Artificial Sequence
  Description of Artificial Sequence: Synthetic oligonucleotide
gaacucaccuccugauuau

SEQ ID NO: 320 - RNA, 19 nt, Artificial Sequence
  Description of Artificial Sequence: Synthetic oligonucleotide
gcuuaguaaugcuacgugu

SEQ ID NO: 321 - RNA, 19 nt, Artificial Sequence
  Description of Artificial Sequence: Synthetic oligonucleotide
ccuuagcagucuuaucuaa

SEQ ID NO: 322 - RNA, 19 nt, Artificial Sequence
  Description of Artificial Sequence: Synthetic oligonucleotide
ggaccaccgcaucucuaca

SEQ ID NO: 323 - RNA, 19 nt, Artificial Sequence
  Description of Artificial Sequence: Synthetic oligonucleotide
ggccaagauccaggauaaa

SEQ ID NO: 324 - RNA, 19 nt, Artificial Sequence
  Description of Artificial Sequence: Synthetic oligonucleotide
ggccaagauccaggauaag

SEQ ID NO: 325 - RNA, 19 nt, Artificial Sequence
  Description of Artificial Sequence: Synthetic oligonucleotide
ggcaaagauccaagauaag

SEQ ID NO: 326 - DNA, 19 bp, Artificial Sequence
  Description of Artificial Sequence: Synthetic oligonucleotide
  Description of Combined DNA/RNA Molecule: Synthetic oligonucleotide
aaatgugaaggccaagauc

SEQ ID NO: 327 - DNA, 19 bp, Artificial Sequence
  Description of Artificial Sequence: Synthetic oligonucleotide
  Description of Combined DNA/RNA Molecule: Synthetic oligonucleotide
aatgugaaggccaagaucc

SEQ ID NO: 328 - DNA, 19 bp, Artificial Sequence
  Description of Artificial Sequence: Synthetic oligonucleotide
  Description of Combined DNA/RNA Molecule: Synthetic oligonucleotide
atgugaaggccaagaucca

SEQ ID NO: 329 - DNA, 19 bp, Artificial Sequence
  Description of Artificial Sequence: Synthetic oligonucleotide
  Description of Combined DNA/RNA Molecule: Synthetic oligonucleotide
tgugaaggccaagauccaa

SEQ ID NO: 330 - RNA, 19 nt, Artificial Sequence
  Description of Artificial Sequence: Synthetic oligonucleotide
gugaaggccaagauccaag

SEQ ID NO: 331 - RNA, 19 nt, Artificial Sequence
  Description of Artificial Sequence: Synthetic oligonucleotide
ugaaggccaagauccaaga

SEQ ID NO: 332 - RNA, 19 nt, Artificial Sequence
  Description of Artificial Sequence: Synthetic oligonucleotide
gaaggccaagauccaagau

SEQ ID NO: 333 - RNA, 19 nt, Artificial Sequence
  Description of Artificial Sequence: Synthetic oligonucleotide
aaggccaagauccaagaua

SEQ ID NO: 334 - RNA, 19 nt, Artificial Sequence
  Description of Artificial Sequence: Synthetic oligonucleotide
aggccaagauccaagauaa

SEQ ID NO: 335 - RNA, 19 nt, Artificial Sequence
  Description of Artificial Sequence: Synthetic oligonucleotide
ggccaagauccaagauaaa

SEQ ID NO: 336 - RNA, 19 nt, Artificial Sequence
  Description of Artificial Sequence: Synthetic oligonucleotide
gccaagauccaagauaaag

SEQ ID NO: 337 - RNA, 19 nt, Artificial Sequence
  Description of Artificial Sequence: Synthetic oligonucleotide
ccaagauccaagauaaaga

SEQ ID NO: 338 - RNA, 19 nt, Artificial Sequence
  Description of Artificial Sequence: Synthetic oligonucleotide
caagauccaagauaaagaa

SEQ ID NO: 339 - RNA, 19 nt, Artificial Sequence
  Description of Artificial Sequence: Synthetic oligonucleotide
aagauccaagauaaagaag

SEQ ID NO: 340 - RNA, 19 nt, Artificial Sequence
  Description of Artificial Sequence: Synthetic oligonucleotide
agauccaagauaaagaagg

SEQ ID NO: 341 - RNA, 19 nt, Artificial Sequence
  Description of Artificial Sequence: Synthetic oligonucleotide
gauccaagauaaagaaggc

SEQ ID NO: 342 - RNA, 19 nt, Artificial Sequence
  Description of Artificial Sequence: Synthetic oligonucleotide
auccaagauaaagaaggca

SEQ ID NO: 343 - DNA, 19 bp, Artificial Sequence
  Description of Artificial Sequence: Synthetic oligonucleotide
  Description of Combined DNA/RNA Molecule: Synthetic oligonucleotide
uccaagauaaagaaggcat

SEQ ID NO: 344 - DNA, 19 bp, Artificial Sequence
  Description of Artificial Sequence: Synthetic oligonucleotide
  Description of Combined DNA/RNA Molecule: Synthetic oligonucleotide
ccaagauaaagaaggcatc

SEQ ID NO: 345 - RNA, 19 nt, Artificial Sequence
  Description of Artificial Sequence: Synthetic oligonucleotide
caagaccaucacccuugag

SEQ ID NO: 346 - RNA, 19 nt, Artificial Sequence
  Description of Artificial Sequence: Synthetic oligonucleotide
ugcagaucuucgugaagac

SEQ ID NO: 347 - RNA, 19 nt, Artificial Sequence
  Description of Artificial Sequence: Synthetic oligonucleotide
agcccagugacaccaucga

SEQ ID NO: 348 - RNA, 19 nt, Artificial Sequence
  Description of Artificial Sequence: Synthetic oligonucleotide
gacuacaacauccagaaag

SEQ ID NO: 349 - RNA, 19 nt, Artificial Sequence
  Description of Artificial Sequence: Synthetic oligonucleotide
cuacaacauccagaaagag

SEQ ID NO: 350 - RNA, 19 nt, Artificial Sequence
  Description of Artificial Sequence: Synthetic oligonucleotide
agugacaccaucgaaaaug

SEQ ID NO: 351 - RNA, 19 nt, Artificial Sequence
  Description of Artificial Sequence: Synthetic oligonucleotide
aggcaaagauccaagauaa

SEQ ID NO: 352 - RNA, 19 nt, Artificial Sequence
  Description of Artificial Sequence: Synthetic oligonucleotide
ggcaaagauccaagacaag

SEQ ID NO: 353 - RNA, 19 nt, Artificial Sequence
  Description of Artificial Sequence: Synthetic oligonucleotide
caaggcaaagauccaagac

SEQ ID NO: 354 - RNA, 19 nt, Artificial Sequence
  Description of Artificial Sequence: Synthetic oligonucleotide
aggcaaagauccaagacaa

SEQ ID NO: 355 - RNA, 19 nt, Artificial Sequence
  Description of Artificial Sequence: Synthetic oligonucleotide
caggauaaggaaggcauuc

SEQ ID NO: 356 - RNA, 19 nt, Artificial Sequence
  Description of Artificial Sequence: Synthetic oligonucleotide
caggacaaggaaggcauuc

SEQ ID NO: 357 - RNA, 19 nt, Artificial Sequence
  Description of Artificial Sequence: Synthetic oligonucleotide
ggcaagcagcuggaagaug

SEQ ID NO: 358 - RNA, 19 nt, Artificial Sequence
  Description of Artificial Sequence: Synthetic oligonucleotide
ggaaagcagcuggaagaug

SEQ ID NO: 359 - RNA, 19 nt, Artificial Sequence
  Description of Artificial Sequence: Synthetic oligonucleotide
gacuacaacauccagaagg

SEQ ID NO: 360 - RNA, 19 nt, Artificial Sequence
  Description of Artificial Sequence: Synthetic oligonucleotide
ugacuacaacauccagaag

SEQ ID NO: 361 - RNA, 19 nt, Artificial Sequence
  Description of Artificial Sequence: Synthetic oligonucleotide
ugacuacaacauccagaaa

SEQ ID NO: 362 - RNA, 19 nt, Artificial Sequence
  Description of Artificial Sequence: Synthetic oligonucleotide
ugcuguguguggggacaac

SEQ ID NO: 363 - RNA, 19 nt, Artificial Sequence
  Description of Artificial Sequence: Synthetic oligonucleotide
gggcugcaagggcuucuuc

SEQ ID NO: 364 - RNA, 19 nt, Artificial Sequence
  Description of Artificial Sequence: Synthetic oligonucleotide
gcgcacagugcagaaaaac

SEQ ID NO: 365 - RNA, 19 nt, Artificial Sequence
  Description of Artificial Sequence: Synthetic oligonucleotide
ccucacgcagagcuccauc

SEQ ID NO: 366 - RNA, 19 nt, Artificial Sequence
  Description of Artificial Sequence: Synthetic oligonucleotide
caugguguacuucaacuuc

SEQ ID NO: 367 - RNA, 19 nt, Artificial Sequence
  Description of Artificial Sequence: Synthetic oligonucleotide
guguacuucaacuucuuug

SEQ ID NO: 368 - RNA, 19 nt, Artificial Sequence
  Description of Artificial Sequence: Synthetic oligonucleotide
aauccggaaccagaucaua

SEQ ID NO: 369 - RNA, 19 nt, Artificial Sequence
  Description of Artificial Sequence: Synthetic oligonucleotide
guacaucgugggcuucuau

SEQ ID NO: 370 - RNA, 19 nt, Artificial Sequence
  Description of Artificial Sequence: Synthetic oligonucleotide
caagcccuccaacauccua

SEQ ID NO: 371 - RNA, 19 nt, Artificial Sequence
  Description of Artificial Sequence: Synthetic oligonucleotide
ucgacuccauggccaacuc

SEQ ID NO: 372 - RNA, 19 nt, Artificial Sequence
  Description of Artificial Sequence: Synthetic oligonucleotide
cauggccaacuccuucgug

SEQ ID NO: 373 - RNA, 19 nt, Artificial Sequence
  Description of Artificial Sequence: Synthetic oligonucleotide
ugagcaaugaccauaucug

SEQ ID NO: 374 - RNA, 19 nt, Artificial Sequence
  Description of Artificial Sequence: Synthetic oligonucleotide
ccaagggcuauaccaaguc

SEQ ID NO: 375 - RNA, 19 nt, Artificial Sequence
  Description of Artificial Sequence: Synthetic oligonucleotide
gucugugggcugcauucug

SEQ ID NO: 376 - RNA, 19 nt, Artificial Sequence
  Description of Artificial Sequence: Synthetic oligonucleotide
ggaggaccugaauuguauc

SEQ ID NO: 377 - RNA, 19 nt, Artificial Sequence
  Description of Artificial Sequence: Synthetic oligonucleotide
ccggcaggagcugaacaag

SEQ ID NO: 378 - RNA, 19 nt, Artificial Sequence
  Description of Artificial Sequence: Synthetic oligonucleotide
aacuggaugcauuacaacc

SEQ ID NO: 379 - RNA, 19 nt, Artificial Sequence
  Description of Artificial Sequence: Synthetic oligonucleotide
caacuggaugcauuacaac

SEQ ID NO: 380 - RNA, 19 nt, Artificial Sequence
  Description of Artificial Sequence: Synthetic oligonucleotide
gaccgagucuugcucuguu

SEQ ID NO: 381 - RNA, 19 nt, Artificial Sequence
  Description of Artificial Sequence: Synthetic oligonucleotide
uacccaggcuggagugcag

SEQ ID NO: 382 - RNA, 19 nt, Artificial Sequence
  Description of Artificial Sequence: Synthetic oligonucleotide
gaccuacaucuccaagaag

SEQ ID NO: 383 - RNA, 19 nt, Artificial Sequence
  Description of Artificial Sequence: Synthetic oligonucleotide
agaaugugcuggucaucug

SEQ ID NO: 384 - RNA, 19 nt, Artificial Sequence
  Description of Artificial Sequence: Synthetic oligonucleotide
ucaacgagcucacauauga

SEQ ID NO: 385 - RNA, 19 nt, Artificial Sequence
  Description of Artificial Sequence: Synthetic oligonucleotide
ugaccgaucuggacccaua

SEQ ID NO: 386 - RNA, 19 nt, Artificial Sequence
  Description of Artificial Sequence: Synthetic oligonucleotide
guacuugggcucauagcua

SEQ ID NO: 387 - RNA, 19 nt, Artificial Sequence
  Description of Artificial Sequence: Synthetic oligonucleotide
gcagaugagucuuacggaa

SEQ ID NO: 388 - RNA, 19 nt, Artificial Sequence
  Description of Artificial Sequence: Synthetic oligonucleotide
ccacauggccagcugacua

SEQ ID NO: 389 - RNA, 19 nt, Artificial Sequence
  Description of Artificial Sequence: Synthetic oligonucleotide
caacccgacuaucagacua

SEQ ID NO: 390 - RNA, 19 nt, Artificial Sequence
  Description of Artificial Sequence: Synthetic oligonucleotide
gaaugaauccucaccuaac

SEQ ID NO: 391 - RNA, 19 nt, Artificial Sequence
  Description of Artificial Sequence: Synthetic oligonucleotide
gcaacaaguuugacgguga

SEQ ID NO: 392 - RNA, 19 nt, Artificial Sequence
  Description of Artificial Sequence: Synthetic oligonucleotide
ggaagugcauuucaagauc

SEQ ID NO: 393 - RNA, 19 nt, Artificial Sequence
  Description of Artificial Sequence: Synthetic oligonucleotide
cgagaacaacgaagacuau

SEQ ID NO: 394 - RNA, 19 nt, Artificial Sequence
  Description of Artificial Sequence: Synthetic oligonucleotide
caagagcacucaggcaaac

SEQ ID NO: 395 - RNA, 19 nt, Artificial Sequence
  Description of Artificial Sequence: Synthetic oligonucleotide
aaagagaccuucagccgac

SEQ ID NO: 396 - RNA, 19 nt, Artificial Sequence
  Description of Artificial Sequence: Synthetic oligonucleotide
gaacgucaccaacuacuuu

SEQ ID NO: 397 - RNA, 19 nt, Artificial Sequence
  Description of Artificial Sequence: Synthetic oligonucleotide
caugcugggugucuauuug

SEQ ID NO: 398 - RNA, 19 nt, Artificial Sequence
  Description of Artificial Sequence: Synthetic oligonucleotide
caacugcggucagccaaag

SEQ ID NO: 399 - RNA, 19 nt, Artificial Sequence
  Description of Artificial Sequence: Synthetic oligonucleotide
ccaaguggccugucucuuu

SEQ ID NO: 400 - RNA, 19 nt, Artificial Sequence
  Description of Artificial Sequence: Synthetic oligonucleotide
ugagcuacaugguauauuu

SEQ ID NO: 401 - RNA, 19 nt, Artificial Sequence
  Description of Artificial Sequence: Synthetic oligonucleotide
gggauggaaccacgaauga

SEQ ID NO: 402 - RNA, 19 nt, Artificial Sequence
  Description of Artificial Sequence: Synthetic oligonucleotide
gauggaaccacgaaugaaa

SEQ ID NO: 403 - RNA, 19 nt, Artificial Sequence
  Description of Artificial Sequence: Synthetic oligonucleotide
gaaccgagacuuccgcuac

SEQ ID NO: 404 - RNA, 19 nt, Artificial Sequence
  Description of Artificial Sequence: Synthetic oligonucleotide
gaccauaccucauaacaag

SEQ ID NO: 405 - RNA, 19 nt, Artificial Sequence
  Description of Artificial Sequence: Synthetic oligonucleotide
ugaaagaccuccaccaauc

SEQ ID NO: 406 - RNA, 19 nt, Artificial Sequence
  Description of Artificial Sequence: Synthetic oligonucleotide
gaugaaggcuccagguguu

SEQ ID NO: 407 - RNA, 19 nt, Artificial Sequence
  Description of Artificial Sequence: Synthetic oligonucleotide
uaucagcauuuacgacuua

SEQ ID NO: 408 - RNA, 19 nt, Artificial Sequence
  Description of Artificial Sequence: Synthetic oligonucleotide
gaaguuguccguacagaua

SEQ ID NO: 409 - RNA, 19 nt, Artificial Sequence
  Description of Artificial Sequence: Synthetic oligonucleotide
cggaauacaccacggagau

SEQ ID NO: 410 - RNA, 19 nt, Artificial Sequence
  Description of Artificial Sequence: Synthetic oligonucleotide
ccuccaaucugcaugauga

SEQ ID NO: 411 - RNA, 19 nt, Artificial Sequence
  Description of Artificial Sequence: Synthetic oligonucleotide
gcacugagaucacggcuac

SEQ ID NO: 412 - RNA, 19 nt, Artificial Sequence
  Description of Artificial Sequence: Synthetic oligonucleotide
ccguauggcucaacuucga

SEQ ID NO: 413 - RNA, 19 nt, Artificial Sequence
  Description of Artificial Sequence: Synthetic oligonucleotide
aggcaggcgucgaagagua

SEQ ID NO: 414 - RNA, 19 nt, Artificial Sequence
  Description of Artificial Sequence: Synthetic oligonucleotide
gcucuacgaagaucuggaa

SEQ ID NO: 415 - RNA, 19 nt, Artificial Sequence
  Description of Artificial Sequence: Synthetic oligonucleotide
ggagcgcauugcacaucaa

SEQ ID NO: 416 - RNA, 19 nt, Artificial Sequence
  Description of Artificial Sequence: Synthetic oligonucleotide
guacagcgcccacgcauuc

SEQ ID NO: 417 - RNA, 19 nt, Artificial Sequence
  Description of Artificial Sequence: Synthetic oligonucleotide
gaaacagaacgcggcucua

SEQ ID NO: 418 - RNA, 19 nt, Artificial Sequence
  Description of Artificial Sequence: Synthetic oligonucleotide
gaacgcggcucuacgcaag

SEQ ID NO: 419 - RNA, 19 nt, Artificial Sequence
  Description of Artificial Sequence: Synthetic oligonucleotide
agaguucagaggagggaga

SEQ ID NO: 420 - RNA, 19 nt, Artificial Sequence
  Description of Artificial Sequence: Synthetic oligonucleotide
gaaacaggauucaguguga

SEQ ID NO: 421 - RNA, 19 nt, Artificial Sequence
  Description of Artificial Sequence: Synthetic oligonucleotide
acaaaggaguuacaucuua

SEQ ID NO: 422 - RNA, 19 nt, Artificial Sequence
  Description of Artificial Sequence: Synthetic oligonucleotide
aaagggagauucuaguaua

SEQ ID NO: 423 - RNA, 19 nt, Artificial Sequence
  Description of Artificial Sequence: Synthetic oligonucleotide
aaacagaaaucgaaacaga

SEQ ID NO: 424 - RNA, 19 nt, Artificial Sequence
  Description of Artificial Sequence: Synthetic oligonucleotide
gaagaaagcccucagaauc

SEQ ID NO: 425 - RNA, 19 nt, Artificial Sequence
  Description of Artificial Sequence: Synthetic oligonucleotide
gugccgagcugcaugacua

SEQ ID NO: 426 - RNA, 19 nt, Artificial Sequence
  Description of Artificial Sequence: Synthetic oligonucleotide
gcacagcagucaucguggu

SEQ ID NO: 427 - RNA, 19 nt, Artificial Sequence
  Description of Artificial Sequence: Synthetic oligonucleotide
ucgcugaccgugaacgaua

SEQ ID NO: 428 - RNA, 19 nt, Artificial Sequence
  Description of Artificial Sequence: Synthetic oligonucleotide
cgacuuuacccuucgacua

SEQ ID NO: 429 - RNA, 19 nt, Artificial Sequence
  Description of Artificial Sequence: Synthetic oligonucleotide
cuacagagauggcuucaac

SEQ ID NO: 430 - RNA, 19 nt, Artificial Sequence
  Description of Artificial Sequence: Synthetic oligonucleotide
caacucccuugacagugua

SEQ ID NO: 431 - RNA, 19 nt, Artificial Sequence
  Description of Artificial Sequence: Synthetic oligonucleotide
ugaggugacuccaguaucu

SEQ ID NO: 432 - RNA, 19 nt, Artificial Sequence
  Description of Artificial Sequence: Synthetic oligonucleotide
aaagggaugugaauuauug

SEQ ID NO: 433 - RNA, 19 nt, Artificial Sequence
  Description of Artificial Sequence: Synthetic oligonucleotide
gaaaacaucuauaccauug

SEQ ID NO: 434 - RNA, 19 nt, Artificial Sequence
  Description of Artificial Sequence: Synthetic oligonucleotide
gagcggaggucggucagaa

SEQ ID NO: 435 - RNA, 19 nt, Artificial Sequence
  Description of Artificial Sequence: Synthetic oligonucleotide
cagcaacccucacaaccuu

SEQ ID NO: 436 - RNA, 19 nt, Artificial Sequence
  Description of Artificial Sequence: Synthetic oligonucleotide
gggcgugacuuccacauga

SEQ ID NO: 437 - RNA, 19 nt, Artificial Sequence
  Description of Artificial Sequence: Synthetic oligonucleotide
cggagagcuucgugcuaaa

SEQ ID NO: 438 - RNA, 19 nt, Artificial Sequence
  Description of Artificial Sequence: Synthetic oligonucleotide
acaauaggagccaggcgca

SEQ ID NO: 439 - RNA, 19 nt, Artificial Sequence
  Description of Artificial Sequence: Synthetic oligonucleotide
gcaaugacagcggcaccua

SEQ ID NO: 440 - RNA, 19 nt, Artificial Sequence
  Description of Artificial Sequence: Synthetic oligonucleotide
gaacccagauuuauguaau

SEQ ID NO: 441 - RNA, 19 nt, Artificial Sequence
  Description of Artificial Sequence: Synthetic oligonucleotide
gaaccucacuauccaagga

SEQ ID NO: 442 - RNA, 19 nt, Artificial Sequence
  Description of Artificial Sequence: Synthetic oligonucleotide
auauaaaguuggaugcgga

SEQ ID NO: 443 - RNA, 19 nt, Artificial Sequence
  Description of Artificial Sequence: Synthetic oligonucleotide
cuaggaagcuccaguucga

SEQ ID NO: 444 - RNA, 19 nt, Artificial Sequence
  Description of Artificial Sequence: Synthetic oligonucleotide
gcagaucgauuugaauaua

SEQ ID NO: 445 - RNA, 19 nt, Artificial Sequence
  Description of Artificial Sequence: Synthetic oligonucleotide
gcgcagaucgauuugaaua

SEQ ID NO: 446 - RNA, 19 nt, Artificial Sequence
  Description of Artificial Sequence: Synthetic oligonucleotide
cuguuauaucagaggagua

SEQ ID NO: 447 - RNA, 19 nt, Artificial Sequence
  Description of Artificial Sequence: Synthetic oligonucleotide
ccuaaacuuaugugcuuaa

SEQ ID NO: 448 - RNA, 19 nt, Artificial Sequence
  Description of Artificial Sequence: Synthetic oligonucleotide
gccguaaacugcuuuguga

SEQ ID NO: 449 - RNA, 19 nt, Artificial Sequence
  Description of Artificial Sequence: Synthetic oligonucleotide
aggagaucacaacgcguua

SEQ ID NO: 450 - RNA, 19 nt, Artificial Sequence
  Description of Artificial Sequence: Synthetic oligonucleotide
ccaaaauuuaucacgagua

SEQ ID NO: 451 - RNA, 19 nt, Artificial Sequence
  Description of Artificial Sequence: Synthetic oligonucleotide
aguaaaaguuugcggacug

SEQ ID NO: 452 - RNA, 19 nt, Artificial Sequence
  Description of Artificial Sequence: Synthetic oligonucleotide
gaaaggaaccgagcuugua

SEQ ID NO: 453 - RNA, 19 nt, Artificial Sequence
  Description of Artificial Sequence: Synthetic oligonucleotide
ucuauuaccuggacgagau

SEQ ID NO: 454 - RNA, 19 nt, Artificial Sequence
  Description of Artificial Sequence: Synthetic oligonucleotide
gcagaacacgucucagaag

SEQ ID NO: 455 - RNA, 19 nt, Artificial Sequence
  Description of Artificial Sequence: Synthetic oligonucleotide
caagggcgagucucuauuc

SEQ ID NO: 456 - RNA, 19 nt, Artificial Sequence
  Description of Artificial Sequence: Synthetic oligonucleotide
gaaccuggcugcuguauug

SEQ ID NO: 457 - RNA, 19 nt, Artificial Sequence
  Description of Artificial Sequence: Synthetic oligonucleotide
ggaagucacugccauggaa

SEQ ID NO: 458 - RNA, 19 nt, Artificial Sequence
  Description of Artificial Sequence: Synthetic oligonucleotide
ugaaaucacugcauuacaa

SEQ ID NO: 459 - RNA, 19 nt, Artificial Sequence
  Description of Artificial Sequence: Synthetic oligonucleotide
ggacuuuauuugccauaua

SEQ ID NO: 460 - RNA, 19 nt, Artificial Sequence
  Description of Artificial Sequence: Synthetic oligonucleotide
ugaaaggacucacuuggua

SEQ ID NO: 461 - RNA, 19 nt, Artificial Sequence
  Description of Artificial Sequence: Synthetic oligonucleotide
cauaguagcuacagacaga

SEQ ID NO: 462 - RNA, 19 nt, Artificial Sequence
  Description of Artificial Sequence: Synthetic oligonucleotide
agaccuggcugcacuaauu

SEQ ID NO: 463 - RNA, 19 nt, Artificial Sequence
  Description of Artificial Sequence: Synthetic oligonucleotide
ggaccuauaugugguagag

SEQ ID NO: 464 - RNA, 21 nt, Artificial Sequence
  Description of Artificial Sequence: Synthetic oligonucleotide
uacgaauauuaaacacuucaa

SEQ ID NO: 465 - RNA, 21 nt, Artificial Sequence
  Description of Artificial Sequence: Synthetic oligonucleotide
auagguauuggugaauuuaaa

SEQ ID NO: 466 - RNA, 21 nt, Artificial Sequence
  Description of Artificial Sequence: Synthetic oligonucleotide
caggaacuggauauucugaaa

SEQ ID NO: 467 - RNA, 21 nt, Artificial Sequence
  Description of Artificial Sequence: Synthetic oligonucleotide
ugccgcaaauuccgagacgaa

SEQ ID NO: 468 - RNA, 21 nt, Artificial Sequence
  Description of Artificial Sequence: Synthetic oligonucleotide
aagaagcaguuugaagaauua

SEQ ID NO: 469 - RNA, 21 nt, Artificial Sequence
  Description of Artificial Sequence: Synthetic oligonucleotide
ccgcaucucuacauucaagaa

SEQ ID NO: 470 - RNA, 21 nt, Artificial Sequence
  Description of Artificial Sequence: Synthetic oligonucleotide
cucggcuguuccugagaaaua

SEQ ID NO: 471 - RNA, 21 nt, Artificial Sequence
  Description of Artificial Sequence: Synthetic oligonucleotide
cuggcguaagaugauggauuu

SEQ ID NO: 472 - RNA, 21 nt, Artificial Sequence
  Description of Artificial Sequence: Synthetic oligonucleotide
aagcauucguccgguugcgcu

SEQ ID NO: 473 - RNA, 21 nt, Artificial Sequence
  Description of Artificial Sequence: Synthetic oligonucleotide
ugcaccacuuccaggguuuau

SEQ ID NO: 474 - RNA, 19 nt, Artificial Sequence
  Description of Artificial Sequence: Synthetic oligonucleotide
ggaccaccgcaucucuaca

SEQ ID NO: 475 - RNA, 21 nt, Artificial Sequence
  Description of Artificial Sequence: Synthetic oligonucleotide
cagagaugaguaugcgcugua

SEQ ID NO: 476 - RNA, 21 nt, Artificial Sequence
  Description of Artificial Sequence: Synthetic oligonucleotide
auccugguuuaaagacauaaa

SEQ ID NO: 477 - skipped

SEQ ID NO: 478 - RNA, 21 nt, Artificial Sequence
  Description of Artificial Sequence: Synthetic oligonucleotide
caacgugaacuuccauaucaa

SEQ ID NO: 479 - RNA, 21 nt, Artificial Sequence
  Description of Artificial Sequence: Synthetic oligonucleotide
cugguugagaacgcaucccaa

SEQ ID NO: 480 - RNA, 21 nt, Artificial Sequence
  Description of Artificial Sequence: Synthetic oligonucleotide
uugccguuacccaguaacaua

SEQ ID NO: 481 - RNA, 21 nt, Artificial Sequence
  Description of Artificial Sequence: Synthetic oligonucleotide
acccaguaacauaauauuuca

SEQ ID NO: 482 - RNA, 21 nt, Artificial Sequence
  Description of Artificial Sequence: Synthetic oligonucleotide
uugccguuacccaguaacaua

SEQ ID NO: 483 - RNA, 21 nt, Artificial Sequence
  Description of Artificial Sequence: Synthetic oligonucleotide
acccaguaacauaauauuuca

SEQ ID NO: 484 - RNA, 21 nt, Artificial Sequence
  Description of Artificial Sequence: Synthetic oligonucleotide
gagguugaucuuugccggaaa

SEQ ID NO: 485 - DNA, 22 bp, Artificial Sequence
  Description of Artificial Sequence: Synthetic oligonucleotide
  Description of Combined DNA/RNA Molecule: Synthetic oligonucleotide
gagguugatcuuugcugggaaa

SEQ ID NO: 486 - RNA, 21 nt, Artificial Sequence
  Description of Artificial Sequence: Synthetic oligonucleotide
aacgucaaagcaaagauccaa

SEQ ID NO: 487 - DNA, 21 bp, Artificial Sequence
  Description of Artificial Sequence: Synthetic oligonucleotide
  Description of Combined DNA/RNA Molecule: Synthetic oligonucleotide
aucgcugugaucgtcacuuga

SEQ ID NO: 488 - RNA, 21 nt, Artificial Sequence
  Description of Artificial Sequence: Synthetic oligonucleotide
aaggagguuuguauaaguaau

SEQ ID NO: 489 - RNA, 21 nt, Artificial Sequence
  Description of Artificial Sequence: Synthetic oligonucleotide
caggagcgcauugcacaucaa

SEQ ID NO: 490 - RNA, 21 nt, Artificial Sequence
  Description of Artificial Sequence: Synthetic oligonucleotide
uucagugggaauugcagcaua

SEQ ID NO: 491 - RNA, 21 nt, Artificial Sequence
  Description of Artificial Sequence: Synthetic oligonucleotide
cugaugagucuugaucuagau

SEQ ID NO: 492 - DNA, 21 bp, Artificial Sequence
  Description of Artificial Sequence: Synthetic oligonucleotide
  Description of Combined DNA/RNA Molecule: Synthetic oligonucleotide
ctccaguggcucugacuacua

SEQ ID NO: 493 - RNA, 21 nt, Artificial Sequence
  Description of Artificial Sequence: Synthetic oligonucleotide
cacaggagaguuugacaccuu

SEQ ID NO: 494 - RNA, 21 nt, Artificial Sequence
  Description of Artificial Sequence: Synthetic oligonucleotide
cagcaccuucauggacggcua

SEQ ID NO: 495 - RNA, 21 nt, Artificial Sequence
  Description of Artificial Sequence: Synthetic oligonucleotide
cagcauuaugguguccgcaca

SEQ ID NO: 496 - RNA, 23 nt, Artificial Sequence
  Description of Artificial Sequence: Synthetic oligonucleotide
cagggugucagaucacaugggcu

SEQ ID NO: 497 - RNA, 21 nt, Artificial Sequence
  Description of Artificial Sequence: Synthetic oligonucleotide
cuccaguggcucugacuacua

SEQ ID NO: 498 - RNA, 25 nt, Artificial Sequence
  Description of Artificial Sequence: Synthetic oligonucleotide
cagcauuaugguguccgcacaugug

SEQ ID NO: 499 - RNA, 25 nt, Artificial Sequence
  Description of Artificial Sequence: Synthetic oligonucleotide
ggccaagauccaagauaaagaaggc

SEQ ID NO: 500 - RNA, 19 nt, Artificial Sequence
  Description of Artificial Sequence: Synthetic oligonucleotide
caccauugaugucuacaug

SEQ ID NO: 501 - RNA, 19 nt, Artificial Sequence
  Description of Artificial Sequence: Synthetic oligonucleotide
cagcaacuucuucaugaac

SEQ ID NO: 502 - RNA, 19 nt, Artificial Sequence
  Description of Artificial Sequence: Synthetic oligonucleotide
caucuucaaggagaacaaa

SEQ ID NO: 503 - RNA, 19 nt, Artificial Sequence
  Description of Artificial Sequence: Synthetic oligonucleotide
ugccaggaggcggcagcuc

SEQ ID NO: 504 - RNA, 19 nt, Artificial Sequence
  Description of Artificial Sequence: Synthetic oligonucleotide
ugcaguaucuggccccaga

SEQ ID NO: 505 - RNA, 19 nt, Artificial Sequence
  Description of Artificial Sequence: Synthetic oligonucleotide
cuacauagggaucugaaac

SEQ ID NO: 506 - RNA, 19 nt, Artificial Sequence
  Description of Artificial Sequence: Synthetic oligonucleotide
uuugccaguggcaguccau

SEQ ID NO: 507 - RNA, 19 nt, Artificial Sequence
  Description of Artificial Sequence: Synthetic oligonucleotide
gaauguaagaaagcucagc

SEQ ID NO: 508 - RNA, 19 nt, Artificial Sequence
  Description of Artificial Sequence: Synthetic oligonucleotide
gucuacuucaacuucuuug

SEQ ID NO: 509 - RNA, 19 nt, Artificial Sequence
  Description of Artificial Sequence: Synthetic oligonucleotide
ccucacgcagagcuccauc

SEQ ID NO: 510 - RNA, 19 nt, Artificial Sequence
  Description of Artificial Sequence: Synthetic oligonucleotide
ccucacccagagcuccauc

SEQ ID NO: 511 - RNA, 19 nt, Artificial Sequence
  Description of Artificial Sequence: Synthetic oligonucleotide
caugguguacuucaacuuc

SEQ ID NO: 512 - RNA, 19 nt, Artificial Sequence
  Description of Artificial Sequence: Synthetic oligonucleotide
cauggucuacuucaacuuc

SEQ ID NO: 513 - RNA, 19 nt, Artificial Sequence
  Description of Artificial Sequence: Synthetic oligonucleotide
guguacuucaacuucuuug

SEQ ID NO: 514 - RNA, 19 nt, Artificial Sequence
  Description of Artificial Sequence: Synthetic oligonucleotide
gucuacuucaacuucuuug

SEQ ID NO: 515 - RNA, 19 nt, Artificial Sequence
  Description of Artificial Sequence: Synthetic oligonucleotide
aauccggaaccagaucaua

SEQ ID NO: 516 - RNA, 19 nt, Artificial Sequence
  Description of Artificial Sequence: Synthetic oligonucleotide
cauccggaaccagaucauc

SEQ ID NO: 517 - RNA, 19 nt, Artificial Sequence
  Description of Artificial Sequence: Synthetic oligonucleotide
guacaucgugggcuucuau

SEQ ID NO: 518 - RNA, 19 nt, Artificial Sequence
  Description of Artificial Sequence: Synthetic oligonucleotide
guacaucgugggcuucuac

SEQ ID NO: 519 - RNA, 19 nt, Artificial Sequence
  Description of Artificial Sequence: Synthetic oligonucleotide
caagcccuccaacauccua

SEQ ID NO: 520 - RNA, 19 nt, Artificial Sequence
  Description of Artificial Sequence: Synthetic oligonucleotide
gaagcccuccaacauccuc

SEQ ID NO: 521 - RNA, 19 nt, Artificial Sequence
  Description of Artificial Sequence: Synthetic oligonucleotide
gaucaagcucugugacuuu

SEQ ID NO: 522 - RNA, 19 nt, Artificial Sequence
  Description of Artificial Sequence: Synthetic oligonucleotide
gaucaagcugugugacuuc

SEQ ID NO: 523 - RNA, 19 nt, Artificial Sequence
  Description of Artificial Sequence: Synthetic oligonucleotide
ucgacuccauggccaacuc

SEQ ID NO: 524 - RNA, 19 nt, Artificial Sequence
  Description of Artificial Sequence: Synthetic oligonucleotide
cauggccaacuccuucgug

SEQ ID NO: 525 - RNA, 19 nt, Artificial Sequence
  Description of Artificial Sequence: Synthetic oligonucleotide
ugagcaaugaccauaucug

SEQ ID NO: 526 - RNA, 19 nt, Artificial Sequence
  Description of Artificial Sequence: Synthetic oligonucleotide
ucagcaaugaccauaucug

SEQ ID NO: 527 - RNA, 19 nt, Artificial Sequence
  Description of Artificial Sequence: Synthetic oligonucleotide
cuacuuccucuaccagauc

SEQ ID NO: 528 - RNA, 19 nt, Artificial Sequence
  Description of Artificial Sequence: Synthetic oligonucleotide
cuauuuucucuaccagauc

SEQ ID NO: 529 - RNA, 19 nt, Artificial Sequence
  Description of Artificial Sequence: Synthetic oligonucleotide
ccaagggcuauaccaaguc

SEQ ID NO: 530 - RNA, 19 nt, Artificial Sequence
  Description of Artificial Sequence: Synthetic oligonucleotide
ccaagggcuacaccaaguc

SEQ ID NO: 531 - RNA, 19 nt, Artificial Sequence
  Description of Artificial Sequence: Synthetic oligonucleotide
gucugugggcugcauucug

SEQ ID NO: 532 - RNA, 19 nt, Artificial Sequence
  Description of Artificial Sequence: Synthetic oligonucleotide
gucuguaggcugcauucug

SEQ ID NO: 533 - RNA, 19 nt, Artificial Sequence
  Description of Artificial Sequence: Synthetic oligonucleotide
cucuaaccggcccaucuuc

SEQ ID NO: 534 - RNA, 19 nt, Artificial Sequence
  Description of Artificial Sequence: Synthetic oligonucleotide
cucuaacaggcccaucuuc

SEQ ID NO: 535 - RNA, 19 nt, Artificial Sequence
  Description of Artificial Sequence: Synthetic oligonucleotide
ggaggaccugaauuguauc

SEQ ID NO: 536 - RNA, 19 nt, Artificial Sequence
  Description of Artificial Sequence: Synthetic oligonucleotide
agaagaccugaauuguaua

SEQ ID NO: 537 - RNA, 19 nt, Artificial Sequence
  Description of Artificial Sequence: Synthetic oligonucleotide
gaaagccuuggaugguuuu

SEQ ID NO: 538 - RNA, 19 nt, Artificial Sequence
  Description of Artificial Sequence: Synthetic oligonucleotide
uacucaggccgccaucaag

SEQ ID NO: 539 - RNA, 19 nt, Artificial Sequence
  Description of Artificial Sequence: Synthetic oligonucleotide
agaccuacaucuccaagaa

SEQ ID NO: 540 - RNA, 19 nt, Artificial Sequence
  Description of Artificial Sequence: Synthetic oligonucleotide
gugggacuccuaauuacau

SEQ ID NO: 541 - RNA, 19 nt, Artificial Sequence
  Description of Artificial Sequence: Synthetic oligonucleotide
caagaaaacagaugaaauu

SEQ ID NO: 542 - RNA, 19 nt, Artificial Sequence
  Description of Artificial Sequence: Synthetic oligonucleotide
guuucgucgaaagccucuc

SEQ ID NO: 543 - RNA, 19 nt, Artificial Sequence
  Description of Artificial Sequence: Synthetic oligonucleotide
ccaagagugccaacuacug

SEQ ID NO: 544 - RNA, 19 nt, Artificial Sequence
  Description of Artificial Sequence: Synthetic oligonucleotide
agcgcacagugcagaaaaa

SEQ ID NO: 545 - RNA, 19 nt, Artificial Sequence
  Description of Artificial Sequence: Synthetic oligonucleotide
ugcuguguguggggacaac

SEQ ID NO: 546 - RNA, 19 nt, Artificial Sequence
  Description of Artificial Sequence: Synthetic oligonucleotide
cgcuguguguggggacaac

SEQ ID NO: 547 - RNA, 19 nt, Artificial Sequence
  Description of Artificial Sequence: Synthetic oligonucleotide
augugagggcugcugcaag

SEQ ID NO: 548 - RNA, 19 nt, Artificial Sequence
  Description of Artificial Sequence: Synthetic oligonucleotide
cugugagggcugcugcaaa

SEQ ID NO: 549 - RNA, 19 nt, Artificial Sequence
  Description of Artificial Sequence: Synthetic oligonucleotide
cugcaagggcuucuucaag

SEQ ID NO: 550 - RNA, 19 nt, Artificial Sequence
  Description of Artificial Sequence: Synthetic oligonucleotide
cugcaaaggcuucuuuaag

SEQ ID NO: 551 - RNA, 19 nt, Artificial Sequence
  Description of Artificial Sequence: Synthetic oligonucleotide
gggcugcaagggcuucuuc

SEQ ID NO: 552 - RNA, 19 nt, Artificial Sequence
  Description of Artificial Sequence: Synthetic oligonucleotide
gggcugcaaaggcuucuuu

SEQ ID NO: 553 - RNA, 19 nt, Artificial Sequence
  Description of Artificial Sequence: Synthetic oligonucleotide
gcgcacagugcagaaaaac

SEQ ID NO: 554 - RNA, 19 nt, Artificial Sequence
  Description of Artificial Sequence: Synthetic oligonucleotide
gcgcacagugcaaaaaaac

SEQ ID NO: 555 - RNA, 19 nt, Artificial Sequence
  Description of Artificial Sequence: Synthetic oligonucleotide
ugcuguguguggggacaac

SEQ ID NO: 556 - RNA, 19 nt, Artificial Sequence
  Description of Artificial Sequence: Synthetic oligonucleotide
cgcuguguguggggacaac

SEQ ID NO: 557 - RNA, 19 nt, Artificial Sequence
  Description of Artificial Sequence: Synthetic oligonucleotide
ugccgugugcggggacaac

SEQ ID NO: 558 - RNA, 19 nt, Artificial Sequence
  Description of Artificial Sequence: Synthetic oligonucleotide
cugcaagggcuucuucaag

SEQ ID NO: 559 - RNA, 19 nt, Artificial Sequence
  Description of Artificial Sequence: Synthetic oligonucleotide
cugcaaaggcuucuuuaag

SEQ ID NO: 560 - RNA, 19 nt, Artificial Sequence
  Description of Artificial Sequence: Synthetic oligonucleotide
cugcaagggcuuuuucaag

SEQ ID NO: 561 - RNA, 19 nt, Artificial Sequence
  Description of Artificial Sequence: Synthetic oligonucleotide
gggcugcaagggcuucuuc

SEQ ID NO: 562 - RNA, 19 nt, Artificial Sequence
  Description of Artificial Sequence: Synthetic oligonucleotide
gggcugcaaaggcuucuuu

SEQ ID NO: 563 - RNA, 19 nt, Artificial Sequence
  Description of Artificial Sequence: Synthetic oligonucleotide
gggcugcaagggcuuuuuc

SEQ ID NO: 564 - RNA, 19 nt, Artificial Sequence
  Description of Artificial Sequence: Synthetic oligonucleotide
gcgcacagugcagaaaaac

SEQ ID NO: 565 - RNA, 19 nt, Artificial Sequence
  Description of Artificial Sequence: Synthetic oligonucleotide
gcgcacagugcaaaaaaac

SEQ ID NO: 566 - RNA, 19 nt, Artificial Sequence
  Description of Artificial Sequence: Synthetic oligonucleotide
gagaacagugcagaaaaau

SEQ ID NO: 567 - RNA, 19 nt, Artificial Sequence
  Description of Artificial Sequence: Synthetic oligonucleotide
acugcccuguggacaagag

SEQ ID NO: 568 - RNA, 19 nt, Artificial Sequence
  Description of Artificial Sequence: Synthetic oligonucleotide
acugcccaguggacaagcg

SEQ ID NO: 569 - RNA, 19 nt, Artificial Sequence
  Description of Artificial Sequence: Synthetic oligonucleotide
acugcccaguagacaagag

SEQ ID NO: 570 - RNA, 19 nt, Artificial Sequence
  Description of Artificial Sequence: Synthetic oligonucleotide
uucaaugaugauauucaag

SEQ ID NO: 571 - RNA, 19 nt, Artificial Sequence
  Description of Artificial Sequence: Synthetic oligonucleotide
auccaacaauagaggauuc

SEQ ID NO: 572 - RNA, 19 nt, Artificial Sequence
  Description of Artificial Sequence: Synthetic oligonucleotide
ucauuuuguggacgaauau

SEQ ID NO: 573 - RNA, 19 nt, Artificial Sequence
  Description of Artificial Sequence: Synthetic oligonucleotide
guggugugagggcuccagc

SEQ ID NO: 574 - RNA, 19 nt, Artificial Sequence
  Description of Artificial Sequence: Synthetic oligonucleotide
cuuacaacacacaacuuug

SEQ ID NO: 575 - DNA, 25 bp, Artificial Sequence
  Description of Artificial Sequence: Synthetic oligonucleotide
  Description of Combined DNA/RNA Molecule: Synthetic oligonucleotide
aauugaggugcuuccuuaauuguga

SEQ ID NO: 576 - RNA, 27 nt, Artificial Sequence
  Description of Artificial Sequence: Synthetic oligonucleotide
ucacaaucagggaagcaucacaauugg

SEQ ID NO: 577 - RNA, 21 nt, Artificial Sequence
  Description of Artificial Sequence: Synthetic oligonucleotide
aauugaggugcuuccuuaauu

SEQ ID NO: 578 - RNA, 21 nt, Artificial Sequence
  Description of Artificial Sequence: Synthetic oligonucleotide
ucagggaagcaucacaauugg

SEQ ID NO: 579 - DNA, 25 bp, Artificial Sequence
  Description of Artificial Sequence: Synthetic oligonucleotide
  Description of Combined DNA/RNA Molecule: Synthetic oligonucleotide
auuugaggcaguuuacauuucggtg

SEQ ID NO: 580 - RNA, 27 nt, Artificial Sequence
  Description of Artificial Sequence: Synthetic oligonucleotide
caccgaaauguucacuuccucaaguuc

SEQ ID NO: 581 - RNA, 21 nt, Artificial Sequence
  Description of Artificial Sequence: Synthetic oligonucleotide
auuugaggcaguuuacauuuc

SEQ ID NO: 582 - RNA, 21 nt, Artificial Sequence
  Description of Artificial Sequence: Synthetic oligonucleotide
aauguucacuuccucaaguuc

SEQ ID NO: 583 - DNA, 25 bp, Artificial Sequence
  Description of Artificial Sequence: Synthetic oligonucleotide
  Description of Combined DNA/RNA Molecule: Synthetic oligonucleotide
uuuccauuugucuuugaucuuugtt

SEQ ID NO: 584 - RNA, 27 nt, Artificial Sequence
  Description of Artificial Sequence: Synthetic oligonucleotide
aacaaagaucacauauaaauggaaggc

SEQ ID NO: 585 - RNA, 21 nt, Artificial Sequence
  Description of Artificial Sequence: Synthetic oligonucleotide
uuuccauuugucuuugaucuu

SEQ ID NO: 586 - RNA, 21 nt, Artificial Sequence
  Description of Artificial Sequence: Synthetic oligonucleotide
gaucacauauaaauggaaggc

SEQ ID NO: 587 - DNA, 25 bp, Artificial Sequence
  Description of Artificial Sequence: Synthetic oligonucleotide
  Description of Combined DNA/RNA Molecule: Synthetic oligonucleotide
uuccucaaguuuggaagcagccutg

SEQ ID NO: 588 - RNA, 27 nt, Artificial Sequence
  Description of Artificial Sequence: Synthetic oligonucleotide
caaggcugauucuaaacuggaagaaaa

SEQ ID NO: 589 - DNA, 25 bp, Artificial Sequence
  Description of Artificial Sequence: Synthetic oligonucleotide
  Description of Combined DNA/RNA Molecule: Synthetic oligonucleotide
accaucagugacguaagcaaaagaa

SEQ ID NO: 590 - RNA, 27 nt, Artificial Sequence
  Description of Artificial Sequence: Synthetic oligonucleotide
uucuuuugcuuaagucagagauggaag

SEQ ID NO: 591 - DNA, 25 bp, Artificial Sequence
  Description of Artificial Sequence: Synthetic oligonucleotide
  Description of Combined DNA/RNA Molecule: Synthetic oligonucleotide
auuccaaugccauccacuuucagtc

SEQ ID NO: 592 - RNA, 27 nt, Artificial Sequence
  Description of Artificial Sequence: Synthetic oligonucleotide
gacugaaagugcuuggaaauggaaugg

SEQ ID NO: 593 - DNA, 25 bp, Artificial Sequence
  Description of Artificial Sequence: Synthetic oligonucleotide
  Description of Combined DNA/RNA Molecule: Synthetic oligonucleotide
uccauuuacagacugacacuuucca

SEQ ID NO: 594 - RNA, 27 nt, Artificial Sequence
  Description of Artificial Sequence: Synthetic oligonucleotide
uggaaaguuacagacuguaaauagagu

SEQ ID NO: 595 - DNA, 19 bp, Artificial Sequence
  Description of Artificial Sequence: Synthetic oligonucleotide
aaatgtgaaggccaagatc

SEQ ID NO: 596 - DNA, 19 bp, Artificial Sequence
  Description of Artificial Sequence: Synthetic oligonucleotide
aatgtgaaggccaagatcc

SEQ ID NO: 597 - DNA, 19 bp, Artificial Sequence
  Description of Artificial Sequence: Synthetic oligonucleotide
atgtgaaggccaagatcca

SEQ ID NO: 598 - DNA, 19 bp, Artificial Sequence
  Description of Artificial Sequence: Synthetic oligonucleotide
tgtgaaggccaagatccaa

SEQ ID NO: 599 - DNA, 19 bp, Artificial Sequence
  Description of Artificial Sequence: Synthetic oligonucleotide
gtgaaggccaagatccaag

SEQ ID NO: 600 - DNA, 19 bp, Artificial Sequence
  Description of Artificial Sequence: Synthetic oligonucleotide
tgaaggccaagatccaaga

SEQ ID NO: 601 - DNA, 19 bp, Artificial Sequence
  Description of Artificial Sequence: Synthetic oligonucleotide
gaaggccaagatccaagat

SEQ ID NO: 602 - DNA, 19 bp, Artificial Sequence
  Description of Artificial Sequence: Synthetic oligonucleotide
aaggccaagatccaagata

SEQ ID NO: 603 - DNA, 19 bp, Artificial Sequence
  Description of Artificial Sequence: Synthetic oligonucleotide
aggccaagatccaagataa

SEQ ID NO: 604 - DNA, 19 bp, Artificial Sequence
  Description of Artificial Sequence: Synthetic oligonucleotide
ggccaagatccaagataaa

SEQ ID NO: 605 - DNA, 19 bp, Artificial Sequence
  Description of Artificial Sequence: Synthetic oligonucleotide
gccaagatccaagataaag

SEQ ID NO: 606 - DNA, 19 bp, Artificial Sequence
  Description of Artificial Sequence: Synthetic oligonucleotide
ccaagatccaagataaaga

SEQ ID NO: 607 - DNA, 19 bp, Artificial Sequence
  Description of Artificial Sequence: Synthetic oligonucleotide
caagatccaagataaagaa

SEQ ID NO: 608 - DNA, 19 bp, Artificial Sequence
  Description of Artificial Sequence: Synthetic oligonucleotide
aagatccaagataaagaag

SEQ ID NO: 609 - DNA, 19 bp, Artificial Sequence
  Description of Artificial Sequence: Synthetic oligonucleotide
agatccaagataaagaagg

SEQ ID NO: 610 - DNA, 19 bp, Artificial Sequence
  Description of Artificial Sequence: Synthetic oligonucleotide
gatccaagataaagaaggc

SEQ ID NO: 611 - DNA, 19 bp, Artificial Sequence
  Description of Artificial Sequence: Synthetic oligonucleotide
atccaagataaagaaggca

SEQ ID NO: 612 - DNA, 19 bp, Artificial Sequence
  Description of Artificial Sequence: Synthetic oligonucleotide
tccaagataaagaaggcat

SEQ ID NO: 613 - DNA, 19 bp, Artificial Sequence
  Description of Artificial Sequence: Synthetic oligonucleotide
ccaagataaagaaggcatc

SEQ ID NO: 614 - DNA, 21 bp, Unknown
  Description of Unknown: UBB_1 sequence
caggatcctggtatccgctaa

SEQ ID NO: 615 - DNA, 21 bp, Unknown
  Description of Unknown: UBB_2 sequence
atggcattactctgcactata

SEQ ID NO: 616 - DNA, 21 bp, Unknown
  Description of Unknown: UBB_3 sequence
ccaacttaagtttagaaatta

SEQ ID NO: 617 - DNA, 21 bp, Unknown
  Description of Unknown: UBB_4 sequence
gaggctcatctttgcaggcaa

SEQ ID NO: 618 - DNA, 19 bp, Artificial Sequence
  Description of Artificial Sequence: Synthetic oligonucleotide
gaacaaccggcaaatagat

SEQ ID NO: 619 - DNA, 19 bp, Artificial Sequence
  Description of Artificial Sequence: Synthetic oligonucleotide
gcaatacgcgaagacataa

SEQ ID NO: 620 - DNA, 19 bp, Artificial Sequence
  Description of Artificial Sequence: Synthetic oligonucleotide
gaaagacggaccataacat

SEQ ID NO: 621 - DNA, 19 bp, Artificial Sequence
  Description of Artificial Sequence: Synthetic oligonucleotide
gaagaaccacgaagactta

SEQ ID NO: 622 - DNA, 19 bp, Artificial Sequence
  Description of Artificial Sequence: Synthetic oligonucleotide
gtaggacgcacaaactaaa

SEQ ID NO: 623 - DNA, 19 bp, Artificial Sequence
  Description of Artificial Sequence: Synthetic oligonucleotide
ggacagatcgctaaacaaa

SEQ ID NO: 624 - DNA, 19 bp, Artificial Sequence
  Description of Artificial Sequence: Synthetic oligonucleotide
ggccaagatccaggataaa

SEQ ID NO: 625 - DNA, 19 bp, Artificial Sequence
  Description of Artificial Sequence: Synthetic oligonucleotide
ggccaagatccaggataag

SEQ ID NO: 626 - DNA, 19 bp, Artificial Sequence
  Description of Artificial Sequence: Synthetic oligonucleotide
ggcaaagatccaagataag

SEQ ID NO: 627 - DNA, 19 bp, Artificial Sequence
  Description of Artificial Sequence: Synthetic oligonucleotide
caagaccatcacccttgag

SEQ ID NO: 628 - DNA, 19 bp, Artificial Sequence
  Description of Artificial Sequence: Synthetic oligonucleotide
tgcagatcttcgtgaagac

SEQ ID NO: 629 - DNA, 19 bp, Artificial Sequence
  Description of Artificial Sequence: Synthetic oligonucleotide
agcccagtgacaccatcga

SEQ ID NO: 630 - DNA, 19 bp, Artificial Sequence
  Description of Artificial Sequence: Synthetic oligonucleotide
gactacaacatccagaaag

SEQ ID NO: 631 - DNA, 19 bp, Artificial Sequence
  Description of Artificial Sequence: Synthetic oligonucleotide
ctacaacatccagaaagag

SEQ ID NO: 632 - DNA, 19 bp, Artificial Sequence
  Description of Artificial Sequence: Synthetic oligonucleotide
tgactacaacatccagaaa

SEQ ID NO: 633 - DNA, 19 bp, Artificial Sequence
  Description of Artificial Sequence: Synthetic oligonucleotide
agtgacaccatcgaaaatg

SEQ ID NO: 634 - DNA, 19 bp, Artificial Sequence
  Description of Artificial Sequence: Synthetic oligonucleotide
aggcaaagatccaagataa

SEQ ID NO: 635 - DNA, 19 bp, Artificial Sequence
  Description of Artificial Sequence: Synthetic oligonucleotide
ggcaaagatccaagacaag

SEQ ID NO: 636 - DNA, 19 bp, Artificial Sequence
  Description of Artificial Sequence: Synthetic oligonucleotide
caaggcaaagatccaagac

SEQ ID NO: 637 - DNA, 19 bp, Artificial Sequence
  Description of Artificial Sequence: Synthetic oligonucleotide
aggcaaagatccaagacaa

SEQ ID NO: 638 - DNA, 19 bp, Artificial Sequence
  Description of Artificial Sequence: Synthetic oligonucleotide
caggataaggaaggcattc

SEQ ID NO: 639 - DNA, 19 bp, Artificial Sequence
  Description of Artificial Sequence: Synthetic oligonucleotide
caggacaaggaaggcattc

SEQ ID NO: 640 - DNA, 19 bp, Artificial Sequence
  Description of Artificial Sequence: Synthetic oligonucleotide
ggcaagcagctggaagatg

SEQ ID NO: 641 - DNA, 19 bp, Artificial Sequence
  Description of Artificial Sequence: Synthetic oligonucleotide
ggaaagcagctggaagatg

SEQ ID NO: 642 - DNA, 19 bp, Artificial Sequence
  Description of Artificial Sequence: Synthetic oligonucleotide
gactacaacatccagaagg

SEQ ID NO: 643 - DNA, 19 bp, Artificial Sequence
  Description of Artificial Sequence: Synthetic oligonucleotide
tgactacaacatccagaag

SEQ ID NO: 644 - RNA, 19 nt, Unknown
  Description of Unknown: U52 sequence
ggcaaagauccaggacaag

SEQ ID NO: 645 - RNA, 25 nt, Artificial Sequence
  Description of Artificial Sequence: Synthetic oligonucleotide
ggccaagauccaagauaaagaaggc

SEQ ID NO: 646 - DNA, 19 bp, Artificial Sequence
  Description of Artificial Sequence: Synthetic oligonucleotide
tgctgtgtgtggggacaac

SEQ ID NO: 647 - DNA, 19 bp, Artificial Sequence
  Description of Artificial Sequence: Synthetic oligonucleotide
gggctgcaagggcttcttc

SEQ ID NO: 648 - DNA, 19 bp, Artificial Sequence
  Description of Artificial Sequence: Synthetic oligonucleotide
gcgcacagtgcagaaaaac

SEQ ID NO: 649 - DNA, 19 bp, Artificial Sequence
  Description of Artificial Sequence: Synthetic oligonucleotide
cctcacgcagagctccatc

SEQ ID NO: 650 - DNA, 19 bp, Artificial Sequence
  Description of Artificial Sequence: Synthetic oligonucleotide
catggtgtacttcaacttc

SEQ ID NO: 651 - DNA, 19 bp, Artificial Sequence
  Description of Artificial Sequence: Synthetic oligonucleotide
gtgtacttcaacttctttg

SEQ ID NO: 652 - DNA, 19 bp, Artificial Sequence
  Description of Artificial Sequence: Synthetic oligonucleotide
aatccggaaccagatcata

SEQ ID NO: 653 - DNA, 19 bp, Artificial Sequence
  Description of Artificial Sequence: Synthetic oligonucleotide
gtacatcgtgggcttctat

SEQ ID NO: 654 - DNA, 19 bp, Artificial Sequence
  Description of Artificial Sequence: Synthetic oligonucleotide
caagccctccaacatccta

SEQ ID NO: 655 - DNA, 19 bp, Artificial Sequence
  Description of Artificial Sequence: Synthetic oligonucleotide
tcgactccatggccaactc

SEQ ID NO: 656 - DNA, 19 bp, Artificial Sequence
  Description of Artificial Sequence: Synthetic oligonucleotide
catggccaactccttcgtg

SEQ ID NO: 657 - DNA, 19 bp, Artificial Sequence
  Description of Artificial Sequence: Synthetic oligonucleotide
tgagcaatgaccatatctg

SEQ ID NO: 658 - DNA, 19 bp, Artificial Sequence
  Description of Artificial Sequence: Synthetic oligonucleotide
ccaagggctataccaagtc

SEQ ID NO: 659 - DNA, 19 bp, Artificial Sequence
  Description of Artificial Sequence: Synthetic oligonucleotide
gtctgtgggctgcattctg

SEQ ID NO: 660 - DNA, 19 bp, Artificial Sequence
  Description of Artificial Sequence: Synthetic oligonucleotide
ggaggacctgaattgtatc

SEQ ID NO: 661 - DNA, 19 bp, Artificial Sequence
  Description of Artificial Sequence: Synthetic oligonucleotide
ccggcaggagctgaacaag

SEQ ID NO: 662 - DNA, 19 bp, Artificial Sequence
  Description of Artificial Sequence: Synthetic oligonucleotide
aactggatgcattacaacc

SEQ ID NO: 663 - DNA, 19 bp, Artificial Sequence
  Description of Artificial Sequence: Synthetic oligonucleotide
caactggatgcattacaac

SEQ ID NO: 664 - DNA, 19 bp, Artificial Sequence
  Description of Artificial Sequence: Synthetic oligonucleotide
gaccgagtcttgctctgtt

SEQ ID NO: 665 - DNA, 19 bp, Artificial Sequence
  Description of Artificial Sequence: Synthetic oligonucleotide
tacccaggctggagtgcag

SEQ ID NO: 666 - DNA, 19 bp, Artificial Sequence
  Description of Artificial Sequence: Synthetic oligonucleotide
gacctacatctccaagaag

SEQ ID NO: 667 - DNA, 19 bp, Artificial Sequence
  Description of Artificial Sequence: Synthetic oligonucleotide
agaatgtgctggtcatctg

SEQ ID NO: 668 - DNA, 19 bp, Artificial Sequence
  Description of Artificial Sequence: Synthetic oligonucleotide
gcacatggatggaggttct

SEQ ID NO: 669 - DNA, 19 bp, Artificial Sequence
  Description of Artificial Sequence: Synthetic oligonucleotide
gcagagagagcagatttga

SEQ ID NO: 670 - DNA, 19 bp, Artificial Sequence
  Description of Artificial Sequence: Synthetic oligonucleotide
gagcagatttgaagcaact

SEQ ID NO: 671 - DNA, 19 bp, Artificial Sequence
  Description of Artificial Sequence: Synthetic oligonucleotide
ccagaaagctaattcatct

SEQ ID NO: 672 - DNA, 19 bp, Artificial Sequence
  Description of Artificial Sequence: Synthetic oligonucleotide
caaagacgatgacttcgaa

SEQ ID NO: 673 - DNA, 19 bp, Artificial Sequence
  Description of Artificial Sequence: Synthetic oligonucleotide
gatcagcatttgcatggaa

SEQ ID NO: 674 - DNA, 19 bp, Artificial Sequence
  Description of Artificial Sequence: Synthetic oligonucleotide
ggaagctgatccaccttga

SEQ ID NO: 675 - DNA, 19 bp, Artificial Sequence
  Description of Artificial Sequence: Synthetic oligonucleotide
gaaagtcagcatcgcggtt

SEQ ID NO: 676 - DNA, 21 bp, Artificial Sequence
  Description of Artificial Sequence: Synthetic oligonucleotide
tacgaatattaaacacttcaa

SEQ ID NO: 677 - DNA, 21 bp, Artificial Sequence
  Description of Artificial Sequence: Synthetic oligonucleotide
ataggtattggtgaatttaaa

SEQ ID NO: 678 - DNA, 21 bp, Artificial Sequence
  Description of Artificial Sequence: Synthetic oligonucleotide
caggaactggatattctgaaa

SEQ ID NO: 679 - DNA, 21 bp, Artificial Sequence
  Description of Artificial Sequence: Synthetic oligonucleotide
tgccgcaaattccgagacgaa

SEQ ID NO: 680 - DNA, 19 bp, Artificial Sequence
  Description of Artificial Sequence: Synthetic oligonucleotide
ccgcaaattccgagacgaa

SEQ ID NO: 681 - DNA, 19 bp, Artificial Sequence
  Description of Artificial Sequence: Synthetic oligonucleotide
caaagtgtgtaacggaata

SEQ ID NO: 682 - DNA, 19 bp, Artificial Sequence
  Description of Artificial Sequence: Synthetic oligonucleotide
gtaacaagctcacgcagtt

SEQ ID NO: 683 - DNA, 19 bp, Artificial Sequence
  Description of Artificial Sequence: Synthetic oligonucleotide
gaggaaatatgtactacga

SEQ ID NO: 684 - DNA, 19 bp, Artificial Sequence
  Description of Artificial Sequence: Synthetic oligonucleotide
ccttagcagtcttatctaa

SEQ ID NO: 685 - DNA, 21 bp, Artificial Sequence
  Description of Artificial Sequence: Synthetic oligonucleotide
aagaagcagtttgaagaatta

SEQ ID NO: 686 - DNA, 21 bp, Artificial Sequence
  Description of Artificial Sequence: Synthetic oligonucleotide
ccgcatctctacattcaagaa

SEQ ID NO: 687 - DNA, 21 bp, Artificial Sequence
  Description of Artificial Sequence: Synthetic oligonucleotide
ctcggctgttcctgagaaata

SEQ ID NO: 688 - DNA, 21 bp, Artificial Sequence
  Description of Artificial Sequence: Synthetic oligonucleotide
ctggcgtaagatgatggattt

SEQ ID NO: 689 - DNA, 21 bp, Artificial Sequence
  Description of Artificial Sequence: Synthetic oligonucleotide
aagcattcgtccggttgcgct

SEQ ID NO: 690 - DNA, 21 bp, Artificial Sequence
  Description of Artificial Sequence: Synthetic oligonucleotide
tgcaccacttccagggtttat

SEQ ID NO: 691 - DNA, 19 bp, Artificial Sequence
  Description of Artificial Sequence: Synthetic oligonucleotide
ggaccaccgcatctctaca

SEQ ID NO: 692 - DNA, 21 bp, Artificial Sequence
  Description of Artificial Sequence: Synthetic oligonucleotide
cagagatgagtatgcgctgta

SEQ ID NO: 693 - DNA, 21 bp, Artificial Sequence
  Description of Artificial Sequence: Synthetic oligonucleotide
atcctggtttaaagacataaa

SEQ ID NO: 694 - DNA, 21 bp, Artificial Sequence
  Description of Artificial Sequence: Synthetic oligonucleotide
caacgtgaacttccatatcaa

SEQ ID NO: 695 - DNA, 21 bp, Artificial Sequence
  Description of Artificial Sequence: Synthetic oligonucleotide
acccagtaacataatatttca

SEQ ID NO: 696 - DNA, 19 bp, Artificial Sequence
  Description of Artificial Sequence: Synthetic oligonucleotide
gaatggagtttcaggatca

SEQ ID NO: 697 - DNA, 19 bp, Artificial Sequence
  Description of Artificial Sequence: Synthetic oligonucleotide
ggaaatctcctgctcgaaa

SEQ ID NO: 698 - DNA, 19 bp, Artificial Sequence
  Description of Artificial Sequence: Synthetic oligonucleotide
tgaagaaggtgacgataat

SEQ ID NO: 699 - DNA, 19 bp, Artificial Sequence
  Description of Artificial Sequence: Synthetic oligonucleotide
ggactctgctaatgatttg

SEQ ID NO: 700 - DNA, 19 bp, Artificial Sequence
  Description of Artificial Sequence: Synthetic oligonucleotide
gcaccttcatggacggcta

SEQ ID NO: 701 - DNA, 19 bp, Artificial Sequence
  Description of Artificial Sequence: Synthetic oligonucleotide
gcattatggtgtccgcaca

SEQ ID NO: 702 - DNA, 19 bp, Artificial Sequence
  Description of Artificial Sequence: Synthetic oligonucleotide
tgaaggaagttgtccgaac

SEQ ID NO: 703 - DNA, 19 bp, Artificial Sequence
  Description of Artificial Sequence: Synthetic oligonucleotide
ctgcagaaccgcatcgcca

SEQ ID NO: 704 - DNA, 19 bp, Artificial Sequence
  Description of Artificial Sequence: Synthetic oligonucleotide
ccacgtgactttcaacaat

SEQ ID NO: 705 - DNA, 19 bp, Artificial Sequence
  Description of Artificial Sequence: Synthetic oligonucleotide
acattcagatgcacaacta

SEQ ID NO: 706 - DNA, 19 bp, Artificial Sequence
  Description of Artificial Sequence: Synthetic oligonucleotide
ggacaagcgtcgccggaat

SEQ ID NO: 707 - DNA, 19 bp, Artificial Sequence
  Description of Artificial Sequence: Synthetic oligonucleotide
ccaccttgcttgtaccaaa

SEQ ID NO: 708 - DNA, 19 bp, Artificial Sequence
  Description of Artificial Sequence: Synthetic oligonucleotide
agaagtgcctcagataata

SEQ ID NO: 709 - DNA, 19 bp, Artificial Sequence
  Description of Artificial Sequence: Synthetic oligonucleotide
tagtaaaggtgtacagtga

SEQ ID NO: 710 - DNA, 19 bp, Artificial Sequence
  Description of Artificial Sequence: Synthetic oligonucleotide
tgcagaaagtgaagcatta

SEQ ID NO: 711 - DNA, 19 bp, Artificial Sequence
  Description of Artificial Sequence: Synthetic oligonucleotide
ggagatagccgcttcgtct

SEQ ID NO: 712 - DNA, 19 bp, Artificial Sequence
  Description of Artificial Sequence: Synthetic oligonucleotide
gagaagaaatggaagaaat

SEQ ID NO: 713 - DNA, 19 bp, Artificial Sequence
  Description of Artificial Sequence: Synthetic oligonucleotide
ccaaagtgctgcagtacta

SEQ ID NO: 714 - DNA, 19 bp, Artificial Sequence
  Description of Artificial Sequence: Synthetic oligonucleotide
ggtctgaactgaatgaaga

SEQ ID NO: 715 - DNA, 19 bp, Artificial Sequence
  Description of Artificial Sequence: Synthetic oligonucleotide
ataaagttctggtgcgaca

SEQ ID NO: 716 - DNA, 19 bp, Artificial Sequence
  Description of Artificial Sequence: Synthetic oligonucleotide
agaaagagttgagaagtta

SEQ ID NO: 717 - DNA, 19 bp, Artificial Sequence
  Description of Artificial Sequence: Synthetic oligonucleotide
gaaatattgctgttcctta

SEQ ID NO: 718 - DNA, 19 bp, Artificial Sequence
  Description of Artificial Sequence: Synthetic oligonucleotide
gaacgggacactttgctaa

SEQ ID NO: 719 - DNA, 19 bp, Artificial Sequence
  Description of Artificial Sequence: Synthetic oligonucleotide
gggcaaaggtcatggagat

SEQ ID NO: 720 - DNA, 19 bp, Artificial Sequence
  Description of Artificial Sequence: Synthetic oligonucleotide
ggagaagcatcgtgagtga

SEQ ID NO: 721 - DNA, 19 bp, Artificial Sequence
  Description of Artificial Sequence: Synthetic oligonucleotide
ccactttggtgtttcataa

SEQ ID NO: 722 - DNA, 19 bp, Artificial Sequence
  Description of Artificial Sequence: Synthetic oligonucleotide
tcaggttgctggtcaaatt

SEQ ID NO: 723 - DNA, 19 bp, Artificial Sequence
  Description of Artificial Sequence: Synthetic oligonucleotide
cgttatataggaaccgtaa

SEQ ID NO: 724 - DNA, 19 bp, Artificial Sequence
  Description of Artificial Sequence: Synthetic oligonucleotide
aacgttagcttcaccaaca

SEQ ID NO: 725 - DNA, 19 bp, Artificial Sequence
  Description of Artificial Sequence: Synthetic oligonucleotide
ggaactatgacctcgacta

SEQ ID NO: 726 - DNA, 19 bp, Artificial Sequence
  Description of Artificial Sequence: Synthetic oligonucleotide
gaacacacaacgtcttgga

SEQ ID NO: 727 - DNA, 19 bp, Artificial Sequence
  Description of Artificial Sequence: Synthetic oligonucleotide
ctaccaggctgcgcgcaaa

SEQ ID NO: 728 - DNA, 19 bp, Artificial Sequence
  Description of Artificial Sequence: Synthetic oligonucleotide
ggataactcggccatgaga

SEQ ID NO: 729 - DNA, 19 bp, Artificial Sequence
  Description of Artificial Sequence: Synthetic oligonucleotide
caagaagagttacctcagc

SEQ ID NO: 730 - DNA, 19 bp, Artificial Sequence
  Description of Artificial Sequence: Synthetic oligonucleotide
gaactcacctcctgattat

SEQ ID NO: 731 - DNA, 19 bp, Artificial Sequence
  Description of Artificial Sequence: Synthetic oligonucleotide
gcttagtaatgctacgtgt

SEQ ID NO: 732 - DNA, 19 bp, Artificial Sequence
  Description of Artificial Sequence: Synthetic oligonucleotide
gccagtatattatgactaa

SEQ ID NO: 733 - DNA, 19 bp, Artificial Sequence
  Description of Artificial Sequence: Synthetic oligonucleotide
gatgagaagcaacaacata

SEQ ID NO: 734 - DNA, 19 bp, Artificial Sequence
  Description of Artificial Sequence: Synthetic oligonucleotide
ccctaggaatttagacaac

SEQ ID NO: 735 - DNA, 19 bp, Artificial Sequence
  Description of Artificial Sequence: Synthetic oligonucleotide
aaagtctgttggtgcacaa

SEQ ID NO: 736 - DNA, 19 bp, Artificial Sequence
  Description of Artificial Sequence: Synthetic oligonucleotide
gcagttaggtgttggaata

SEQ ID NO: 737 - DNA, 19 bp, Artificial Sequence
  Description of Artificial Sequence: Synthetic oligonucleotide
tgataccgatgtagaggtt

SEQ ID NO: 738 - DNA, 19 bp, Artificial Sequence
  Description of Artificial Sequence: Synthetic oligonucleotide
gcataatggtagtacgaac

SEQ ID NO: 739 - DNA, 19 bp, Artificial Sequence
  Description of Artificial Sequence: Synthetic oligonucleotide
ccacgagacgggaacatta

SEQ ID NO: 740 - DNA, 19 bp, Artificial Sequence
  Description of Artificial Sequence: Synthetic oligonucleotide
gaccatacctcataacaag

SEQ ID NO: 741 - DNA, 19 bp, Artificial Sequence
  Description of Artificial Sequence: Synthetic oligonucleotide
tgaaagacctccaccaatc

SEQ ID NO: 742 - DNA, 19 bp, Artificial Sequence
  Description of Artificial Sequence: Synthetic oligonucleotide
gatgaaggctccaggtgtt

SEQ ID NO: 743 - DNA, 19 bp, Artificial Sequence
  Description of Artificial Sequence: Synthetic oligonucleotide
tatcagcatttacgactta

SEQ ID NO: 744 - DNA, 19 bp, Artificial Sequence
  Description of Artificial Sequence: Synthetic oligonucleotide
ccacatggccagctgacta

SEQ ID NO: 745 - DNA, 19 bp, Artificial Sequence
  Description of Artificial Sequence: Synthetic oligonucleotide
caacccgactatcagacta

SEQ ID NO: 746 - DNA, 19 bp, Artificial Sequence
  Description of Artificial Sequence: Synthetic oligonucleotide
gaatgaatcctcacctaac

SEQ ID NO: 747 - DNA, 19 bp, Artificial Sequence
  Description of Artificial Sequence: Synthetic oligonucleotide
gcaacaagtttgacggtga

SEQ ID NO: 748 - DNA, 19 bp, Artificial Sequence
  Description of Artificial Sequence: Synthetic oligonucleotide
tcaacgagctcacatatga

SEQ ID NO: 749 - DNA, 19 bp, Artificial Sequence
  Description of Artificial Sequence: Synthetic oligonucleotide
tgaccgatctggacccata

SEQ ID NO: 750 - DNA, 19 bp, Artificial Sequence
  Description of Artificial Sequence: Synthetic oligonucleotide
gtacttgggctcatagcta

SEQ ID NO: 751 - DNA, 19 bp, Artificial Sequence
  Description of Artificial Sequence: Synthetic oligonucleotide
gcagatgagtcttacggaa

SEQ ID NO: 752 - DNA, 19 bp, Artificial Sequence
  Description of Artificial Sequence: Synthetic oligonucleotide
ggaagtgcatttcaagatc

SEQ ID NO: 753 - DNA, 19 bp, Artificial Sequence
  Description of Artificial Sequence: Synthetic oligonucleotide
cgagaacaacgaagactat

SEQ ID NO: 754 - DNA, 19 bp, Artificial Sequence
  Description of Artificial Sequence: Synthetic oligonucleotide
caagagcactcaggcaaac

SEQ ID NO: 755 - DNA, 19 bp, Artificial Sequence
  Description of Artificial Sequence: Synthetic oligonucleotide
aaagagaccttcagccgac

SEQ ID NO: 756 - DNA, 19 bp, Artificial Sequence
  Description of Artificial Sequence: Synthetic oligonucleotide
ggactcagttggcagacta

SEQ ID NO: 757 - DNA, 19 bp, Artificial Sequence
  Description of Artificial Sequence: Synthetic oligonucleotide
aggaagggctgcgcgaata

SEQ ID NO: 758 - DNA, 19 bp, Artificial Sequence
  Description of Artificial Sequence: Synthetic oligonucleotide
ataaagtgctggcgcaatc

SEQ ID NO: 759 - DNA, 19 bp, Artificial Sequence
  Description of Artificial Sequence: Synthetic oligonucleotide
gagtcatgccctacggaat

SEQ ID NO: 760 - DNA, 19 bp, Artificial Sequence
  Description of Artificial Sequence: Synthetic oligonucleotide
caatgctgatgtttgataa

SEQ ID NO: 761 - DNA, 19 bp, Artificial Sequence
  Description of Artificial Sequence: Synthetic oligonucleotide
ccagatagccttagagact

SEQ ID NO: 762 - DNA, 19 bp, Artificial Sequence
  Description of Artificial Sequence: Synthetic oligonucleotide
gagttagattccaagacga

SEQ ID NO: 763 - DNA, 19 bp, Artificial Sequence
  Description of Artificial Sequence: Synthetic oligonucleotide
ccaacttcgtggcgaccta

SEQ ID NO: 764 - DNA, 21 bp, Artificial Sequence
  Description of Artificial Sequence: Synthetic oligonucleotide
gaggttgatctttgccggaaa

SEQ ID NO: 765 - DNA, 22 bp, Artificial Sequence
  Description of Artificial Sequence: Synthetic oligonucleotide
gaggttgatctttgctgggaaa

SEQ ID NO: 766 - DNA, 21 bp, Artificial Sequence
  Description of Artificial Sequence: Synthetic oligonucleotide
aacgtcaaagcaaagatccaa

SEQ ID NO: 767 - DNA, 21 bp, Artificial Sequence
  Description of Artificial Sequence: Synthetic oligonucleotide
atcgctgtgatcgtcacttga

SEQ ID NO: 768 - DNA, 21 bp, Artificial Sequence
  Description of Artificial Sequence: Synthetic oligonucleotide
aaggaggtttgtataagtaat

SEQ ID NO: 769 - DNA, 21 bp, Artificial Sequence
  Description of Artificial Sequence: Synthetic oligonucleotide
caggagcgcattgcacatcaa

SEQ ID NO: 770 - DNA, 21 bp, Artificial Sequence
  Description of Artificial Sequence: Synthetic oligonucleotide
ttcagtgggaattgcagcata

SEQ ID NO: 771 - DNA, 21 bp, Artificial Sequence
  Description of Artificial Sequence: Synthetic oligonucleotide
ctgatgagtcttgatctagat

SEQ ID NO: 772 - DNA, 37 bp, Artificial Sequence
  Description of Artificial Sequence: Synthetic oligonucleotide
taatacgactcactatagcgactggttacccggtcgt

SEQ ID NO: 773 - DNA, 28 bp, Artificial Sequence
  Description of Artificial Sequence: Synthetic primer
taatacgactcactatagcgactggtta

SEQ ID NO: 774 - DNA, 20 bp, Artificial Sequence
  Description of Artificial Sequence: Synthetic primer
acgaccgggtaaccagtcgc

SEQ ID NO: 775 - DNA, 64 bp, Artificial Sequence
  Description of Artificial Sequence: Synthetic oligonucleotide
gtaatacgactcactataggcgactggttacccggtcgcaattggccaagatccaagataaatt

SEQ ID NO: 776 - DNA, 64 bp, Artificial Sequence
  Description of Artificial Sequence: Synthetic oligonucleotide
  Description of Combined DNA/RNA Molecule: Synthetic oligonucleotide
aatttatcttggaucttggccaattgcgaccgggtaaccagtcgcctatagtgagtcgtattac

SEQ ID NO: 777 - DNA, 64 bp, Artificial Sequence
  Description of Artificial Sequence: Synthetic oligonucleotide
gtaatacgactcactataggcgactggttacccggtcgcaaaatttatcttggatcttggcctt

SEQ ID NO: 778 - DNA, 64 bp, Artificial Sequence
  Description of Artificial Sequence: Synthetic oligonucleotide
aaggccaagatccaagataaattttgcgaccgggtaaccagtcgcctatagtgagtcgtattac

SEQ ID NO: 779 - RNA, 89 nt, Artificial Sequence
  Description of Artificial Sequence: Synthetic oligonucleotide
gggagacaaacaaagagcgacaagggcagauagcuuuuaguugugcaaugcucugcaccgucgaguucccacccagaagaagccagaag

SEQ ID NO: 780 - RNA, 89 nt, Artificial Sequence
  Description of Artificial Sequence: Synthetic oligonucleotide
gggagacaaacaaagagcgacaagggcagugagauaguagacgaggagguuccauuagaaugcaaauaucacccagaagaagccagaag

SEQ ID NO: 781 - RNA, 88 nt, Artificial Sequence
  Description of Artificial Sequence: Synthetic oligonucleotide
gggagacaaacaaagagcgacaagggcagagguaccaagcagagggucuaaggguagcccggacgagucacccagaagaagccagaag

SEQ ID NO: 782 - RNA, 89 nt, Artificial Sequence
  Description of Artificial Sequence: Synthetic oligonucleotide
gggagacaaacaaagagcgacaagggcagcaucugcuaguaauguucggcggucgaacucuacuuggaacacccagaagaagccagaag

SEQ ID NO: 783 - RNA, 89 nt, Artificial Sequence
  Description of Artificial Sequence: Synthetic oligonucleotide
gggagacaaacaaagagcgacaagggcagagacagguuaggggaaaguguguuaaacuuuaaaguaauucacccagaagaagccagaag

SEQ ID NO: 784 - RNA, 89 nt, Artificial Sequence
  Description of Artificial Sequence: Synthetic oligonucleotide
gggagacaaacaaagagcgacaagggcagguaggacgucaguuuaacgcaagcgcguuacaccuagauccacccagaagaagccagaag

SEQ ID NO: 785 - RNA, 89 nt, Artificial Sequence
  Description of Artificial Sequence: Synthetic oligonucleotide
gggagacaaacaaagagcgacaagggcagccuauuuuggggcugugacauauugucaaaugcuaaacggcacccagaagaagccagaag

SEQ ID NO: 786 - RNA, 89 nt, Artificial Sequence
  Description of Artificial Sequence: Synthetic oligonucleotide
gggagacaaacaaagagcgacaagggcagagcaguuucguugggcgucggucuaauagacugacuggggcacccagaagaagccagaag

SEQ ID NO: 787 - RNA, 89 nt, Artificial Sequence
  Description of Artificial Sequence: Synthetic oligonucleotide
gggagacaaacaaagagcgacaagggcagguccaaucugagugaugucugucaagaccuagagaaguaccacccagaagaagccagaag

SEQ ID NO: 788 - RNA, 89 nt, Artificial Sequence
  Description of Artificial Sequence: Synthetic oligonucleotide
gggagacaaacaaagagcgacaagggcagagugaugucugucaagaccgggcucuaccgcugguucaagcacccagaagaagccagaag

SEQ ID NO: 789 - RNA, 89 nt, Artificial Sequence
  Description of Artificial Sequence: Synthetic oligonucleotide
gggagacaaacaaagagcgacaagggcagugggagcacuagaauggucuauauuaauguuagcgcagugcacccagaagaagccagaag

SEQ ID NO: 790 - RNA, 51 nt, Artificial Sequence
  Description of Artificial Sequence: Synthetic oligonucleotide
aggagaugcguaggguggacugagugaugucugucaagaccucguccaaac

SEQ ID NO: 791 - RNA, 51 nt, Artificial Sequence
  Description of Artificial Sequence: Synthetic oligonucleotide
aggagugaugucugucaagaccgauugucugccacucaaucgggaccaaac

SEQ ID NO: 792 - RNA, 51 nt, Artificial Sequence
  Description of Artificial Sequence: Synthetic oligonucleotide
aggagugaugucugucaagaccaggugcugaggugacucuguaauucaaac

SEQ ID NO: 793 - RNA, 40 nt, Artificial Sequence
  Description of Artificial Sequence: Synthetic oligonucleotide
gggaggacgaugcggaucagccauguuuacgucacuccua

SEQ ID NO: 794 - RNA, 67 nt, Artificial Sequence
  Description of Artificial Sequence: Synthetic oligonucleotide
gcccagcaugcauuacugaucgugguguuugcuuagcccaaaggccaagauccaagauaaagaaggc

SEQ ID NO: 795 - RNA, 69 nt, Artificial Sequence
  Description of Artificial Sequence: Synthetic oligonucleotide
gcccagcaugcauuacugaucgugguguuugcuuagcccaaagccuucuuuaucuuggaucuuggccuu

SEQ ID NO: 796 - RNA, 40 nt, Artificial Sequence
  Description of Artificial Sequence: Synthetic oligonucleotide
gcccagcaugcauuacugaucgugguguuugcuuagccca

SEQ ID NO: 797 - RNA, 41 nt, Artificial Sequence
  Description of Artificial Sequence: Synthetic oligonucleotide
ggcuggggugcgacauuauguucguuaaggaucaaucgcuc

SEQ ID NO: 798 - RNA, 40 nt, Artificial Sequence
  Description of Artificial Sequence: Synthetic oligonucleotide
uuaugaugcaaaaacgaacuggaauggccaugcagguaca

SEQ ID NO: 799 - RNA, 41 nt, Artificial Sequence
  Description of Artificial Sequence: Synthetic oligonucleotide
gguuggggugcgacauuauguucguuaaggaucaaucgcuc

SEQ ID NO: 800 - RNA, 41 nt, Artificial Sequence
  Description of Artificial Sequence: Synthetic oligonucleotide
gauuuggagagcauuauguuagguuaaggaucaaucuucua

SEQ ID NO: 801 - RNA, 41 nt, Artificial Sequence
  Description of Artificial Sequence: Synthetic oligonucleotide
ggcuggggugcgacauuauguucguuaaggaucaaucgcuu

SEQ ID NO: 802 - RNA, 83 nt, Artificial Sequence
  Description of Artificial Sequence: Synthetic oligonucleotide
gggguaaacgaacaggauuuuuuuaacgagcuauauuguuuccuguugcccguccgucuagucgugaagagagcaagguuacu

SEQ ID NO: 803 - RNA, 83 nt, Artificial Sequence
  Description of Artificial Sequence: Synthetic oligonucleotide
gggguaaacgaacaggacgacgggucgaagcugaauagguaaccaaucacggcauaacuagucgugaagagagcaagguuacu

SEQ ID NO: 804 - RNA, 83 nt, Artificial Sequence
  Description of Artificial Sequence: Synthetic oligonucleotide
gggguaaacgaacaggaugagggagcaaaaagggcgaaaaugcaguaacuaaacguucuagucgugaagagagcaagguuacu

SEQ ID NO: 805 - RNA, 83 nt, Artificial Sequence
  Description of Artificial Sequence: Synthetic oligonucleotide
gggguaaacgaacaggauuuuuuuaacgagcuauauuauuuccuguugcccguccgucuagucgugaagagagcaagguuacu

SEQ ID NO: 806 - RNA, 84 nt, Artificial Sequence
  Description of Artificial Sequence: Synthetic oligonucleotide
gggguaaacgaacaggaaccauuaaaucauaaggagaaagaugaugugcgcgacauaacuagucgugaagagagcaagguuacu

SEQ ID NO: 807 - RNA, 89 nt, Artificial Sequence
  Description of Artificial Sequence: Synthetic oligonucleotide
gggagacaaacaaagagcgacaagggcagcggguuaucacguugggaacgggccaucaacucuucucaccacccagaagaagccagaag

SEQ ID NO: 808 - RNA, 89 nt, Artificial Sequence
  Description of Artificial Sequence: Synthetic oligonucleotide
gggagacaaacaaagagcgacaagggcaguagugagggauucaccagagugaaugcgcuccucggaaaucacccagaagaagccagaag

SEQ ID NO: 809 - RNA, 89 nt, Artificial Sequence
  Description of Artificial Sequence: Synthetic oligonucleotide
gggagacaaacaaagagcgacaagggcagaacgggcaauguccaaggugaggcaguuuguauggacacacacccagaagaagccagaag

SEQ ID NO: 810 - RNA, 89 nt, Artificial Sequence
  Description of Artificial Sequence: Synthetic oligonucleotide
gggagacaaacaaagagcgacaagggcaguugagauugaggagucagaccugcgucucuaguaacaaugcacccagaagaagccagaag

SEQ ID NO: 811 - RNA, 89 nt, Artificial Sequence
  Description of Artificial Sequence: Synthetic oligonucleotide
gggagacaaacaaagagcgacaagggcagaguggacggucggcuagagccgggaggaauuccuugugaccacccagaagaagccagaag

SEQ ID NO: 812 - RNA, 89 nt, Artificial Sequence
  Description of Artificial Sequence: Synthetic oligonucleotide
gggagacaaacaaagagcgacaagggcaguuugacaauguaccuuuaauuacggauuguaccuugggcgcacccagaagaagccagaag

SEQ ID NO: 813 - RNA, 35 nt, Artificial Sequence
  Description of Artificial Sequence: Synthetic oligonucleotide
gggagagaggaagagggaugggccgacgugccgca

SEQ ID NO: 814 - RNA, 89 nt, Artificial Sequence
  Description of Artificial Sequence: Synthetic oligonucleotide
gggagacaaacaaagagcgacaagggcagaauuacaauagcuauaguccgggcaccaugcuuguaaauccacccagaagaagccagaag

SEQ ID NO: 815 - RNA, 89 nt, Artificial Sequence
  Description of Artificial Sequence: Synthetic oligonucleotide
gggagacaaacaaagagcgacaagggcagguaggacgcuagcagacuagaauguaucuaugcuuagauccacccagaagaagccagaag

SEQ ID NO: 816 - RNA, 89 nt, Artificial Sequence
  Description of Artificial Sequence: Synthetic oligonucleotide
gggagacaaacaaagagcgacaagggcaggcuaguauuacaaugucguggaaaagccgugcgggguauccacccagaagaagccagaag

SEQ ID NO: 817 - RNA, 89 nt, Artificial Sequence
  Description of Artificial Sequence: Synthetic oligonucleotide
gggagacaaacaaagagcgacaagggcagggagccauucuugaaauugucaguuugauugugcucaggucacccagaagaagccagaag

SEQ ID NO: 818 - RNA, 89 nt, Artificial Sequence
  Description of Artificial Sequence: Synthetic oligonucleotide
gggagacaaacaaagagcgacaagggcagaaaguacaaugguugacauauaccgucgguuuauccuaugcacccagaagaagccagaag

SEQ ID NO: 819 - RNA, 89 nt, Artificial Sequence
  Description of Artificial Sequence: Synthetic oligonucleotide
gggagacaaacaaagagcgacaagggcagguaggcuaucgcugcuugaucgucugaucagagccuauaccacccagaagaagccagaag

SEQ ID NO: 820 - RNA, 83 nt, Artificial Sequence
  Description of Artificial Sequence: Synthetic oligonucleotide
gggguaaacgaacaggaaacagauggccaacacaggcgaagcauagacuaggaacggcuagucgugaagagagcaagguuacu

SEQ ID NO: 821 - RNA, 81 nt, Artificial Sequence
  Description of Artificial Sequence: Synthetic oligonucleotide
gggguaaacgaacaggacuugaugugaaaaggcgacgcgaugagacgaagggcuucuagucgugaagagagcaagguuacu

SEQ ID NO: 822 - RNA, 83 nt, Artificial Sequence
  Description of Artificial Sequence: Synthetic oligonucleotide
gggguaaacgaacaggaaguagacuagacggcggcgauaaccagauaacgacauucucuagucgugaagagagcaagguuacu

SEQ ID NO: 823 - RNA, 83 nt, Artificial Sequence
  Description of Artificial Sequence: Synthetic oligonucleotide
gggguaaacgaacaggaccgagugagacggguaguggacaaaugaaguagugugguccuagucgugaagagagcaagguuacu

SEQ ID NO: 824 - RNA, 83 nt, Artificial Sequence
  Description of Artificial Sequence: Synthetic oligonucleotide
gggguaaacgaacaggacuuuuaauuucacgccgcacgauccggaaaaacgacuugacuagucgugaagagagcaagguuacu

SEQ ID NO: 825 - RNA, 83 nt, Artificial Sequence
  Description of Artificial Sequence: Synthetic oligonucleotide
gggguaaacgaacaggaagggagucgauuugaguuguaauuugaccuauguuauaaucuagucgugaagagagcaagguuacu

SEQ ID NO: 826 - RNA, 83 nt, Artificial Sequence
  Description of Artificial Sequence: Synthetic oligonucleotide
gggguaaacgaacaggaauggcuacaguaucgaugcaguuuucgaaugaaguagaaacuagucgugaagagagcaagguuacu

SEQ ID NO: 827 - RNA, 83 nt, Artificial Sequence
  Description of Artificial Sequence: Synthetic oligonucleotide
gggguaaacgaacaggacaggacagcaagcaguagaaaacaagccacgaaggggacucuagucgugaagagagcaagguuacu

SEQ ID NO: 828 - RNA, 83 nt, Artificial Sequence
  Description of Artificial Sequence: Synthetic oligonucleotide
gggguaaacgaacaggauuuuggacugucuagccgauguacuuaaguuuaucauuuucuagucgugaagagagcaagguuacu

SEQ ID NO: 829 - RNA, 83 nt, Artificial Sequence
  Description of Artificial Sequence: Synthetic oligonucleotide
gggguaaacgaacaggagcagucgcuggcuucauuuuuuuuuuuuuuuugugcucaacuagucgugaagagagcaagguuacu

SEQ ID NO: 830 - RNA, 51 nt, Artificial Sequence
  Description of Artificial Sequence: Synthetic oligonucleotide
aggagacacaugugacaagaggcuaugauccugaaugcauccuuggcaaac

SEQ ID NO: 831 - RNA, 51 nt, Artificial Sequence
  Description of Artificial Sequence: Synthetic oligonucleotide
aggagaaucaugaguuaucuguguaaggaaccaaagccaugcuuaucaaac

SEQ ID NO: 832 - DNA, 21 bp, Unknown
  Description of Unknown: target sequence
ctgattaatatatttaatata

SEQ ID NO: 833 - DNA, 21 bp, Unknown
  Description of Unknown: target sequence
ctccttccacatgtacataaa

SEQ ID NO: 834 - DNA, 21 bp, Unknown
  Description of Unknown: target sequence
cagcattatggtgtccgcaca

SEQ ID NO: 835 - DNA, 21 bp, Unknown
  Description of Unknown: target sequence
cagcaccttcatggacggcta

SEQ ID NO: 836 - DNA, 19 bp, Unknown
  Description of Unknown: target sequence
gcaccttcatggacggcta

SEQ ID NO: 837 - DNA, 18 bp, Unknown
  Description of Unknown: target sequence
gcatatggtgtccgcaca

SEQ ID NO: 838 - DNA, 18 bp, Unknown
  Description of Unknown: target sequence
tgaaggaagtgtccgaac

SEQ ID NO: 839 - DNA, 19 bp, Unknown
  Description of Unknown: target sequence
ctgcagaaccgcatcgcca

SEQ ID NO: 840 - DNA, 19 bp, Unknown
  Description of Unknown: target sequence
tgctgtgtgtggggacaac

SEQ ID NO: 841 - DNA, 19 bp, Unknown
  Description of Unknown: target sequence
gggctgcaagggcttcttc

SEQ ID NO: 842 - DNA, 19 bp, Unknown
  Description of Unknown: target sequence
gcgcacagtgcagaaaaac

SEQ ID NO: 843 - DNA, 17 bp, Unknown
  Description of Unknown: target sequence
cagtggctctgactact

SEQ ID NO: 844 - DNA, 19 bp, Unknown
  Description of Unknown: target sequence
ccacttctccacaccttga

SEQ ID NO: 845 - DNA, 18 bp, Unknown
  Description of Unknown: target sequence
ggcttgagctgcagaatg

SEQ ID NO: 846 - DNA, 21 bp, Unknown
  Description of Unknown: target sequence
cacagcttgcttgtcgatgtc

SEQ ID NO: 847 - DNA, 25 bp, Unknown
  Description of Unknown: target sequence
ggtccctgcacagcttgcttgtcga

SEQ ID NO: 848 - DNA, 25 bp, Unknown
  Description of Unknown: target sequence
ccggttctctggaggtcatccgcaa

SEQ ID NO: 849 - DNA, 25 bp, Unknown
  Description of Unknown: target sequence
cagcattatggtgtccgcacatgtg

SEQ ID NO: 850 - DNA, 21 bp, Unknown
  Description of Unknown: target sequence
aatgttgacggacagcctatt

SEQ ID NO: 851 - DNA, 21 bp, Unknown
  Description of Unknown: target sequence
aagagtacggccctgaagaag

SEQ ID NO: 852 - DNA, 21 bp, Unknown
  Description of Unknown: target sequence
aaggaggtttgtataagtaat

SEQ ID NO: 853 - DNA, 21 bp, Unknown
  Description of Unknown: target sequence
caggagcgcattgcacatcaa

SEQ ID NO: 854 - DNA, 21 bp, Unknown
  Description of Unknown: target sequence
ccctattagatacacttctta

SEQ ID NO: 855 - DNA, 21 bp, Unknown
  Description of Unknown: target sequence
taaggaggtttgtataagtaa

SEQ ID NO: 856 - DNA, 21 bp, Unknown
  Description of Unknown: target sequence
cctagtcaagcctgagaatta

SEQ ID NO: 857 - DNA, 19 bp, Unknown
  Description of Unknown: target sequence
ctgccagtgtatactctga

SEQ ID NO: 858 - DNA, 19 bp, Unknown
  Description of Unknown: target sequence
atacactcggtagctgtgg

SEQ ID NO: 859 - RNA, 70 nt, Artificial Sequence
  Description of Artificial Sequence: Synthetic oligonucleotide
gggagagagauauaagggccuccugauacccgcugcuaucuggaccgaucccauuaccaaauucucuccc

SEQ ID NO: 860 - RNA, 83 nt, Artificial Sequence
  Description of Artificial Sequence: Synthetic oligonucleotide
gggguaaacgaacaggaagacggcgcaauaagacagacuaggacacgauuagagguacuagucgugaagagagcaagguuacu

SEQ ID NO: 861 - RNA, 83 nt, Artificial Sequence
  Description of Artificial Sequence: Synthetic oligonucleotide
gggguaaacgaacaggauaaaagaaaacaacuagcgcgacgagagaauaaaaugaaacuagucgugaagagagcaagguuacu

SEQ ID NO: 862 - RNA, 83 nt, Artificial Sequence
  Description of Artificial Sequence: Synthetic oligonucleotide
gggguaaacgaacaggauaauuguuggggaaauaaauugcugggaacgacuuaaaagcuagucgugaagagagcaagguuacu

SEQ ID NO: 863 - RNA, 83 nt, Artificial Sequence
  Description of Artificial Sequence: Synthetic oligonucleotide
gggguaaacgaacaggaguuaaucaugagguagguaacaaaaggcaacggccaauaacuagucgugaagagagcaagguuacu

SEQ ID NO: 864 - RNA, 83 nt, Artificial Sequence
  Description of Artificial Sequence: Synthetic oligonucleotide
gggguaaacgaacaggauaaccaugcaaauaacaagcaaacagagaacucacgccagcuagucgugaagagagcaagguuacu

SEQ ID NO: 865 - RNA, 41 nt, Artificial Sequence
  Description of Artificial Sequence: Synthetic oligonucleotide
uauacauucuugguucauaaaggauaaggccuaagucgggu

SEQ ID NO: 866 - RNA, 51 nt, Artificial Sequence
  Description of Artificial Sequence: Synthetic oligonucleotide
aggaguauacauucuugguucauaaaggauaaggccuaagucgggucaaac

SEQ ID NO: 867 - RNA, 87 nt, Artificial Sequence
  Description of Artificial Sequence: Synthetic oligonucleotide
gggagacaagaauaaacgcucaagacacggauacauaaugcugucuugauuuacaaacugagcuucgacaggaggcucacaacaggc

SEQ ID NO: 868 - RNA, 87 nt, Artificial Sequence
  Description of Artificial Sequence: Synthetic oligonucleotide
gggagacaagaauaaacgcucaaugagcuuacagcggccauugauuuacuaacggacugagcauucgacaggaggcucacaacaggc

SEQ ID NO: 869 - RNA, 79 nt, Artificial Sequence
  Description of Artificial Sequence: Synthetic oligonucleotide
gaauuccgcgugugccagcgaaaguugcguaugggucacaucgcaggcacaugucaucugggcgguccguucgggaucc

SEQ ID NO: 870 - RNA, 51 nt, Artificial Sequence
  Description of Artificial Sequence: Synthetic oligonucleotide
aggaggguugcguugcaaguaacagaaaggaauuugaaaauuguggcaaac

SEQ ID NO: 871 - RNA, 51 nt, Artificial Sequence
  Description of Artificial Sequence: Synthetic oligonucleotide
aggagguuggcaaucccggauugaggaaucgcaugacgcuauuaaccaaac

SEQ ID NO: 872 - RNA, 41 nt, Artificial Sequence
  Description of Artificial Sequence: Synthetic oligonucleotide
uaguaaaugagagaugaaaucuguaugcgccgcacugauug

SEQ ID NO: 873 - RNA, 51 nt, Artificial Sequence
  Description of Artificial Sequence: Synthetic oligonucleotide
aggaguagugcagcauugacugaaugucauacggcauaagcaucuacaaac

SEQ ID NO: 874 - RNA, 51 nt, Artificial Sequence
  Description of Artificial Sequence: Synthetic oligonucleotide
aggaggacaucggaaacgcugaucuuaauagugaauuaacaugcgacaaac

SEQ ID NO: 875 - RNA, 51 nt, Artificial Sequence
  Description of Artificial Sequence: Synthetic oligonucleotide
aggaguaguaaaugagagaugaaaucuguaugcgccgcacugauugcaaac

SEQ ID NO: 876 - RNA, 89 nt, Artificial Sequence
  Description of Artificial Sequence: Synthetic oligonucleotide
gggagacaaacaaagagcgacaagggcagggaugaccgguguauuaaggucuaacuuaacucggugauacacccagaagaagccagaag

SEQ ID NO: 877 - RNA, 89 nt, Artificial Sequence
  Description of Artificial Sequence: Synthetic oligonucleotide
gggagacaaacaaagagcgacaagggcagcucaaaaaggguagugugugguauagucuaaucguacccacacccagaagaagccagaag

SEQ ID NO: 878 - RNA, 89 nt, Artificial Sequence
  Description of Artificial Sequence: Synthetic oligonucleotide
gggagacaaacaaagagcgacaagggcaguagugugguauuguguaauaauacccuacugaggucaaaacacccagaagaagccagaag

SEQ ID NO: 879 - RNA, 89 nt, Artificial Sequence
  Description of Artificial Sequence: Synthetic oligonucleotide
gggagacaaacaaagagcgacaagggcagcggguugucaagaugggaacgggcccggaucuuuagcgcacacccagaagaagccagaag

SEQ ID NO: 880 - RNA, 89 nt, Artificial Sequence
  Description of Artificial Sequence: Synthetic oligonucleotide
gggagacaaacaaagagcgacaagggcagugggcugugcgcgcgaugagaucacguuagcguaauugugcacccagaagaagccagaag

SEQ ID NO: 881 - RNA, 89 nt, Artificial Sequence
  Description of Artificial Sequence: Synthetic oligonucleotide
gggagacaaacaaagagcgacaagggcagacagguccaucaggcagaaccgagggagagugcgcgucgucacccagaagaagccagaag

SEQ ID NO: 882 - RNA, 89 nt, Artificial Sequence
  Description of Artificial Sequence: Synthetic oligonucleotide
gggagacaaacaaagagcgacaagggcagaaguuuggauuucaagaugcucaucacgcucaaacuuucacacccagaagaagccagaag

SEQ ID NO: 883 - RNA, 89 nt, Artificial Sequence
  Description of Artificial Sequence: Synthetic oligonucleotide
gggagacaaacaaagagcgacaagggcaggacgcugcgaaaagugcgaaguuugcauccuggccuaguucacccagaagaagccagaag

SEQ ID NO: 884 - RNA, 89 nt, Artificial Sequence
  Description of Artificial Sequence: Synthetic oligonucleotide
gggagacaaacaaagagcgacaagggcaguucucuccacaaaguuuagauuucaagcgugagcagggaucacccagaagaagccagaag

SEQ ID NO: 885 - RNA, 89 nt, Artificial Sequence
  Description of Artificial Sequence: Synthetic oligonucleotide
gggagacaaacaaagagcgacaagggcagguguugagaugaaggaguucuagcccuucgaauggugugacacccagaagaagccagaag

SEQ ID NO: 886 - RNA, 89 nt, Artificial Sequence
  Description of Artificial Sequence: Synthetic oligonucleotide
gggagacaaacaaagagcgacaagggcagaguuggguagguugugacaggaaugugauugguaagauaccacccagaagaagccagaag

SEQ ID NO: 887 - RNA, 89 nt, Artificial Sequence
  Description of Artificial Sequence: Synthetic oligonucleotide
gggagacaaacaaagagcgacaagggcaguacuugagucauuguauagaucuaauucgcgcagaauugacacccagaagaagccagaag

SEQ ID NO: 888 - RNA, 92 nt, Artificial Sequence
  Description of Artificial Sequence: Synthetic oligonucleotide
gggagacaaacaaagagcgacaagggcagcccuugucgcucuuuguuugucucccuauagugagucguauuacacccagaagaagccagaag

SEQ ID NO: 889 - RNA, 89 nt, Artificial Sequence
  Description of Artificial Sequence: Synthetic oligonucleotide
gggagacaaacaaagagcgacaagggcagguaggacgcuagcagacuagaaugaaucuaugcuuagauccacccagaagaagccagaag

SEQ ID NO: 890 - RNA, 89 nt, Artificial Sequence
  Description of Artificial Sequence: Synthetic oligonucleotide
gggagacaaacaaagagcgacaagggcagcgguuaaaauauaguucuaaguuagucuggugaauccacucacccagaagaagccagaag

SEQ ID NO: 891 - RNA, 89 nt, Artificial Sequence
  Description of Artificial Sequence: Synthetic oligonucleotide
gggagacaaacaaagagcgacaagggcagucugggggauggcugugaggucccaauguaucgcaucucacacccagaagaagccagaag

SEQ ID NO: 892 - RNA, 86 nt, Artificial Sequence
  Description of Artificial Sequence: Synthetic oligonucleotide
gggagacaaacaaagagcgacaagggcaguuaaaauauaguucuaaguuagucuggugaauccacucacccagaagaagccagaag

SEQ ID NO: 893 - RNA, 19 nt, Artificial Sequence
  Description of Artificial Sequence: Synthetic oligonucleotide
ggcaaagauccaagauaag

SEQ ID NO: 894 - RNA, 19 nt, Artificial Sequence
  Description of Artificial Sequence: Synthetic oligonucleotide
ggccaagauccaagauaaa

SEQ ID NO: 895 - RNA, 19 nt, Artificial Sequence
  Description of Artificial Sequence: Synthetic oligonucleotide
aggccaagauccaagauaa

SEQ ID NO: 896 - DNA, 21 bp, Artificial Sequence
  Description of Artificial Sequence: Synthetic oligonucleotide
aaggccaagatccaagataaa

SEQ ID NO: 897 - DNA, 21 bp, Artificial Sequence
  Description of Artificial Sequence: Synthetic oligonucleotide
aagaggtggtatgcagatctt

SEQ ID NO: 898 - DNA, 19 bp, Homo sapiens
ggccaagatccaagataaa

SEQ ID NO: 899 - DNA, 19 bp, Homo sapiens
ggccaagatccaggataaa

SEQ ID NO: 900 - DNA, 18 bp, Artificial Sequence
  Description of Artificial Sequence: Synthetic oligonucleotide
ggccaagatccaagataa

SEQ ID NO: 901 - DNA, 18 bp, Homo sapiens
ggccaagatccaggataa

SEQ ID NO: 902 - DNA, 21 bp, Artificial Sequence
  Description of Artificial Sequence: Synthetic oligonucleotide
  Description of Combined DNA/RNA Molecule: Synthetic oligonucleotide
ggccaagauccaagauaaatt

SEQ ID NO: 903 - DNA, 21 bp, Artificial Sequence
  Description of Artificial Sequence: Synthetic oligonucleotide
  Description of Combined DNA/RNA Molecule: Synthetic oligonucleotide
uuuaucuuggaucuuggcctt

SEQ ID NO: 904 - DNA, 14 bp, Artificial Sequence
  Description of Artificial Sequence: Synthetic oligonucleotide
aagatccaagataa

SEQ ID NO: 905 - DNA, 14 bp, Homo sapiens
aagatccaagataa

SEQ ID NO: 906 - DNA, 15 bp, Artificial Sequence
  Description of Artificial Sequence: Synthetic oligonucleotide
aagatccaagataaa

SEQ ID NO: 907 - DNA, 15 bp, Homo sapiens
aagatccaagataaa

SEQ ID NO: 908 - DNA, 15 bp, Artificial Sequence
  Description of Artificial Sequence: Synthetic oligonucleotide
ggccaagatccaaga

SEQ ID NO: 909 - DNA, 15 bp, Homo sapiens
ggccaagatccaaga

SEQ ID NO: 910 - DNA, 15 bp, Artificial Sequence
  Description of Artificial Sequence: Synthetic oligonucleotide
gccaagatccaagat

SEQ ID NO: 911 - DNA, 15 bp, Homo sapiens
gccaagatccaagat

SEQ ID NO: 912 - DNA, 933 bp, Homo sapiens
atttaggggcggttggctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaaggtttcgttgcatggta

SEQ ID NO: 913 - DNA, 2193 bp, Homo sapiens
agttccgtcgcagccgggatttgggtcgcagttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaa

SEQ ID NO: 914 - RNA, 19 nt, Artificial Sequence
  Description of Artificial Sequence: Synthetic oligonucleotide
gcgacugguuacccggucg

SEQ ID NO: 915 - RNA, 45 nt, Artificial Sequence
  Description of Artificial Sequence: Synthetic oligonucleotide
gcgacugguuacccggucguaaaaggccaagauccaagauaaauu

SEQ ID NO: 916 - RNA, 45 nt, Artificial Sequence
  Description of Artificial Sequence: Synthetic oligonucleotide
gcgacugguuacccggucguaaaauuuaucuuggaucuuggccuu

SEQ ID NO: 917 - DNA, 240 bp, Homo sapiens
atcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaag

SEQ ID NO: 918 - DNA, 240 bp, Homo sapiens
atcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaag

SEQ ID NO: 919 - DNA, 251 bp, Homo sapiens
aaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggt

SEQ ID NO: 920 - DNA, 300 bp, Homo sapiens
aaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgcccaagacaaggaaggcattcctcctgaccagcagaggttgatctttgccggaaagcagctggaagatgggcgcaccctgtctgactacaacatccagaaagagtctaccctgcacctggtg

SEQ ID NO: 921 - DNA, 233 bp, Homo sapiens
ggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtgg

SEQ ID NO: 922 - DNA, 240 bp, Homo sapiens
ggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcag

SEQ ID NO: 923 - DNA, 180 bp, Homo sapiens
ccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgc

SEQ ID NO: 924 - DNA, 180 bp, Mus musculus
ccatcaccttggaggtggagcccagtgacaccatcgagaatgtgaaggccaagatccaggataaagagggcatcccccctgaccagcagaggctgatctttgccggcaagcagctagaagatggccgcactctctctgattacaacatccagaaagagtcgaccctgcacctggtcctcc

SEQ ID NO: 925 - DNA, 19 bp, Mus musculus
aggccaagatccaggataa

SEQ ID NO: 926 - DNA, 180 bp, Homo sapiens
ccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctcc

SEQ ID NO: 927 - DNA, 180 bp, Mus musculus
ccatcaccctggaggtcgagcccagtgacaccattgagaatgtcaaggcaaagatccaggacaaggagggcatcccccctgaccagcagaggctgatctttgcaggcaagcagctggaagatggccgcaccctgtcagactacaacatccagaaagagtccaccctgcacctggtccttc

SEQ ID NO: 928 - DNA, 19 bp, Mus musculus
ggcaaagatccaggacaag

SEQ ID NO: 929 - DNA, 19 bp, Mus musculus
aggcaaagatccaggacaa

SEQ ID NO: 930 - RNA, 21 nt, Artificial Sequence
  Description of Artificial Sequence: Synthetic oligonucleotide
aggccaagauccaagauaauu

SEQ ID NO: 931 - RNA, 21 nt, Artificial Sequence
  Description of Artificial Sequence: Synthetic oligonucleotide
uuaucuuggaucuuggccuuu

SEQ ID NO: 932 - RNA, 27 nt, Artificial Sequence
  Description of Artificial Sequence: Synthetic oligonucleotide
ggccaagauccaagauaaagaaggcuu

SEQ ID NO: 933 - RNA, 27 nt, Artificial Sequence
  Description of Artificial Sequence: Synthetic oligonucleotide
gccuucuuuaucuuggaucuuggccuu

SEQ ID NO: 934 - DNA, 120 bp, Homo sapiens
acgtgtgccgtgtgcggggacaacgccgcctgccagcactacggcgtgcgaacctgcgagggctgcaagggctttttcaagagaacagtgcagaaaaatgcaaaatatgtttgcctggca

SEQ ID NO: 935 - DNA, 120 bp, Homo sapiens
cgctgtgctgtgtgtggggacaacgcttcatgccagcattatggtgtccgcacatgtgagggctgcaagggcttcttcaagcgcacagtgcagaaaaacgccaagtacatctgcctggct

SEQ ID NO: 936 - DNA, 120 bp, Homo sapiens
ctgtgcgctgtgtgtggggacaacgcggcctgccaacactacggcgtgcgcacctgtgagggctgcaaaggcttctttaagcgcacagtgcaaaaaaatgcaaaatacgtgtgtttagca

SEQ ID NO: 937 - DNA, 19 bp, Artificial Sequence
  Description of Artificial Sequence: Synthetic oligonucleotide
cgctgtgtgtggggacaac

SEQ ID NO: 938 - DNA, 19 bp, Artificial Sequence
  Description of Artificial Sequence: Synthetic oligonucleotide
tgccgtgtgcggggacaac

SEQ ID NO: 939 - DNA, 19 bp, Artificial Sequence
  Description of Artificial Sequence: Synthetic oligonucleotide
gggctgcaaaggcttcttt

SEQ ID NO: 940 - DNA, 19 bp, Artificial Sequence
  Description of Artificial Sequence: Synthetic oligonucleotide
gggctgcaagggctttttc

SEQ ID NO: 941 - DNA, 19 bp, Artificial Sequence
  Description of Artificial Sequence: Synthetic oligonucleotide
gcgcacagtgcaaaaaaat

SEQ ID NO: 942 - DNA, 19 bp, Artificial Sequence
  Description of Artificial Sequence: Synthetic oligonucleotide
gagaacagtgcagaaaaat

SEQ ID NO: 943 - DNA, 50 bp, Homo sapiens
ctgcctcttcattgcctgcttcgtcctggtcctcacgcagagctccatct

SEQ ID NO: 944 - DNA, 49 bp, Homo sapiens
ctgcctcatggttgcctgtccggtcctcatcctcacccagagctccatc

SEQ ID NO: 945 - DNA, 19 bp, Artificial Sequence
  Description of Artificial Sequence: Synthetic oligonucleotide
cctcacccagagctccatc

SEQ ID NO: 946 - DNA, 19 bp, Artificial Sequence
  Description of Artificial Sequence: Synthetic oligonucleotide
catggtctacttcaacttc

SEQ ID NO: 947 - DNA, 19 bp, Artificial Sequence
  Description of Artificial Sequence: Synthetic oligonucleotide
gtctacttcaacttctttg

SEQ ID NO: 948 - DNA, 120 bp, Homo sapiens
aagctaattcatctggagatcaaacccgcaatccggaaccagatcataagggagctgcaggttctgcatgagtgcaactctccgtacatcgtgggcttctatggtgcgttctacagcgat

SEQ ID NO: 949 - DNA, 120 bp, Homo sapiens
aagctgatccaccttgagatcaagccggccatccggaaccagatcatccgcgagctgcaggtcctgcacgaatgcaactcgccgtacatcgtgggcttctacggggccttctacagtgac

SEQ ID NO: 950 - DNA, 19 bp, Artificial Sequence
  Description of Artificial Sequence: Synthetic oligonucleotide
catccggaaccagatcatc

SEQ ID NO: 951 - DNA, 19 bp, Artificial Sequence
  Description of Artificial Sequence: Synthetic oligonucleotide
gtacatcgtgggcttctac

SEQ ID NO: 952 - DNA, 19 bp, Artificial Sequence
  Description of Artificial Sequence: Synthetic oligonucleotide
gaagccctccaacatcctc

SEQ ID NO: 953 - DNA, 60 bp, Homo sapiens
ttgctgaaaagccagcagctgagcaatgaccatatctgctacttcctctaccagatcctg

SEQ ID NO: 954 - DNA, 60 bp, Homo sapiens
ctcttgaagacacaacacctcagcaatgaccatatctgctattttctctaccagatcctc

SEQ ID NO: 955 - DNA, 19 bp, Artificial Sequence
  Description of Artificial Sequence: Synthetic oligonucleotide
tcagcaatgaccatatctg

SEQ ID NO: 956 - DNA, 19 bp, Artificial Sequence
  Description of Artificial Sequence: Synthetic oligonucleotide
ccaagggctacaccaagtc

SEQ ID NO: 957 - DNA, 19 bp, Artificial Sequence
  Description of Artificial Sequence: Synthetic oligonucleotide
gtctgtaggctgcattctg

SEQ ID NO: 958 - DNA, 19 bp, Artificial Sequence
  Description of Artificial Sequence: Synthetic oligonucleotide
agaagacctgaattgtata

SEQ ID NO: 959 - DNA, 60 bp, Homo sapiens
cgggccctcgcgccggcttctaccggcaggagctgaacaagaccgtgtgggaggtgccgc

SEQ ID NO: 960 - DNA, 51 bp, Homo sapiens
ggcccacgttctaccggcaggagctgaacaagacaatctgggaggtgcccg

SEQ ID NO: 961 - DNA, 19 bp, Artificial Sequence
  Description of Artificial Sequence: Synthetic oligonucleotide
gaactggatgcattacaac

SEQ ID NO: 962 - DNA, 54 bp, Homo sapiens
ttattttttttgagaccgagtcttgctctgttacccaggctggagtgcagtggc

SEQ ID NO: 963 - DNA, 60 bp, Homo sapiens
gttttgttttgttttttgagacagggtcttgctctgtcgcccaggctggagtgcactggc

SEQ ID NO: 964 - DNA, 19 bp, Artificial Sequence
  Description of Artificial Sequence: Synthetic oligonucleotide
gacagggtcttgctctgtc

SEQ ID NO: 965 - DNA, 19 bp, Artificial Sequence
  Description of Artificial Sequence: Synthetic oligonucleotide
cgcccaggctggagtgcac

SEQ ID NO: 966 - DNA, 60 bp, Homo sapiens
tcatcgtcatggtgggcctccccgcccggggcaagacctacatctccaagaagctgactc

SEQ ID NO: 967 - DNA, 60 bp, Homo sapiens
tcattgtcatggtgggcctgcccgccaggggcaagacctacatctccaagaagctgactc

SEQ ID NO: 968 - RNA, 45 nt, Artificial Sequence
  Description of Artificial Sequence: Synthetic oligonucleotide
gcgacugguuacccggucgcaaaauuuaucuuggaucuuggccuu

SEQ ID NO: 969 - RNA, 45 nt, Artificial Sequence
  Description of Artificial Sequence: Synthetic oligonucleotide
gcgacugguuacccggucgcaauuggccaagauccaagauaaauu

SEQ ID NO: 970 - RNA, 104 nt, Artificial Sequence
  Description of Artificial Sequence: Synthetic polynucleotide
gaauuccgcgugugccagcgaaaguugcguaugggucacaucgcaggcacaugucaucugggcgguccguucgggauccaaaauuaucuuggaucuuggccuca

SEQ ID NO: 971 - RNA, 59 nt, Artificial Sequence
  Description of Artificial Sequence: Synthetic oligonucleotide
agccgcgaggggagggauaggguagggcgcggcuaaaaaggccaagauccaagauaauu

SEQ ID NO: 972 - RNA, 47 nt, Artificial Sequence
  Description of Artificial Sequence: Synthetic oligonucleotide
acguaucccuuuucgcguaagccuucuuuaucuuggaucuuggccuu

SEQ ID NO: 973 - RNA, 106 nt, Artificial Sequence
  Description of Artificial Sequence: Synthetic polynucleotide
gaauuccgcgugugccagcgaaaguugcguaugggucacaucgcaggcacaugucaucugggcgguccguucgggauccuuggccaagauccaagauaaagaaggc

SEQ ID NO: 974 - RNA, 43 nt, Artificial Sequence
  Description of Artificial Sequence: Synthetic oligonucleotide
acguaucccuuuucgcguaaaaucgaaguacucagcguaaguu

SEQ ID NO: 975 - RNA, 104 nt, Artificial Sequence
  Description of Artificial Sequence: Synthetic polynucleotide
gaauuccgcgugugccagcgaaaguugcguaugggucacaucgcaggcacaugucaucugggcgguccguucgggauccuuuucuuacgcugaguacuucgauu

SEQ ID NO: 976 - RNA, 45 nt, Artificial Sequence
  Description of Artificial Sequence: Synthetic oligonucleotide
acguaucccuuuucgcguuuggccaagauccaagauaaagaaggc

SEQ ID NO: 977 - RNA, 108 nt, Artificial Sequence
  Description of Artificial Sequence: Synthetic polynucleotide
gaauuccgcgugugccagcgaaaguugcguaugggucacaucgcaggcacaugucaucugggcgguccguucgggauccaagccuucuuuaucuuggaucuuggccuu

SEQ ID NO: 978 - RNA, 43 nt, Artificial Sequence
  Description of Artificial Sequence: Synthetic oligonucleotide
acguaucccuuuucgcguuuuucuuacgcugaguacuucgauu

SEQ ID NO: 979 - RNA, 64 nt, Artificial Sequence
  Description of Artificial Sequence: Synthetic oligonucleotide
gggaggacgaugcggaucagccauguuuacgucacuccuaaaauguagagaugcggugguccuu

SEQ ID NO: 980 - RNA, 21 nt, Artificial Sequence
  Description of Artificial Sequence: Synthetic oligonucleotide
uuaucuuggaucuuggccuuc

SEQ ID NO: 981 - RNA, 87 nt, Artificial Sequence
  Description of Artificial Sequence: Synthetic oligonucleotide
gggaggacgaugcggaucagccauguuuacgucacuccuaaaaaggccaagauccaagauaauuuuggaccaccgcaucucuacauu

SEQ ID NO: 982 - RNA, 21 nt, Artificial Sequence
  Description of Artificial Sequence: Synthetic oligonucleotide
cuuacgcugaguacuucgauu

SEQ ID NO: 983 - DNA, 27 bp, Artificial Sequence
  Description of Artificial Sequence: Synthetic oligonucleotide
  Description of Combined DNA/RNA Molecule: Synthetic oligonucleotide
ggccaagauccaagauaaagaaggctt

SEQ ID NO: 984 - RNA, 13 nt, Artificial Sequence
  Description of Artificial Sequence: Synthetic oligonucleotide
uugauuuacaaac

SEQ ID NO: 985 - RNA, 13 nt, Artificial Sequence
  Description of Artificial Sequence: Synthetic oligonucleotide
uugauuuacuaac

SEQ ID NO: 986 - RNA, 84 nt, Artificial Sequence
  Description of Artificial Sequence: Synthetic oligonucleotide
gccuucuuuaucuuggaucuuggccuuaaaaaaaggagaugcguaggguggacugagugaugucugucaagaccucguccaaac

SEQ ID NO: 987 - RNA, 84 nt, Artificial Sequence
  Description of Artificial Sequence: Synthetic oligonucleotide
gccuucuuuaucuuggaucuuggccuuaaaaaaaggaggguugcguugcaaguaacagaaaggaauuugaaaauuguggcaaac

SEQ ID NO: 988 - RNA, 84 nt, Artificial Sequence
  Description of Artificial Sequence: Synthetic oligonucleotide
gccuucuuuaucuuggaucuuggccuuaaaaaaaggagacacaugugacaagaggcuaugauccugaaugcauccuuggcaaac

SEQ ID NO: 989 - RNA, 84 nt, Artificial Sequence
  Description of Artificial Sequence: Synthetic oligonucleotide
gccuucuuuaucuuggaucuuggccuuaaaaaaaggaguagugcagcauugacugaaugucauacggcauaagcaucuacaaac

SEQ ID NO: 990 - RNA, 84 nt, Artificial Sequence
  Description of Artificial Sequence: Synthetic oligonucleotide
gccuucuuuaucuuggaucuuggccuuaaaaaaaggaguauacauucuugguucauaaaggauaaggccuaagucgggucaaac

SEQ ID NO: 991 - RNA, 50 nt, Artificial Sequence
  Description of Artificial Sequence: Synthetic oligonucleotide
ggagaugcguaggguggacugagugaugucugucaagaccucguccaaac

SEQ ID NO: 992 - RNA, 50 nt, Artificial Sequence
  Description of Artificial Sequence: Synthetic oligonucleotide
ggaggguugcguugcaaguaacagaaaggaauuugaaaauuguggcaaac

SEQ ID NO: 993 - RNA, 50 nt, Artificial Sequence
  Description of Artificial Sequence: Synthetic oligonucleotide
ggagacacaugugacaagaggcuaugauccugaaugcauccuuggcaaac

SEQ ID NO: 994 - RNA, 50 nt, Artificial Sequence
  Description of Artificial Sequence: Synthetic oligonucleotide
ggaguagugcagcauugacugaaugucauacggcauaagcaucuacaaac

SEQ ID NO: 995 - RNA, 50 nt, Artificial Sequence
  Description of Artificial Sequence: Synthetic oligonucleotide
ggaguauacauucuugguucauaaaggauaaggccuaagucgggucaaac

SEQ ID NO: 996 - DNA, 89 bp, Artificial Sequence
  Description of Artificial Sequence: Synthetic oligonucleotide
gggagacaaacaaagagcgacaagggcagagtggacggtcggctagagccgggaggaattccttgtgaccacccagaagaagccagaag

SEQ ID NO: 997 - DNA, 50 bp, Homo sapiens
ccatgaactacatggtgtacttcaacttctttgcctgtgtgctggtgccc

SEQ ID NO: 998 - DNA, 47 bp, Homo sapiens
gcatggagtacatggtctacttcaacttctttgtgtgggtgctgccc

SEQ ID NO: 999 - DNA, 180 bp, Homo sapiens
gcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactcc

SEQ ID NO: 1000 - DNA, 180 bp, Homo sapiens
gcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgc

SEQ ID NO: 1001 - DNA, 240 bp, Homo sapiens
gccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggct

SEQ ID NO: 1002 - DNA, 240 bp, Homo sapiens
gctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgcca

SEQ ID NO: 1003 - DNA, 120 bp, Homo sapiens
cgaggagatgaccggctatgtggccacgcgctggtaccgggcacctgagatcatgctcaactggatgcattacaaccaaacagtggatatctggtccgtgggctgcatcatggctgagct

SEQ ID NO: 1004 - DNA, 120 bp, Homo sapiens
tgatgaaatgacaggctacgtggccactaggtggtacagggctcctgagatcatgctgaactggatgcattacaaccagacagttgatatttggtcagtgggatgcataatggccgagct

SEQ ID NO: 1005 - DNA, 60 bp, Homo sapiens
tggagcggcaggagaatgtgctggtcatctgccaccaggccgtcctgcgctgcctgcttg

SEQ ID NO: 1006 - DNA, 60 bp, Homo sapiens
tggagaggcaagagaatgtgctggtcatctgccaccaggctgtgatgcgctgcctgctgg

(19) United States
(12) Patent Application Publication
(10) Pub. No.: US 2024/0240178 A1
(43) Pub. Date: Jul. 18, 2024

(54) SIRNA CONSTRUCTS FOR INHIBITING GENE EXPRESSION IN TARGETED CANCER CELLS

(71) Applicant: SYSTEMS ONCOLOGY, LLC, Scotsdale, AZ (US)

(72) Inventors: Spyro Mousses, Scotsdale, AZ (US); David Azorsa, Scotsdale, AZ (US); Daniel Feldheim, Boulder, CO (US); James Heil, Boulder, CO (US); Necky Tran, London, Ontario (CA); Gregory Allen Penner, Toronto, Ontario (CA)

(21) Appl. No.: 18/289,519

(22) PCT Filed: May 5, 2022
(86) PCT No.: PCT/US2022/027930

§ 371 Date: Nov. 3, 2023

Related U.S. Application Data
(60) Provisional application No. 63/185,359, filed on May 6, 2021.
(60) Provisional application No. 63/231,234, filed on Aug. 9, 2021.
(60) Provisional application No. 63/242,865, filed on Sep. 10, 2021.
(60) Provisional application No. 63/250,548, filed on Sep. 30, 2021.
(60) Provisional application No. 63/287,037, filed on Dec. 7, 2021.
(60) Provisional application No. 63/287,040, filed on Dec. 7, 2021.
(60) Provisional application No. 63/323,997, filed on Mar. 25, 2022.

(51) Int. Cl. C12N 15/113; C12N 15/115

(52) U.S. Cl. CPC C12N 15/113; C12N 15/115; C12N 2310/14; C12N 2310/16; C12N 2310/3519; C12N 2320/32

(57) ABSTRACT

The invention provides siRNA compositions for inhibiting gene expression in targeted cells. A multi-targeting siR-NA-aptamer platform is provided that is efficiently delivered and is processed by cellular RNAi machinery to produce one, two or more siRNAs. Methods of using the multi-targeting siRNA-aptamer for selectively targeting cancer cells to downregulate the expression of multiple genes are also provided.

### RELATED AND PRIORITY APPLICATIONS

This application is a National Stage of International Application No. PCT/US2022/027930, filed May 5, 2022, which claims priority to U.S. Provisional Patent Application No. 63/185,359 filed May 6, 2021, U.S. Provisional Patent Application No. 63/231,234 filed Aug. 9, 2021, U.S. Provisional Patent Application No. 63/242,865 filed Sep. 10, 2021, U.S. Provisional Patent Application No. 63/250,548 filed Sep. 30, 2021, U.S. Provisional Patent Application No. 63/287,037 filed Dec. 7, 2021, U.S. Provisional Patent Application No. 63/287,040 filed Dec. 7, 2021 and U.S. Provisional Patent Application No. 63/323,997 filed Mar. 25, 2022. All of the above applications are incorporated herein by reference in their entirety.

### FIELD OF THE INVENTION

The invention is generally directed to siRNA compositions for inhibiting gene expression in targeted cancer cells.

### SEQUENCE LISTING

The instant application contains a Sequence Listing which has been submitted electronically in ASCII format and is hereby incorporated by reference in its entirety. Said ASCII copy, created on May 26, 2022, is named 10911_011098-WO0_SL.txt and is 248,895 bytes in size.

### BACKGROUND OF THE INVENTION

RNA interference (RNAi), also known as RNA silencing, has been extensively explored for therapeutic use in reducing gene expression but in the decades since its discovery few therapeutics have been approved. The traditional design pattern for RNA inhibition is that one piece of siRNA aims at one specific sequence (Reynolds et al., Nat Biotechnol, 22:326-330 (2004)). There remains a need in the art for compositions and methods of delivering siRNA to cells (e.g., malignant cells, tumor-associated T cells, effector T cells) to inhibit diseases such as cancer, metastasis or metabolic diseases. The nucleic acid compounds and methods of using the same as provided herein solve these and other problems in the art.

Recent work has expanded the RNA constructs to include joining two siRNAs to inhibit two different targets (Liu et al., Sci Reports, 6: (2016)). SiRNA's processed by cellular RNAi machinery to produce two siRNAs as opposed to dual administration offers a number of benefits including increased circulating half-life and reduced renal excretion (Liu et al., Sci Reports, 6: (2016)).

Dual targeting of genes by a single siRNA through targeting conserved homologous regions has been shown to be effective to inhibit the expression of gene families by diminishing the function of escape pathways. In vitro, a multi-target siRNA targeting the conserved homology region of DNMT3 family members effectively inhibited expression (Du et al., Gen and Mol Bio, 35:164-171(2012)).

Delivery to tissues other than the liver has remained a complication and hinderance for RNAi therapies. Aptamer-siRNA chimeras have been used to effectively deliver siRNA's to downregulate expression of oncological genes targets (Liu et al., Sci Reports, 6: (2016)).

U.S. Pat. No. 6,506,559 discloses a method to inhibit expression of a target gene in a cell, the method comprising the introduction of a double-stranded RNA into the cell in an amount sufficient to inhibit expression of the target gene, wherein the RNA is a double-stranded molecule with a first ribonucleic acid strand consisting essentially of a ribonucleotide sequence which corresponds to a nucleotide sequence of the target gene and a second ribonucleic acid strand consisting essentially of a ribonucleotide sequence which is complementary to the nucleotide sequence of the target gene. Furthermore, the first and the second ribonucleotide strands are separately complementary strands that hybridize to each other to form the said double-stranded construct, and the double-stranded construct inhibits expression of the target gene.

U.S. Pat. No. 5,475,096 discloses nucleic acid molecules each having a unique sequence, each of which has the property of binding specifically to a desired target compound or molecule. Each nucleic acid molecule is a specific ligand of a given target compound or molecule. The process, known as SELEX, is based on the idea that nucleic acids have sufficient capacity to form a variety of two- and three-dimensional structures with sufficient chemical versatility available within their monomers to act as ligands (form specific binding pairs) with virtually any chemical compound, whether monomeric or polymeric. Molecules of potentially any size can serve as targets.

The SELEX method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

U.S. Pat. No. 9,953,131 discloses a method for designing a dual-targeting short interfering RNAs (siRNAs) in which both strands are deliberately designed to separately target different mRNA transcripts with complete complementarity.

U.S. Pat. No. 9,777,278 discloses an interfering nucleic acid (iNA) duplex comprised of a sense strand of nucleotides having a 5′ end and a 3′ end annealed onto an antisense strand of nucleotides having a 5′ end and a 3′ end wherein the antisense strand has at least two segments, wherein one segment of the antisense strand can target a first RNA and another segment of the antisense strand can target a second RNA, or one segment of the antisense strand can target a first portion of an RNA and another segment of the antisense strand can target a second non-contiguous portion of said RNA.

U.S. Pat. No. 9,695,425 discloses an siRNA molecule that, when internalized by a B cell, suppresses expression of BAFF-R and one other target oncogene selected from: Bcl6, Bcl2, STAT3, Cyclin D1, Cyclin E2 and c-myc.

U.S. Pat. No. 10,689,654 discloses a bivalent siRNA chimera capable of silencing two or more genes. Methods of using the bivalent siRNA chimeras for selectively targeting cells to down-regulate the expression of multiple genes is also disclosed

Du et al., Gen and Mol Bio, 35:164-171(2012) discloses a siRNA targeting the conserved homologous region of DNMT3 family members.

U.S. Pat. No. 10,689,654 discloses a bivalent siRNA chimera platform that incorporates two aptamers for increase efficiency of delivering siRNAs to the targeted cell. Furthermore, those aptamers are conjugated to an siRNA construct that is processed by cellular RNAi machinery to produce at least two different siRNAs to inhibit expression of two or more different genes.

U.S. patent application Ser. No. 15/899,473 discloses bispecific aptamers.

U.S. Pat. No. 9,567,586 discloses an EPCAM aptamer coupled to an siRNA.

U.S. Pat. No. 10,385,343 discloses a method of treating cancer by administering a chimeric molecule comprising an EPCAM binding aptamer domain and an inhibitory nucleic acid domain that targets Plk1.

Patent Application PCT/US2020/038355 discloses an EpCAM-binding aptamer domain conjugated to an siRNA that inhibits the expression of a gene selected from the group consisting of: UPF2; PARP1; APE1; PD-L1; MCL1; PTPN2; SMG1; TREX1; CMAS; and CD47 for the purpose of treating cancer.

U.S. Pat. No. 10,960,086 discloses an siRNA-aptamer chimera that utilizes two aptamers targeting HER2 and HER3 and an siRNA targeting EGFR.

U.S. Pat. No. 8,828,956 N-acetylgalactosamine (GalNAc)-siRNA conjugates that enables subcutaneous dosing of RNAi therapeutics with potent and durable effects and a wide therapeutic index. This delivery systems is only effective for delivering to the liver as GalNAc binds to the Asialoglycoprotein receptor (ASGPR) that is predominantly expressed on liver hepatocytes.

U.S. Pat. No. 8,058,069 discloses lipid nanoparticle (LNP) delivery technology. LNP technology (formerly referred to as stable nucleic acid-lipid particles or SNALP) encapsulates siRNAs with high efficiency in uniform lipid nanoparticles that are claimed to be effective in delivering RNAi therapeutics to disease sites in various preclinical models.

U.S. Pat. No. 10,278,986 discloses an antibody conjugated to an siRNA as a delivery mechanism. The antibody targets C5aR and the siRNA targets C5 expression for the treatment of rheumatoid arthritis. Patent Application PCT/US2020/036307 discloses a method of preparing an antibody covalently linked to one or more oligonucleotides.

Aptamers are single-stranded RNA or DNA oligonucleotides that are capable of binding with high affinity and specificity and are cost effective to produce. Aptamers are of great interest as an antibody-like replacement and are being investigate for their ability to selectively bind to a specific target, including proteins, peptides, carbohydrates, etc., as well as function as a ligand for directed drug delivery. However, there are two primary hurdles for aptamers reaching clinical significance, their need to be stabilized for in vivo use against nuclease degradation which results in a short half life, and their rapid renal clearance due to their small size.

Native DNA aptamers are more stable than RNA aptamers as RNA is a transient messenger. The in vitro half-life of an RNA aptamer in plasma is a few seconds, while a DNA aptamer has a half-life of up to hour (2000 White et al, 2002 Takei et al, 1991 Shaw et al). The 2′ hydroxyl group of RNA makes it chemically unstable, susceptible to hydrolysis, and allows for the catalysis of RNA strand scission by endoribonucleases (2009 Houseley et al). For these reasons, RNA aptamers are commonly chemically modified primarily at the 2′-position of pyrimidines to enhance stability.

U.S. Pat. No. 5,660,985 describes oligonucleotides containing nucleotide derivatives chemically modified at the 5- and 2′-positions of pyrimidines and purines including 2′-fluoro and 2′-amino modifications.

U.S. Pat. No. 5,580,737, describes highly specific nucleic acid ligands containing one or more nucleotides modified with 2′-amino (2′-NH_{#}), 2′-fluoro (2′-F), and/or 2′-O-methyl (2′-OMe).

U.S. patent application Ser. No. 08/264,029 describes oligonucleotides containing various 2′-modified pyrimidines.

U.S. patent application Ser. No. 10/524,817 describes a 4′-thioribonucleotide modified aptamer which was later developed in Kato, Y. et al. (2005) into an aptamer against human α-thrombin.

U.S. Pat. No. 9,914,914 describes six different modifications where the canonical ribofuranose ring of DNA and RNA is replaced by five- or six-membered congeners comprising HNA (1,5 anhydrohexitol nucleic acids), CeNA (cyclohexenyl nucleic acids), LNA (2′-O,4′-C-methylene-β-D-ribonucleic acids; locked nucleic acids), ANA (arabinonucleic acids), FANA (2′-fluoro-arabinonucleic acid) and TNA (a-L-threofuranosyl nucleic acids).

U.S. patent application Ser. No. 61/748,834 describes Threose nucleic acid (TNA) modified aptamers.

U.S. patent application Ser. No. 60/905,461 describes double-stranded locked nucleic acid modifications (2′-O,4′-C-methylene-β-D-ribonucleic acids).

Lato, S. M. (2002) Nucleic Acids Res., describes Ribonucleoside 5′-(alpha-P-borano)-triphosphates (BH₃-RNA) modified aptamers.

PCT Publication No. 1997/004726 describes spiegelmers which are mirror images of the natural aptamers in which the D-ribose (the natural ribose) are replaced with the unnatural L-ribose. PCT Publication NO. 2001/006014 describes one of the first SELEX generated spiegelmers developed against D-adenosine.

Jhaveri, S. et al. (1998) Bioorg. Med. Chem. Lett., describes Ribonucleoside 5′-(alpha-thio) triphosphates (S-RNA) modified aptamers.

PCT Publication No. 1994/010562 describes RNA aptamers containing photoreactive chromophore 5-iodouridine using crosslinking SELEX.

In spite of recent advances, there is a need in the art for compositions and methods of delivering modulators of cell activity (e.g., anti-tumor agents, anti-obesity agents) to cells (e.g., malignant cells, tumor-associated T cells, effector T cells) to inhibit diseases such as cancer, metastasis or metabolic diseases. The nucleic acid compounds and methods of using the same as provided herein solve these and other problems in the art.

### SUMMARY OF THE INVENTION

A multi-targeting siRNA-aptamer platform is provided that is efficiently delivered and is processed by cellular RNAi machinery to produce one, two or more siRNAs. Methods of using the multi-targeting siRNA-aptamer for selectively targeting cancer cells to down-regulate the expression of multiple genes are also provided.

### BRIEF DESCRIPTION OF THE DRAWINGS

FIG. 1: Depicts the sequence alignment of UBBs1 to various targets, non-binding regions are highlighted.

FIG. 1A: Depicts BLAST results of UBBs1 showing potential homologous regions to UBB mRNA at three regions with 19/19, 18/19 and 17/19 identity over the 19 nt stretch. Plus/Plus indicated that the guide strand of UBBs1 would bind the the mRNA of UBB. FIG. 1A discloses SEQ ID NOS 604, 898, 604 and 899-903, respectively, in order of appearance.

FIG. 1B: Depicts BLAST results of UBBs1 showing potential homologous regions to UBC mRNA at three regions with 14/14 identity over the 19 nt stretch. Results for UBBs1 BLAST showing potential binding to UBC mRNA with 14/14 identity. Further examination showed 3 of 4 nt were identical and overall 17/19 identity to UBBs1. FIG. 1B discloses SEQ ID NOS 904-905, 904-905, 904-905 and 902-903, respectively, in order of appearance.

FIG. 1C: Depicts BLAST results of UBBs1 showing potential homologous regions to DCP2 mRNA at one region with 15/15 identity. FIG. 1C discloses SEQ ID NOS 906-907 and 902-903, respectively, in order of appearance.

FIG. 1D: Depicts BLAST results of UBBs1 showing potential homologous regions to FAM83F mRNA at one region with 15/15 identity. FIG. 1D discloses SEQ ID NOS 908-909 and 902-903, respectively, in order of appearance.

FIG. 1E: Depicts BLAST results of UBBs1 showing potential homologous regions to LOC646588 mRNA at one region with 15/15 identity. FIG. 1E discloses SEQ ID NOS 908-909 and 902-903, respectively, in order of appearance.

FIG. 1F: Depicts BLAST results of UBBs1 showing potential homologous regions to NACA2 mRNA at one region with 15/15 identity. FIG. 1F discloses SEQ ID NOS 910-911 and 902-903, respectively, in order of appearance.

FIG. 1G: Depicts BLAST results of UBBs1 showing potential homologous regions to RNF17 mRNA at one region with 15/15 identity. FIG. 1G discloses SEQ ID NOS 906-907 and 902-903, respectively, in order of appearance.

FIG. 2A: Depicts the potential UBBs1 siRNA targeting sites (highlighted in yellow) on the UBB sequence. Nucleotide differences are highlighted in red and similar repeat sequences are in blue. FIG. 2A discloses SEQ ID NO: 912.

FIG. 2B: Depicts the potential UBBs1 siRNA targeting sites (highlighted in yellow) on the UBC sequence. Nucleotide differences are highlighted in red and similar repeat sequences are in blue. FIG. 2B discloses SEQ ID NO: 913.

FIG. 3A: Schematic of a potential dual UBB/UBC siRNA aptamer. FIG. 3A discloses SEQ ID NOS 914, 902-903 and 914, respectively, in order of appearance.

FIG. 3B: Schematic of aptamer depicting UBBs1 siRNA and EPCAM aptamer. FIG. 3B discloses SEQ ID NOS 915-916, respectively, in order of appearance.

FIG. 4A: Depicts effect of siRNA on HCT-116 colon cancer cell viability.

FIG. 4B: Depicts effect of siRNA on SW480 colon cancer cell viability.

FIG. 5A: Depicts effect of siRNA on HT-29 colon cancer cell viability.

FIG. 5B: Depicts effect of siRNA on RKO colon cancer cell viability.

FIG. 6A: Depicts effect of siRNA on MCF-7 breast cancer cell viability.

FIG. 6B: Depicts effect of siRNA on SK-BR-3 breast cancer cell viability.

FIG. 7A: Dose response curve of UBB targeting siRNA on HCT-116 colon cancer cells.

FIG. 7B: Dose response curve of UBB targeting siRNA on SW480 colon cancer cells.

FIG. 8A: Depicts effect of U22 siRNA treatment of colon cancer cells on UBB expression normalized to b-Actin.

FIG. 8B: Depicts effect of U22 siRNA treatment of colon cancer cells on UBC expression normalized to b-Actin.

FIG. 8C: Depicts effect of U22 siRNA treatment of colon cancer cells on UBB expression normalized to GAPDH.

FIG. 8D: Depicts effect of U22 siRNA treatment of colon cancer cells on UBC expression normalized to GAPDH.

FIG. 9: Depicts effect of UBB targeting siRNA treatment of HCT-116 colon cancer cells on UBB and UBC expression normalized to GAPDH.

FIG. 10: Depicts effect of UBC targeting siRNA treatment of HCT-116 colon cancer cells on UBB and UBC expression normalized to GAPDH.

FIG. 11A: Depicts alignment of UBB and UBC gene sequences to identify dual targeting siRNA. FIG. 11A discloses SEQ ID NOS 917-918, 627, 627, 638-642 and 630, respectively, in order of appearance.

FIG. 11B: Depicts alignment of UBB and UBC gene sequences to identify dual targeting siRNA. FIG. 11B discloses SEQ ID NOS 919-920, 628, 628, 629, 629, 640-641, 643 and 632, respectively, in order of appearance.

FIG. 11C: Depicts alignment of UBB and UBC gene sequences to identify dual targeting siRNA. FIG. 11C discloses SEQ ID NOS 921-922, 628, 628, 603, 637, 604, 635, 630, 630, 631 and 631, respectively, in order of appearance.

FIG. 12A: Depicts effect of siRNA on HCT-116 colon cancer cell viability.

FIG. 12B: Depicts effect of siRNA on SK-BR-3 colon cancer cell viability.

FIG. 13A: Depicts alignment of HsUBB and MmUBB to identify dual targeting sequences. FIG. 13A discloses SEQ ID NOS 923-924, 624, and 925, respectively, in order of appearance.

FIG. 13B: Depicts alignment of HsUBC and MmUBC to identify dual targeting sequences. FIG. 13B discloses SEQ ID NOS 926-927, 635 and 928-929, respectively, in order of appearance.

FIG. 14: Depicts effect of UBB targeting siRNA treatment of HCT-116 colon cancer cells on UBB and UBC expression normalized to GAPDH.

FIGS. 15A-D: Depicts modifications of UBB and UBC targeting siRNA.

FIG. 15A discloses SEQ ID NOS 930 and 931, respectively, in order of appearance.

FIG. 15B discloses SEQ ID NOS 930 and 931, respectively, in order of appearance.

FIG. 15C discloses SEQ ID NOS 932 and 933, respectively, in order of appearance.

FIG. 15D discloses SEQ ID NOS 499 and 933, respectively, in order of appearance.

FIG. 16: Depicts effect of treatment of HCT-116 colon cancer cells with modified UBB targeting siRNA on UBB and UBC expression.

FIG. 17: Depicts effect of treatment of HCT-116 colon cancer cells with modified UBB targeting siRNA on cell viability.

FIG. 18A: Depicts alignment of NR4A1, NR4A2 and NR4A3 gene sequences to identify multitargeting siRNA. FIG. 18A discloses SEQ ID NOS 934-936, 646, 937-938, 647, 939-940, 648 and 941-942, respectively, in order of appearance.

FIG. 18B: Depicts alignment of ADORA2A and ADORA2B gene sequences to identify dual targeting siRNA. FIG. 18B discloses SEQ ID NOS 943-944, 997-998, 649, 945, 650, 946, 651 and 947, respectively, in order of appearance.

FIG. 18C: Depicts alignment of MAP2K1 and MAP2K2 gene sequences to identify dual targeting siRNA. FIG. 18C discloses SEQ ID NOS 948-949, 999-1000, 652, 950, 653, 951, 654, 952, 655, 655, 656 and 656, respectively, in order of appearance.

FIG. 18D: Depicts alignment of MAPK1 and MAPK3 gene sequences to identify dual targeting siRNA. FIG. 18D discloses SEQ ID NOS 953-954, 1001-1002, 657, 955, 658, 956, 659, 957, 660 and 958, respectively, in order of appearance.

FIG. 18E: Depicts alignment of MAPK11 and MAPK14 gene sequences to identify dual targeting siRNA. FIG. 18E discloses SEQ ID NOS 959-960, 1003-1004, 661, 661, 662, 662-663 and 961, respectively, in order of appearance.

FIG. 18F: Depicts alignment of MDM2 and MDM4 gene sequences to identify dual targeting siRNA. FIG. 18F discloses SEQ ID NOS 962-963, 664, 964, 665 and 965, respectively, in order of appearance.

FIG. 18G: Depicts alignment of PFKFB3 and PFKFB4 gene sequences to identify dual targeting siRNA. FIG. 18G discloses SEQ ID NOS 966-967, 1005-1006, 666, 666, 667 and 667, respectively, in order of appearance.

FIG. 19A: Depicts effect of dual targeting siRNA treatment of cancer cells on MAP2K1 and MAP2K2 expression normalized to GAPDH.

FIG. 19B: Depicts effect of dual targeting siRNA treatment of cancer cells on MAPK1 and MAPK3 expression normalized to GAPDH.

FIG. 20A: Depicts effect of dual targeting siRNA treatment of cancer cells on ADORA2A and ADORA2B expression.

FIG. 20B: Depicts effect of dual targeting siRNA treatment of cancer cells on MAPK11 and MAPK14 expression.

FIG. 21: Depicts effect of gene specific siRNA treatment of cancer cells on MAP2K1 and MAP2K2 expression normalized to GAPDH.

FIG. 22A: Depicts effect of siRNA treatment on EGFR expression in cancer cells normalized to GAPDH.

FIG. 22B: Depicts effect of siRNA treatment on EGFR expression in cancer cells normalized to GAPDH.

FIG. 23: Depicts effect of siRNA treatment on BIRC5 expression in cancer cells normalized to GAPDH.

FIG. 24: Depicts effect of siRNA treatment on PIKFYVE expression in cancer cells normalized to GAPDH.

FIG. 25A: Depicts effect of gene specific siRNA treatment of cancer cells on NR4A1 expression normalized to GAPDH.

FIG. 25B: Depicts effect of gene specific siRNA treatment of cancer cells on NR4A2 expression normalized to GAPDH.

FIG. 25C: Depicts effect of gene specific siRNA treatment of cancer cells on NR4A3 expression normalized to GAPDH.

FIG. 26A: Depicts effect of gene specific siRNA treatment of cancer cells on MTOR and GRB7 expression normalized to GAPDH.

FIG. 26B: Depicts effect of gene specific siRNA treatment of cancer cells on IDO1 and STAT3 expression normalized to GAPDH.

FIG. 27A: Depicts effect of gene specific siRNA treatment of cancer cells on c-MYC and YY1 expression normalized to GAPDH.

FIG. 27B: Depicts effect of gene specific siRNA treatment of cancer cells on MDM2 and MDM4 expression normalized to GAPDH.

FIG. 28A: Depicts effect of gene specific siRNA treatment of cancer cells on CBLB and TOX expression normalized to GAPDH.

FIG. 28B: Depicts effect of gene specific siRNA treatment of cancer cells on CBLB and TOX expression normalized to GAPDH.

FIG. 29: Depicts effect of gene specific siRNA treatment of cancer cells on RICTOR and TOX2 expression normalized to GAPDH.

FIG. 30A: Depicts effect of gene specific siRNA treatment of cancer cells on MSI1 and MSI2 expression normalized to GAPDH.

FIG. 30B: Depicts effect of gene specific siRNA treatment of cancer cells on UBC and VHL expression normalized to GAPDH.

FIG. 31: Depicts effect of gene specific siRNA treatment of cancer cells on ADORA2A and ADORA2B expression normalized to GAPDH.

FIG. 32A: Depicts effect of gene specific siRNA treatment of cancer cells on PTPN2 and VHL expression normalized to GAPDH.

FIG. 32B: Depicts effect of gene specific siRNA treatment of cancer cells on UBB and UBC expression normalized to GAPDH.

FIG. 33A: Depicts effect of gene specific siRNA treatment of cancer cells on AKT1 and BATF expression normalized to GAPDH.

FIG. 33B: Depicts effect of gene specific siRNA treatment of cancer cells on ME2 and ME3 expression normalized to GAPDH.

FIG. 34: Schematic depicting dual binding properties of bivalent aptamer-siRNA chimera.

FIG. 35A: Schematic depicting the annealed bivalent EPCAM aptamer-UBB siRNA chimera. FIG. 35A discloses SEQ ID NOS 969 and 968, respectively, in order of appearance.

FIG. 35B: Gel showing comparison of RNA1, RNA2, RNA1 and RNA2 and the annealed EpCAM-directed aptamers-siRNA chimera.

FIG. 36A: Schematic depicting the annealed Her2/Her3 dual targeting aptamer-UBB siRNA chimera. FIG. 36A discloses SEQ ID NOS 970-971, respectively, in order of appearance.

FIG. 36B: Gel showing comparison of RNA1, RNA2, and the annealed Her2/Her3 dual targeting aptamer-UBB siRNA chimera.

FIG. 37A: Schematic depicting the annealed EPCAM/Her3 dual targeting aptamer-UBB siRNA chimera. FIG. 37A discloses SEQ ID NOS 972-973, respectively, in order of appearance.

FIG. 37B: Schematic depicting the annealed EPCAM/Her3 dual targeting aptamer-Luc siRNA chimera. FIG. 37B discloses SEQ ID NOS 974-975, respectively, in order of appearance.

FIG. 37C: Schematic depicting the annealed EPCAM/Her3 dual targeting aptamer-UBB siRNA chimera. FIG. 37C discloses SEQ ID NOS 976-977, respectively, in order of appearance.

FIG. 37D: Gel showing comparison of RNA 1, RNA2, and the annealed EPCAM/Her3 dual targeting aptamer-UBB siRNA chimera; RNA3, RNA 4, and the annealed EPCAM/Her3 dual targeting aptamer-Luc siRNA chimera; RNA5, RNA6, and the annealed EPCAM/Her3 dual targeting aptamer-UBB siRNA chimera.

FIG. 37E: Schematic depicting the annealed bivalent EPCAM aptamer-UBB siRNA chimera. FIG. 37E discloses SEQ ID NOS 972 and 976, respectively, in order of appearance.

FIG. 37F: Schematic depicting the annealed bivalent EPCAM aptamer-Luc siRNA chimera. FIG. 37F discloses SEQ ID NOS 974 and 978, respectively, in order of appearance.

FIG. 37G: Gel showing comparison of RNA 7, RNA8, and the annealed bivalent EPCAM aptamer-UBB siRNA chimera; RNA9, RNA10, and the annealed bivalent EPCAM aptamer-Luc siRNA chimera.

FIG. 38A: Schematic depicting the annealed bivalent PSMA aptamer-dual BIRC5 and UBB siRNA chimera. FIG. 38A discloses SEQ ID NOS 979-981, respectively, in order of appearance.

FIG. 38B: Gel showing comparison of RNA 1, RNA2, RNA3 and the annealed bivalent PSMA aptamer-dual BIRC5 and UBB siRNA chimera.

FIG. 39: Depicts the effect of dicer treatment on the PSMA aptamer-dual BIRC5 and UBB siRNA chimera.

FIG. 40A: Schematic depicting annealed EPCAM aptamer-UBB siRNA chimera. FIG. 40A discloses SEQ ID NOS 972 and 932, respectively, in order of appearance.

FIG. 40B: Schematic depicting annealed EPCAM aptamer-Luc siRNA chimera. FIG. 40B discloses SEQ ID NOS 974 and 982, respectively, in order of appearance.

FIG. 40C: Schematic depicting annealed EPCAM aptamer-UBB siRNA chimera. FIG. 40C discloses SEQ ID NOS 972 and 983, respectively, in order of appearance.

FIG. 40D: Depicts the effect of transfection of siRNA or aptamer/siRNA chimeras on UBB expression in cancer cells normalized to GAPDH.

FIG. 41: Depicts the effect of transfection of aptamer/siRNA chimeras on viability of cancer cells normalized to control.

FIG. 42: Depicts the effect of transfection of siRNA on viability of cancer cells normalized to control.

FIG. 43: Depicts the effect of transfection of aptamer/siRNA chimeras on viability of cancer cells normalized to control.

FIG. 44: Depicts predicted folding structures of potential PD1 binding RNA aptamers. FIG. 44 discloses SEQ ID NOS 807-810, 812, 996, 805-806 and 802-804, respectively, in order of appearance.

FIG. 45: Depicts predicted folding structures of potential CTLA4 binding RNA aptamers. FIG. 45 discloses SEQ ID NOS 820-824, respectively, in order of appearance.

FIG. 46: Depicts predicted folding structures of potential TIM3 binding RNA aptamers. FIG. 46 discloses SEQ ID NOS 825-829, respectively, in order of appearance.

FIG. 47: Depicts predicted folding structures of potential LAG3 binding RNA aptamers. FIG. 47 discloses SEQ ID NOS 860-864, respectively, in order of appearance.

FIG. 48: Depicts predicted folding structures of potential TROP2 binding RNA aptamers. FIG. 48 discloses SEQ ID NOS 867, 984, 868 and 985, respectively, in order of appearance.

FIG. 49A: Provides a table of 5-benzyl Uridine modified RNA aptamer sequences linked to UBB siRNA sequence via nucleotide linker. FIG. 49A discloses SEQ ID NOS 986-990, respectively, in order of appearance.

FIG. 49B: Provides a table of 5-benzyl Uridine modified RNA aptamer sequences linked to UBB siRNA sequence via chemical linker. FIG. 49B discloses SEQ ID NOS 933, 991, 933, 992, 933, 993, 933, 994, 933 and 995, respectively, in order of appearance.

FIG. 49C: Schematic of chemical linker for aptamer/siRNA chimera.

### DETAILED DESCRIPTION OF THE INVENTION

Cancer drugs are most effective when given in combination. One rationale for combination therapy is to use drugs that work by different mechanisms, thereby decreasing the likelihood that resistant cancer cells will develop. When drugs with different effects are combined, each drug can be used at its optimal dose, without intolerable side effects. See for example, https://www.merckmanuals.com/en-ca/home/cancer/prevention-and-treatment-of-cancer/combination-cancer-therapy, accessed May 3, 2021.

Combination therapy may also operate by simultaneously blocking two or more signaling pathways, Wu et al., Nat Biotechnol, 25:1290-1297 (2007). In addition, tumor progression and metastasis may be suppressed by overcoming the functional redundancy or synergistic action of targeted molecules (van der Veeken, et al., Current Cancer Drug Targets, 9:748-760 (2009)).

Zhao, et al. (Cancer discovery. 4. 10.1158/2159-8290.CD-13-0465, 2013) discuss the problem of intra-tumor heterogeneity and the approach of using computationally predictive combination therapy to address this problem.

NSCLC is any type of epithelial lung cancer other than small cell lung cancer (SCLC). NSCLC includes squamous cell carcinoma, large cell carcinoma, and adenocarcinoma, but there are other types also. NSCLCs are associated with cigarette smoke, however, adenocarcinomas are also found in patients who have never smoked.

NSCLC is generally less sensitive to chemotherapy and radiation therapy compared with SCLC. There are approximately 240,000 new cases and 130,000 deaths from lung cancer (NSCLC and SCLC combined) in the United States per year and lung cancer is the leading cause of cancer-related mortality in the United States.

Patients with advanced non-small cell lung cancer (NSCLC) have a very poor prognosis. TROP2 expression is associated with a poor prognosis, particularly in patients with adenocarcinoma histology, and offers a promising target for treatments. See https://www.onclive.com/view/novel-adc-appears-to-leverage-trop2-expression-in-nsclc accessed Apr. 27, 2022.

In certain embodiments of the instant invention NSCLC is treated with a chimeric aptamer siRNA construct comprising aptamers against Trop2 and Her3 plus siRNAs that inhibit a synthetic lethal pair of genes. In one preferred embodiment of a NSCLC treatment the synthetic lethal gene pair include UBB and UBC.

Colorectal cancer (CRC), including bowel cancer, colon cancer, or rectal cancer, colorectal cancer is the third most common cancer diagnosed in the United States. The American Cancer Society's estimates that in the United States there are 106,180 new cases of colon cancer.

In certain embodiments of the instant invention colon cancer is treated with a chimeric aptamer siRNA construct comprising aptamers against Epcam and Her3 plus siRNAs that inhibit a synthetic lethal pair of genes. In one preferred embodiment of a colon cancer treatment the synthetic lethal gene pair include UBB and UBC.

Prostate cancer is the second most common cancer globally. In 2018 there an estimated 1.2 million new cases with 359,000 deaths. Bray F, Ferlay J, Soerjomataram I, Siegel R L, Torre L A, Jemal A (November 2018). “Global cancer statistics 2018: GLOBOCAN estimates of incidence and mortality worldwide for 36 cancers in 185 countries”. CA: A Cancer Journal for Clinicians. 68 (6): 394-424. doi:10.3322/caac.21492. PMID 30207593. S2CID 52188256.

In certain embodiments of the instant invention prostate cancer is treated with a chimeric aptamer siRNA construct comprising aptamers against Trop2 and PSMA plus siRNAs that inhibit a synthetic lethal pair of genes. In one preferred embodiment of a NSCLC treatment the synthetic lethal gene pair include UBB and UBC.

As used herein, the term “oncogene” refers to a gene that can in some circumstances transform a cell into a cancerous cell or a gene that promotes the survival of a cancer cell.

As used herein, the term “effective amount” in the context of the administration of a therapy to a subject refers to the amount of a therapy that achieves a desired prophylactic or therapeutic effect.

A “siRNA,” “small interfering RNA,” “small RNA,” or “RNAi” as provided herein refers to a nucleic acid that forms a double stranded RNA, which double stranded RNA has the ability to reduce or inhibit expression of a gene or target gene (e.g., when expressed in the same cell as the gene or target gene). The complementary portions of the nucleic acid that hybridize to form the double stranded molecule typically have substantial or complete identity. In certain embodiments, a siRNA or RNAi is a nucleic acid that has substantial or complete identity to a target gene and forms a double stranded siRNA.

In certain embodiments, the instant invention comprises a chimeric molecule including a cancer marker-binding domain and an inhibitory nucleic acid domain. As used herein, “cancer marker-binding domain” refers to a domain and/or molecule that can bind specifically to a molecule more highly expressed on the surface of a cancer cell as compared to a healthy cell of the same type (a “cancer marker”). As used herein, “inhibitory nucleic acid domain” refers to a domain comprising an inhibitory nucleic acid. In some embodiments, the inhibitory nucleic acid can be a siRNA.

Certain embodiments of the instant invention comprise multi- and multi-multi-targeting siRNA and siRNA-aptamer chimeric molecules in treating cancer and other diseases which can be treated by genetic inhibition. The compounds and methods in certain embodiments of the instant invention may utilize one or more aptamers that target the therapeutic constructs specifically to cancer cells, providing effective and on-target suppression of the gene or genes targeted by the siRNA.

As used herein “multi-targeting siRNA or construct” refers to a set of unique and novel synthetic molecules for efficacious anti-tumor activity. These constructs each include siRNA molecules that each engage a cell's RNA inhibition system to inhibit more than one different gene (for example UBB and UBC).

As used herein “multi-multi-targeting siRNA or construct” refers to a set of unique and novel synthetic molecules for efficacious anti-tumor activity. These constructs each include siRNA molecules that each engage cell's RNA inhibition system to inhibit more than one different gene and that also include sequences found multiple times within each gene. Such multi-multi-targeting siRNA can be utilized alone or in constructs comprising multiple such siRNAs as well as one or more aptamers. Simple examples of such constructs can be targeted to one or more cancer cells and can inhibit or silence three or four genes although more exotic constructs can readily be envisioned by one skilled in the art once the instant invention is understood.

Ubiquitin B (UBB) is one of the two genes that encode for Ubiquitin. Silencing of UBB results in dependence on the second gene, Ubiquitin C (UBC) (Tsherniak et al., Cell, 170: 564-576(2017)). In certain embodiments described herein UBB and UBC can be effectively targeted with a single siRNA.

UBB and UBC also contain multiple conserved regions that could be exploited as a means to target both genes in multiple locations with one siRNA. Targeting multiple genes in multiple locations will be defined as multi-multi-targeting. Thus, a UBB/UBC siRNA can be designed as a multi-multi-targeting siRNA construct. When included in an siRNA/aptamer chimera including more than one aptamer, the construct actually can be thought of as a multi-multi-multi-targeting molecule.

A preferred aptamer for conjugation to a multi-targeting siRNA is an epithelial cell adhesion molecule (EpCAM) aptamer, EpCAM is a glycosylated membrane protein that is expressed in most organs and glands, with the highest expression in colon. (Schnell et al., BBA—Biomembranes, 1828: 1989-2001(2013)). Sequences for EpCAM are known for a variety of species, e.g., human EpCAM (see, e.g., NCBI Gene ID:4072; protein sequence: NCBI Ref Seq: NP_002345.2). A single EpCAM aptamer consisting of 19-nt RNA possesses similar binding affinity as antibodies and is efficiently internalized through receptor-mediated endocytosis (Shigdar, et al., Cancer Sci, 102:991-998 (2011); Wang, et al., Theranostics, 5:1456-1472 (2015)). Additionally, EpCAM is highly expressed in colon cancers and associated with colon cancer cell migration, proliferation, metastasis, and poor prognosis (Liang et al., Cancer Letters, 433: 165-175(2018)). For these reasons EpCAM has been used in certain embodiments of the instant invention as an aptamer target for targeted delivery of therapeutic siRNAs for colon cancer.

In certain embodiments, the aptamers described herein, for example those targeting EpCAM, permit the therapy to target tumor-initiating cells (also referred to as cancer stem cells). These cells are responsible not only for tumor initiation, relapse, and metastasis, but are also relatively resistant to conventional cytotoxic therapy. Thus, the compositions and methods described herein permit effective treatment of the underlying pathology in a novel way that existing therapies fail to do.

Moreover, the compounds according to certain embodiments of the instant invention are expected to be surprisingly efficacious in the treatment of colon cancers.

The compounds according to the instant invention are effective to inhibit gene expression in tumor cells.

The instant invention is also designed for targeted delivery of the therapeutic constructs and thus rapid tumor regression.

In certain embodiments, the cancer marker can be a protein and/or polypeptide. In certain embodiments, one cancer marker can be EpCAM. In certain preferred embodiments, the cancer marker-binding domain can be an aptamer.

In certain embodiments each siRNA inhibits two or more different genes.

One embodiment provides a bivalent siRNA chimera that contains two siRNAs where one siRNA inhibits the expression of UBB and UBC.

One embodiment provides a bivalent siRNA chimera that contains two siRNAs where one siRNA inhibits the expression of MAP2K1 and MAP2K2.

One embodiment provides a bivalent siRNA chimera that contains two siRNAs where one siRNA inhibits the expression of ERK1 (MAPK3) and ERK2 (MAPK1).

One embodiment provides a bivalent siRNA chimera that contains two siRNAs where one siRNA inhibits the expression of MAPK11 and MAPK14.

One embodiment provides a bivalent siRNA chimera that contains two siRNAs where one siRNA inhibits the expression of MDM2 and MDM4.

One embodiment provides a bivalent siRNA chimera that contains two siRNAs where one siRNA inhibits the expression of PFKFB3 and PFKFB4.

In certain embodiments siRNAs have been experimentally verified by real-time RT-PCR analysis and shown to provide at least 70% target knockdown at the mRNA level when used under optimal delivery conditions (confirmed using validated positive control and measured at the mRNA level 24 to 48 hours after transfection using 100 nM siRNA).

In certain other embodiments, siRNAs have been demonstrated to silence target gene expression by at least 75% at the mRNA level when used under optimal delivery conditions as validated by positive controls and measured at the mRNA level 24 to 48 hours after transfection using 100 nM siRNA.

Another embodiment provides a siRNA-aptamer chimera with two aptamers.

In certain embodiments, an aptamer of the siRNA chimeras binds to a cell surface protein expressed on cancer cells.

In certain embodiments, an aptamer of the siRNA chimeras specifically bind to epithelial cell adhesion molecules (EpCAM), a glycosylated membrane protein.

In certain embodiments, an aptamer of the siRNA chimeras specifically bind to DExH-Box Helicase 9, DHXP ((NCBI Gene ID: 1660). DHX9 protein is involved in transcriptional and translational regulation, DNA replication/repair, and maintenance of genome stability. DHX9 has been shown to shuttle between the nucleus and the cytoplasm

In certain embodiments, a method is provided which includes administering to a subject in need thereof and effective amount of bivalent siRNA chimera having aptamers that specifically bind to EPCAM and siRNA constructs that are processed to produce siRNA that inhibits expression of UBB and UBC; NR4A1, NR4A2 and NR4A3; ADORA2A and ADORA2B; MAP2K1 and MAP2K2; ERK1 (MAPK3) and ERK2 (MAPK1); MAPK11 and MAPK14; MDM2 and MDM4; PFKFB3 and PFKFB4; TOX and TOX2.

Another embodiment provides a pharmaceutical composition containing one or more different bivalent siRNA chimeras in an amount effective to down down-regulate at least three different genes in a target cell.

The method includes administering a dual targeting siRNA agent to the subject to be treated. When the organism to be treated is a mammal such as a human, the composition can be administered by any means known in the art including, but not limited to oral or parenteral routes, including intravenous, intramuscular, subcutaneous, transdermal, and airway (aerosol) administration. In some embodiments, the compositions are administered by intravenous infusion or injection.

Additional cancer markers that may be targeted by the aptamer portion of certain embodiments of the instant invention include, but are not limited to, ERBB2, ERBB3, PSMA, FOLH1, CD44, FOLH1, PSCA, PDCD1, TACSTD2, NT5E, PDCD1, CTLA4, LAG3, DHX9, or HAVCR2.

In certain embodiments, the aptamer-siRNA chimera of the instant invention includes an aptamer targeting ERBB2 (HER2) (NCBI Gene ID: 2064). HER2, a membrane tyrosine kinase, is overexpressed in 20%-30% of breast cancer and correlates with poor prognosis, high aggressiveness, and extensive drug resistance. U.S. Pat. No. 10,960,086 discloses an aptamer targeting HER2 as part of an siRNA-aptamer chimera.

In certain embodiments, the aptamer-siRNA chimera of the instant invention includes an aptamer targeting ERBB3 (HER3) (NCBI Gene ID: 2065). HER3, a membrane tyrosine kinase, is involved in the resistance against EGFR- and HER2-targeted therapies through activation of a compensatory survival pathway. U.S. Pat. No. 10,960,086 discloses an aptamer targeting HER3 as part of an siRNA-aptamer chimera.

In certain embodiments, the aptamer-siRNA chimera of the instant invention includes an aptamer targeting PSMA (NCBI Gene ID: 2346). Prostate-specific membrane antigen is a transmembrane protein expressed in all types of prostatic tissue. PSMA expression correlates

In certain embodiments, the aptamer-siRNA chimera of the instant invention includes an aptamer targeting CD44 (NCBI Gene ID: 960). CD44 is a transmembrane glycoprotein whose aberrant expression and dysregulation contributes to tumor initiation and progression. CD44 is involved in many processes including T cell differentiation, branching morphogenesis, proliferation, adhesion and migration. CD44 is a common biomarker of cancer stem cells.

In certain embodiments, the aptamer-siRNA chimera of the instant invention includes an aptamer targeting EPCAM (NCBI Gene ID: 4072). EPCAM is a glycosylated membrane protein that is expressed in most organs and glands, with the highest expression in colon and is associated with colon cancer cell migration, proliferation, metastasis, and poor prognosis. A single EpCAM aptamer consisting of 19-nt RNA possesses similar binding affinity as antibodies and is efficiently internalized through receptor-mediated endocytosis (Shigdar, et al., Cancer Sci, 102:991-998 (2011).

In certain embodiments, the aptamer-siRNA chimera of the instant invention includes an aptamer targeting PSCA, prostate stem cell antigen (NCBI Gene ID: 8000). PSCA is a membrane glycoprotein predominantly expressed in the prostate with a possible role in cell adhesion, proliferation control and cell survival. PSCA can have a tumor promoting or a tumor suppressive effect depending on the cell type.

In certain embodiments, the aptamer-siRNA chimera of the instant invention includes an aptamer targeting TROP2 (NCBI Gene ID: 4070). TROP2, a cell-surface glycoprotein, is a paralog of epithelial-specific cell adhesion molecule (EpCAM). It is overexpressed in adenocarcinomas, minimally expressed in normal tissues, and expression level is correlated with tumor invasiveness and poor prognosis.

The inhibitory nucleic acid domain of constructs according to the instant invention can inhibit the expression of a gene product that is upregulated in a cancer cell and/or the expression of a gene that is required for cell growth and/or survival. In some embodiments, the inhibitory nucleic acid domain can inhibit the expression of a gene selected from UBB (e.g. “Ubiquitin B”; NCBI Gene ID: 7314); UBC (e.g. “Ubiquitin C”; NCBI Gene ID: 7316), BCL2, STAT3, MYC, SYK, CCNE2, CCND1, CCND2, BIRC5, EGFR, UBB, UBC, NR4A1, NR4A2, NR4A1, NR4A3, ADORA2a, ADORA2b, ADORA1, MAP2K1, MAP2K2, MAPK3 (ERK1), MAPK1 (ERK2), HIF1, HIF2, PFKFB3, PFKFB4, PLK1, PLK4, CDK11A, CDK11B, CDK4, CDK6, PARP1, or PARP2. Sequences of these genes, e.g., the human mRNAs, may be obtained from the NCBI database and can be used according to the instant invention to inhibitory nucleic acids.

Furthermore, provided herein are exemplary inhibitory nucleic acid domains, e.g., a nucleic acid having the sequence of SEQ ID NO: 604.

Ubiquitin B (UBB) is one of the two genes that encode for Ubiquitin. Silencing of UBB results in dependence on the second gene, Ubiquitin C (UBC) (Tsherniak et al., Cell, 170: 564-576(2017)). Targeting of UBC in high-grade serous ovarian cancer (HGSOC), a cancer known for chronic UBB repression, demonstrated tumor regression and long term survival benefits. This suggests dual targeting UBB and UBC as a potential therapeutic strategy for cancer (Kedves, et al., Clin Invest, 127: 4554-4568 (2017)).

In certain embodiments, a siRNA according to the invention targets BCL2 (NCBI Gene ID:596) which is a regulator of apoptosis that is triggered in response to stress signals. BCL-2 was the first gene shown to promote prolonged cell survival rather than increased proliferation leading to the concept that inhibition of apoptosis is an important step in tumorigenesis.

In certain embodiments, a dual-targeting siRNA targets BCL2 and STAT3 (NCBI Gene ID: 6774) which is a cytoplasmic transcription factor that regulates cell proliferation, differentiation, survival, angiogenesis, and immune response.

In certain embodiments, a dual-targeting siRNA targets BCL2 and MYC (NCBI Gene ID: 4609) which is a proto-oncogene and encodes a nuclear phosphoprotein that plays a role in cell cycle progression, apoptosis and cellular transformation. Reregulated expression of MYC causally contributes to tumorigenesis and tumor growth maintenance.

In certain embodiments, a dual-targeting siRNA targets BCL2 and SYK (NCBI Gene ID: 6850), Spleen Associated Tyrosine Kinase, which has a cancer dependent therapeutic function. In many hematopoietic malignancies, SYK provides a survival function and inhibition or silencing of SYK can promote apoptosis. In cancers of non-immune cells, SYK can suppress tumorigenesis by enhancing cell-cell interactions and inhibiting migration.

In certain embodiments, a dual-targeting siRNA targets BCL2 and Cyclin E2 (NCBI Gene ID: 9134), a member of the cyclin family that assists in regulating the cell cycle and whose expression has been associated with chemotherapy resistance of tumor cells and poor prognosis.

In certain embodiments, a dual-targeting siRNA targets Cyclin E2 and Cyclin D1 (NCBI Gene ID: 595). Cyclin D1 overexpression is predominantly correlated with early cancer onset, tumor progression, shorter cancer patient survival and increased metastases.

In certain embodiments, a dual-targeting siRNA targets Cyclin D1 and EGFR (NCBI Gene ID: 1956), epidermal growth factor receptor, a cell surface protein whose expression modulates growth, signaling, differentiation, adhesion, migration and survival of cancer cells.

In certain embodiments, a dual-targeting siRNA targets Survivin (BIRC5) (NCBI Gene ID: 332) and Cyclin D2 (NCBI Gene ID: 895). Expression of Survivin in tumors correlates with inhibition of apoptosis, resistance to chemotherapy, and tumor progression. Cyclin D2 overexpression has a critical role in cell cycle progression and the tumorigenicity and suppression of cyclin D2 expression has been linked to G1 arrest in vitro.

CD45.1+CD45.2+(B6SJL×C57BL6) congenic mice were subcutaneously injected with OVA-expressing EL4 cells (E.G7 lymphoma) cells (5×10⁵ cells per mouse) in one flank. Six days later, PBS, wild-type or Nr4a1^{-/-} OT-I cells (3×10⁶ cells per mouse) were adoptively transferred into mice intravenously. Tumor sizes were monitored after adoptive transfer. To assess tumor-infiltrating donor T cells, mice were euthanized 6 days after T cell transfer. Donor-derived T cells were collected from tumor, draining lymph nodes and spleens, and subjected to flow cytometry analysis. Adoptive transfer of Nr4a1^{-/-} transgenic CD8⁺ T recognizing OVA₂₅₇₋₂₆₄ peptide (OT-I) cells into E.G7 tumor cell-bearing mice nearly eliminated tumors, in contrast with wild-type OT-I cells and a PBS control group. This data demonstrates that NR4A1 is linked to CD8⁺ T cell dysfunction (Liu, X., et al. Nature 2019).

Dysfunctional, or exhausted CD8⁺ T cells arise in the settings of chronic viral infection or cancer when persistent exposure to antigen leads to prolonged T cell receptor (TCR) signaling. In the exhausted state, T cell effector functions are impaired and manifest as decreased proliferative capacity, reduced cytolytic function and effector cytokine production, and altered in gene expression and metabolism. Notably, exhausted T cells upregulate multiple inhibitory receptors that include but are not limited to these immune checkpoint proteins: PD-1, CTLA-4, TIM-3, LAG-3, TIGIT, 2B4/CD244 and others. While activated effector T cells also transiently express immune checkpoint proteins, expression level increase and are sustained on exhausted T cell subsets. Transcription factors such as TOX and NR4A1 have been described as master regulators of exhaust.

In certain embodiments, these first-in-class, bivalent aptamer-dual siRNA chimeras harnesses the immune stimulatory potential of CTLA-4 and PD-1 within one RNA molecule. The results of the Phase III Checkmate 227 clinical trial in advanced non-small cell lung cancer recently demonstrated the longer duration of overall survival compared with chemotherapy in patients with NSCLC (Hellmann et al., N Engl J Med, 2019). In addition to delivering CTLA-4 and PD-1 antagonists selectively to T cells, this bivalent aptamer carries siRNA silencers that knock down expression of NR4A1, which reinvigorates exhausted T cells and VHL, which enables cells to adapt to hypoxic conditions in the TME.

In certain embodiments, a dual-targeting siRNA targets NR4A1 (NCBI Gene ID: 3164) and NR4A2 (NCBI Gene ID: 4929). When T cells encounter sustained T cell stimulation through exposure to self-antigens, to chronic infections or to the tumor microenvironment, then effector T cells may become dysfunctional to avoid excessive immune responses, which is known as T-cell exhaustion. NR4A1, a driver of cancer cell survival, has been identified as a key mediator of T cell dysfunction and contributor of regulatory T-cell-mediated suppression of anti-tumor immunity in the tumor microenvironment. Nr4a2 is highly expressed in tumor-infiltrating cells than in bystander cells. Furthermore, mice lacking Nr4a1 and Nr4a2 genes specifically in Tregs showed resistance to tumor growth in transplantation models.

In certain embodiments, a dual-targeting siRNA targets NR4A1 and NR4A3 (NCBI Gene ID: 8013), which is expressed similarly to NR4A1.

In certain embodiments, a multi-targeting siRNA targets NR4A1, NR4A2, and NR4A3.

In certain embodiments, a dual-targeting siRNA targets ADORA2a (NCBI Gene ID: 135) and ADORA2b (NCBI Gene ID: 136). ADORA2a signaling during T cell activation strongly inhibited development of cytotoxicity and cytokine-producing activity in T cells, whereas the inhibition of T cell proliferation was only marginal. While an adenosine-rich environment may allow for the expansion of T cell, it impairs the functional activation of T cells. Targeting the ADORA2a immunosuppressive pathway restores both effector function and metabolic fitness of peripheral and tumor-derived CD8⁺ T cells. ADORA2b promotes the expansion of myeloid-deriver suppressor cells which are immunosuppressive cells that promote tumor progression by impairing antitumor T-cell responses and/or modulating angiogenesis. Inhibition may be effective in delaying the growth of melanoma and perhaps other cancer as they improve local immunosurveillance. Experiments targeting both ADORA2a and aADORA2b have shown greater infiltration by CD8⁺ T cells as well as NK cells, and they encompass fewer Tregs.

In certain embodiments, a dual-targeting siRNA targets ADORA2a and ADORA1 (NCBI Gene ID: 134). ADORA1 and ADORA2A are paralogues and high-affinity receptors responding to low concentrations of extracellular adenosine.

In certain embodiments, a dual-targeting siRNA targets MAP2K1 (NCBI Gene ID: 5604), MEK1, and MAP2K2 (NCBI Gene ID: 5605), MEK2. MEK1 and MEK2 are closely related and participate in the Ras/Raf/MEK/ERK signal transduction cascade. MEK1 and MEK2 are the exclusively specific activators of ERK1/2, and their inhibition could result in the clinical benefits for treatment of cancers with RAS/RAF dysfunction.

In certain embodiments, a dual-targeting siRNA targets MAPK3 (NCBI Gene ID: 5595), ERK1, and MAPK1 (NCBI Gene ID: 5594) ERK2. ERK1 and ERK2, which are homologous by 85%, are part of the MAPK pathway, and the only substrate or MEK. The Ras-dependent extracellular signal-regulated kinase (ERK)1/2 mitogen-activated protein (MAP) kinase pathway plays a central role in cell proliferation control. ERK1/2 inhibitors can reverse the abnormal activation of MAPK pathway induced by upstream mutations including RAS mutation (Liu et al).

In certain embodiments, a dual-targeting siRNA target HIF1 (NCBI Gene ID: 3091) and HIF-2 (NCBI Gene ID: 2034). Hypoxia inducible factor (HIF)-1 and HIF-2 are heterodimeric transcription factors mediating the cellular response to hypoxia.

In certain embodiments, a dual-targeting siRNA target TOX(NCBI Gene ID: 9760) and TOX2 (NCBI Gene ID: 84968). High mobility group (HMG)-box transcription factors, TOX and TOX2, are critical for the transcriptional program of CD8; T cell exhaustion downstream of NFAT.

In certain embodiments, a dual-targeting siRNA targets PFKFB2 (NCBI Gene ID: 5208) and PFKFB3 (NCBI Gene ID: 5209). PFKFB2 is overexpressed in pancreatic adenocarcinomas and functions to regulate glycolysis and proliferation in pancreatic cancer cells. PFKFB3 is important for maintaining metabolic functions in pancreatic cancers and may be involved in providing a localized ATP supply at the plasma membrane.

In certain embodiments, a dual-targeting siRNA targets PFKFB3 and PFKFB4 (NCBI Gene ID: 5210). PFKFB4 is regulatory enzyme synthesizes a potent stimulator of glycolysis and is over expressed in many types of cancer such as in glioma, lung, and prostate cancers.

In certain embodiments, a dual-targeting siRNA targets PLK1 (NCBI Gene ID: 5347) and PLK4 (NCBI Gene ID: 10733). Polo-like kinase 1 and 4 play an important role in the initiation, maintenance, and completion of mitosis. Dysfunction of PLK1/4 promotes tumorigenesis. PLK1/4's role in cellular growth and proliferation and overexpression in multiple types of human cancer and has made them an attractive dual target.

In certain embodiments, a dual-targeting siRNA targets CDK11A (NCBI Gene ID: 728642) and CDK11B (NCBI Gene ID: 984). Recent studies have found that the overexpression and activation of CDK11 is crucial in the growth and proliferation of cancer cells, including breast cancer, multiple myeloma, osteosarcoma, and other types of cancer. Both of genes contain 20 exons and 19 introns that encode almost identical protein kinases, CDK11A and CDK11B.

In certain embodiments, a dual-targeting siRNA targets CDK6 (NCBI Gene ID: 1021) and CDK4 (NCBI Gene ID: 1432). CDK4/6 is highly expressed in the majority of human cancers through a multitude of genomic alterations. Sustained activation of CDK4/6 encourages cancer cells to enter the cell cycle continuously by shortening the duration of the G1 phase. CDK4/6 is highly expressed in the majority of human cancers through a multitude of genomic alterations. Sustained activation of CDK4/6 encourages cancer cells to enter the cell cycle continuously by shortening the duration of the G1 phase.

In certain embodiments, a dual-targeting siRNA targets MAPK11 (NCBI Gene ID: 5600) and MAPK14 (NCBI Gene ID: 1019). Mitogen activated protein kinases are involved in signaling transduction pathways, cell survival, differentiation, proliferation and apoptosis. MAPK11 has been found to be hypermethylated with a slight increase of expression in Breast, Uterine Endometrial, Cervical, Ovarian and Uterine Carcinosarcoma cell samples. MAPK11'S functions are mostly redundant to MAPK14 making these genes a strong dual target.

In certain embodiments, a dual-targeting siRNA targets MDM2 (NCBI Gene ID: 4193) and MDM4 (NCBI Gene ID: 4194). MDM2 and MDM4 are inhibitors of p53 expression. Dual inhibition of these genes has been shown to inhibit cellular proliferation by inducing cell cycle arrest and apoptosis in certain cancers.

In certain embodiments, a dual-targeting siRNA targets PARP1 (NCBI Gene ID: 142) and PARP2 (NCBI Gene ID: 10038). PARP is an important player in the DNA repair pathway which decreases cytotoxicity of chemotherapies and other. Targeted inhibition of PARP in cancerous cells assists in promoting cytotoxicity especially in combination with another therapy.

In certain embodiments, a dual-targeting construct targets PIKFYVE (NCBI Gene ID: 200576) as one of the targets. PIKFYVE is a lipid kinase and is involved in oncogenesis and cancer cell migration. Inhibition of this target has demonstrated slowed growth in prostate tumor cells.

In certain embodiments, a dual-targeting construct targets MTOR (NCBI Gene ID: 2475) as one of the targets. mTOR is a phosphatidylinositol kinase-related kinase and plays a key role in tumorigenesis. The AKT/mTOR signaling pathway is often upregulated in tumors.

In certain embodiments, a dual-targeting construct targets GRB7 (NCBI Gene ID: 2886) as one of the targets. GRB7, growth factor receptor bound protein-7, is a critical mediator of EGFR/ErbB signaling and the cancers associated.

In certain embodiments, a dual-targeting construct targets IDO1 (NCBI Gene ID: 3620) as one of the targets. Indoleamine 2, 3-dioxygenase, IDO1, is a tryptophan catabolic enzymes that catalyze the conversion of tryptophan into kynurenine which has the effect of suppressing the functions of effector T and natural killer cells, and promotes neovascularization of solid tumors.

In certain embodiments, a dual-targeting construct targets c-MYC (NCBI Gene ID: 4609) as one of the targets. C-MYC is a proto-oncogene and overexpression of the c-Myc gene is responsible for many of the changes that induce malignant changes.

In certain embodiments, a dual-targeting construct targets YY1 (NCBI Gene ID: 7528) as one of the targets. Yin Yang 1, YY1 is a transcription factor that regulates transcriptional activation and repression of many genes associated malignant transformation. YY1 is known to be pro-tumorigenic in colon cancer.

In certain embodiments, a dual-targeting construct targets CBLB (NCBI Gene ID: 868) as one of the targets. Cbl-b is expressed in all leukocyte subsets and regulates several signaling pathways in T cells, NK cells, B cells, and different types of myeloid cells

In certain embodiments, a dual-targeting construct targets RICTOR (NCBI Gene ID: 253260) as one of the targets. RICTOR is a member of the protein complex mTORC2 that functions in the regulation of actin organization, cell proliferation and survival.

In certain embodiments, a dual-targeting construct targets MSI1 (NCBI Gene ID: 4440) as one of the targets. Musashi RNA binding protein is a member of the protein complex mTORC2 that functions in the regulation of actin organization, cell proliferation and survival.

In certain embodiments, a dual-targeting construct targets AKT1 (NCBI Gene ID: 207) as one of the targets. AKT is a key element of the PI3K/AKT signaling pathway and regulates tumor growth, survival and invasiveness of tumor cells.

In certain embodiments, a dual-targeting construct targets BATF (NCBI Gene ID: 10538) as one of the targets. BATF, Basic Leucine Zipper ATF-Like Transcription Factor, may play an important role in the development of different types of cancer, including colon cancer, lymphoma and multiple myeloma

In certain embodiments, a dual-targeting construct targets ME2 (NCBI Gene ID: 4200) as one of the targets. Malic Enzyme 2 expression increases as tumor progression, cell migration, and invasion capabilities of cells are increased.

In certain embodiments, a dual-targeting construct targets ME3 (NCBI Gene ID: 10873) as one of the targets. Malic Enzyme 3 can promote proliferation, migration and invasion in pancreatic cancer cells.

Certain embodiment this invention include dual-targeting siRNA targeting two genes selected from a list consisting of: AKT1, ASCL1, BRAF, CD155, CDCP1, CTLA4, CTNNB1, CUX1, DHODH, EHMT1, ELK1, ERBB2, EZH2, FLT3, GLI1, GRB2, TOP1, GRB7, IDO1, KRAS, FGFR1, FGFR2, FKBP52, UBB, UBC, NUAK1, ONECUT2, PSMA, PDL1, PDL2, SON, NR4A1, NR4A, NR4A2, NR4A3, ADORA2a, ADORA2B, ADORA1, MAP2K1, MAP2K2, MAPK3 (ERK1), MAPK1 (ERK2), MAPK14, MDM2, MDM4, ME2, ME3, MSI1, MSI2, MTOR, RICTOR, RPTOR, MYCN, HIF1, HIF2, PFKFB3, PFKFB4, PFKFB2, PIK3CA, PIKFYVE, PTPN11, SKP2, SOAT1, SREBP2, SULT2B1, YAP, YY1, TEAD2, TERTM TMPRSS2, TSPAN3, ULK1, PLK1, PLK4, CDK11A, CDK11B, CDK4, CDK6, PARP1, PARP2, SYK, STAT3, MYC, BCL2, BCLXL, BMI1, FGFR3, FGFR4, PDGFRA, PDGFRB, IGF1R, IGF2R, ABCC3, IKBKB, IKBKA, FOXM1, RORC, PAK1, CXCR4, CCNE2, CCND2, BIRC5, CCND1, EGFR, EPCAM, HER2, HER3, GSK3B, RAB11, RAB1, Mir652, TAGN2, DUSP1, RPL10, FPRL1, miR29B, MUC12, miR200a, HSF1, TSPX, TRAF6, GATA2, ABDH2, AR, CCL5, PIKFYVE, MTOR, GRB7, IDO1, c-MYC, YY1, RICTOR, CBLB, MSI1, MSI2, and TPX2.

TABLE 1
Numerous siRNAs are useful in certain embodiments
of the instant invention. siRNAs that target
the listed gene are disclosed which are used in
certain embodiments in a double-stranded format
with their complementary (guide) strands.

| | | SEQ ID |
|---|---|---|
| Gene Symbol | siRNA (Sense Strand) | NO. |
| ABCC3 | CAAAGAAUGUCGACCCUAA | 1 |
| ABCC3 | GCUCUUUGCUGCACUAUUU | 2 |
| ABCC3 | GCACGCCGCUCUUCACUGU | 3 |
| ABCC3 | GAUCAUGGGUGUCAUCUAC | 4 |
| AKT1 | GACAAGGACGGGCACAUUA | 5 |
| AKT1 | GCUACUUCCUCCUCAAGAA | 6 |
| AKT1 | GACCGCCUCUGCUUUGUCA | 7 |
| AKT1 | GGCAGCACGUGUACGAGAA | 8 |
| ASCL1 | CAACUACUCCAACGACUUG | 9 |
| ASCL1 | CCAACAAGAAGAUGAGUAA | 10 |
| ASCL1 | GCCCGCAGCCUGUUUCUUU | 11 |
| ASCL1 | GCAGGAGCUUCUCGACUUC | 12 |
| BCL2 | GGGAGAUAGUGAUGAAGUA | 13 |
| BCL2 | GAAGUACAUCCAUUAUAAG | 14 |
| BCL2 | GGAGGAUUGUGGCCUUCUU | 15 |
| BCL2 | UCAUGUGUGUGGAGAGCGU | 16 |
| BCL2L1 | CCUACAAGCUUUCCCAGAA | 17 |
| BCL2L1 | GGAGGCAGGCGACGAGUUU | 18 |
| BCL2L1 | GAUUCAGGCUGCUUGGGAU | 19 |
| BCL2L1 | GAAAGGGCCAGGAACGCUU | 20 |
| BIRC5 | CAAAGGAAACCAACAAUAA | 21 |
| BIRC5 | GCAAAGGAAACCAACAAUA | 22 |
| BIRC5 | CACCGCAUCUCUACAUUCA | 23 |
| BIRC5 | CCACUGAGAACGAGCCAGA | 24 |
| BMI1 | GAACAGAUUGGAUCGGAAA | 25 |
| BMI1 | GAGAUGGACUGACAAAUGC | 26 |
| BMI1 | GGAUAUUGCCUACAUUUAU | 27 |
| BMI1 | GAACAACGAGAAUCAAGAU | 28 |
| BRAF | ACAGAGACCUCAAGAGUAA | 29 |
| BRAF | GAAUCGGGCUGGUUUCCAA | 30 |
| BRAF | CAACAACAGGGACCAGAUA | 31 |
| BRAF | GAGAUGAUCAAACUUAUAG | 32 |
| CD274 | UGAAAGGACUCACUUGGUA | 33 |
| CD274 | CAUAGUAGCUACAGACAGA | 34 |
| CD274 | AGACCUGGCUGCACUAAUU | 35 |
| CD274 | GGACCUAUAUGUGGUAGAG | 36 |
| CDCP1 | GCAAACCGCUCAUCUAUAA | 37 |
| CDCP1 | CAUCAAGUCUGGAGAAAGA | 38 |
| CDCP1 | CAACAUUACAGUUCUCAUA | 39 |
| CDCP1 | GAGAAUCACUUUGUCAUAG | 40 |
| CHUK | CAAAGAAGCUGACAAUACU | 41 |
| CHUK | CCAGAUACUUUCUUUACUA | 42 |
| CHUK | GAAGUUCGGUUUAGUAGCC | 43 |
| CHUK | AAAUAUGGCAUCUCCUUAA | 44 |
| CTLA4 | GAACCCAGAUUUAUGUAAU | 45 |
| CTLA4 | GAACCUCACUAUCCAAGGA | 46 |
| CTLA4 | AUAUAAAGUUGGAUGCGGA | 47 |
| CTLA4 | CUAGGAAGCUCCAGUUCGA | 48 |
| CTNNB1 | GCUGAAACAUGCAGUUGUA | 49 |
| CTNNB1 | GAUAAAGGCUACUGUUGGA | 50 |
| CTNNB1 | CCACUAAUGUCCAGCGUUU | 51 |
| CTNNB1 | ACAAGUAGCUGAUAUUGAU | 52 |
| CUX1 | GCAUAAGGUUCAGAGCCUA | 53 |
| CUX1 | GAGAUUGAUGCACUGAGUA | 54 |
| CUX1 | CCGACGAGAUUGAAAUGAU | 55 |
| CUX1 | CCGCAACGGUAUUGGCGAA | 56 |
| CXCR4 | GAAGCAUGACGGACAAGUA | 57 |
| CXCR4 | UAACUACACCGAGGAAAUG | 58 |
| CXCR4 | GAGUCUGAGUCUUCAAGUU | 59 |
| CXCR4 | CAAGCAAGGGUGUGAGUUU | 60 |
| DHODH | GGACGGACUUUAUAAGAUG | 61 |
| DHODH | GGACCAAGCUGUCAUUAAC | 62 |
| DHODH | GGCCAUAAAUUCCGAAAUC | 63 |
| DHODH | AGGAAUUGCUGCAGGAUUU | 64 |
| EGFR | CCGCAAAUUCCGAGACGAA | 65 |
| EGFR | CAAAGUGUGUAACGGAAUA | 66 |
| EGFR | GUAACAAGCUCACGCAGUU | 67 |
| EGFR | GAGGAAAUAUGUACUACGA | 68 |
| EHMT1 | CAAACAGCGUGGUCAAGUA | 69 |
| EHMT1 | CAAGAAAGGCCACUACGAA | 70 |
| EHMT1 | GGAAUUCUGUCUUCACAAG | 71 |
| EHMT1 | CAAAGCAGCUGUACUUCUC | 72 |
| ELK1 | GCCAGAAGUUCGUCUACAA | 73 |
| ELK1 | GCAAGAACAAGACCAACAU | 74 |
| ELK1 | ACGGGAUGGUGGUGAAUUC | 75 |
| ELK1 | UUUAAUGGGUUGGGAGUCU | 76 |
| ERBB2 | GGACGAAUUCUGCACAAUG | 77 |
| ERBB2 | GACGAAUUCUGCACAAUGG | 78 |
| ERBB2 | CUACAACACAGACACGUUU | 79 |
| ERBB2 | AGACGAAGCAUACGUGAUG | 80 |
| EZH2 | CAAAGAAUCUAGCAUCAUA | 81 |
| EZH2 | GAGGACGGCUUCCCAAUAA | 82 |
| EZH2 | GCUGAAGCCUCAAUGUUUA | 83 |
| EZH2 | GAAUGGAAACAGCGAAGGA | 84 |
| FGFR1 | AUUCAAACCUGACCACAGA | 85 |
| FGFR1 | CCACAGAAUUGGAGGCUAC | 86 |
| FGFR1 | CCGCACAUCCAGUGGCUAA | 87 |
| FGFR1 | ACGUGGAGUUCAUGUGUAA | 88 |
| FGFR2 | CCAAAUCUCUCAACCAGAA | 89 |
| FGFR2 | GAACAGUAUUCACCUAGUU | 90 |
| FGFR2 | GGCCAACACUGUCAAGUUU | 91 |
| FGFR2 | GUGAAGAUGUUGAAAGAUG | 92 |
| FGFR3 | UGUCGGACCUGGUGUCUGA | 93 |
| FGFR3 | GCAUCAAGCUGCGGCAUCA | 94 |
| FGFR3 | GGACGGCACACCCUACGUU | 95 |
| FGFR3 | UGCACAACCUCGACUACUA | 96 |
| FGFR4 | GCACUGGAGUCUCGUGAUG | 97 |
| FGFR4 | CCUCGAAUAGGCACAGUUA | 98 |
| FGFR4 | AUAACUACCUGCUAGAUGU | 99 |
| FGFR4 | GCAUUCGGCUGCGCCAUCA | 100 |
| FKBP4 | GCGGUGAAGGCUAUGCUAA | 101 |
| FKBP4 | GCUCUAUGCCAAUAUGUUU | 102 |
| FKBP4 | GAGAACAUUCCAUCGUGUA | 103 |
| FKBP4 | AGGGAGAAGAUCUGACGGA | 104 |
| FLT3 | GAAGGCAUCUACACCAUUA | 105 |
| FLT3 | GAAGGAGUCUGGAAUAGAA | 106 |
| FLT3 | GAAUUUAAGUCGUGUGUUC | 107 |
| FLT3 | GGAAUUCAUUUCACUCUGA | 108 |
| FOXM1 | CAACAGGAGUCUAAUCAAG | 109 |
| FOXM1 | GGACCACUUUCCCUACUUU | 110 |
| FOXM1 | GUAGUGGGCCCAACAAAUU | 111 |
| FOXM1 | GCUGGGAUCAAGAUUAUUA | 112 |
| GLI1 | GGAAAUGACUGGCAAUGCA | 113 |
| GLI1 | GCACUGGUCUGUCCACUCU | 114 |
| GLI1 | GUCCUCGACUUGAACAUUA | 115 |
| GLI1 | AGGCUCAGCUUGUGUGUAA | 116 |
| GRB7 | AGAAGUGCCUCAGAUAAUA | 117 |
| GRB7 | UAGUAAAGGUGUACAGUGA | 118 |
| GRB7 | UGCAGAAAGUGAAGCAUUA | 119 |
| GRB7 | GGAGAUAGCCGCUUCGUCU | 120 |
| IDO1 | AGAAAGAGUUGAGAAGUUA | 121 |
| IDO1 | GAAAUAUUGCUGUUCCUUA | 122 |
| IDO1 | GAACGGGACACUUUGCUAA | 123 |
| IDO1 | GGGCAAAGGUCAUGGAGAU | 124 |
| IGF1R | GGCCAGAAAUGGAGAAUAA | 125 |
| IGF1R | GCAGACACCUACAACAUCA | 126 |
| IGF1R | GGACUCAGUACGCCGUUUA | 127 |
| IGF1R | GUGGGAGGGUUGGUGAUUA | 128 |
| IGF2R | GAAUUGACCUGUACAAAGA | 129 |
| IGF2R | GAAAUUGAGUGGAUUACUG | 130 |
| IGF2R | GAAGUGGUUUCCAUCAGUA | 131 |
| IGF2R | GUGGAGAGAUGGCAUAAUU | 132 |
| IKBKB | GGAAGUACCUGAACCAGUU | 133 |
| IKBKB | CCAAUAAUCUUAACAGUGU | 134 |
| IKBKB | GGAUUCAGCUUCUCCUAAA | 135 |
| IKBKB | GUGGUGAGCUUAAUGAAUG | 136 |
| KRAS | CGAAUAUGAUCCAACAAUA | 137 |
| KRAS | UAAGGACUCUGAAGAUGUA | 138 |
| KRAS | GACAAAGUGUGUAAUUAUG | 139 |
| KRAS | GCUCAGGACUUAGCAAGAA | 140 |
| MAP2K1 | GCACAUGGAUGGAGGUUCU | 141 |
| MAP2K1 | GCAGAGAGAGCAGAUUUGA | 142 |
| MAP2K1 | GAGCAGAUUUGAAGCAACU | 143 |
| MAP2K1 | CCAGAAAGCUAAUUCAUCU | 144 |
| MAP2K2 | CAAAGACGAUGACUUCGAA | 145 |
| MAP2K2 | GAUCAGCAUUUGCAUGGAA | 146 |
| MAP2K2 | GGAAGCUGAUCCACCUUGA | 147 |
| MAP2K2 | GAAAGUCAGCAUCGCGGUU | 148 |
| MAP3K1 | GCUGAAAGGUCAACAGAUA | 149 |
| MAP3K1 | AGAAGAAGCUUUAGCAAUU | 150 |
| MAP3K1 | GAGCUCAACUCCAGUAUUG | 151 |
| MAP3K1 | GAACAGAUGUGUCCUAUUU | 152 |
| MAPK1 | CCAAAGCUCUGGACUUAUU | 153 |
| MAPK1 | AAACAGAUCUUUACAAGCU | 154 |
| MAPK1 | CAAGAGGAUUGAAGUAGAA | 155 |
| MAPK1 | GUACAGGGCUCCAGAAAUU | 156 |
| MAPK14 | CAAGGUCUCUGGAGGAAUU | 157 |
| MAPK14 | GUCAGAAGCUUACAGAUGA | 158 |
| MAPK14 | GUCCAUCAUUCAUGCGAAA | 159 |
| MAPK14 | CUACAGAGAACUGCGGUUA | 160 |
| MAPK3 | GACCGGAUGUUAACCUUUA | 161 |
| MAPK3 | AGACUGACCUGUACAAGUU | 162 |
| MAPK3 | GCUACACGCAGUUGCAGUA | 163 |
| MAPK3 | CCGGCUUCCUGACGGAGUA | 164 |
| MCL1 | CGAAGGAAGUAUCGAAUUU | 165 |
| MCL1 | AGAACGAAUUGAUGUGUAA | 166 |
| MCL1 | GGACCAACUACAAAUUAAU | 167 |
| MCL1 | GCUACGUAGUUCGGGCAAA | 168 |
| MDM2 | GCCAGUAUAUUAUGACUAA | 169 |
| MDM2 | GAUGAGAAGCAACAACAUA | 170 |
| MDM2 | CCCUAGGAAUUUAGACAAC | 171 |
| MDM2 | AAAGUCUGUUGGUGCACAA | 172 |
| MDM4 | GCAGUUAGGUGUUGGAAUA | 173 |
| MDM4 | UGAUACCGAUGUAGAGGUU | 174 |
| MDM4 | GCAUAAUGGUAGUACGAAC | 175 |
| MDM4 | CCACGAGACGGGAACAUUA | 176 |
| ME2 | GAAGAAGCAUAUACACUUA | 177 |
| ME2 | UGAAAGGCCUGUAAUAUUU | 178 |
| ME2 | GAACAUGGCGGAGUGAAUA | 179 |
| ME2 | AUUAGUUAAGGGACGGAAA | 180 |
| ME3 | CAACAAUGCUGAAUUCUUG | 181 |
| ME3 | ACAAAUACCGUAACAAGUA | 182 |
| ME3 | GGAGCCACCUGAACCAUGA | 183 |
| ME3 | CUAAAGGGCUCAUUGUCAA | 184 |
| MSI1 | GGACUCAGUUGGCAGACUA | 185 |
| MSI1 | AGGAAGGGCUGCGCGAAUA | 186 |
| MSI1 | AUAAAGUGCUGGCGCAAUC | 187 |
| MSI1 | GAGUCAUGCCCUACGGAAU | 188 |
| MSI2 | CAAUGCUGAUGUUUGAUAA | 189 |
| MSI2 | CCAGAUAGCCUUAGAGACU | 190 |
| MSI2 | GAGUUAGAUUCCAAGACGA | 191 |
| MSI2 | CCAACUUCGUGGCGACCUA | 192 |
| MTOR | GAGAAGAAAUGGAAGAAAU | 193 |
| MTOR | CCAAAGUGCUGCAGUACUA | 194 |
| MTOR | GGUCUGAACUGAAUGAAGA | 195 |
| MTOR | AUAAAGUUCUGGUGCGACA | 196 |
| MYC | AACGUUAGCUUCACCAACA | 197 |
| MYC | GGAACUAUGACCUCGACUA | 198 |
| MYC | GAACACACAACGUCUUGGA | 199 |
| MYC | CUACCAGGCUGCGCGCAAA | 200 |
| MYCN | GAACCCAGACCUCGAGUUU | 201 |
| MYCN | GAAGAAAUCGACGUGGUCA | 202 |
| MYCN | GGACAGUGAGCGUCGCAGA | 203 |
| MYCN | GAGAGGACACCCUGAGCGA | 204 |
| NR4A1 | GCACCUUCAUGGACGGCUA | 205 |
| NR4A1 | GCAUUAUGGUGUCCGCACA | 206 |
| NR4A1 | UGAAGGAAGUUGUCCGAAC | 207 |
| NR4A1 | CUGCAGAACCGCAUCGCCA | 208 |
| NR4A2 | CCACGUGACUUUCAACAAU | 209 |
| NR4A2 | ACAUUCAGAUGCACAACUA | 210 |
| NR4A2 | GGACAAGCGUCGCCGGAAU | 211 |
| NR4A2 | CCACCUUGCUUGUACCAAA | 212 |
| NUAK1 | GCAGGACUCUUAUCUUAAA | 213 |
| NUAK1 | AAACUUGACUGCACAUUAA | 214 |
| NUAK1 | UGAAUAAGGUGUUGAAAGA | 215 |
| NUAK1 | GAGAUUCACUGUCAGCUUU | 216 |
| ONECUT2 | GAACAAACGCCCGUCAAAG | 217 |
| ONECUT2 | GAACACUCUUCGCCAUCUU | 218 |
| ONECUT2 | ACAAAGACAGGAACAAUUC | 219 |
| ONECUT2 | GGUGUUCACUGACCUCCAA | 220 |
| PAK1 | CAUCAAAUAUCACUAAGUC | 221 |
| PAK1 | CAACAAAGAACAAUCACUA | 222 |
| PAK1 | AGAAAUACCAGCACUAUGA | 223 |
| PAK1 | GUGAAAUGCUCUCGGCUAU | 224 |
| PDCD1LG2 | GGACAGUACCAAUGCAUAA | 225 |
| PDCD1LG2 | GAACUUACUUUGGCCAGCA | 226 |
| PDCD1LG2 | CCUAAGGAACUGUACAUAA | 227 |
| PDCD1LG2 | UGUGAACCUUGGAGCAAUA | 228 |
| PDGFRA | CAUCAGAGCUGGAUCUAGA | 229 |
| PDGFRA | GGCCUUACUUUAUUGGAUU | 230 |
| PDGFRA | GAGCUUCACCUAUCAAGUU | 231 |
| PDGFRA | CCUCUAGGAAUGACGGAUU | 232 |
| PDGFRB | GAAAGGAGACGUCAAAUAU | 233 |
| PDGFRB | GGAAUGAGGUGGUCAACUU | 234 |
| PDGFRB | CAACGAGUCUCCAGUGCUA | 235 |
| PDGFRB | UGACAACGACUAUAUCAUC | 236 |
| PFKFB3 | GGACCUAACCCGCUCAUGA | 237 |
| PFKFB3 | GAGGAUCAGUUGCUAUGAA | 238 |
| PFKFB3 | AAAGCUACCUGGCGAAAGA | 239 |
| PFKFB3 | ACAAGUACUAUUACCGCUA | 240 |
| PFKFB4 | GAGCGACCAUCUUUAAUUU | 241 |
| PFKFB4 | GAAAUGACCUACGAGGAAA | 242 |
| PFKFB4 | CAUCGUAUAUUACCUCAUG | 243 |
| PFKFB4 | GGGACAGGCCUCAGAACGU | 244 |
| PIK3CA | GGACAACUGUUUCAUAUAG | 245 |
| PIK3CA | GCCAGUACCUCAUGGAUUA | 246 |
| PIK3CA | CUUGAAGAGUGUCGAAUUA | 247 |
| PIK3CA | GCUUGAAGAGUGUCGAAUU | 248 |
| PIKFYVE | GAAUGGAGUUUCAGGAUCA | 249 |
| PIKFYVE | GGAAAUCUCCUGCUCGAAA | 250 |
| PIKFYVE | UGAAGAAGGUGACGAUAAU | 251 |
| PIKFYVE | GGACUCUGCUAAUGAUUUG | 252 |
| PTPN11 | GAACAUCACGGGCAAUUAA | 253 |
| PTPN11 | GAACACUGGUGAUUACUAU | 254 |
| PTPN11 | GAAACCAAGUGCAACAAUU | 255 |
| PTPN11 | GAAGCACAGUACCGAUUUA | 256 |
| PVR | CAACACAACUUUAAUCUGC | 257 |
| PVR | GGAUCGGGAUUUAUUUCUA | 258 |
| PVR | CCAAACGGCUGGAAUUCGU | 259 |
| PVR | UAAUGGGCAUGUCUCCUAU | 260 |
| RORC | UAGAACAGCUGCAGUACAA | 261 |
| RORC | UCACCGAGGCCAUUCAGUA | 262 |
| RORC | GAACAGCUGCAGUACAAUC | 263 |
| RORC | CCUCAUGCCACCUUGAAUA | 264 |
| SKP2 | CAUCUAGACUUAAGUGAUA | 265 |
| SKP2 | CUAAAGGUCUCUGGUGUUU | 266 |
| SKP2 | GGUAUCGCCUAGCGUCUGA | 267 |
| SKP2 | UGUCAAUACUCUCGCAAAA | 268 |
| SOAT1 | GAAUAUGCCUUGGCUGUUU | 269 |
| SOAT1 | GAACGUGCCUCGGGUACUA | 270 |
| SOAT1 | GCACACUUGUAGUAGAUUA | 271 |
| SOAT1 | GAUUGGAGUUCUAGGUUUU | 272 |
| SREBF2 | GAGCGGAGCUGGUCUGUGA | 273 |
| SREBF2 | GAAGAGAGCUGUGAAUUCU | 274 |
| SREBF2 | GCACAAGUCUGGCGUUCUG | 275 |
| SREBF2 | AAACUCAGCUGCAACAACA | 276 |
| STAT3 | GGAGAAGCAUCGUGAGUGA | 277 |
| STAT3 | CCACUUUGGUGUUUCAUAA | 278 |
| STAT3 | UCAGGUUGCUGGUCAAAUU | 279 |
| STAT3 | CGUUAUAUAGGAACCGUAA | 280 |
| SULT2B1 | CCAACACCAUGUCCAACUA | 281 |
| SULT2B1 | GGGCAAAGACAACUUCCUA | 282 |
| SULT2B1 | GGGACUUCCUCAAAGGCGA | 283 |
| SULT2B1 | CAUAAUAAACACGUCGAUU | 284 |
| TEAD2 | CGAAGGAAAUCAAGGGAAA | 285 |
| TEAD2 | GCAGUUGAUUCUUACCAGA | 286 |
| TEAD2 | GGAAUGAACUGAUCGCCCG | 287 |
| TEAD2 | GGAAGACCCGAACUCGAAA | 288 |
| TERT | GGUAUGCCGUGGUCCAGAA | 289 |
| TERT | CCACGUCUCUACCUUGACA | 290 |
| TERT | UCACGGAGACCACGUUUCA | 291 |
| TERT | GCGUGGUGAACUUGCGGAA | 292 |
| TMPRSS2 | GCAAUGUCGAUAUCUAUAA | 293 |
| TMPRSS2 | GCUAUUGGACCUUACUAUG | 294 |
| TMPRSS2 | ACGGGAAUGUGAUGGUAUU | 295 |
| TMPRSS2 | CGGACUGGAUUUAUCGACA | 296 |
| TSPAN3 | AGAGACAGCUGCAUUGUUG | 297 |
| TSPAN3 | GUAGAGAUCCUGCUUACGA | 298 |
| TSPAN3 | CCUAAGAUUUUAAGUACGA | 299 |
| TSPAN3 | UGUAGGAUCAUAUGGCGUA | 300 |
| UBB | GCCGUACUCUUUCUGACUA | 301 |
| UBB | GUAUGCAGAUCUUCGUGAA | 302 |
| UBB | GACCAUCACUCUGGAGGUG | 303 |
| UBB | CCCAGUGACACCAUCGAAA | 304 |
| UBC | GUGAAGACCCUGACUGGUA | 305 |
| UBC | AAGCAAAGAUCCAGGACAA | 306 |
| UBC | GAAGAUGGACGCACCCUGU | 307 |
| UBC | GUAAGACCAUCACUCUCGA | 308 |
| ULK1 | CCUAAAACGUGUCUUAUUU | 309 |
| ULK1 | ACUUGUAGGUGUUUAAGAA | 310 |
| ULK1 | GGUUAGCCCUGCCUGAAUC | 311 |
| ULK1 | UGUAGGUGUUUAAGAAUUG | 312 |
| YAP1 | GGUCAGAGAUACUUCUUAA | 313 |
| YAP1 | CCACCAAGCUAGAUAAAGA | 314 |
| YAP1 | GAACAUAGAAGGAGAGGAG | 315 |
| YAP1 | GCACCUAUCACUCUCGAGA | 316 |
| YY1 | GGAUAACUCGGCCAUGAGA | 317 |
| YY1 | CAAGAAGAGUUACCUCAGC | 318 |
| YY1 | GAACUCACCUCCUGAUUAU | 319 |
| YY1 | GCUUAGUAAUGCUACGUGU | 320 |
| EGFR | CCUUAGCAGUCUUAUCUAA | 321 |
| BIRC5 | GGACCACCGCAUCUCUACA | 322 |
| UBB | GGCCAAGAUCCAGGAUAAA | 323 |
| UBB | GGCCAAGAUCCAGGAUAAG | 324 |
| UBC | GGCAAAGAUCCAAGAUAAG | 325 |
| UBB | AAATGUGAAGGCCAAGAUC | 326 |
| UBB | AATGUGAAGGCCAAGAUCC | 327 |
| UBB | ATGUGAAGGCCAAGAUCCA | 328 |
| UBB | TGUGAAGGCCAAGAUCCAA | 329 |
| UBB | GUGAAGGCCAAGAUCCAAG | 330 |
| UBB | UGAAGGCCAAGAUCCAAGA | 331 |
| UBB | GAAGGCCAAGAUCCAAGAU | 332 |
| UBB | AAGGCCAAGAUCCAAGAUA | 333 |
| UBB | AGGCCAAGAUCCAAGAUAA | 334 |
| UBB | GGCCAAGAUCCAAGAUAAA | 335 |
| UBB | GCCAAGAUCCAAGAUAAAG | 336 |
| UBB | CCAAGAUCCAAGAUAAAGA | 337 |
| UBB | CAAGAUCCAAGAUAAAGAA | 338 |
| UBB | AAGAUCCAAGAUAAAGAAG | 339 |
| UBB | AGAUCCAAGAUAAAGAAGG | 340 |
| UBB | GAUCCAAGAUAAAGAAGGC | 341 |
| UBB | AUCCAAGAUAAAGAAGGCA | 342 |
| UBB | UCCAAGAUAAAGAAGGCAT | 343 |
| UBB | CCAAGAUAAAGAAGGCATC | 344 |
| UBB/UBC | CAAGACCAUCACCCUUGAG | 345 |
| UBB/UBC | UGCAGAUCUUCGUGAAGAC | 346 |
| UBB/UBC | AGCCCAGUGACACCAUCGA | 347 |
| UBB/UBC | GACUACAACAUCCAGAAAG | 348 |
| UBB/UBC | CUACAACAUCCAGAAAGAG | 349 |
| UBB | AGUGACACCAUCGAAAAUG | 350 |
| UBC | AGGCAAAGAUCCAAGAUAA | 351 |
| UBC | GGCAAAGAUCCAAGACAAG | 352 |
| UBC | CAAGGCAAAGAUCCAAGAC | 353 |
| UBC | AGGCAAAGAUCCAAGACAA | 354 |
| UBB | CAGGAUAAGGAAGGCAUUC | 355 |
| UBC | CAGGACAAGGAAGGCAUUC | 356 |
| UBB | GGCAAGCAGCUGGAAGAUG | 357 |
| UBC | GGAAAGCAGCUGGAAGAUG | 358 |
| UBB | GACUACAACAUCCAGAAGG | 359 |
| UBB | UGACUACAACAUCCAGAAG | 360 |
| UBB/UBC | UGACUACAACAUCCAGAAA | 361 |
| NR4A1 | UGCUGUGUGUGGGGACAAC | 362 |
| NR4A1 | GGGCUGCAAGGGCUUCUUC | 363 |
| NR4A1 | GCGCACAGUGCAGAAAAAC | 364 |
| ADORA2A | CCUCACGCAGAGCUCCAUC | 365 |
| ADORA2A | CAUGGUGUACUUCAACUUC | 366 |
| ADORA2A | GUGUACUUCAACUUCUUUG | 367 |
| MAP2K1 | AAUCCGGAACCAGAUCAUA | 368 |
| MAP2K1 | GUACAUCGUGGGCUUCUAU | 369 |
| MAP2K1 | CAAGCCCUCCAACAUCCUA | 370 |
| MAP2K1/2 | UCGACUCCAUGGCCAACUC | 371 |
| MAP2K1/2 | CAUGGCCAACUCCUUCGUG | 372 |
| MAPK3 | UGAGCAAUGACCAUAUCUG | 373 |
| MAPK3 | CCAAGGGCUAUACCAAGUC | 374 |
| MAPK3 | GUCUGUGGGCUGCAUUCUG | 375 |
| MAPK3 | GGAGGACCUGAAUUGUAUC | 376 |
| MAPK11/14 | CCGGCAGGAGCUGAACAAG | 377 |
| MAPK11/14 | AACUGGAUGCAUUACAACC | 378 |
| MAPK14 | CAACUGGAUGCAUUACAAC | 379 |
| MDM2 | GACCGAGUCUUGCUCUGUU | 380 |
| MDM2 | UACCCAGGCUGGAGUGCAG | 381 |
| PFKFB3/4 | GACCUACAUCUCCAAGAAG | 382 |
| PFKFB3/4 | AGAAUGUGCUGGUCAUCUG | 383 |
| RICTOR | UCAACGAGCUCACAUAUGA | 384 |
| RICTOR | UGACCGAUCUGGACCCAUA | 385 |
| RICTOR | GUACUUGGGCUCAUAGCUA | 386 |
| RICTOR | GCAGAUGAGUCUUACGGAA | 387 |
| TOX | CCACAUGGCCAGCUGACUA | 388 |
| TOX | CAACCCGACUAUCAGACUA | 389 |
| TOX | GAAUGAAUCCUCACCUAAC | 390 |
| TOX | GCAACAAGUUUGACGGUGA | 391 |
| TOX2 | GGAAGUGCAUUUCAAGAUC | 392 |
| TOX2 | CGAGAACAACGAAGACUAU | 393 |
| TOX2 | CAAGAGCACUCAGGCAAAC | 394 |
| TOX2 | AAAGAGACCUUCAGCCGAC | 395 |
| ADORA2A | GAACGUCACCAACUACUUU | 396 |
| ADORA2A | CAUGCUGGGUGUCUAUUUG | 397 |
| ADORA2A | CAACUGCGGUCAGCCAAAG | 398 |
| ADORA2A | CCAAGUGGCCUGUCUCUUU | 399 |
| ADORA2B | UGAGCUACAUGGUAUAUUU | 400 |
| ADORA2B | GGGAUGGAACCACGAAUGA | 401 |
| ADORA2B | GAUGGAACCACGAAUGAAA | 402 |
| ADORA2B | GAACCGAGACUUCCGCUAC | 403 |
| CBLB | GACCAUACCUCAUAACAAG | 404 |
| CBLB | UGAAAGACCUCCACCAAUC | 405 |
| CBLB | GAUGAAGGCUCCAGGUGUU | 406 |
| CBLB | UAUCAGCAUUUACGACUUA | 407 |
| NR4A3 | GAAGUUGUCCGUACAGAUA | 408 |
| NR4A3 | CGGAAUACACCACGGAGAU | 409 |
| NR4A3 | CCUCCAAUCUGCAUGAUGA | 410 |
| NR4A3 | GCACUGAGAUCACGGCUAC | 411 |
| VHL | CCGUAUGGCUCAACUUCGA | 412 |
| VHL | AGGCAGGCGUCGAAGAGUA | 413 |
| VHL | GCUCUACGAAGAUCUGGAA | 414 |
| VHL | GGAGCGCAUUGCACAUCAA | 415 |
| BATF | GUACAGCGCCCACGCAUUC | 416 |
| BATF | GAAACAGAACGCGGCUCUA | 417 |
| BATF | GAACGCGGCUCUACGCAAG | 418 |
| BATF | AGAGUUCAGAGGAGGGAGA | 419 |
| PTPN2 | GAAACAGGAUUCAGUGUGA | 420 |
| PTPN2 | ACAAAGGAGUUACAUCUUA | 421 |
| PTPN2 | AAAGGGAGAUUCUAGUAUA | 422 |
| PTPN2 | AAACAGAAAUCGAAACAGA | 423 |
| TIGIT | GAAGAAAGCCCUCAGAAUC | 424 |
| TIGIT | GUGCCGAGCUGCAUGACUA | 425 |
| TIGIT | GCACAGCAGUCAUCGUGGU | 426 |
| TIGIT | UCGCUGACCGUGAACGAUA | 427 |
| LAG3 | CGACUUUACCCUUCGACUA | 428 |
| LAG3 | CUACAGAGAUGGCUUCAAC | 429 |
| LAG3 | CAACUCCCUUGACAGUGUA | 430 |
| LAG3 | UGAGGUGACUCCAGUAUCU | 431 |
| HAVCR2 | AAAGGGAUGUGAAUUAUUG | 432 |
| HAVCR2 | GAAAACAUCUAUACCAUUG | 433 |
| HAVCR2 | GAGCGGAGGUCGGUCAGAA | 434 |
| HAVCR2 | CAGCAACCCUCACAACCUU | 435 |
| PDCD1 | GGGCGUGACUUCCACAUGA | 436 |
| PDCD1 | CGGAGAGCUUCGUGCUAAA | 437 |
| PDCD1 | ACAAUAGGAGCCAGGCGCA | 438 |
| PDCD1 | GCAAUGACAGCGGCACCUA | 439 |
| CTLA4 | GAACCCAGAUUUAUGUAAU | 440 |
| CTLA4 | GAACCUCACUAUCCAAGGA | 441 |
| CTLA4 | AUAUAAAGUUGGAUGCGGA | 442 |
| CTLA4 | CUAGGAAGCUCCAGUUCGA | 443 |
| CD44 | GCAGAUCGAUUUGAAUAUA | 444 |
| CD44 | GCGCAGAUCGAUUUGAAUA | 445 |
| CD44 | CUGUUAUAUCAGAGGAGUA | 446 |
| CD44 | CCUAAACUUAUGUGCUUAA | 447 |
| EPCAM | GCCGUAAACUGCUUUGUGA | 448 |
| EPCAM | AGGAGAUCACAACGCGUUA | 449 |
| EPCAM | CCAAAAUUUAUCACGAGUA | 450 |
| EPCAM | AGUAAAAGUUUGCGGACUG | 451 |
| TACSTD2 | GAAAGGAACCGAGCUUGUA | 452 |
| TACSTD2 | UCUAUUACCUGGACGAGAU | 453 |
| TACSTD2 | GCAGAACACGUCUCAGAAG | 454 |
| TACSTD2 | CAAGGGCGAGUCUCUAUUC | 455 |
| NT5E | GAACCUGGCUGCUGUAUUG | 456 |
| NT5E | GGAAGUCACUGCCAUGGAA | 457 |
| NT5E | UGAAAUCACUGCAUUACAA | 458 |
| NT5E | GGACUUUAUUUGCCAUAUA | 459 |
| CD274 | UGAAAGGACUCACUUGGUA | 460 |
| CD274 | CAUAGUAGCUACAGACAGA | 461 |
| CD274 | AGACCUGGCUGCACUAAUU | 462 |
| CD274 | GGACCUAUAUGUGGUAGAG | 463 |
| EGFR | UACGAAUAUUAAACACUUCAA | 464 |
| EGFR | AUAGGUAUUGGUGAAUUUAAA | 465 |
| EGFR | CAGGAACUGGAUAUUCUGAAA | 466 |
| EGFR | UGCCGCAAAUUCCGAGACGAA | 467 |
| BIRC5 | AAGAAGCAGUUUGAAGAAUUA | 468 |
| BIRC5 | CCGCAUCUCUACAUUCAAGAA | 469 |
| BIRC5 | CUCGGCUGUUCCUGAGAAAUA | 470 |
| BIRC5 | CUGGCGUAAGAUGAUGGAUUU | 471 |
| BIRC5 | AAGCAUUCGUCCGGUUGCGCU | 472 |
| BIRC5 | UGCACCACUUCCAGGGUUUAU | 473 |
| BIRC5 | GGACCACCGCAUCUCUACA | 474 |
| PIKFYVE | CAGAGAUGAGUAUGCGCUGUA | 475 |
| PIKFYVE | AUCCUGGUUUAAAGACAUAAA | 476 |
| PIKFYVE | CAACGUGAACUUCCAUAUCAA | 478 |
| PIKFYVE | CUGGUUGAGAACGCAUCCCAA | 479 |
| PIKFYVE | UUGCCGUUACCCAGUAACAUA | 480 |
| PIKFYVE | ACCCAGUAACAUAAUAUUUCA | 481 |
| PIKFYVE | UUGCCGUUACCCAGUAACAUA | 482 |
| PIKFYVE | ACCCAGUAACAUAAUAUUUCA | 483 |
| UBC | GAGGUUGAUCUUUGCCGGAAA | 484 |
| UBC | GAGGUUGATCUUUGCUGGGAAA | 485 |
| UBC | AACGUCAAAGCAAAGAUCCAA | 486 |
| UBC | AUCGCUGUGAUCGTCACUUGA | 487 |
| VHL | AAGGAGGUUUGUAUAAGUAAU | 488 |
| VHL | CAGGAGCGCAUUGCACAUCAA | 489 |
| VHL | UUCAGUGGGAAUUGCAGCAUA | 490 |
| VHL | CUGAUGAGUCUUGAUCUAGAU | 491 |
| NR4A1 | CTCCAGUGGCUCUGACUACUA | 492 |
| NR4A1 | CACAGGAGAGUUUGACACCUU | 493 |
| NR4A1 | CAGCACCUUCAUGGACGGCUA | 494 |
| NR4A1 | CAGCAUUAUGGUGUCCGCACA | 495 |
| STAT3 | CAGGGUGUCAGAUCACAUGGGCU | 496 |
| NR4A1 | CUCCAGUGGCUCUGACUACUA | 497 |
| NR4A1 | CAGCAUUAUGGUGUCCGCACAUGUG | 498 |
| UBB | GGCCAAGAUCCAAGAUAAAGAAGGC | 499 |
| ERBB2ERBB3 | CACCAUUGAUGUCUACAUG | 500 |
| OC1OC2 | CAGCAACUUCUUCAUGAAC | 501 |
| OC2OC3 | CAUCUUCAAGGAGAACAAA | 502 |
| GRB2GRB7 | UGCCAGGAGGCGGCAGCUC | 503 |
| IKBKAIKBKA | UGCAGUAUCUGGCCCCAGA | 504 |
| CDK7CDK20 | CUACAUAGGGAUCUGAAAC | 505 |
| ME2ME3 | UUUGCCAGUGGCAGUCCAU | 506 |
| MSI1MSI2 | GAAUGUAAGAAAGCUCAGC | 507 |
| ADORA2AADORA1 | GUCUACUUCAACUUCUUUG | 508 |
| ADORA2AADORA2B | CCUCACGCAGAGCUCCAUC | 509 |
| ADORA2AADORA2B | CCUCACCCAGAGCUCCAUC | 510 |
| ADORA2AADORA2B | CAUGGUGUACUUCAACUUC | 511 |
| ADORA2AADORA2B | CAUGGUCUACUUCAACUUC | 512 |
| ADORA2AADORA2B | GUGUACUUCAACUUCUUUG | 513 |
| ADORA2AADORA2B | GUCUACUUCAACUUCUUUG | 514 |
| MAP2K1 (MEK1) | AAUCCGGAACCAGAUCAUA | 515 |
| MAP2K2 (MEK2) | | |
| MAP2K1 (MEK1) | CAUCCGGAACCAGAUCAUC | 516 |
| MAP2K2 (MEK2) | | |
| MAP2K1 (MEK1) | GUACAUCGUGGGCUUCUAU | 517 |
| MAP2K2 (MEK2) | | |
| MAP2K1 (MEK1) | GUACAUCGUGGGCUUCUAC | 518 |
| MAP2K2 (MEK2) | | |
| MAP2K1 (MEK1) | CAAGCCCUCCAACAUCCUA | 519 |
| MAP2K2 (MEK2) | | |
| MAP2K1 (MEK1) | GAAGCCCUCCAACAUCCUC | 520 |
| MAP2K2 (MEK2) | | |
| MAP2K1 (MEK1) | GAUCAAGCUCUGUGACUUU | 521 |
| MAP2K2 (MEK2) | | |
| MAP2K1 (MEK1) | GAUCAAGCUGUGUGACUUC | 522 |
| MAP2K2 (MEK2) | | |
| MAP2K1 (MEK1) | UCGACUCCAUGGCCAACUC | 523 |
| MAP2K2 (MEK2) | | |
| MAP2K1 (MEK1) | CAUGGCCAACUCCUUCGUG | 524 |
| MAP2K2 (MEK2) | | |
| MAPK3 (ERK1) | UGAGCAAUGACCAUAUCUG | 525 |
| MAPK1 (ERK2) | | |
| MAPK3 (ERK1) | UCAGCAAUGACCAUAUCUG | 526 |
| MAPK1 (ERK2) | | |
| MAPK3 (ERK1) | CUACUUCCUCUACCAGAUC | 527 |
| MAPK1 (ERK2) | | |
| MAPK3 (ERK1) | CUAUUUUCUCUACCAGAUC | 528 |
| MAPK1 (ERK2) | | |
| MAPK3 (ERK1) | CCAAGGGCUAUACCAAGUC | 529 |
| MAPK1 (ERK2) | | |
| MAPK3 (ERK1) | CCAAGGGCUACACCAAGUC | 530 |
| MAPK1 (ERK2) | | |
| MAPK3 (ERK1) | GUCUGUGGGCUGCAUUCUG | 531 |
| MAPK1 (ERK2) | | |
| MAPK3 (ERK1) | GUCUGUAGGCUGCAUUCUG | 532 |
| MAPK1 (ERK2) | | |
| MAPK3 (ERK1) | CUCUAACCGGCCCAUCUUC | 533 |
| MAPK1 (ERK2) | | |
| MAPK3 (ERK1) | CUCUAACAGGCCCAUCUUC | 534 |
| MAPK1 (ERK2) | | |
| MAPK3 (ERK1) | GGAGGACCUGAAUUGUAUC | 535 |
| MAPK1 (ERK2) | | |
| MAPK3 (ERK1) | AGAAGACCUGAAUUGUAUA | 536 |
| MAPK1 (ERK2) | | |
| HIF1HIF2 | GAAAGCCUUGGAUGGUUUU | 537 |
| TOXTOX2 | UACUCAGGCCGCCAUCAAG | 538 |
| PFKFB3PFKFB4 | AGACCUACAUCUCCAAGAA | 539 |
| PLK1PLK4 | GUGGGACUCCUAAUUACAU | 540 |
| CDK11ACDK11B | CAAGAAAACAGAUGAAAUU | 541 |
| CDK4CDK6 | GUUUCGUCGAAAGCCUCUC | 542 |
| PARP1PARP2 | CCAAGAGUGCCAACUACUG | 543 |
| NR4A1NR4A2 | AGCGCACAGUGCAGAAAAA | 544 |
| NR4A1NR4A2 | UGCUGUGUGUGGGGACAAC | 545 |
| NR4A1NR4A2 | CGCUGUGUGUGGGGACAAC | 546 |
| NR4A1NR4A2 | AUGUGAGGGCUGCUGCAAG | 547 |
| NR4A1NR4A2 | CUGUGAGGGCUGCUGCAAA | 548 |
| NR4A1NR4A2 | CUGCAAGGGCUUCUUCAAG | 549 |
| NR4A1NR4A2 | CUGCAAAGGCUUCUUUAAG | 550 |
| NR4A1NR4A2 | GGGCUGCAAGGGCUUCUUC | 551 |
| NR4A1NR4A2 | GGGCUGCAAAGGCUUCUUU | 552 |
| NR4A1NR4A2 | GCGCACAGUGCAGAAAAAC | 553 |
| NR4A1NR4A2 | GCGCACAGUGCAAAAAAAC | 554 |
| NR4A1, | UGCUGUGUGUGGGGACAAC | 555 |
| NR4A2, NR4A31 | | |
| NR4A1, | CGCUGUGUGUGGGGACAAC | 556 |
| NR4A2, NR4A32 | | |
| NR4A1, | UGCCGUGUGCGGGGACAAC | 557 |
| NR4A2, NR4A33 | | |
| NR4A1, | CUGCAAGGGCUUCUUCAAG | 558 |
| NR4A2, NR4A31 | | |
| NR4A1, | CUGCAAAGGCUUCUUUAAG | 559 |
| NR4A2, NR4A32 | | |
| NR4A1, | CUGCAAGGGCUUUUUCAAG | 560 |
| NR4A2, NR4A33 | | |
| NR4A1, | GGGCUGCAAGGGCUUCUUC | 561 |
| NR4A2, NR4A31 | | |
| NR4A1, | GGGCUGCAAAGGCUUCUUU | 562 |
| NR4A2, NR4A32 | | |
| NR4A1, | GGGCUGCAAGGGCUUUUUC | 563 |
| NR4A2, NR4A33 | | |
| NR4A1, | GCGCACAGUGCAGAAAAAC | 564 |
| NR4A2, NR4A31 | | |
| NR4A1, | GCGCACAGUGCAAAAAAAC | 565 |
| NR4A2, NR4A32 | | |
| NR4A1, | GAGAACAGUGCAGAAAAAU | 566 |
| NR4A2, NR4A33 | | |
| NR4A1, | ACUGCCCUGUGGACAAGAG | 567 |
| NR4A2, NR4A31 | | |
| NR4A1, | ACUGCCCAGUGGACAAGCG | 568 |
| NR4A2, NR4A32 | | |
| NR4A1, | ACUGCCCAGUAGACAAGAG | 569 |
| NR4A2, NR4A33 | | |
| ME1ME2 | UUCAAUGAUGAUAUUCAAG | 570 |
| KRASNRAS | AUCCAACAAUAGAGGAUUC | 571 |
| KRASHRAS | UCAUUUUGUGGACGAAUAU | 572 |
| BRAFARAF | GUGGUGUGAGGGCUCCAGC | 573 |
| BRAFRAF1 | CUUACAACACACAACUUUG | 574 |
| BCL2STAT3 | AAUUGAGGUGCUUCCUUAAUUGUga | 575 |
| BCL2STAT3 | UCACAAUCAGGGAAGCAUCACAAUU | 576 |
| | GG | |
| BCL2STAT3 | AAUUGAGGUGCUUCCUUAAUU | 577 |
| BCL2STAT3 | UCAGGGAAGCAUCACAAUUGG | 578 |
| BCL2MYC | AUUUGAGGCAGUUUACAUUUCGGtg | 579 |
| BCL2MYC | CACCGAAAUGUUCACUUCCUCAAGU | 580 |
| | UC | |
| BCL2MYC | AUUUGAGGCAGUUUACAUUUC | 581 |
| BCL2MYC | AAUGUUCACUUCCUCAAGUUC | 582 |
| BCL2SYK | UUUCCAUUUGUCUUUGAUCUUUGtt | 583 |
| BCL2SYK | AACAAAGAUCACAUAUAAAUGGAAG | 584 |
| | GC | |
| BCL2SYK | UUUCCAUUUGUCUUUGAUCUU | 585 |
| BCL2SYK | GAUCACAUAUAAAUGGAAGGC | 586 |
| BCL2CCNE2 | UUCCUCAAGUUUGGAAGCAGCCUtg | 587 |
| BCL2CCNE2 | CAAGGCUGAUUCUAAACUGGAAGAA | 588 |
| | AA | |
| CCNE2CCND1 | ACCAUCAGUGACGUAAGCAAAAGaa | 589 |
| CCNE2CCND1 | UUCUUUUGCUUAAGUCAGAGAUGGA | 590 |
| | AG | |
| CCND1EGFR | AUUCCAAUGCCAUCCACUUUCAGtc | 591 |
| CCND1EGFR | GACUGAAAGUGCUUGGAAAUGGAAU | 592 |
| | GG | |
| BIRC5CCND2 | UCCAUUUACAGACUGACACUUUCca | 593 |
| BIRC5CCND2 | UGGAAAGUUACAGACUGUAAAUAGA | 594 |
| | GU | |

In some embodiments, the methods described herein relate to treating a subject having or diagnosed as having cancer with a composition as described herein. Subjects having cancer can be identified by a physician using current methods of diagnosing cancer.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

In further embodiments, administration of a dual targeting siRNA agent is administered in combination an additional therapeutic agent. The dual targeting siRNA agent and an additional therapeutic agent can be administered in combination in the same composition, e.g., parenterally, or the additional therapeutic agent can be administered as part of a separate composition or by another method described herein.

The following non-limiting examples illustrate embodiments of the invention in operation. Having read the specification claims and Figures in their entirety, one skilled in the art will reasonably appreciate that numerous modifications and substitutions of the embodiments are possible and can be carried out without requiring undue experimentation. Such modifications and substitutions constitute part of the present invention.

### EXAMPLES

### Example 1: Identifying Target Gene with Multiple Target Regions

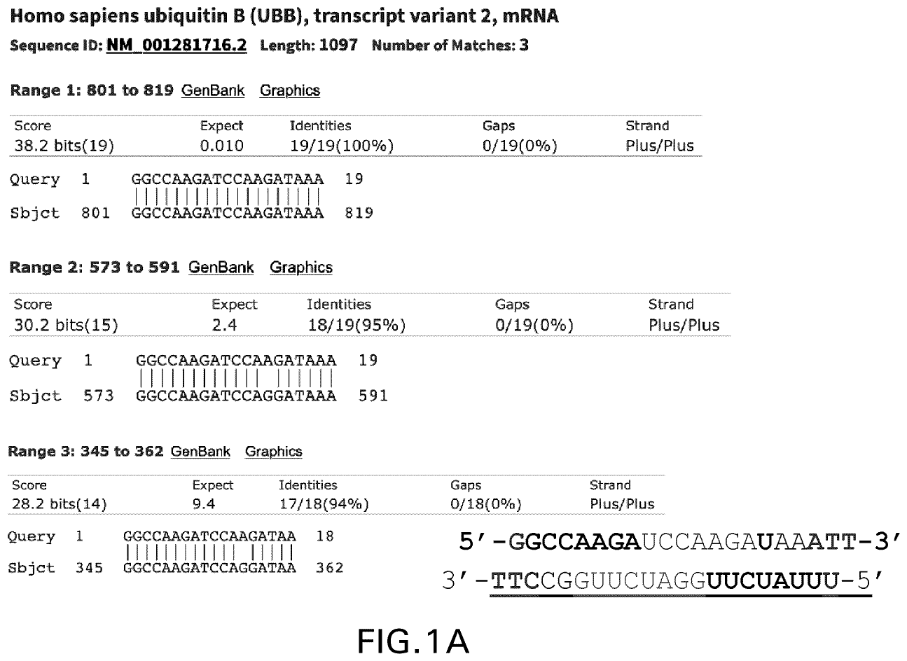

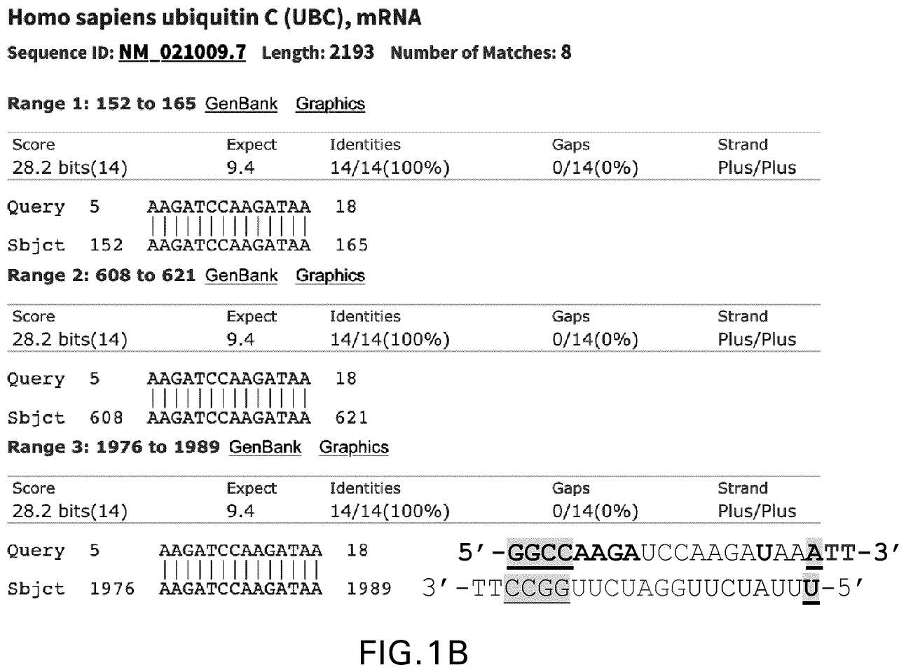

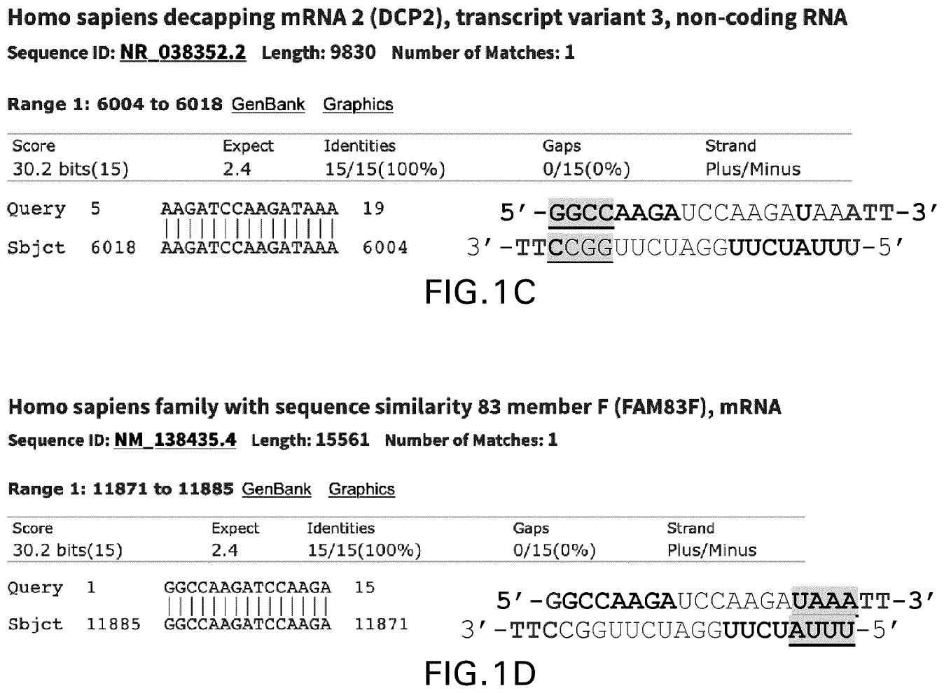

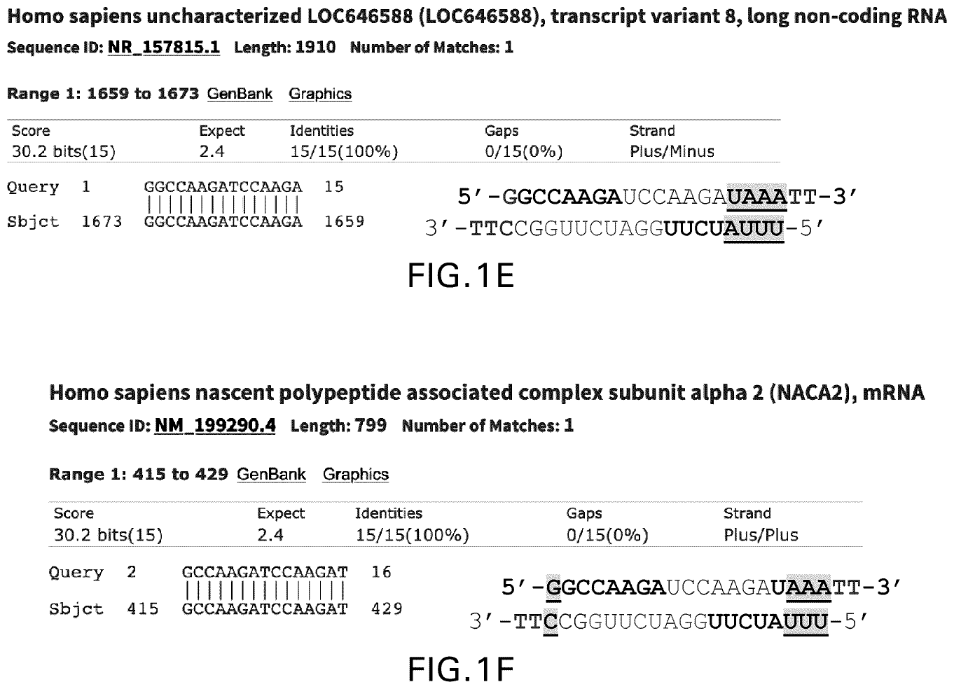

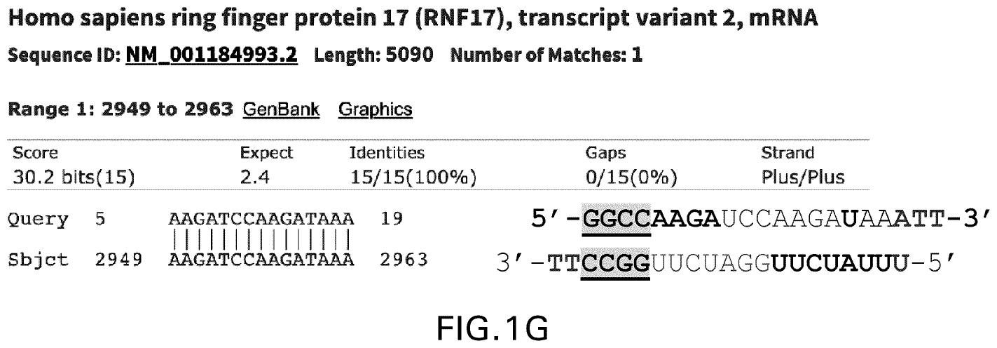

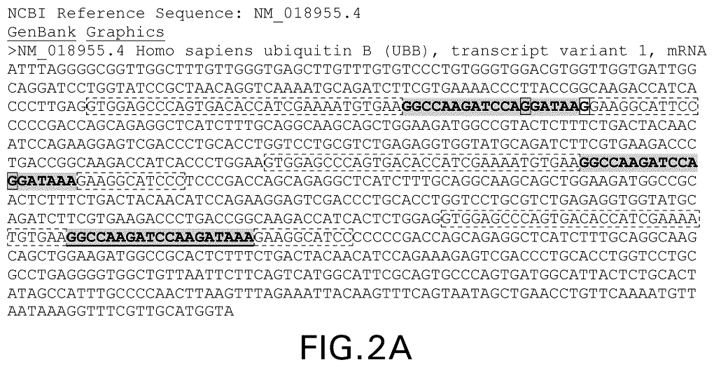

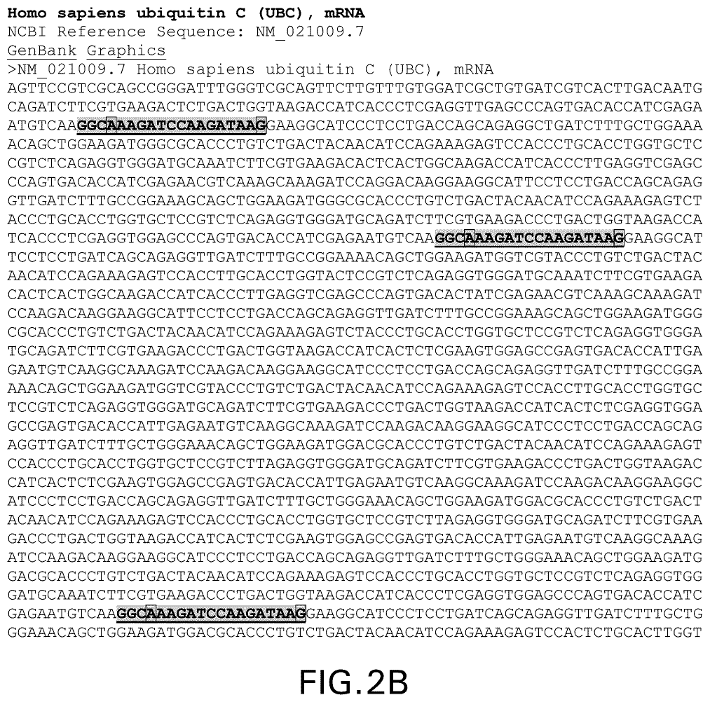

siRNA targeting sequences UBBs1- (SEQ ID NO: 896): AAGGCCAAGATCCAAGATAAA (U.S. Pat. No. 8,470,998) and UBBs2- (SEQ ID NO: 897): AAGAGGTGGTATGCAGATCTT. Analysis of UBB revealed three potential targeting regions for UBBs1 with 19/19, 18/19, and 17/19 conserved identities (FIG. 1A and FIG. 2A). Based on this analysis UBB is, surprisingly, a potential gene for a siRNA to target in multiple regions.

FIG. 1: depicts the sequence alignment of UBBs1 to various targets, non-binding regions are highlighted.

FIG. 1a: Depicts BLAST results of UBBs1 showing potential homologous regions to UBB mRNA at three regions with 19/19, 18/19 and 17/19 identity over the 19 nt stretch. Plus/Plus indicated that the guide strand of UBBs1 would bind the the mRNA of UBB.

FIG. 1b: Depicts BLAST results of UBBs1 showing potential homologous regions to UBC mRNA at three regions with 14/14 identity over the 19 nt stretch. Results for UBBs1 BLAST showing potential binding to UBC mRNA with 14/14 identity. Further examination showed 3 of 4 nt were identical and overall 17/19 identity to UBBs1.

FIG. 1c: Depicts BLAST results of UBBs1 showing potential homologous regions to DCP2 mRNA at one region with 15/15 identity.

FIG. 1d. Depicts BLAST results of UBBs1 showing potential homologous regions to FAM83F mRNA at one region with 15/15 identity.

FIG. 1e. Depicts BLAST results of UBBs1 showing potential homologous regions to LOC646588 mRNA at one region with 15/15 identity.

FIG. 1f. Depicts BLAST results of UBBs1 showing potential homologous regions to NACA2 mRNA at one region with 15/15 identity.

FIG. 1g. Depicts BLAST results of UBBs1 showing potential homologous regions to RNF17 mRNA at one region with 15/15 identity.

### Example 2: Identification of a UBBs1 Dual Target

After identifying a siRNA that bound to the UBB gene in three regions, 6 genes were identified to have conserved homology with UBB and to be potential dual target partners to the siRNA inhibition. These targets (DCP2, FAM83F, LOC646588, RNF17, NACA2, and UBC, FIG. 1) were analyzed with the goal of finding key cancer dependencies. Analysis revealed that all but one were non-essential. UBC was found to be essential and a dual target of siRNA targeting UBBs1.

FIG. 2a: depicts the potential UBBs1 siRNA targeting sites (highlighted in yellow) on the UBB sequence. Nucleotide differences are highlighted in red and similar repeat sequences are in blue. FIG. 2b: depicts the potential UBBs1 siRNA targeting sites (highlighted in yellow) on the UBC sequence. Nucleotide differences are highlighted in red and similar repeat sequences are in blue.

### Example 3: Characterization of UBB/UBC Dual Inhibition

BLAST results of UBC (FIG. 1b) reveal three potential targeting regions for siRNA targeting UBBs1 all with 18/19 identify with a 14/14 identity stretch (FIG. 1b and FIG. 2b). These results point to one siRNA potentially targeting multiple genes and multiple regions within each gene (multi-multi-targeting).

### Example 4: A Schematic of a Potential Dual UBB/UBC siRNA with Aptamers Depicting UBBs1 siRNA and EPCAM Aptamers

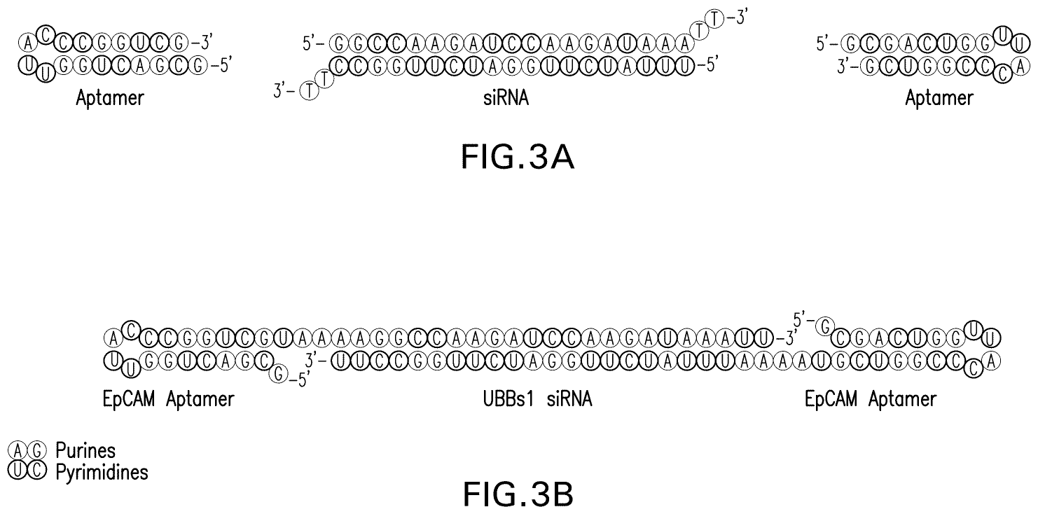

A lead siRNA or aptamer compound could be substituted in this template (FIG. 3). A depiction of an aptamer-siRNA chimera with EPCAM aptamers and UBBs1 siRNA combined with an example of an acceptable linker, for example as disclosed in U.S. Pat. No. 10,960,086 (FIG. 3). Alternative linkers can be substituted. 2-4 unpaired bases have been demonstrated to be necessary to retain aptamer function. However, U's can be substituted in place of the A's. Additionally, a streptavidin disulfide linker can be used (Ted et al., Nucleic Acid Research, 2006). The aptamers and siRNAs can be tethered to complementary linker sequences and hybridized together through Watson-Crick base pairing (Pastor et al., Mol Ther, 2011). Additionally, siRNA and aptamers can be tethered through a 4 nt (CUCU) linker or covalently fused through 2 nt linker (UU) (Zhou et al, Mol Ther, 2008) (Zhou et el., Theranostics, 2018). The aptamers and siRNAs can also be bound through a “sticky bridge” of 16 nt repeating GC with a three carbon spacer on either side of the sticky bridge (Zhou et al., Nucleic Acids, 2009). The aptamers and siRNAs can be conjugated with an acid-labile linkage or a kissing loop interaction (Huang et al., Chembiochem. 2009) (Guo et al., Human Gene Therapy, 2005).

### Example 5: Library Development of UBBs1 Variations

A siRNA library was developed containing 19 compounds of 19mer siRNA's targeting UBB Sequences:

(SEQ ID NO: 595):
5′-AAATGTGAAGGCCAAGATC-3′
(SO_U13)
(SEQ ID NO: 596:
5′-AATGTGAAGGCCAAGATCC-3′
(SO_U14)
(SEQ ID NO: 597):
5′-ATGTGAAGGCCAAGATCCA-3′
(SO_U15)
(SEQ ID NO: 598):
5′-TGTGAAGGCCAAGATCCAA-3′
(SO_U16)
(SEQ ID NO: 599):
5′-GTGAAGGCCAAGATCCAAG-3′
(SO_U17)
(SEQ ID NO: 600):
5′-TGAAGGCCAAGATCCAAGA-3′
(SO_U18)
(SEQ ID NO: 601):
5′-GAAGGCCAAGATCCAAGAT-3′
(SO_U19)
(SEQ ID NO: 602):
5′-AAGGCCAAGATCCAAGATA-3′
(SO_U20)
(SEQ ID NO: 603):
5′-AGGCCAAGATCCAAGATAA-3′
(SO_U21)
(SEQ ID NO: 604):
5′-GGCCAAGATCCAAGATAAA-3′
(SO_U22)
(SEQ ID NO: 605):
5′-GCCAAGATCCAAGATAAAG-3′
(SO_U23)
(SEQ ID NO: 606):
5′-CCAAGATCCAAGATAAAGA-3′
(SO_U24)
(SEQ ID NO: 607):
5′-CAAGATCCAAGATAAAGAA-3′
(SO_U25)
(SEQ ID NO: 608):
5′-AAGATCCAAGATAAAGAAG-3′
(SO_U26)
(SEQ ID NO: 609):
5′-AGATCCAAGATAAAGAAGG-3′
(SO_U27)
(SEQ ID NO: 610):
5′-GATCCAAGATAAAGAAGGC-3′
(SO_U28)
(SEQ ID NO: 611):
5′-ATCCAAGATAAAGAAGGCA-3′
(SO_U29)
(SEQ ID NO: 612):
5′-TCCAAGATAAAGAAGGCAT-3′
(SO_U30)
(SEQ ID NO: 613):
5′-CCAAGATAAAGAAGGCATC-3′
(SO_U31)
(SEQ ID NO: 614):
5′-CAGGATCCTGGTATCCGCTAA-3′
(UBB_1)
(SEQ ID NO: 615):
5′-ATGGCATTACTCTGCACTATA-3′
(UBB_2)
(SEQ ID NO: 616):
5′-CCAACTTAAGTTTAGAAATTA-3′
(UBB_3)
(SEQ ID NO: 617):
5′-GAGGCTCATCTTTGCAGGCAA-3′
(UBB_4)

6 scrambled UBBs1 targeting sequences were developed as controls:

(SEQ ID NO: 618):
5′- GAACAACCGGCAAATAGAT-3′
(SO_U07)
(SEQ ID NO: 619):
5′- GCAATACGCGAAGACATAA-3′
(SO_U08)
(SEQ ID NO: 620):
5′- GAAAGACGGACCATAACAT-3′
(SO_U09)
(SEQ ID NO: 621):
5′- GAAGAACCACGAAGACTTA-3′
(SO_U10)
(SEQ ID NO: 622):
5′- GTAGGACGCACAAACTAAA-3′
(SO_U11)
(SEQ ID NO: 623):
5′- GGACAGATCGCTAAACAAA-3′
(SO_U12)

Three UBBs1-like targeting compounds were developed including one that is designed to target UBC in a conserved location to target both UBB and UBC.

(SEQ ID NO: 624):
5′- GGCCAAGATCCAGGATAAA -3′
(SO_U04)
(SEQ ID NO: 625):
5′-GGCCAAGATCCAGGATAAG-3′
(SO_U05)
(SEQ ID NO: 626):
5′-GGCAAAGATCCAAGATAAG-3′
(SO_U06)

### Example 6: In Vitro Validation of UBB siRNA Library

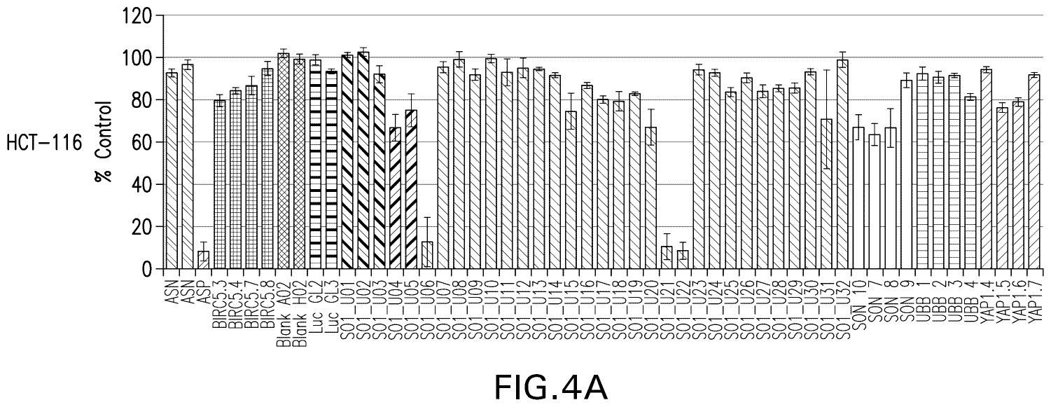

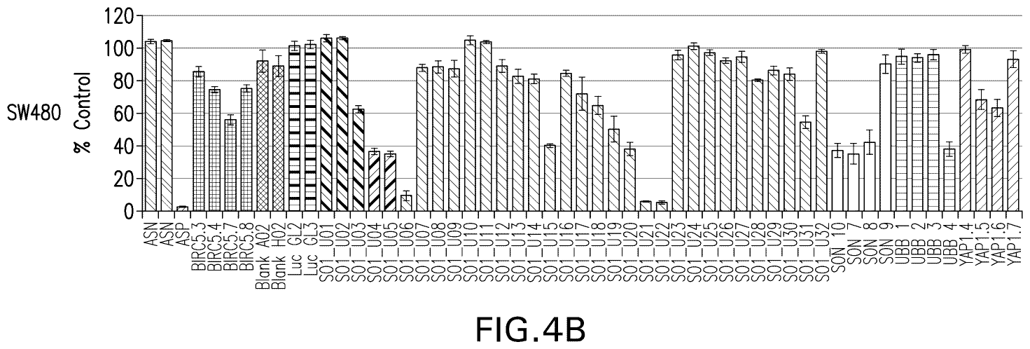

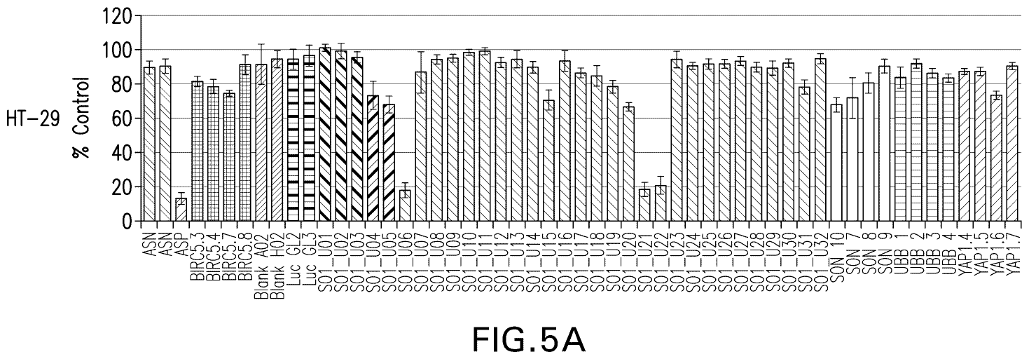

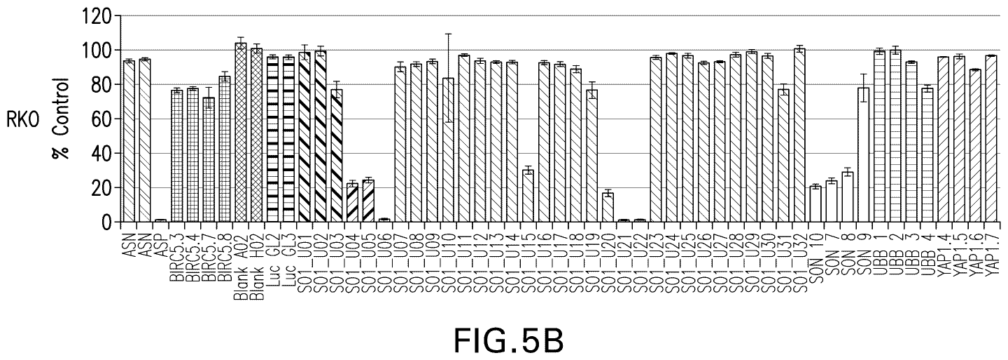

HCT-116, SW480, RKO, and HT-29 colon cancer cells were treated under standard siRNA transfection conditions with various siRNA compounds including those previously listed as well as ASN (negative control) and ASP (positive control) (16.7 nM; 96 hr) (FIGS. 4 and 5).

The siRNA targeting UBBs1 (SEQ ID NO: 604) is cytotoxic to SW480 and HCT-116. The siRNA targeting sequence (SEQ ID NO: 624) and (SEQ ID NO: 625) also inhibit UBB. The siRNA developed to target UBC (SEQ ID NO: 626) is as potent as the siRNA targeting UBBs1 (SEQ ID NO: 604). A UBBs1 scrambled siRNA targeting sequence (SEQ ID NO: 621) does not have a cytotoxic effect and could be used as a negative control. A novel siRNA targeting sequence (SEQ ID NO: 603) is surprisingly more potent than UBBs1 (SEQ ID NO: 604).

### Example 7: Validation of UBB siRNA Library

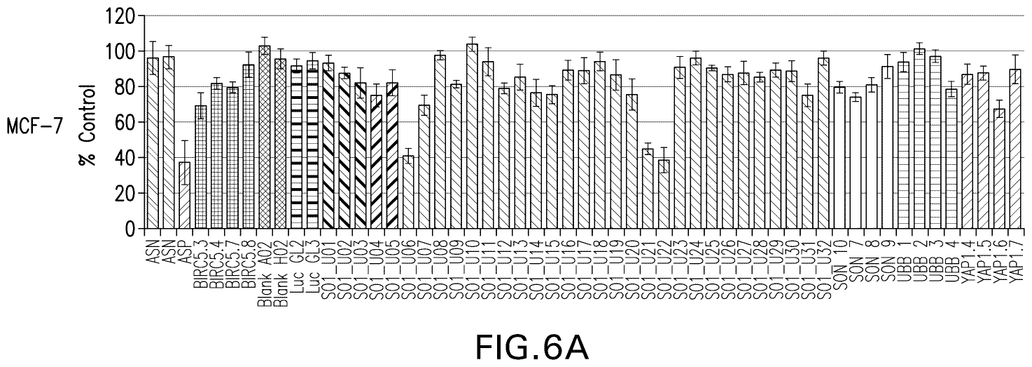

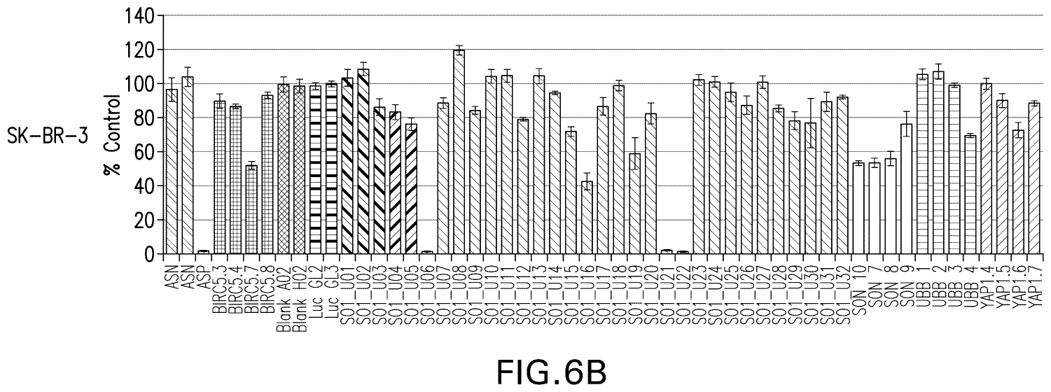

MCF-7 and SK-BR-3 breast cancer cells were treated under standard siRNA transfection conditions with various siRNA compounds including those previously listed as well as controls: ASN siRNA (negative), ASP siRNA (positive) (16.7 nM; 96 hr) (FIG. 6).

The siRNA targeting UBBs1 (SEQ ID NO: 604) is cytotoxic to MCF-7 and SK-BR-3. The siRNA targeting (SEQ ID NO: 626) is as potent as the siRNA to UBBs1 (SEQ ID NO: 604) and the siRNA targeting (SEQ ID NO: 603) appears to be more potent than UBBs1 (SEQ ID NO: 604). This experiment demonstrated surprising efficacy of dual UBB and UBC siRNA inhibition on breast cancer cells.

### Example 8: Dose Response of Various siRNA Sequences on Colon Cancer Cells

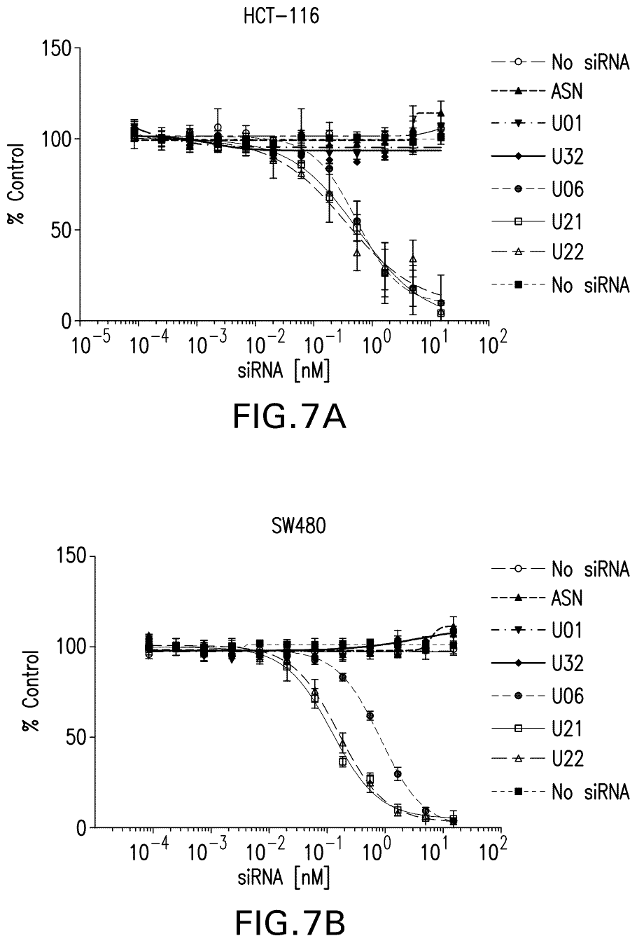

Dose response curve of HCT-116 and various siRNA sequences. Cells were grown to 2,000 cells/well in a 384-well plate, and treated with 62 pM-15 nM of compounds for 96 hours (FIG. 7A).

FIG. 7B: Dose response curve of SW480 and various siRNA sequences. Cells were grown to 2,000 cells/well in a 384-well plate, and treated with 62 pM-15 nM of compounds for 96 hours (FIG. 7B).

These results demonstrate that high concentrations of siRNA-aptamer dual targeting chimeras is not necessary to see efficacy in cancer cells.

### Example 9: Silencing of UBB and UBC

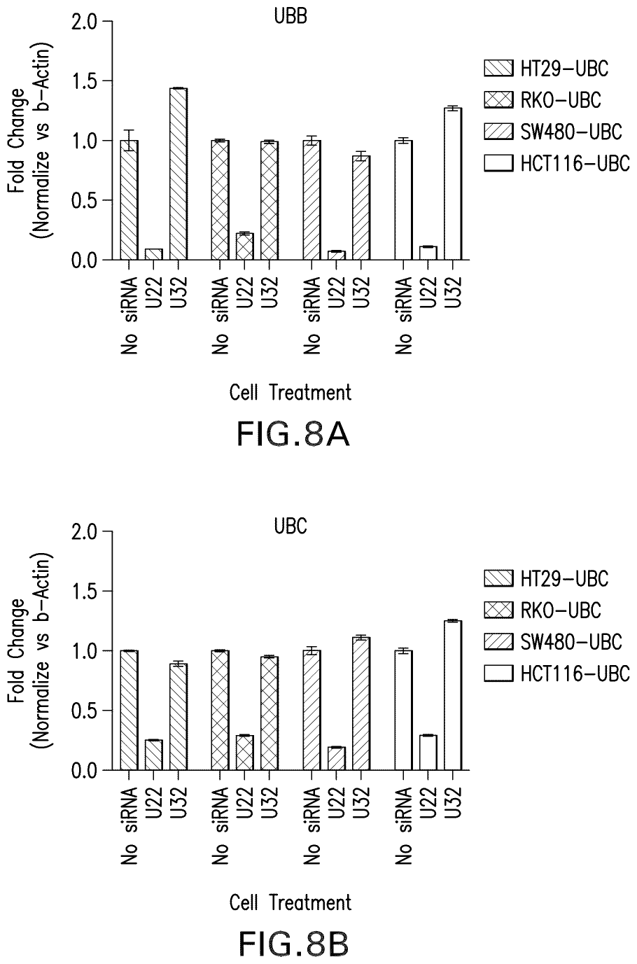

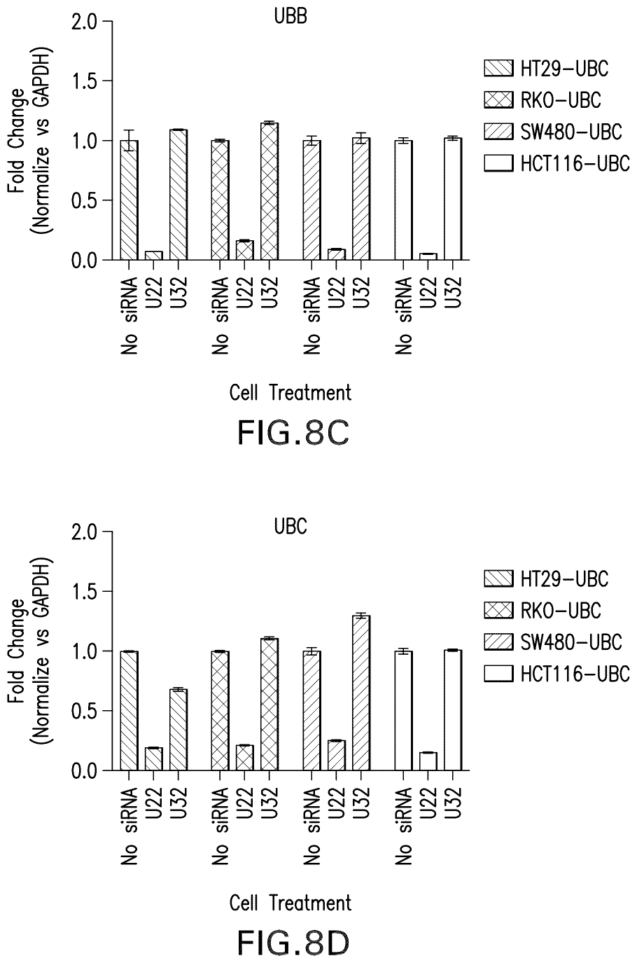

In order to demonstrate that active siRNA targeting (SEQ ID NO: 626), (SEQ ID NO: 6-03), and (SEQ ID NO: 604) silence both UBB and UBC and other UBB targeting siRNA's do not, a cell assay was performed using HT29, RKO, SW480, and HCT116 cells. Cells were treated with siRNA or control (15 nM siRNA; 20 hr). UBB (FIGS. 8a and 8c) or UBC levels (FIG. 8b or 8d) were measured and normalized by b-Actin (FIGS. 8a and 8b) or GAPDH (FIGS. 8c and 8d).

Results indicate the dual targeting capability of siRNA's to (SEQ ID NO: 604) across multiple cell types.

### Example 10: UBB-UBC Expression in HCT116 Cells Following siRNA Knockdown

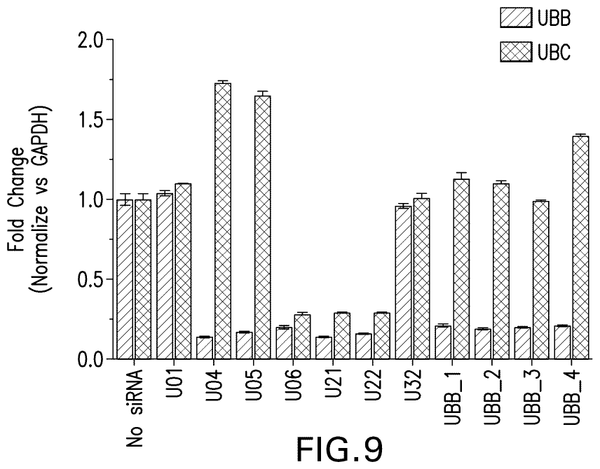

HCT116 cells were treated with the specified siRNA including U01, a Luciferase GL3 siRNA (15 nM siRNA; 20 hr). qPCR results were normalized to GAPDH. Results demonstrate the ability of siRNA's targeting (SEQ ID NO: 626), (SEQ ID NO: 603) and (SEQ ID NO: 604) to dual inhibit UBB and UBC. Control UBB inhibitors are not able to inhibit UBC (FIG. 9).

(SEQ ID NO: 893):
GGCAAAGAUCCAAGAUAAG
(SEQ ID NO: 894):
GGCCAAGAUCCAAGAUAAA
(SEQ ID NO: 895):
AGGCCAAGAUCCAAGAUAA

Additionally, HCT116 cells were treated with another set of UBB/UBC targeted siRNAs.

(SEQ ID NO: 301):
GCCGUACUCUUUCUGACUA
(UBB_1G2)
(SEQ ID NO: 302):
GUAUGCAGAUCUUCGUGAA
(UBB_2G2)
(SEQ ID NO: 303):
GACCAUCACUCUGGAGGUG
(UBB_3G2)
(SEQ ID NO: 304):
CCCAGUGACACCAUCGAAA
(UBB_4G2)
(SEQ ID NO: 305):
GUGAAGACCCUGACUGGUA
(UBC_1G6)
(SEQ ID NO: 306):
AAGCAAAGAUCCAGGACAA
(UBC_2G6)
(SEQ ID NO: 307):
GAAGAUGGACGCACCCUGU
(UBC_3G6)
(SEQ ID NO: 308):
GUAAGACCAUCACUCUCGA
(UBC_4G6)

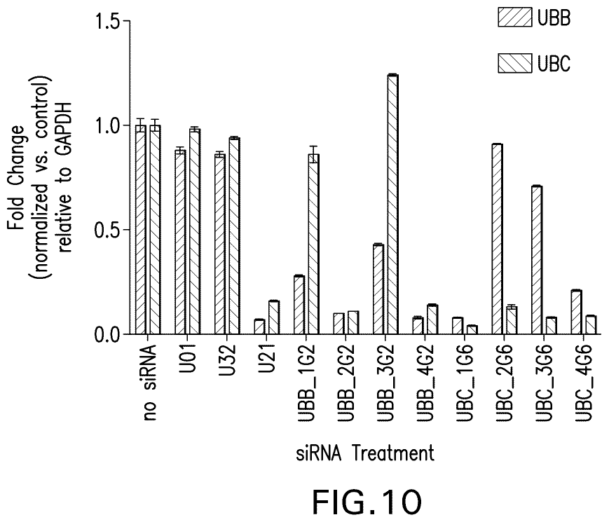

siRNA targeting (SEQ ID NO: 302) and (SEQ ID NO: 304) and (SEQ ID NO: 305) and (SEQ ID NO: 308) demonstrated significantly diminished UBB and UBC expression levels. (FIG. 10).

### Example 11

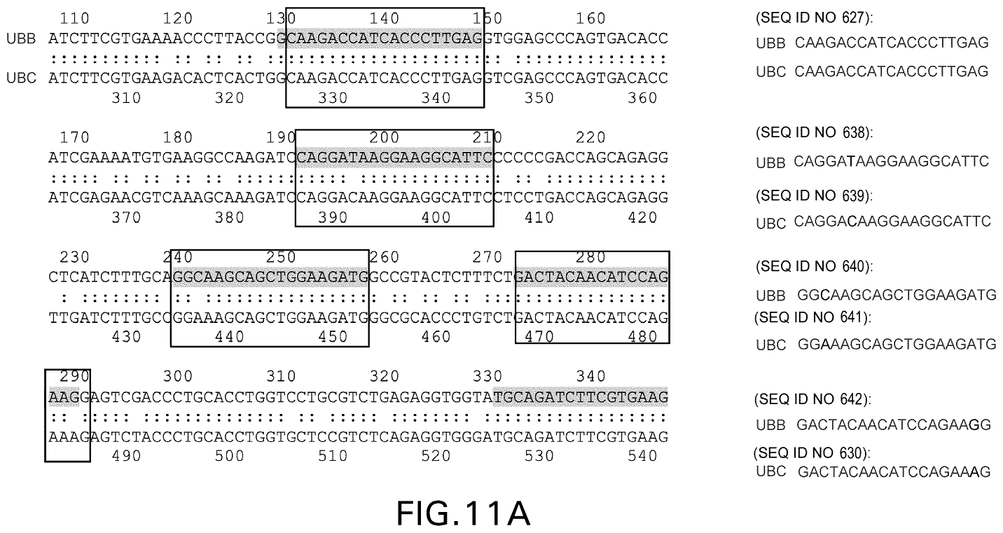

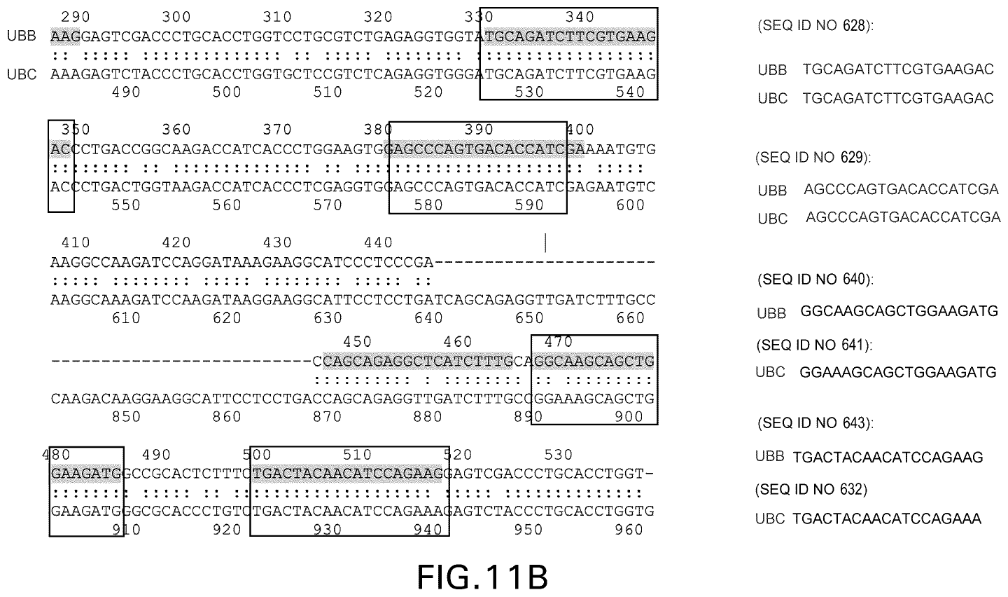

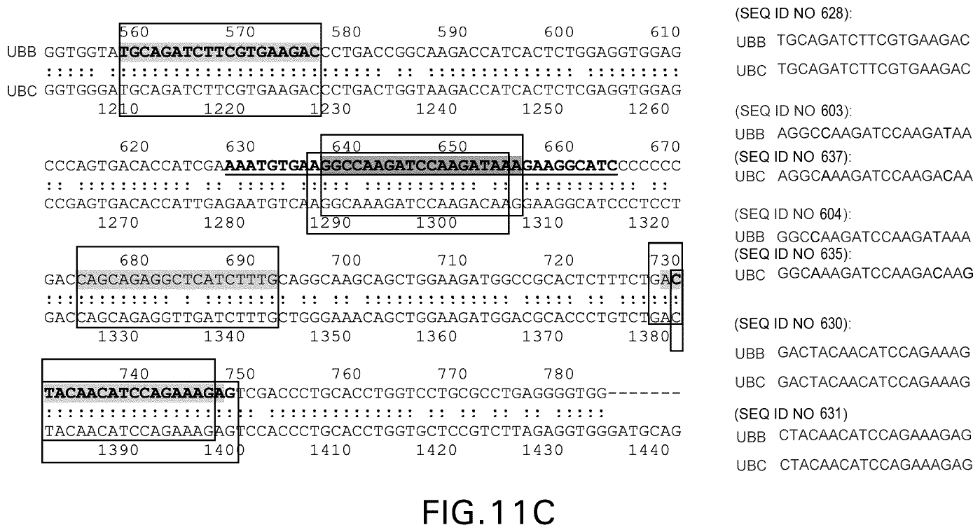

Depicts BLAST results of homologous regions between UBB and UBC mRNA at regions with 19/19, 18/19 and 17/19 identity over the 19 nt stretch (FIG. 11)

Dual UBB and UBC siRNA Targeting Sequences:

(SEQ ID NO: 627):
5′-CAAGACCATCACCCTTGAG-3′
(SO_U33)
(SEQ ID NO: 628):
5′-TGCAGATCTTCGTGAAGAC-3′
(SO_U34)
(SEQ ID NO: 629):
5′-AGCCCAGTGACACCATCGA-3′
(SO_U35)
(SEQ ID NO: 630):
5′-GACTACAACATCCAGAAAG-3′
(SO_U36)
(SEQ ID NO: 631):
5′-CTACAACATCCAGAAAGAG-3′
(SO_U37)
(SEQ ID NO: 632):
5′-TGACTACAACATCCAGAAA-3′
(SO_U49)

UBB Similar to UBC siRNA Targeting Sequences:

(SEQ ID NO: 633):
5′-AGTGACACCATCGAAAATG-3′
(SO_U38)
(SEQ ID NO: 634):
5′-AGGCAAAGATCCAAGATAA-3′
(SO_U39)
(SEQ ID NO: 635):
5′-GGCAAAGATCCAAGACAAG-3′
(SO_U40)
(SEQ ID NO: 636):
5′-CAAGGCAAAGATCCAAGAC-3′
(SO_U41)
(SEQ ID NO: 637):
5′-AGGCAAAGATCCAAGACAA-3′
(SO_U42)
(SEQ ID NO: 638):
5′-CAGGATAAGGAAGGCATTC-3′
(SO_U43)
(SEQ ID NO: 639):
5′-CAGGACAAGGAAGGCATTC-3′
(SO_U44)
(SEQ ID NO: 640):
5′-GGCAAGCAGCTGGAAGATG-3′
(SO_U45)
(SEQ ID NO: 641):
5′-GGAAAGCAGCTGGAAGATG-3′
(SO_U46)
(SEQ ID NO: 642):
5′-GACTACAACATCCAGAAGG-3′
(SO_U47)
(SEQ ID NO: 643):
5′-TGACTACAACATCCAGAAG-3′
(SO_U48)

(SEQ ID NO: 627) was identified 2× in UBC and 1× in UBB. (SEQ ID NO: 628) was identified 4× in UBC and 1× in UBB. (SEQ ID NO: 629) was identified 2× in UBC and 3× in UBB. (SEQ ID NO: 630) was identified 7× in UBC and 1× in UBB. (SEQ ID NO: 631) was identified 7× in UBC and 1× in UBB.

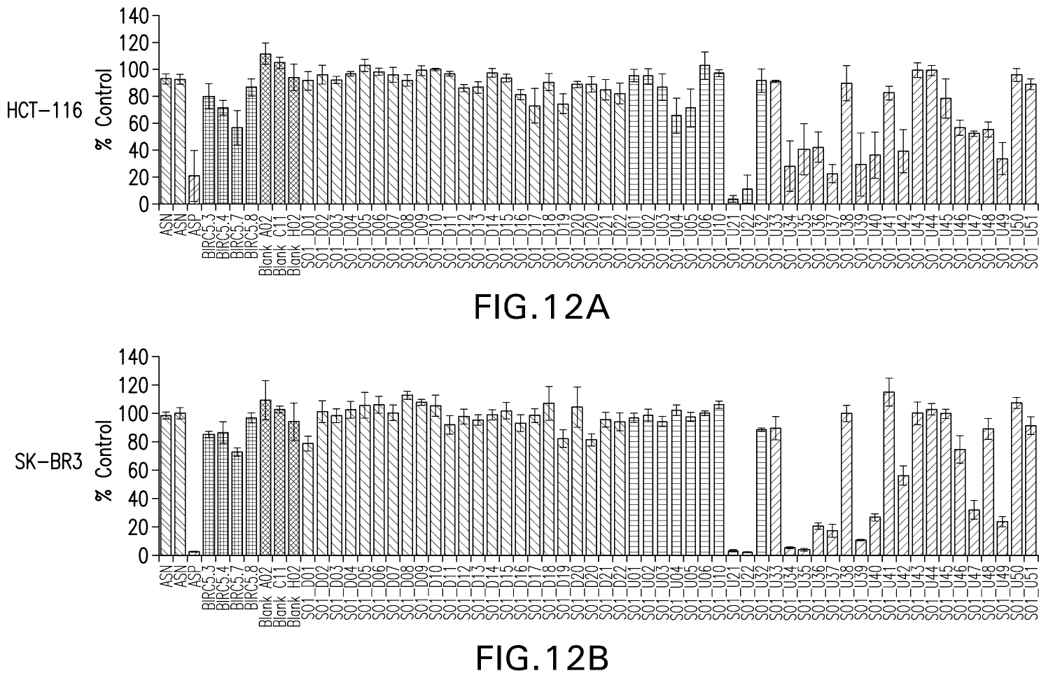

HCT-116 (FIG. 12a), a colon cancer cell line, and SK-BR3 (FIG. 12b), a breast cancer cell line, were treated under standard siRNA transfection conditions with siRNA compounds targeting mRNA sequences previously listed as well as ASN (negative control) and ASP (positive control) (16.7 nM; 96 hr). U32, U50, U51 are negative control siRNAs.

These results identify (SEQ ID NO: 628), (SEQ ID NO: 629), (SEQ ID NO: 630), (SEQ ID NO: 631), and (SEQ ID NO: 641) as siRNA targets with the ability to inhibit both UBB and UBC.

### Example 12: UBB-UBC Species

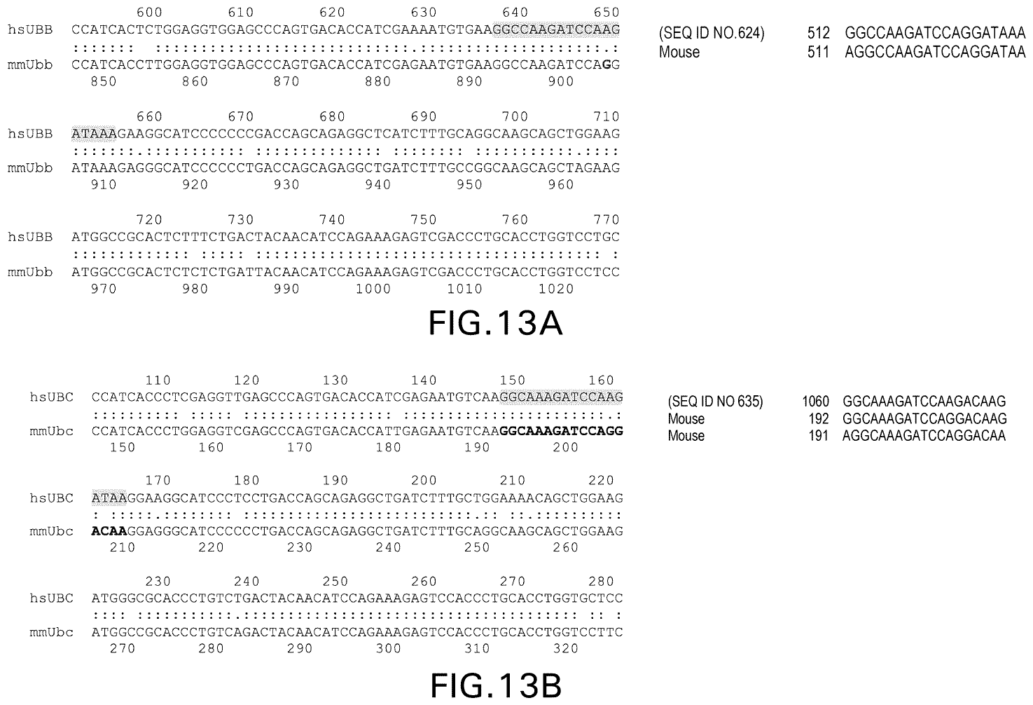

Human UBB and UBC sequences were compared to mouse in order to find potential homologous regions for in vivo drug development studies. (SEQ ID NO: 624) and (SEQ ID NO: 43) sequences were found to be effective siRNA targeting regions for human and contain high homology to mouse. (SEQ ID NO: 62) with minimal nucleotide differences was identified 4× in mouse UBB and (SEQ ID NO: 43) with minimal nucleotide differences was identified 9× in mouse UBC. These sequences will be effective for multi-species in vivo pre-clinical studies (FIG. 13).

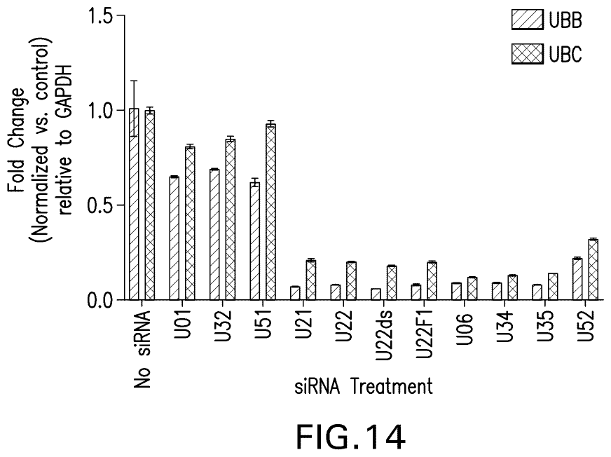

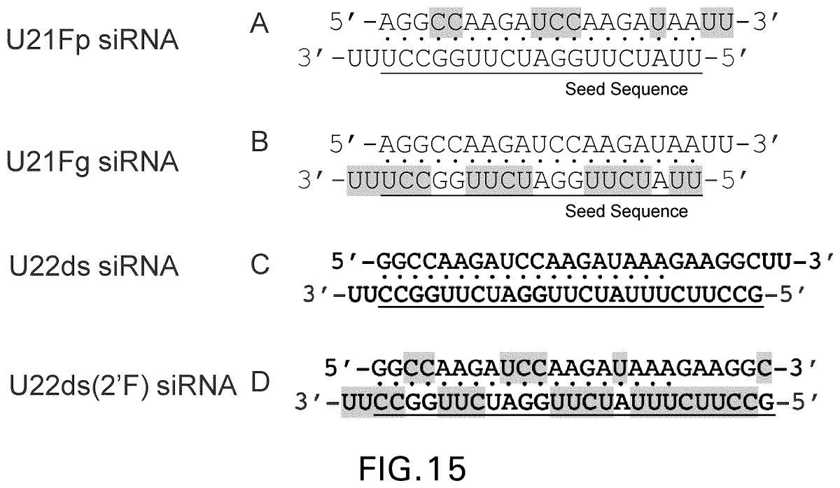

An additional mouse sequence (SEQ ID NO: 644: U52) was tested against UBB and UBC. Additionally, a dicer substrate siRNA (SEQ ID NO: 645: U22ds (FIG. 15C)) and a 2′F pyrimidine modified siRNA (SEQ ID NO: 895: U21F) were included in this experiment. Gene expression of HCT116 cells was measured by qPCR following siRNA treatment and these siRNAs were found to effectively decrease expression of both UBB and UBC (FIG. 14)

(SEQ ID NO: 644):
5′-GGCAAAGAUCCAGGACAAG-3′
(U52)
(SEQ ID NO: 645):
5′-GGCCAAGAUCCAAGAUAAAGAAGGC-3′
(U22ds)

### Example 13: UBB-UBC Modifications

2′F pyrimidine modifications of the siRNA targeting SEQ ID NO: 895 are depicted in FIG. 14. The modifications can either be on the passenger strand, U21Fp (FIG. 15A) or the guide strand, U21Fg (FIG. 15B). The guide strand is underlined. A dicer substrate as well as a 2′F modified dicer substrate are also provided (FIGS. 15C and 15D) (SEQ ID NO: 644).

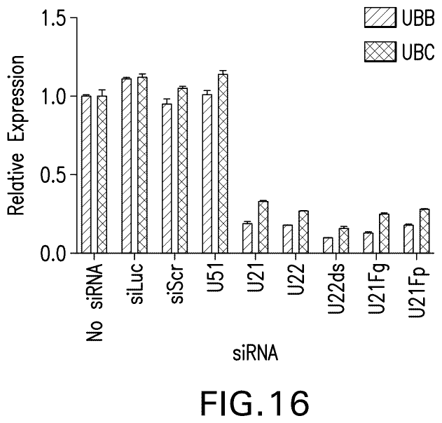

HCT-116 cells were treated with UBB-UBC targeting siRNAs. Modified and unmodified versions of SEQ ID NO: 895 are able to silence UBB and UBC with similar activity to unmodified (FIG. 16).

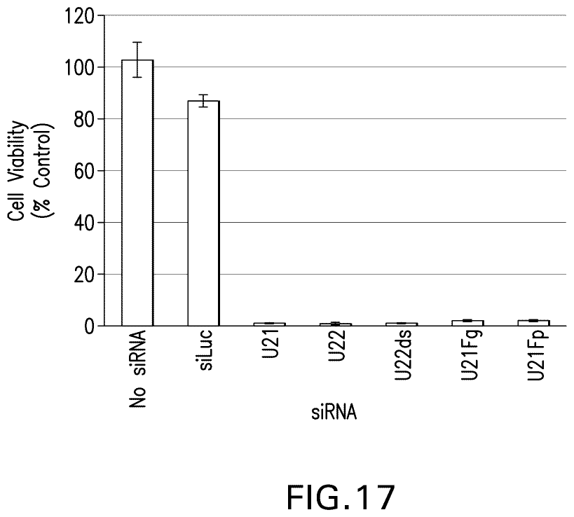

Cell viability was measured, and the silencing of these genes demonstrated >98% cytotoxicity at 96 hours (FIG. 17).

### Example 14: Identification of Multi-Targeting Domains

Utilizing Basic Local Alignment Search Tool (Blast), new sequences were identified with highly conserved homology to dual or triple targets.

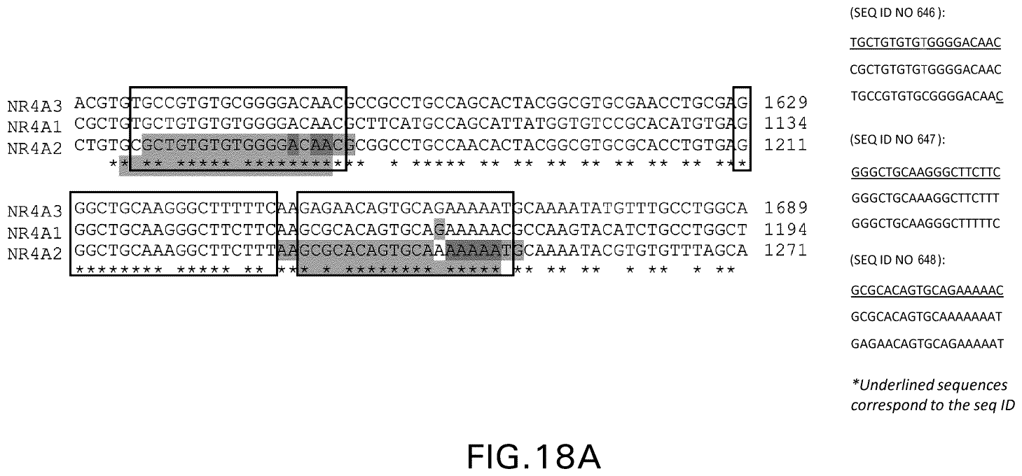

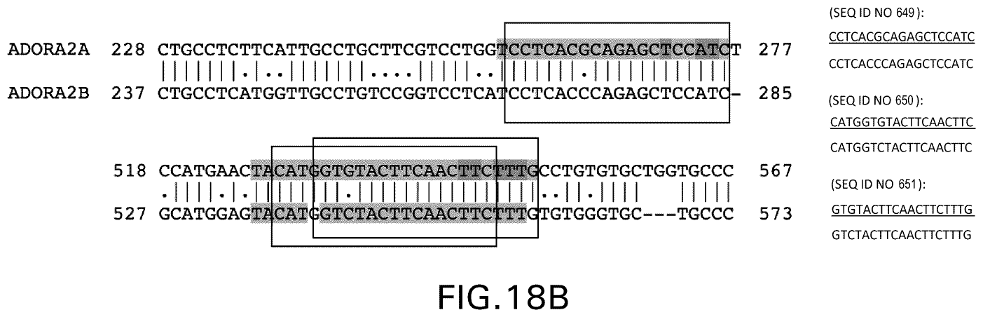

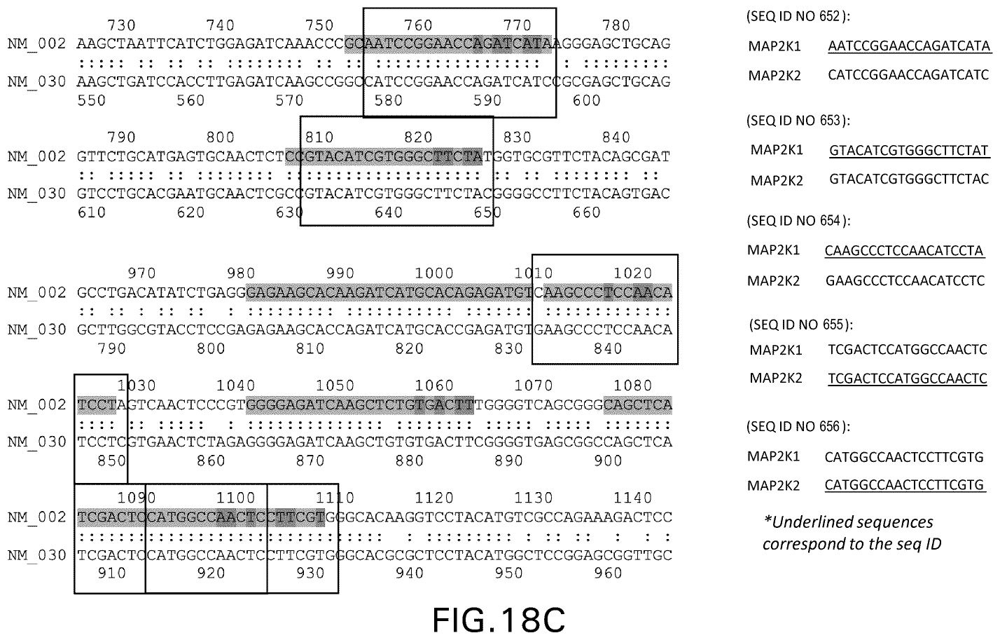

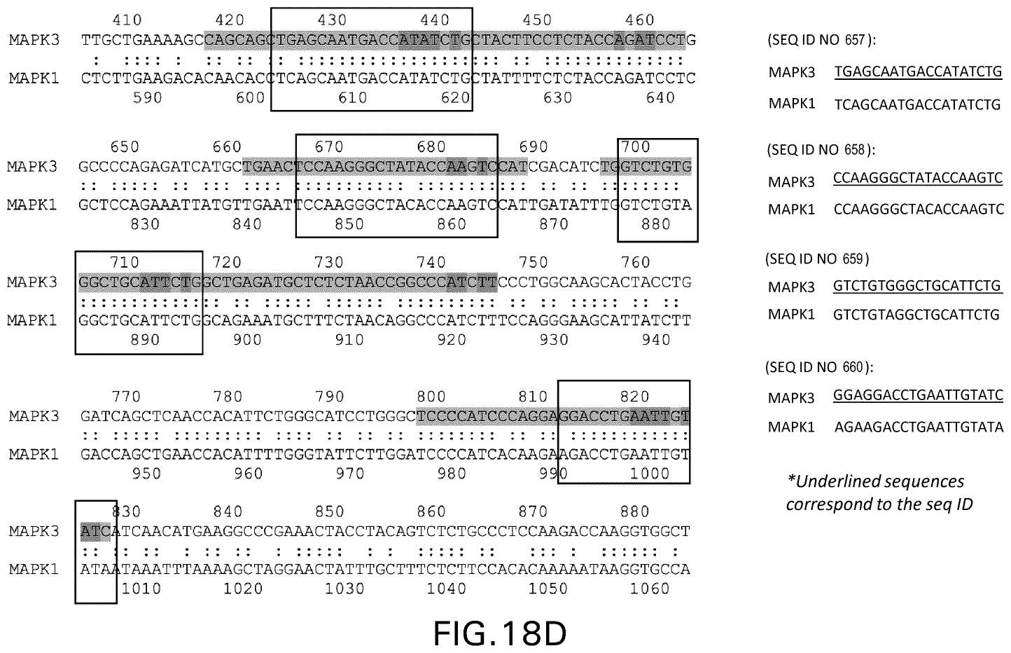

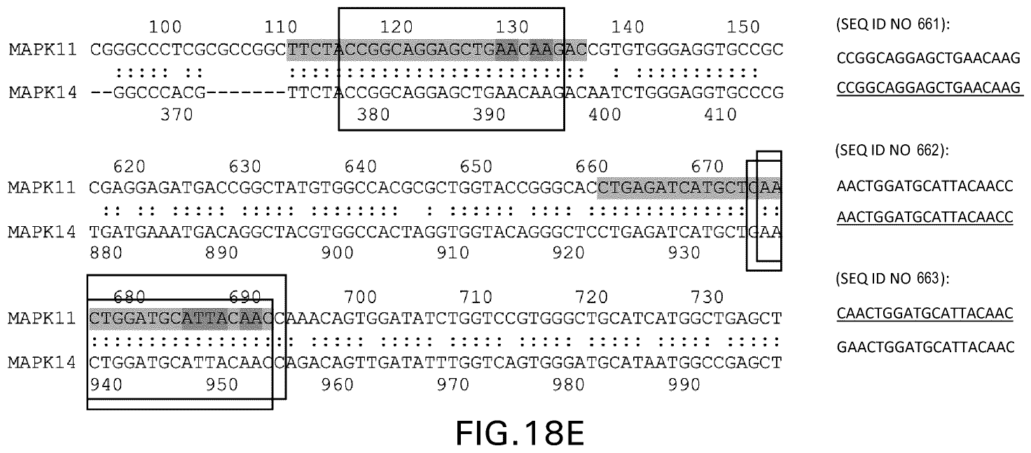

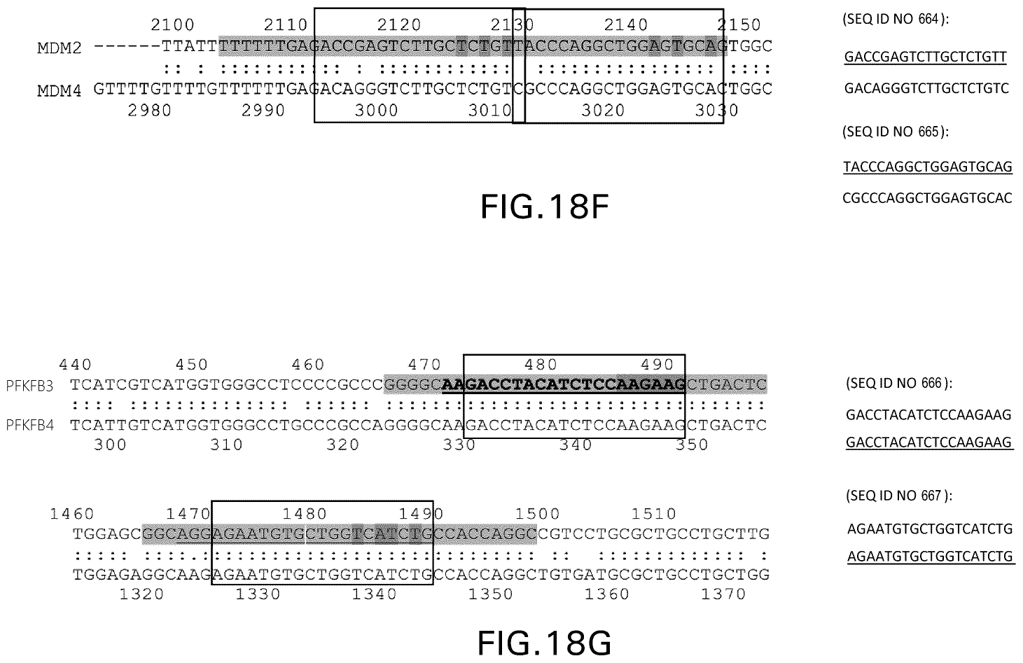

NR4A3 was found to have three potential targeting regions which have 18/19 conserved identities across all three sequences with NR4A1, and 18/19, 18/19, and 17/19 conserved identities with NR4A2 (FIG. 18A).

NR4A1, NR4A2, and NR4A3 siRNA Targeting Sequences:

(SEQ ID NO: 646):
5′-TGCTGTGTGTGGGGACAAC-3′
(SEQ ID NO: 647):
5′-GGGCTGCAAGGGCTTCTTC-3′
(SEQ ID NO: 648):
5′-GCGCACAGTGCAGAAAAAC-3′

ADORA2A was found to have three potential targeting regions which have 18/19 conserved identities across all three sequences with ADORA2B (FIG. 18B).

ADORA2A and ADORA2B siRNA Targeting Sequences:

(SEQ ID NO: 649):
5′-CCTCACGCAGAGCTCCATC-3′
(D04)
(SEQ ID NO: 650):
5′-CATGGTGTACTTCAACTTC-3′
(D05)
(SEQ ID NO: 651):
5′-GTGTACTTCAACTTCTTTG-3′
(D06)

MAP2K1 was found to have five potential targeting regions which have 19/19, 19/19, 17/19, 18/19, and 17/19 conserved identities with MAP2K2 (FIG. 18C).

MAP2K1 and MAP2K2 siRNA Targeting Sequences:

(SEQ ID NO: 652):
5′-AATCCGGAACCAGATCATA-3′
(D07)
(SEQ ID NO: 653):
5′-GTACATCGTGGGCTTCTAT-3′
(D08)
(SEQ ID NO: 654):
5′-CAAGCCCTCCAACATCCTA-3′
(D09)
(SEQ ID NO: 655):
5′-TCGACTCCATGGCCAACTC-3′
(D10)
(SEQ ID NO: 656):
5′-CATGGCCAACTCCTTCGTG-3′
(D11)

ERK1 (MAPK3) was found to have four potential targeting regions which have 18/19, 18/19, 16/19 conserved identities with ERK2 (MAPK1) (FIG. 18D).

MAPK3 and MAPK1 siRNA Targeting Sequences:

(SEQ ID NO: 657):
5′-TGAGCAATGACCATATCTG-3′
(D12)
(SEQ ID NO: 658:
5′-CCAAGGGCTATACCAAGTC-3′
(D13)
(SEQ ID NO: 659):
5′-GTCTGTGGGCTGCATTCTG-3′
(D14)
(SEQ ID NO: 660:
5′-GGAGGACCTGAATTGTATC-3′
(D15)

MAPK11 was found to have three potential targeting regions which have 19/19, 19/19, and 18/19 conserved identities with MAPK14 (FIG. 18E).

MAPK11 and MAPK14 siRNA Targeting Sequences:

(SEQ ID NO: 661):
5′-CCGGCAGGAGCTGAACAAG-3′
(D16)
(SEQ ID NO: 662):
5′-AACTGGATGCATTACAACC-3′
(D17)
(SEQ ID NO: 663):
5′-CAACTGGATGCATTACAAC-3′
(D18)

MDM2 was found to have two potential targeting regions which have 16/19 and 16/19 conserved identities with MDM4 (FIG. 18F).

MDM2 and MDM4 siRNA Targeting Sequences:

(SEQ ID NO: 664):
5′-GACCGAGTCTTGCTCTGTT-3′
(SEQ ID NO: 665):
5′-TACCCAGGCTGGAGTGCAG-3′

PFKFB3 was found to have two potential targeting regions which both had 19/19 conserved identities with PFKFB4 (FIG. 18G).

PFKFB3 and PFKFB4 siRNA Targeting Sequences:

(SEQ ID NO: 666):
5′-GACCTACATCTCCAAGAAG-3′
(SEQ ID NO: 667):
5′-AGAATGTGCTGGTCATCTG-3′

Based on this work these sequences are potential siRNA targets for dual or triple inhibition of gene expression.

### Example 15: siRNA Target Validation-Multiple Gene Expression Following siRNA Treatment

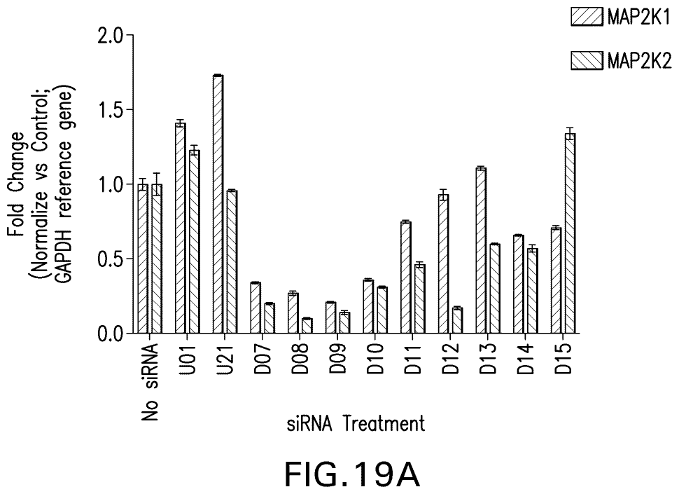

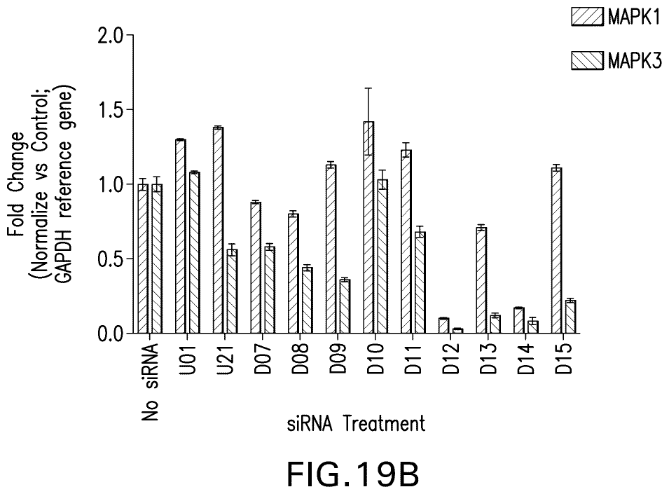

HCT116 cells were treated with siRNA and the expression levels of MAP2K1 and MAP2K2 (FIG. 19A) and MAPK1 and MAPK3 were measured (FIG. 19B).

SiRNA targeting sequences (SEQ ID NOS: 652-654) reduced MAP2K1 and MAP2K2 expression. SiRNA targeting sequences (SEQ ID NOS: 657-659) effectively reduced expression of MAPK1 and MAPK3. The siRNA targeting sequence (SEQ ID NO: 657) knocked down expression of MAPK1, MAPK3, and MAP2K2.

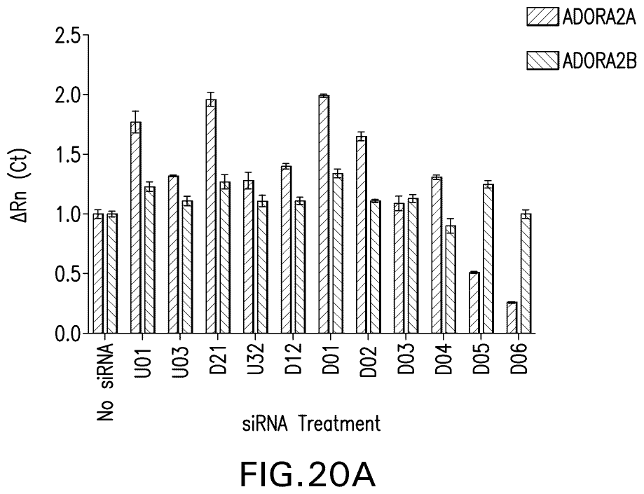

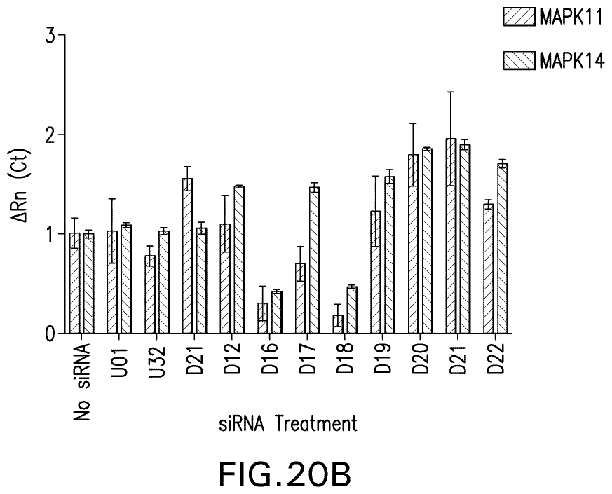

SKBR3 cells were treated with siRNA and the expression levels of ADORA2A/ADORA2B (FIG. 20A) were measured. siRNA targeting (SEQ ID NO: 650) and (SEQ ID NO: 651) demonstrated the largest decrease in ADORA2A expression.

HCT116 cells were treated with siRNA and the expression levels of MAPK11/MAPK14 (FIG. 20B) were measured. siRNA targeting (SEQ ID NO: 661) and (SEQ ID NO: 663) targeting siRNA demonstrates efficacy in decreasing expression of MAPK11 and MAPK14.

### Example 16: siRNA Target Validation-Single Gene Expression Following siRNA Treatment

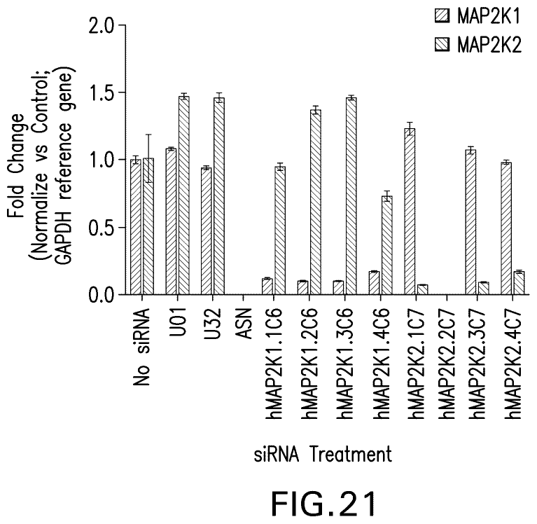

HCT116 cells were treated with siRNA that targeted either the expression levels of MAP2K1 or of MAP2K2, the expression of both were measured after treatment (FIG. 21).

### Target Sequences of MAP2K1:

(SEQ ID NO: 668):
5′-GCACATGGATGGAGGTTCT-3′
(hMAP2K1.1C6)
(SEQ ID NO: 669):
5′-GCAGAGAGAGCAGATTTGA-3′
(hMAP2K1.2C6)
(SEQ ID NO: 670):
5′-GAGCAGATTTGAAGCAACT-3′
(hMAP2K1.3C6)
(SEQ ID NO: 671):
5′-CCAGAAAGCTAATTCATCT-3′
(hMAP2K1.4C6)

All four siRNAs effectively and selectively inhibited expression of MAP2K1.

### Target Sequences of MAP2K2:

(SEQ ID NO: 672):
5′-CAAAGACGATGACTTCGAA-3′
(hMAP2K2.1C7)
(SEQ ID NO: 673):
5′-GATCAGCATTTGCATGGAA-3′
(hMAP2K2.2C7)
(SEQ ID NO: 674):
5′-GGAAGCTGATCCACCTTGA-3′
(hMAP2K2.3C7)
(SEQ ID NO: 675):
5′-GAAAGTCAGCATCGCGGTT-3′
(hMAP2K2.4C7)

(SEQ ID NO: 672), (SEQ ID NO: 674), and (SEQ ID NO: 675) effectively and selectively inhibited expression of MAP2K2.

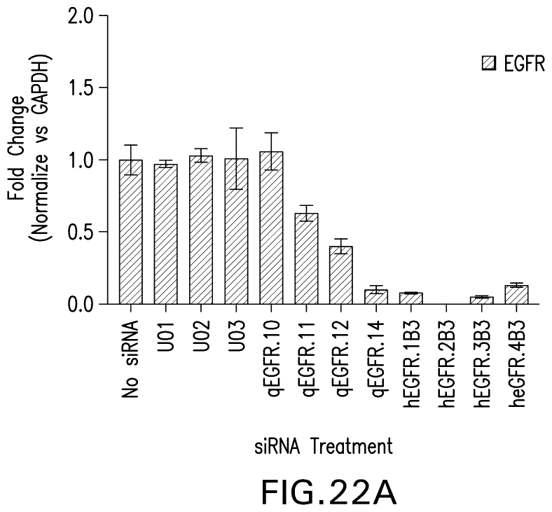

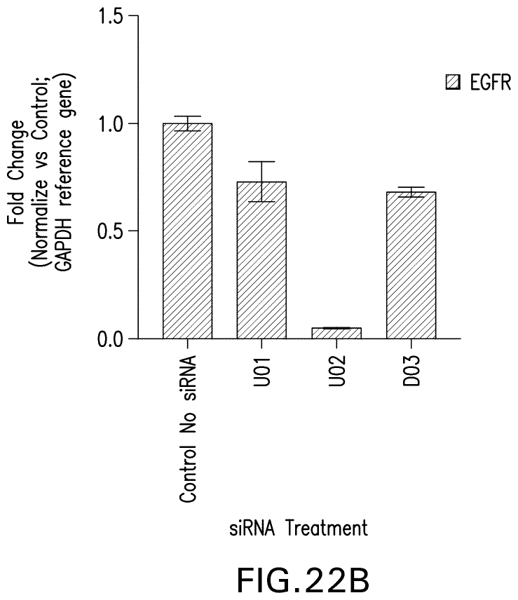

SK-BR3 cells were treated with siRNA and the expression of EGFR was measured after treatment (FIG. 22).

### Target Sequences of EGFR:

(SEQ ID NO: 676):
5′-TACGAATATTAAACACTTCAA-3′
(qEGFR.10)
(SEQ ID NO: 677):
5′-ATAGGTATTGGTGAATTTAAA-3′
(qEGFR.11)
(SEQ ID NO: 678):
5′-CAGGAACTGGATATTCTGAAA-3′
(qEGFR.12)
(SEQ ID NO: 679):
5′-TGCCGCAAATTCCGAGACGAA-3′
(qEGFR.14)
(SEQ ID NO: 680):
5′-CCGCAAATTCCGAGACGAA-3′
(hEGFR.1b3)
(SEQ ID NO: 681):
5′-CAAAGTGTGTAACGGAATA-3′
(hEGFR.2b3)
(SEQ ID NO: 682):
5′-GTAACAAGCTCACGCAGTT-3′
(hEGFR.3b3)
(SEQ ID NO: 683):
5′-GAGGAAATATGTACTACGA-3′
(hEGFR.4b3)

Previously Disclosed in U.S. Pat. No. 10,689,654:

(SEQ ID NO: 684):
5′-CCTTAGCAGTCTTATCTAA-3′
(U02)

All of the siRNAs targeting the sequences above demonstrated significant decrease in target expression, with SEQ ID NO: 682 and SEQ ID NO: 684 showing the most promising inhibition.

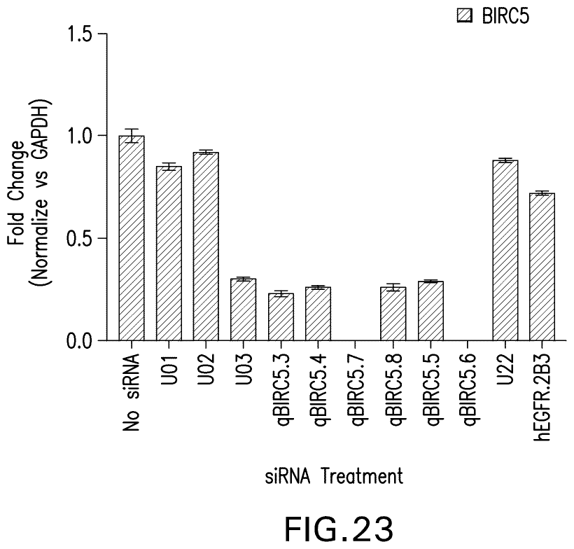

SW-480 cells were treated with siRNA and the expression of BIRC5 was measured after treatment (FIG. 23).

### Target Sequences of BIRC5:

(SEQ ID NO: 685):
5′-AAGAAGCAGTTTGAAGAATTA-3′
(qBIRC5.3)
(SEQ ID NO: 686):
5′-CCGCATCTCTACATTCAAGAA-3′
(qBIRC5.4)
(SEQ ID NO: 687):
5′-CTCGGCTGTTCCTGAGAAATA-3′
(qBIRC5.7)
(SEQ ID NO: 688):
5′-CTGGCGTAAGATGATGGATTT-3′
(qBIRC5.8)
(SEQ ID NO: 689):
5′-AAGCATTCGTCCGGTTGCGCT-3′
(qBIRC5.5)
(SEQ ID NO: 690):
5′-TGCACCACTTCCAGGGTTTAT-3′
(qBIRC5.6)

Previously Disclosed in U.S. Pat. No. 10,689,654:

(SEQ ID NO: 691):
5′-GGACCACCGCATCTCTACA-3′
(U03)

(SEQ ID NO: 691) decreased BIRC5 expression 70% and (SEQ ID NO: 685) decreased expression 76%.

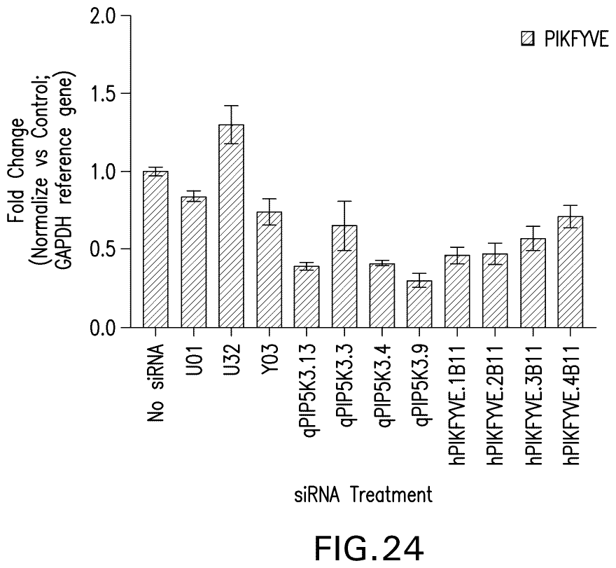

HCT116 cells were treated with siRNA and the expression of PIKFYVE was measured after treatment (FIG. 24).

### Target Sequences of PIKFYVE:

(SEQ ID NO: 692):
5′-CAGAGATGAGTATGCGCTGTA-3′
(qPIK5k3.13)
(SEQ ID NO: 693):
5′-ATCCTGGTTTAAAGACATAAA-3′
(qPIK5k3.4)
(SEQ ID NO: 694):
5′-CAACGTGAACTTCCATATCAA-3′
(qPIK5k3.3)
(SEQ ID NO: 695):
5′-ACCCAGTAACATAATATTTCA-3′
(qPIK5k3.9)
(SEQ ID NO: 696):
5′-GAATGGAGTTTCAGGATCA-3′
(hPIKFYVE.1B11)
(SEQ ID NO: 697):
5′-GGAAATCTCCTGCTCGAAA-3′
(hPIKFYVE.2B11)
(SEQ ID NO: 698):
5′-TGAAGAAGGTGACGATAAT-3′
(hPIKFYVE.3B11)
(SEQ ID NO: 699):
5′-GGACTCTGCTAATGATTTG-3′
(hPIKFYVE.4B11)

SEQ ID NO: 695 decreased PIKFYVE expression 69%.

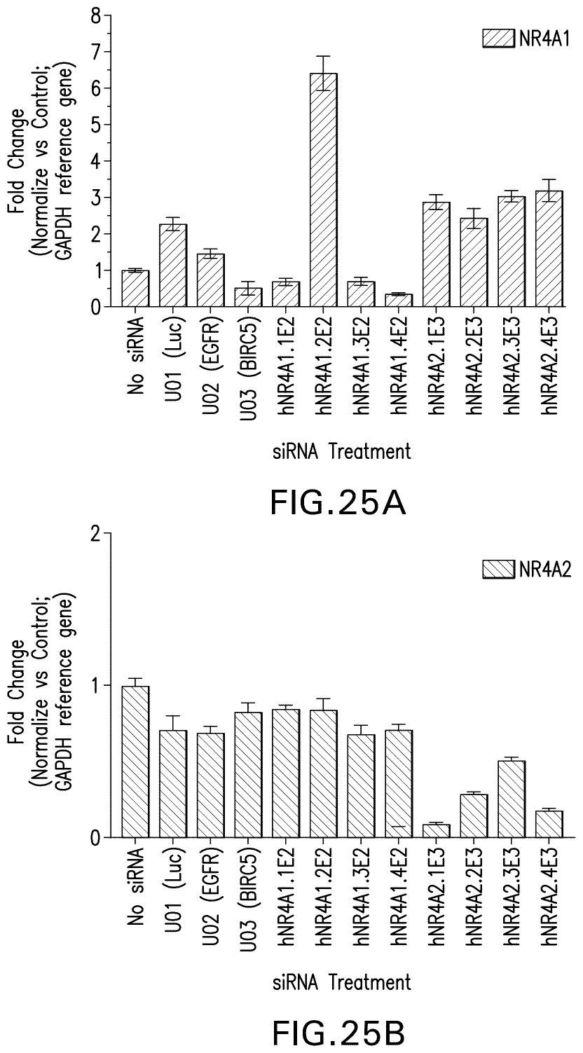

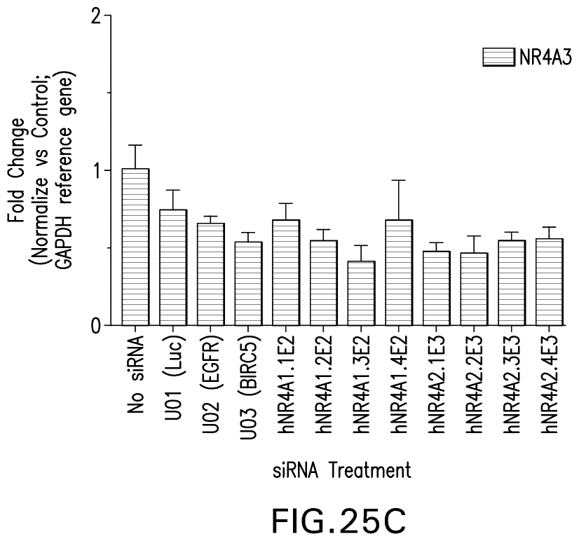

SK-BR3 cells were treated with siRNA and the expression of NR4A1 (FIG. 25A), NR4A2 (FIG. 25B), and NR4A3 (FIG. 25C) was measured after treatment.

### Target Sequences of NR4A1:

(SEQ ID NO: 700):
5′-GCACCTTCATGGACGGCTA-3′
(hNR4A1.1E2)
(SEQ ID NO: 701):
5′-GCATTATGGTGTCCGCACA-3′
(hNR4A1.2E2)
(SEQ ID NO: 702):
5′-TGAAGGAAGTTGTCCGAAC-3′
(hNR4A1.3E2)
(SEQ ID NO: 703):
5′-CTGCAGAACCGCATCGCCA-3′
(hNR4A1.4E2)

### Target Sequences of NR4A2:

(SEQ ID NO: 704):
5′-CCACGTGACTTTCAACAAT-3′ (hNR4A2.1E3)
(SEQ ID NO: 705):
5′-ACATTCAGATGCACAACTA-3′ (hNR4A2.2E3)
(SEQ ID NO: 706):
5′-GGACAAGCGTCGCCGGAAT-3′ (hNR4A2.3E3)
(SEQ ID NO: 707):
5′-CCACCTTGCTTGTACCAAA-3′ (hNR4A2.4E3)

siRNA targeting (SEQ ID NO: 701) induced NR4A1 expression while (SEQ ID NO: 700), (SEQ ID NO: 702) and (SEQ ID NO: 703) reduced it. All four siRNAs targeting NR4A2 sequences reduced NR4A2 expression with (SEQ ID NO: 704) decreasing expression 91%. Sequences were found to moderately reduce NR4A3 expression.

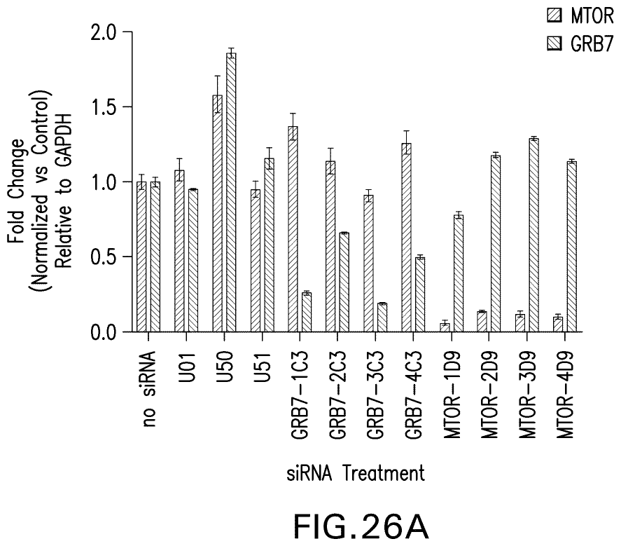

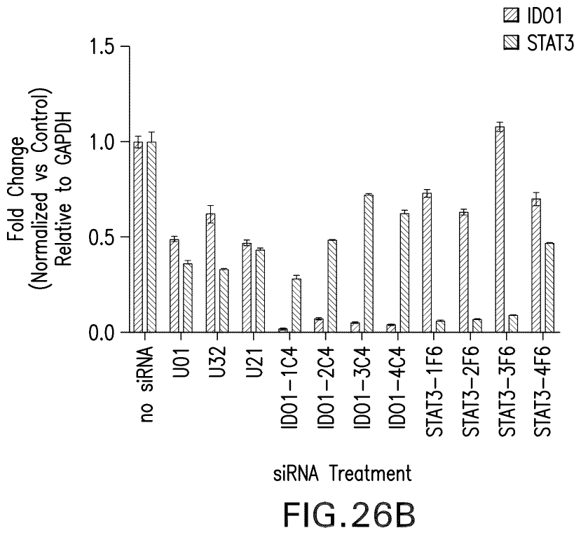

SK-BR3 cells were treated with siRNA and the expression of MTOR and GRB7 (FIG. 26A) was measured after treatment.

### Target Sequences of GRB7:

(SEQ ID NO: 708):
5′-AGAAGTGCCTCAGATAATA-3′ (GRB7-1C3)
(SEQ ID NO: 709):
5′-TAGTAAAGGTGTACAGTGA-3′ (GRB7-2C3)
(SEQ ID NO: 710):
5′-TGCAGAAAGTGAAGCATTA-3′ (GRB7-3C3)
(SEQ ID NO: 711):
5′-GGAGATAGCCGCTTCGTCT-3′ (GRB7-4C3)

### Target Sequences of MTOR:

(SEQ ID NO: 712):
5′-GAGAAGAAATGGAAGAAAT-3′ (MTOR-1D9)
(SEQ ID NO: 713):
5′-CCAAAGTGCTGCAGTACTA-3′ (MTOR-2D9)
(SEQ ID NO: 714):
5′-GGTCTGAACTGAATGAAGA-3′ (MTOR-3D9)
(SEQ ID NO: 715):
5′-ATAAAGTTCTGGTGCGACA-3′ (MTOR-4D9)

siRNA targeting (SEQ ID NO: 708) and (SEQ ID NO: 710) reduced GRB7 expression and all four siRNAs targeting MTOR greatly reduced MTOR expression.

BT549 cells were treated with siRNA and the expression of IDO1 and STAT3 (FIG. 26B) was measured after treatment.

### Target Sequences of IDO1:

(SEQ ID NO: 716):
5′-AGAAAGAGTTGAGAAGTTA-3′ (IDO1-1C4)
(SEQ ID NO: 717):
5′-GAAATATTGCTGTTCCTTA-3′ (IDO1-2C4)
(SEQ ID NO: 718):
5′-GAACGGGACACTTTGCTAA-3′ (IDO1-3C4)
(SEQ ID NO: 719):
5′-GGGCAAAGGTCATGGAGAT-3′ (IDO1-4C4)

### Target Sequences of STAT3:

(SEQ ID NO: 720):
5′-GGAGAAGCATCGTGAGTGA-3′ (STAT3-1F6)
(SEQ ID NO: 721):
5′-CCACTTTGGTGTTTCATAA-3′ (STAT3-2F6)
(SEQ ID NO: 722):
5′-TCAGGTTGCTGGTCAAATT-3′ (STAT3-3F6)
(SEQ ID NO: 723):
5′-CGTTATATAGGAACCGTAA-3′ (STAT3-4F6)

All four siRNAs targeting IDO1 sequences above demonstrated significant decrease in expression, while (SEQ ID NO: 720), (SEQ ID NO: 721), and (SEQ ID NO: 722) demonstrated decrease in STAT3 expression.

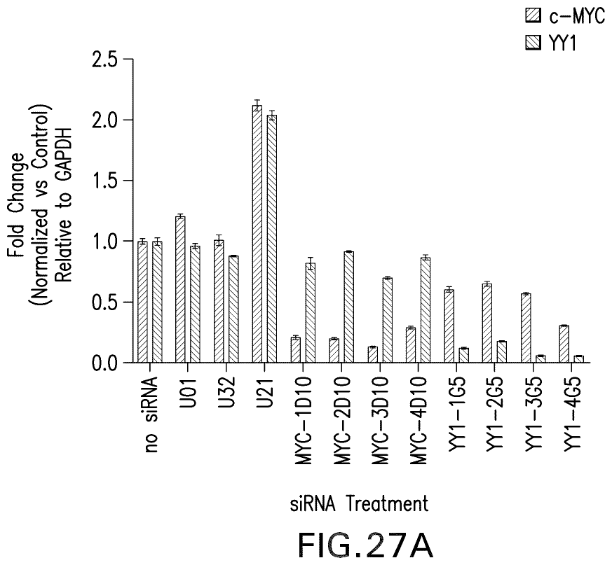

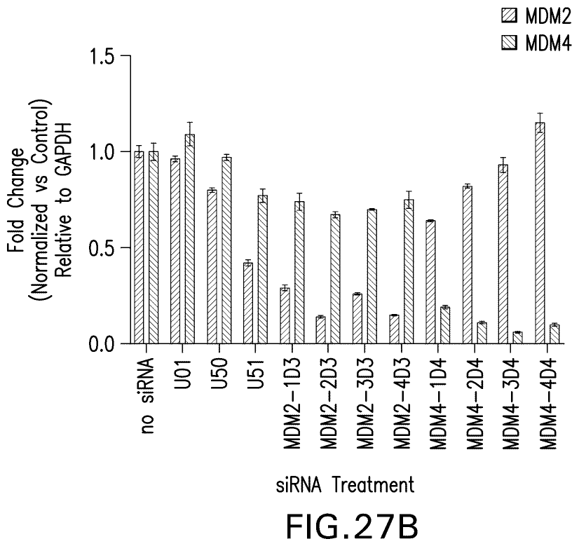

HCT116 cells were treated with siRNA and the expression of c-MYC and YY1 (FIG. 27A) was measured after treatment.

Target Sequences of c-MYC:

(SEQ ID NO: 724):
5′-AACGTTAGCTTCACCAACA-3′ (MYC-1D10)
(SEQ ID NO: 725):
5′-GGAACTATGACCTCGACTA-3′ (MYC-2D10)
(SEQ ID NO: 726):
5′-GAACACACAACGTCTTGGA-3′ (MYC-3D10)
(SEQ ID NO: 727):
5′-CTACCAGGCTGCGCGCAAA-3′ (MYC-4D10)

### Target Sequences of YY1:

(SEQ ID NO: 728):
5′-GGATAACTCGGCCATGAGA-3′ (YY1-1G5)
(SEQ ID NO: 729):
5′-CAAGAAGAGTTACCTCAGC-3′ (YY1-2G5)
(SEQ ID NO: 730):
5′-GAACTCACCTCCTGATTAT-3′ (YY1-3G5)
(SEQ ID NO: 731):
5′-GCTTAGTAATGCTACGTGT-3′ (YY1-4G5)

All four siRNAs targeting c-MYC demonstrated decrease in expression levels, with SEQ ID NO: 725 and SEQ ID NO: 726 showing the largest reduction in expression. All four siRNAs targeting YY1 also demonstrated decrease in expression levels, with SEQ ID NO: 730 and SEQ ID NO: 731 showing the largest reduction in expression.

HCT116 cells were treated with siRNA and the expression of MDM2 and MDM4 (FIG. 27B) was measured after treatment.

### Target Sequences of MDM2:

(SEQ ID NO: 732):
5′-GCCAGTATATTATGACTAA-3′ (MDM2-1D3)
(SEQ ID NO: 733):
5′-GATGAGAAGCAACAACATA-3′ (MDM2-2D3)
(SEQ ID NO: 734):
5′-CCCTAGGAATTTAGACAAC-3′ (MDM2-3D3)
(SEQ ID NO: 735):
5′-AAAGTCTGTTGGTGCACAA-3′ (MDM2-4D3)

### Target Sequences of MDM4:

(SEQ ID NO: 736):
5′-GCAGTTAGGTGTTGGAATA-3′ (MDM4-1D4)
(SEQ ID NO: 737):
5′-TGATACCGATGTAGAGGTT-3′ (MDM4-2D4)
(SEQ ID NO: 738):
5′-GCATAATGGTAGTACGAAC-3′ (MDM4-3D4)
(SEQ ID NO: 739):
5′-CCACGAGACGGGAACATTA-3′ (MDM4-4D4)

siRNAs targeting (SEQ ID NO: 733) and (SEQ ID NO: 735) demonstrated significant reduction in MDM2 expression. And all four siRNAs targeting MDM4 demonstrated decreases in expression levels with (SEQ ID NO: 738) and (SEQ ID NO: 739) exhibiting the greatest expression decrease.

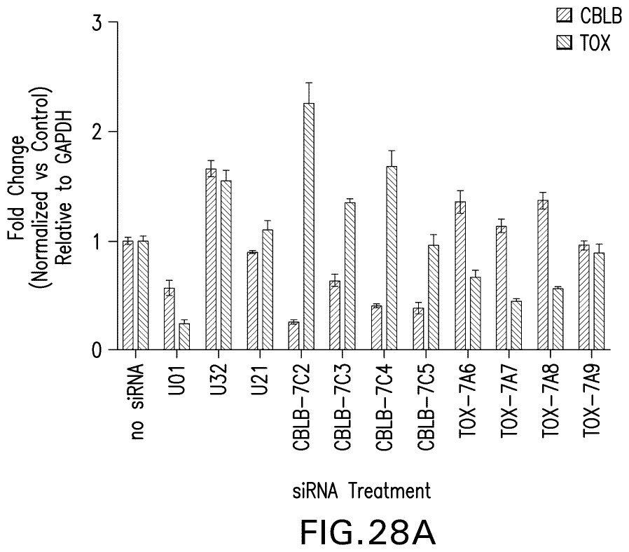

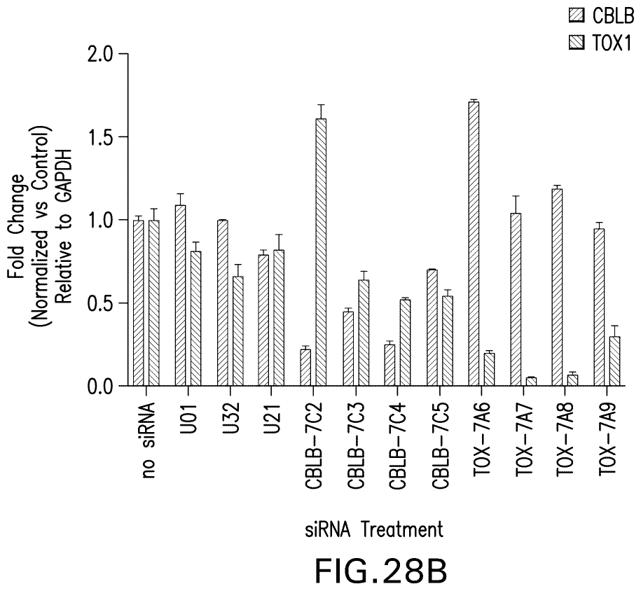

U2OS (FIG. 28A) and ES-2 (FIG. 28B) cells were treated with siRNA and the expression of CBLB and TOX was measured after treatment.

### Target Sequences of CBLB:

(SEQ ID NO: 740):
5′-GACCATACCTCATAACAAG-3′ (CBLB-7C2)
(SEQ ID NO: 741):
5′-TGAAAGACCTCCACCAATC-3′ (CBLB-7C3)
(SEQ ID NO: 742):
5′-GATGAAGGCTCCAGGTGTT-3′ (CBLB-7C4)
(SEQ ID NO: 743):
5′-TATCAGCATTTACGACTTA-3′ (CBLB-7C5)

### Target Sequences of TOX:

(SEQ ID NO: 744):
5′-CCACATGGCCAGCTGACTA-3′
(SEQ ID NO: 745):
5′-CAACCCGACTATCAGACTA-3′
(SEQ ID NO: 746):
5′-GAATGAATCCTCACCTAAC-3′
(SEQ ID NO: 747):
5′-GCAACAAGTTTGACGGTGA-3′

siRNAs targeting (SEQ ID NO: 740):) demonstrated significant reduction in CBLB expression, but all four siRNAs showed efficacy. All four siRNAs targeting TOX demonstrated decreases in expression levels with (SEQ ID NO: 745) exhibiting the greatest expression decrease.

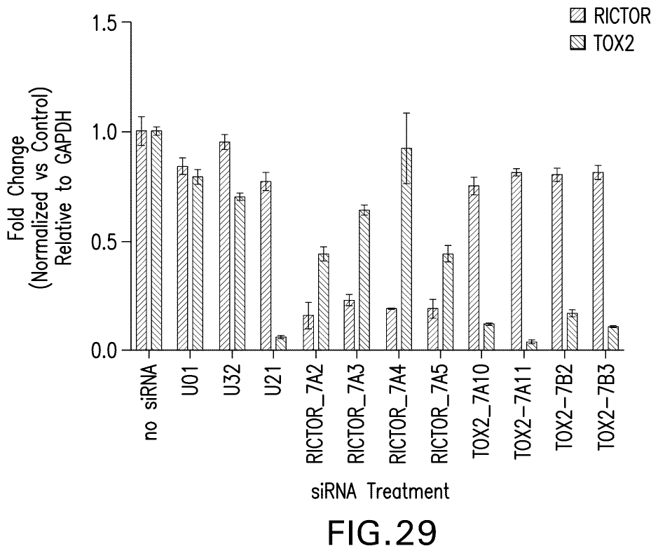

HCT116 cells were treated with siRNA and the expression of RICTOR and TOX2 (FIG. 29) was measured after treatment.

### Target Sequences of RICTOR:

(SEQ ID NO: 748:
5′-TCAACGAGCTCACATATGA-3′ (RICTOR_7A2)
(SEQ ID NO: 749):
5′-TGACCGATCTGGACCCATA-3′ (RICTOR_7A3)
(SEQ ID NO: 750):
5′-GTACTTGGGCTCATAGCTA-3′ (RICTOR_7A4)
(SEQ ID NO: 751):
5′-GCAGATGAGTCTTACGGAA-3′ (RICTOR_7A5)

### Target Sequences of TOX2:

(SEQ ID NO: 752):
5′-GGAAGTGCATTTCAAGATC-3′ (TOX2_7A10)
(SEQ ID NO: 753):
5′-CGAGAACAACGAAGACTAT-3′ (TOX2_7A11)
(SEQ ID NO: 754):
5′-CAAGAGCACTCAGGCAAAC-3′ (TOX2_7B2)
(SEQ ID NO: 755):
5′-AAAGAGACCTTCAGCCGAC-3′ (TOX2_7B3)

All four siRNAs targeting RICTOR demonstrated significant reduction in RICTOR expression. All four siRNAs targeting TOX2 also demonstrated decreases in expression levels of TOX2 with (SEQ ID NO: 753) exhibiting the greatest expression decrease.

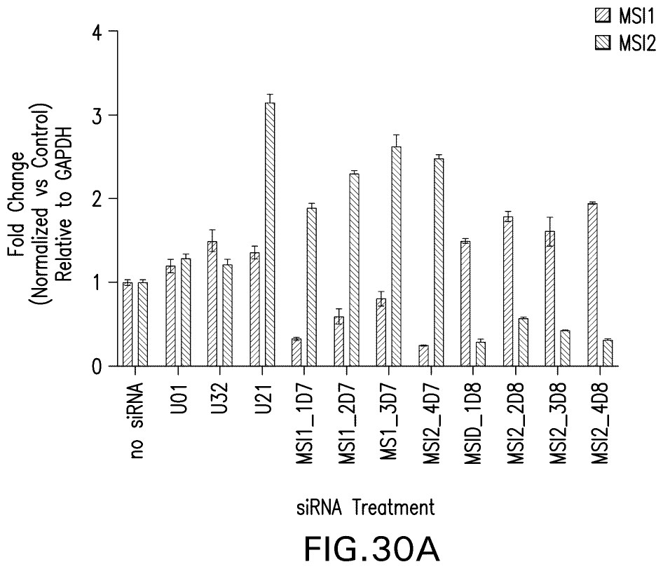

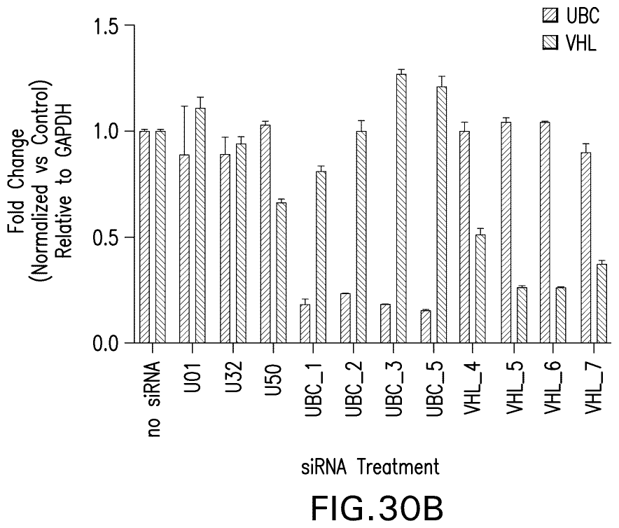

HCT116 cells were treated with siRNA and the expression of MSI1 and MSI2 (FIG. 30A) was measured after treatment.

### Target Sequences of MSI1:

(SEQ ID NO: 756):
5′-GGACTCAGTTGGCAGACTA-3′ (MSI1_1D7)
(SEQ ID NO: 757):
5′-AGGAAGGGCTGCGCGAATA-3′ (MSI1_2D7)
(SEQ ID NO: 758):
5′-ATAAAGTGCTGGCGCAATC-3′ (MSI1_3D7)
(SEQ ID NO: 759):
5′-GAGTCATGCCCTACGGAAT-3′ (MSI1_4D7)

### Target Sequences of MSI2:

(SEQ ID NO: 760):
5′-CAATGCTGATGTTTGATAA-3′ (MSI2_1D8)
(SEQ ID NO: 761):
5′-CCAGATAGCCTTAGAGACT-3′ (MSI2_2D8)
(SEQ ID NO: 762):
5′-GAGTTAGATTCCAAGACGA-3′ (MSI2_3D8)
(SEQ ID NO: 763):
5′-CCAACTTCGTGGCGACCTA-3′ (MSI2_4D8)

All four siRNAs targeting MSI1 targeting siRNAs demonstrated significant reduction in MSI1 expression but (SEQ ID NO: 759) showed the most significant decrease in target expression. All four siRNAs targeting MSI2 also demonstrated decreases in expression levels of MSI2 with (SEQ ID NO: 760) exhibiting the greatest expression decrease.

HCT116 cells were treated with siRNA and the expression of UBC and VHL (FIG. 30B) was measured after treatment.

### Target Sequences of UBC:

(SEQ ID NO: 764):
5′-GAGGTTGATCTTTGCCGGAAA-3′ (UBC_1)
(SEQ ID NO: 765):
5′-GAGGTTGATCTTTGCTGGGAAA-3′ (UBC_2)
(SEQ ID NO: 766):
5′-AACGTCAAAGCAAAGATCCAA-3′ (UBC_3)
(SEQ ID NO: 767):
5′-ATCGCTGTGATCGTCACTTGA-3′ (UBC_5)

### Target Sequences of VHL:

(SEQ ID NO: 768):
5′-AAGGAGGTTTGTATAAGTAAT-3′ (VHL_4)
(SEQ ID NO: 769):
5′-CAGGAGCGCATTGCACATCAA-3′ (VHL_5)
(SEQ ID NO: 770):
5′-TTCAGTGGGAATTGCAGCATA-3′ (VHL_6)
(SEQ ID NO: 771):
5′-CTGATGAGTCTTGATCTAGAT-3′ (VHL_7)

All four siRNAs targeting UBC targeting siRNAs demonstrated significant reduction in UBC expression. All four siRNAs targeting VHL also demonstrated decreases in expression levels of VHL particularly (SEQ ID NO: 769) and (SEQ ID NO: 770).

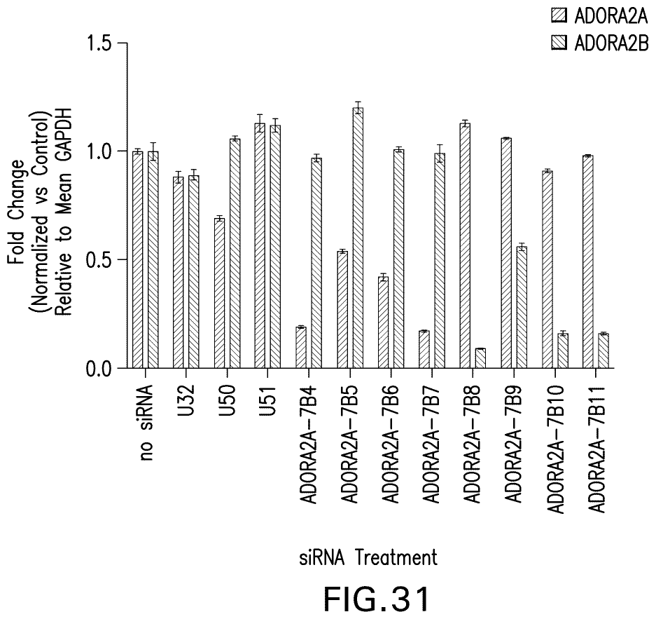

SKBR3 cells were treated with siRNA and the expression of ADORA2A and ADORA2B (FIG. 31) was measured after treatment.

### Target Sequences of ADORA2A:

(SEQ ID NO: 396):
5′-GAACGUCACCAACUACUUU-3′ (ADORA2A-7B4)
(SEQ ID NO: 397):
5′-CAUGCUGGGUGUCUAUUUG-3′ (ADORA2A-7B5)
(SEQ ID NO: 398):
5′-CAACUGCGGUCAGCCAAAG-3′ (ADORA2A-7B6)
(SEQ ID NO: 399):
5′-CCAAGUGGCCUGUCUCUUU-3′ (ADORA2A-7B7)

### Target Sequences of ADORA2B:

(SEQ ID NO: 400):
5′-UGAGCUACAUGGUAUAUUU-3′ (ADORA2b-7B8)
(SEQ ID NO: 401):
5′-GGGAUGGAACCACGAAUGA-3′ (ADORA2b-7B9)
(SEQ ID NO: 402):
5′-GAUGGAACCACGAAUGAAA-3′ (ADORA2b-7B10)
(SEQ ID NO: 403):
5′-GAACCGAGACUUCCGCUAC-3′ (ADORA2b-7B11)

All four siRNAs targeting ADORA2A demonstrated significant reduction in ADORA2A expression, with (SEQ ID NO: 396) and (SEQ ID NO: 398) demonstrating the most significant reduction in expression. All four siRNAs targeting ADORA2B also demonstrated decreases in expression levels of ADORA2B particularly (SEQ ID NO: 400).

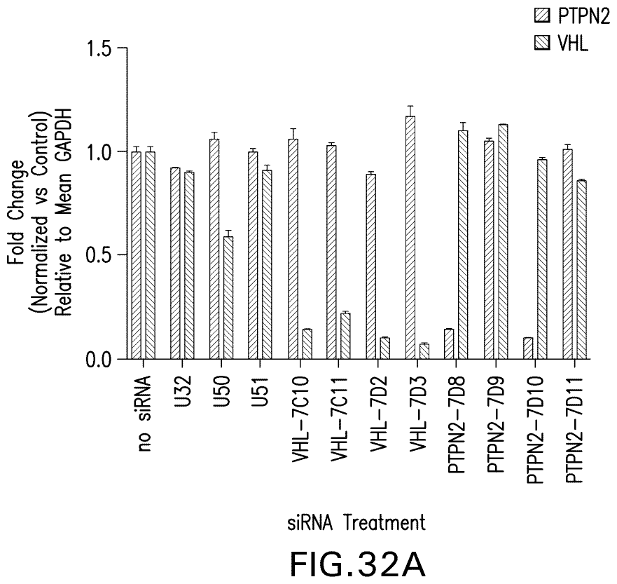

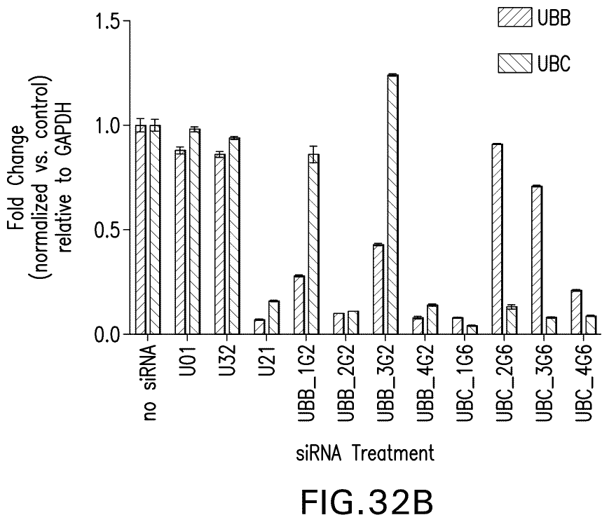

HCT116 cells were treated with siRNA and the expression of PTPN2 and VHL (FIG. 32A) was measured after treatment.

### Target Sequences of PTPN2:

(SEQ ID NO: 420):
5′-GAAACAGGAUUCAGUGUGA-3′ (PTPN2-7D8)
(SEQ ID NO: 421):
5′-ACAAAGGAGUUACAUCUUA-3′ (PTPN2-7D9)
(SEQ ID NO: 422):
5′-AAAGGGAGAUUCUAGUAUA-3′ (PTPN2-7D10)
(SEQ ID NO: 423):
5′-AAACAGAAAUCGAAACAGA-3′ (PTPN2-7D11)

### Target Sequences of VHL:

(SEQ ID NO: 412):
5′-CCGUAUGGCUCAACUUCGA-3′ (VHL-7C10)
(SEQ ID NO: 413):
5′-AGGCAGGCGUCGAAGAGUA-3′ (VHL-7C11)
(SEQ ID NO: 414):
5′-GCUCUACGAAGAUCUGGAA-3′ (VHL-7D2)
(SEQ ID NO: 415):
5′-GGAGCGCAUUGCACAUCAA-3′ (VHL-7D3)

All four siRNAs targeting VHL demonstrated significant reduction in VHL expression with (SEQ ID NO: 415) demonstrating the most significant reduction in expression. Two siRNAs targeting PTPN2 also demonstrated significant in expression levels of PTPN2 particularly (SEQ ID NO: 422).

HCT116 cells were treated with siRNA and the expression of UBB and UBC (FIG. 32B) was measured after treatment.

### Target Sequences of UBB:

(SEQ ID NO: 301):
5′-GCCGUACUCUUUCUGACUA-3′ (UBB_1G2)
(SEQ ID NO: 302):
5′-GUAUGCAGAUCUUCGUGAA-3′ (UBB_2G2)
(SEQ ID NO: 303):
5′-GACCAUCACUCUGGAGGUG-3′ (UBB_3G2)
(SEQ ID NO: 304):
5′-CCCAGUGACACCAUCGAAA-3′ (UBB_4G2)

### Target Sequences of UBC:

(SEQ ID NO: 305):
5′-GUGAAGACCCUGACUGGUA-3′ (UBC_1G6)
(SEQ ID NO: 306):
5′-AAGCAAAGAUCCAGGACAA-3′ (UBC_2G6)
(SEQ ID NO: 307):
5′-GAAGAUGGACGCACCCUGU-3′ (UBC_3G6)
(SEQ ID NO: 308):
5′-GUAAGACCAUCACUCUCGA-3′ (UBC_4G6)

All four siRNAs targeting UBB alone demonstrated reduction in UBB expression, with (SEQ ID NO: 303) and (SEQ ID NO: 304) demonstrating significant reduction in expression. All four siRNAs targeting UBC demonstrated significant decreases in expression levels of UBC. (SEQ ID NO: 302), (SEQ ID NO: 304), and (SEQ ID NO: 305) demonstrated comparable dual action inhibition to U21.

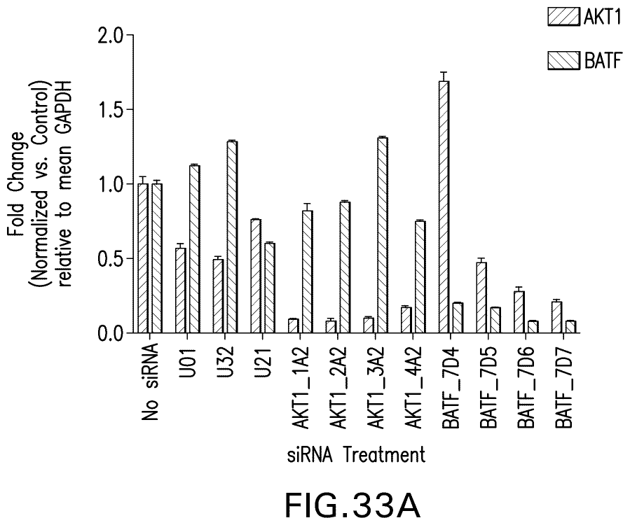

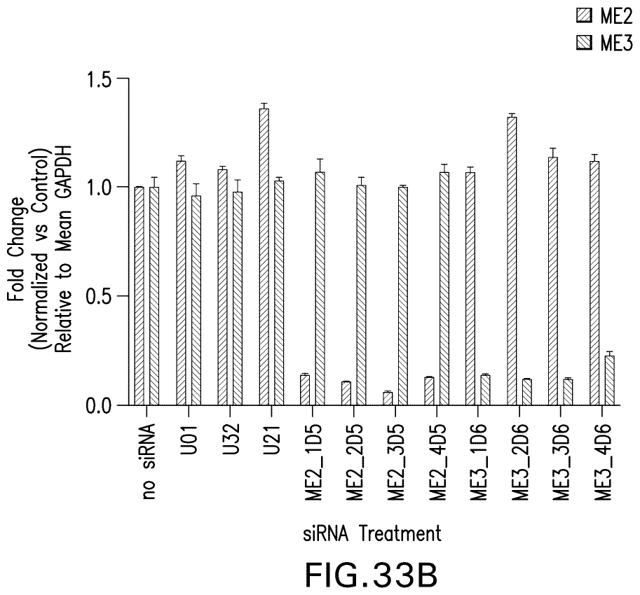

SKBR3 cells were treated with siRNA and the expression of AKT1 and BATF (FIG. 33A) was measured after treatment.

### Target Sequences of AKT1:

(SEQ ID NO: 5):
5′-GACAAGGACGGGCACAUUA-3′ (AKT1_1A2)
(SEQ ID NO: 6):
5′-GCUACUUCCUCCUCAAGAA-3′ (AKT1_2A2)
(SEQ ID NO: 7):
5′-GACCGCCUCUGCUUUGUCA-3′ (AKT1_3A2)
(SEQ ID NO: 8):
5′-GGCAGCACGUGUACGAGAA-3′ (AKT1_4A2)

### Target Sequences of BATF:

(SEQ ID NO: 416):
5′-GUACAGCGCCCACGCAUUC-3′ (BATF_7D4)
(SEQ ID NO: 417):
5′-GAAACAGAACGCGGCUCUA-3′ (BATF_7D5)
(SEQ ID NO: 418):
5′-GAACGCGGCUCUACGCAAG-3′ (BATF_7D6)
(SEQ ID NO: 419):
5′-AGAGUUCAGAGGAGGGAGA-3′ (BATF_7D7)

All four siRNAs targeting AKT1 alone demonstrated reduction in AKT1 expression, with minimal off-target effects. All four BATF targeting siRNAs exhibited significant reduction in BATF expression with possible dual action inhibition to AKT1.

22Rv1 cells were treated with siRNA and the expression of ME2 and ME3 (FIG. 33B) was measured after treatment.

### Target Sequences of ME2:

(SEQ ID NO: 177):
5′-GAAGAAGCAUAUACACUUA-3′ (ME2_1D5)
(SEQ ID NO: 178):
5′-UGAAAGGCCUGUAAUAUUU-3′ (ME2_2D5)
(SEQ ID NO: 179):
5′-GAACAUGGCGGAGUGAAUA-3′ (ME2_3D5)
(SEQ ID NO: 180):
5′-AUUAGUUAAGGGACGGAAA-3′ (ME2_4D5)

### Target Sequences of ME3:

(SEQ ID NO: 181):
5′-CAACAAUGCUGAAUUCUUG-3′ (ME3_1D6)
(SEQ ID NO: 182):
5′-ACAAAUACCGUAACAAGUA-3′ (ME3_2D6)
(SEQ ID NO: 183):
5′-GGAGCCACCUGAACCAUGA-3′ (ME3_3D6)
(SEQ ID NO: 184):
5′-CUAAAGGGCUCAUUGUCAA-3′ (ME3_4D6)

All four siRNAs targeting ME2 alone demonstrated significant reduction in ME2 expression, with (SEQ ID NO: 237) exhibiting the most significant reduction. All four ME3 targeting siRNAs also exhibited significant reduction in ME3.

### Example 17: EPCAM Aptamer Construction

EpCAM aptamers were individually synthesized by in vitro transcription with PCR products as templates. The ssDNA of EpCAM aptamer containing T7 RNA polymerase promoter site (underlined) and adaptor sequence (5′-TAATACGACTCACTATAGCGACTGGTTACCCGGTCGT-3′) (SEQ ID NO: 772) was synthesized from IDT as a PCR template. PCR was performed with forward primer (5′-TAATACGACTCACTATA GCGACTGGTTA-3) (SEQ ID NO: 773) and reverse primer (5′-ACGACCGGGTAACCAGTCGC-3′) (SEQ ID NO: 774). The PCR products were put into T-A cloning pCR 2.1 vector (Invitrogen) and sequenced. Transcription was performed with PCR product as templates using DuraScript transcription kits following manufacturer's instruction.

### Example 18: Bivalent Aptamer-Driven Delivery of Two siRNAs

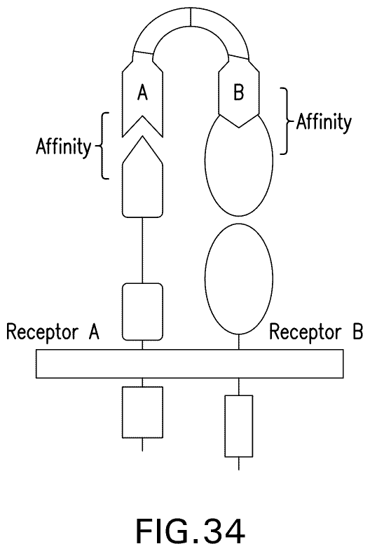

Bivalent aptamers support increased cargo internalization and specificity. Moreover, experiments for increasing ligand valency to augment cargo delivery has been demonstrated by the use of nanoparticle-based carriers (Pardella et al., Cancers 2020. 12(10), 2799) (FIG. 34).

### Example 19: EPCAM-UBB Aptamer-siRNA Chimera Construction

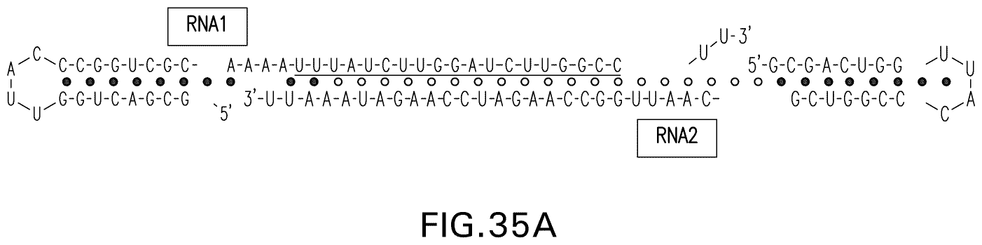

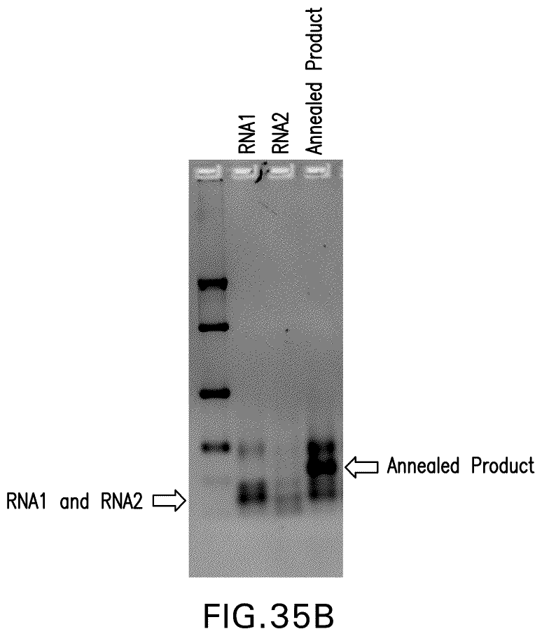

EpCAM-directed aptamers-siRNA chimeras were individually synthesized by in vitro transcription from an annealed DNA templates (FIG. 35A). For RNA 1, two ssDNA containing T7 RNA polymerase promoter site (underlined) and adaptor sequence (5′-GTAATACGACTCACTATAGGCGACTGGTTACCCGGTCGCAATTGGCCAAGATCCAA GATAAATT-3′) (SEQ ID NO: 775) and (5′-AATTTATCTTGGAUCTTGGCCAATTGCGACCGGGTAACCAGTCGCCTATAGTGAGT CGTATTAC-3′) (SEQ ID NO: 776) were synthesized by IDT as a T7 template. For RNA 2, two ssDNA containing T7 RNA polymerase promoter site (underlined) and adaptor sequence (5′-GTAATACGACTCACTATAGGCGACTGGTTACCCGGTCGCAAAATTTATCTTGGATCT TGGCCTT-3′) (SEQ ID NO: 777) and (5′-AAGGCCAAGATCCAAGATAAATTTTGCGACCGGGTAACCAGTCGCCTATAGTGAGT CGTATTAC-3′) (SEQ ID NO: 778) were synthesized by IDT as a T7 template. The annealed double stranded DNA for each RNA1 and RNA2 were used as templates for T7 polymerase using DuraScript transcription kits following manufacturer's instruction. The two RNAs were further purified and mixed at molar ratio 1:1 and annealed to form the chimeric molecule by heating at 94° C. for 3 min followed by slowly cooling to room temperature within 1 h. Resulting products were run on a gel for confirmation (FIG. 35B)

### Example 20: HER3-U21(UBB)-HER2 Aptamer-siRNA Chimera Construction

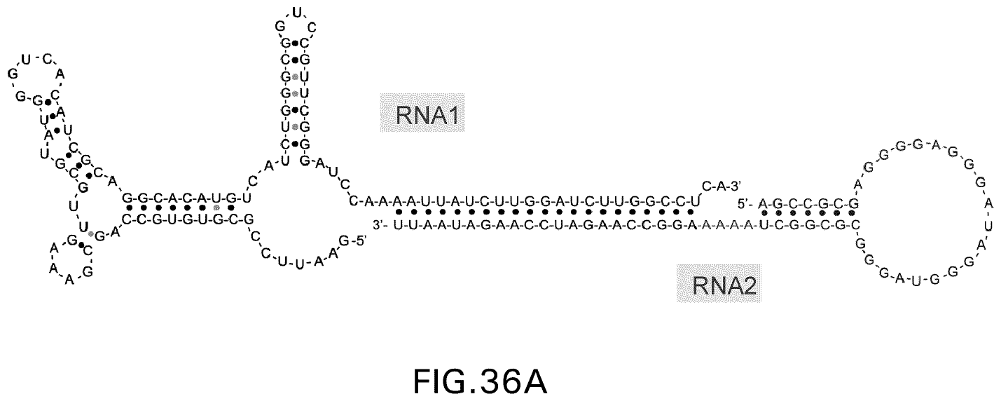

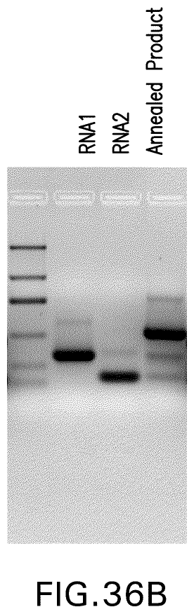

Utilizing the same synthesis method above, RNA1 and RNA2 is synthesized, purified, mixed, and annealed. The resulting products were run on a gel for confirmation (FIG. 36B)
RNA1: HER3 Aptamer-UBB antisense RNA
RNA2: HER2 Aptamer-UBB sense RNA

### Example 21: EPCAM-UBB Chimeras Construction

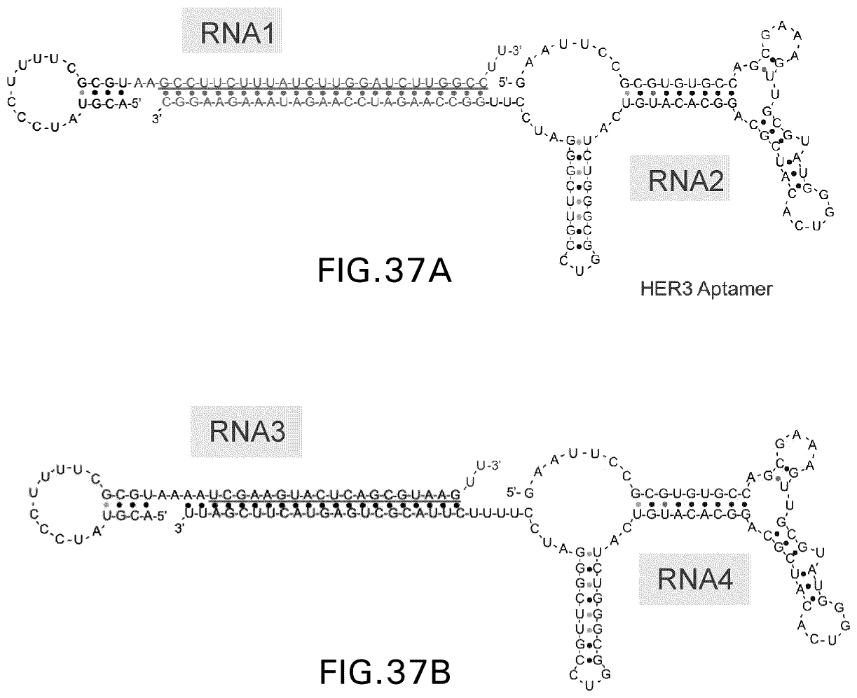

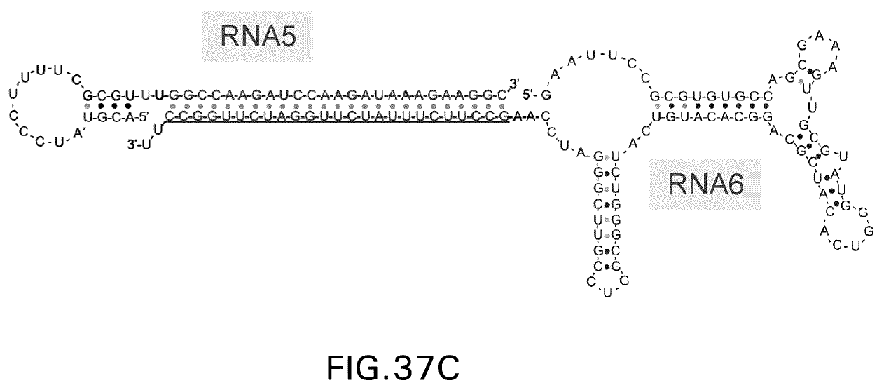

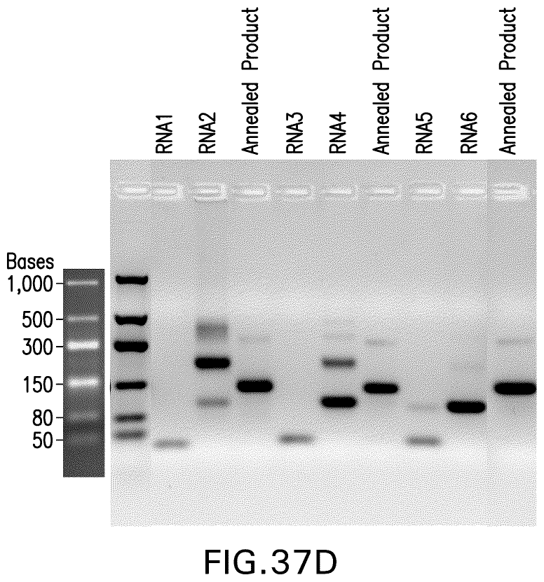

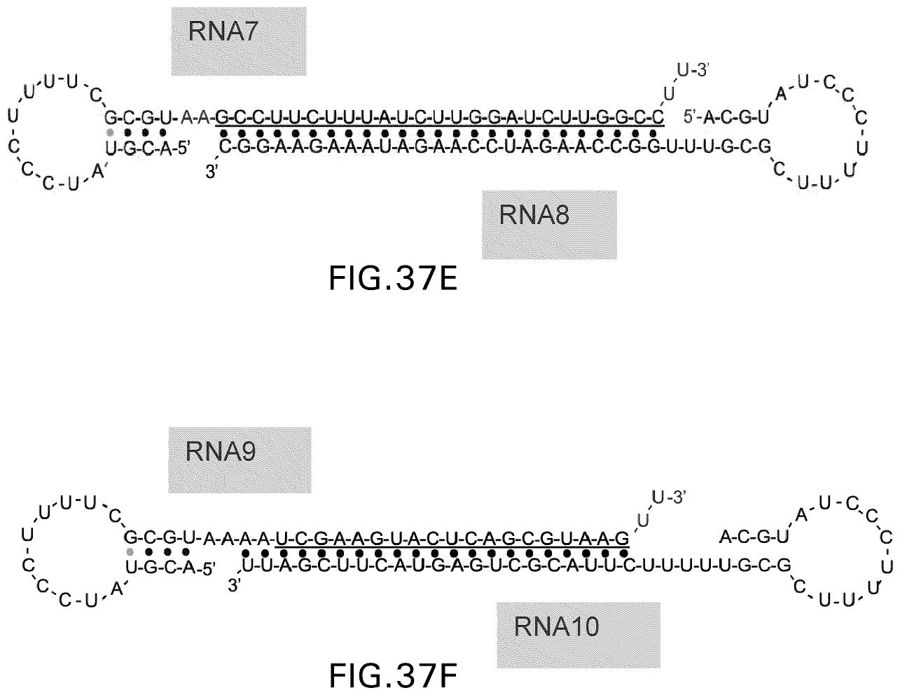

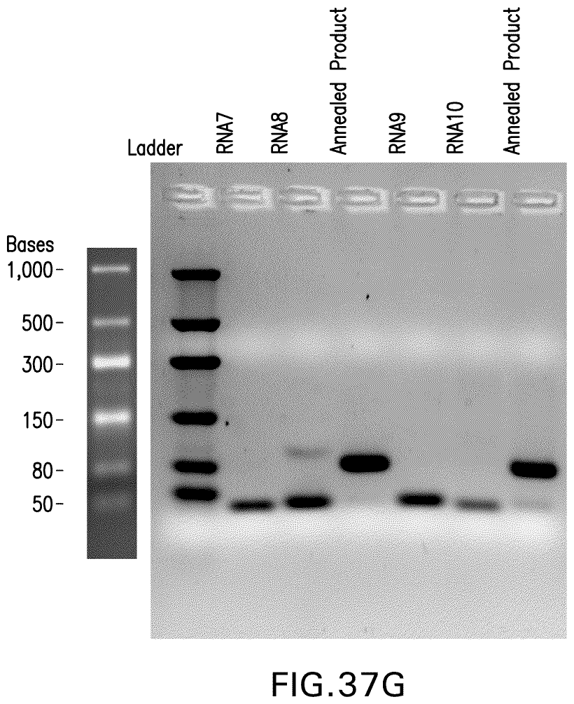

Utilizing the same synthesis method above, two RNA's are synthesized, purified, mixed, and annealed.
EPCAM-UBB-HER3 (FIG. 37A):
RNA1: EPCAM aptamer-U22ds Antisense RNA
RNA2: HER 3 Aptamer-U22ds Sense RNA
EPCAM-LUC-HER3 (FIG. 37B):
RNA3: EPCAM aptamer-Luc Antisense RNA
RNA4: HER 3 Aptamer-Luc Sense RNA
HER3-UBB-EPCAM (FIG. 37C)
RNA5: EPCAM aptamer-U22ds Sense RNA
RNA6: HER 3 Aptamer-U22ds anti-sense RNA
EPCAM-U22ds (UBB)-EPCAM (FIG. 37E)
RNA7: EPCAM aptamer with anti-sense U22ds siRNA
RNA8: EPCAM aptamer with sense U22ds siRNA
EPCAM-Luc-EPCAM (FIG. 37F)
RNA7: EPCAM aptamer with anti-sense Luc siRNA
RNA8: EPCAM aptamer with sense Luc siRNA

Alternative EPCAM aptamer sequences to be used in this construct or in other constructs of this application include:

2′F Cytidine with 2-Pyridyl at the 5 Position Uridine:

(SEQ ID NO: 779):
5′-GGGAGACAAACAAAGAGCGACAAGGGCAGAUAGCUUUUAGUUGUGCAA
UGCUCUGCACCGUCGAGUUCCCACCCAGAAGAAGCCAGAAG-3′
(SEQ ID NO: 780):
5′-GGGAGACAAACAAAGAGCGACAAGGGCAGUGAGAUAGUAGACGAGGAG
GUUCCAUUAGAAUGCAAAUAUCACCCAGAAGAAGCCAGAAG-3′
2′F cytidine with Benzyl at the 5 position uridine:
(SEQ ID NO: 781):
5′-GGGAGACAAACAAAGAGCGACAAGGGCAGAGGUACCAAGCAGAGGGUC
UAAGGGUAGCCCGGACGAGUCACCCAGAAGAAGCCAGAAG-3′
(SEQ ID NO: 782):
5′-GGGAGACAAACAAAGAGCGACAAGGGCAGCAUCUGCUAGUAAUGUUCG
GCGGUCGAACUCUACUUGGAACACCCAGAAGAAGCCAGAAG-3′
(SEQ ID NO: 783):
5′-GGGAGACAAACAAAGAGCGACAAGGGCAGAGACAGGUUAGGGGAAAGU
GUGUUAAACUUUAAAGUAAUUCACCCAGAAGAAGCCAGAAG-3′

Alternative HER2 aptamer sequences to be used in this construct or in other constructs of this application include:

2′F Cytidine with 2-Pyridyl at the 5 Position Uridine:

(SEQ ID NO: 784):
5′-GGGAGACAAACAAAGAGCGACAAGGGCAGGUAGGACGUCAGUUUAA
CGCAAGCGCGUUACACCUAGAUCCACCCAGAAGAAGCCAGAAG-3′
(SEQ ID NO: 785):
5′-GGGAGACAAACAAAGAGCGACAAGGGCAGCCUAUUUUGGGGCUGUG
ACAUAUUGUCAAAUGCUAAACGGCACCCAGAAGAAGCCAGAAG-3′
(SEQ ID NO: 786):
5′-GGGAGACAAACAAAGAGCGACAAGGGCAGAGCAGUUUCGUUGGGCG
UCGGUCUAAUAGACUGACUGGGGCACCCAGAAGAAGCCAGAAG-3′

2′F Cytidine with Benzyl at the 5 Position Uridine:

(SEQ ID NO: 787):
5′-GGGAGACAAACAAAGAGCGACAAGGGCAGGUCCAAUCUGAGUGAUG
UCUGUCAAGACCUAGAGAAGUACCACCCAGAAGAAGCCAGAAG-3′
(SEQ ID NO: 788):
5′-GGGAGACAAACAAAGAGCGACAAGGGCAGAGUGAUGUCUGUCAAGA
CCGGGCUCUACCGCUGGUUCAAGCACCCAGAAGAAGCCAGAAG-3′
(SEQ ID NO: 789):
5′-GGGAGACAAACAAAGAGCGACAAGGGCAGUGGGAGCACUAGAAUGG
UCUAUAUUAAUGUUAGCGCAGUGCACCCAGAAGAAGCCAGAAG-3′
(SEQ ID NO: 790):
5′-AGGAGAUGCGUAGGGUGGACUGAGUGAUGUCUGUCAAGACCUCGUC
CAAAC-3′
(SEQ ID NO: 791):
5′-AGGAGUGAUGUCUGUCAAGACCGAUUGUCUGCCACUCAAUCGGGAC
CAAAC-3′
(SEQ ID NO: 792):
5′-AGGAGUGAUGUCUGUCAAGACCAGGUGCUGAGGUGACUCUGUAAUU
CAAAC-3′

Resulting products were run on a gel for confirmation (FIGS. 37D and 37G)

### Example 22: Building and Testing Bispecific Aptamer-siRNA: PSMA-BIRC5-UBB/UBC-PSMA

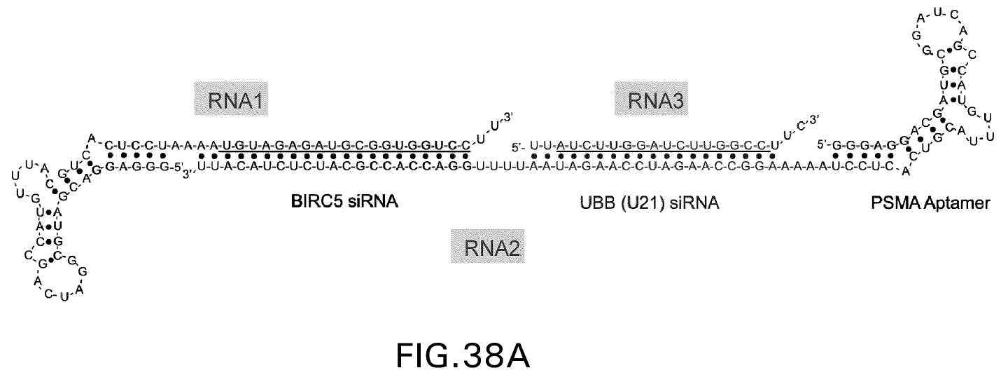

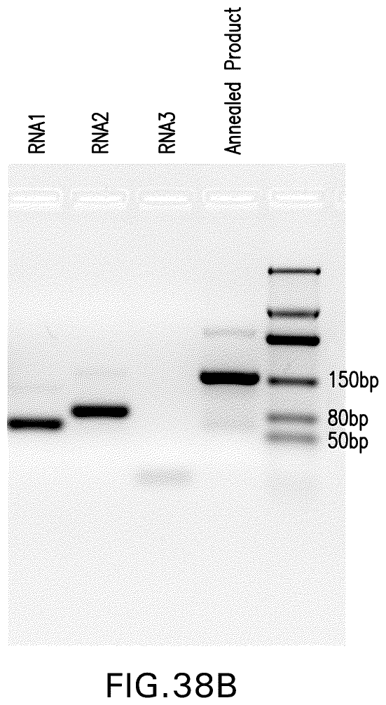

(FIG. 38A) Three RNAs are generated by in vitro transcription, with PCR products as templates.
RNA1: PSMA aptamer-BIRC5 antisense RNA
RNA2: PSMA aptamer-UBB/UBC sense siRNA and BIRC5 sense siRNA
RNA3: UBB/UBC anti-sense strand

### PSMA Aptamer Sequence:

(SEQ ID NO: 793):
5′-GGGAGGACGAUGCGGAUCAGCCAUGUUUACGUCACUCCUA-3′

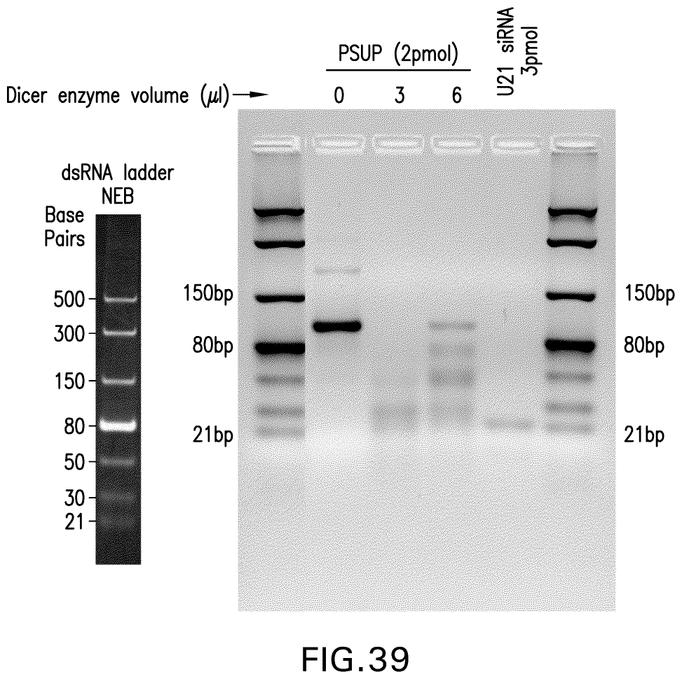

The PCR products are sequenced or put into T-A cloning pCR2.1 vector (Invitrogen) and sequenced. Transcription is performed with TranscriptAid T7 High Yield Transcription Kit following manufacturer's instruction. 2′F-modified pyrimidines (TriLink, San Diego, CA) are incorporated into RNA to replace CTP and UTP. The transcribed RNAs are purified with phenol/chloroform/isoamyl alcohol (25:24:1) (Sigma-Aldrich), precipitated with isopropanol (Sigma-Aldrich) followed by cold 70% ethanol wash. The RNA pellets are dissolved in nuclease free water (IDT). The three RNAs are mixed at molar ratio 1:1:1 and annealed to form one entity by heated at 94° C. for 3 min followed by slowly cooling to room temperature within 1 h. Resulting products were run on a gel for confirmation (FIG. 38B). 2 pmol of product was treated with 0, 3, or 6 μmol of dicer enzyme for 16 hours in order to confirm that the product is able to be cleaved by the enzyme. A gel was run on the resulting product for confirmation (FIG. 39).

### Example 23: Building Bispecific Aptamer-siRNA: DHX9-UBB-DHX9

Two RNAs are generated by in vitro transcription, with PCR products as templates

### RNA1: DHX9 Aptamer-UBB Sense RNA

SEQ ID NO: 794:
5′-GCCCAGCAUGCAUUACUGAUCGUGGUGUUU
GCUUAGCCCAAAGGCCAAGAUCCAAGAUAAAGAAGGC-3′

RNA2: DHX9 Aptamer-UBB Anti-Sense siRNA

SEQ ID NO: 795:
5′-GCCCAGCAUGCAUUACUGAUCGUGGUGUUU
GCUUAGCCCAAAGCCUUCUUUAUCUUGGAUCUUGGCCUU-3′

### DHX9 Aptamer Sequence:

(SEQ ID NO: 796):
5-GCCCAGCAUGCAUUACUGAUCGUGGUGUUUGCUUAGCCCA-3′

U22ds (SEQ ID NO: 645) is utilized as the UBB targeting sequence.

The PCR products are sequenced or put into T-A cloning pCR2.1 vector (Invitrogen) and sequenced. Transcription is performed with TranscriptAid T7 High Yield Transcription Kit following manufacturer's instruction. 2′F-modified pyrimidines (TriLink, San Diego, CA) are incorporated into RNA to replace CTP and UTP. The transcribed RNAs are purified with phenol/chloroform/isoamyl alcohol (25:24:1) (Sigma-Aldrich), precipitated with isopropanol (Sigma-Aldrich) followed by cold 70% ethanol wash. The RNA pellets are dissolved in nuclease free water (IDT). The RNAs are mixed at molar ratio 1:1 and annealed to form one entity by heated at 94° C. for 3 min followed by slowly cooling to room temperature within 1 h. Resulting products were run on a gel for confirmation.

### Example 24 In Vitro Aptamer-siRNA Inhibition of UBB Expression

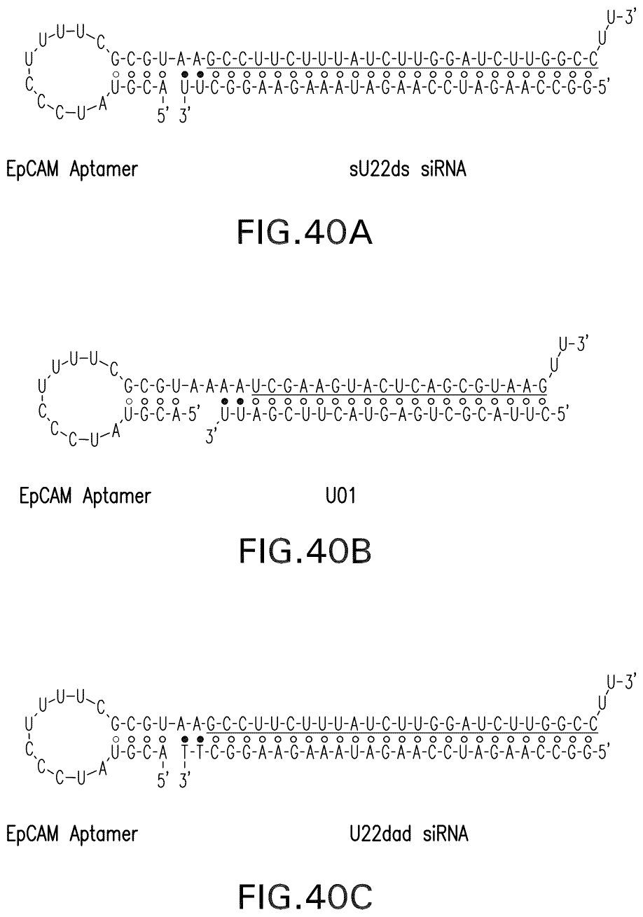

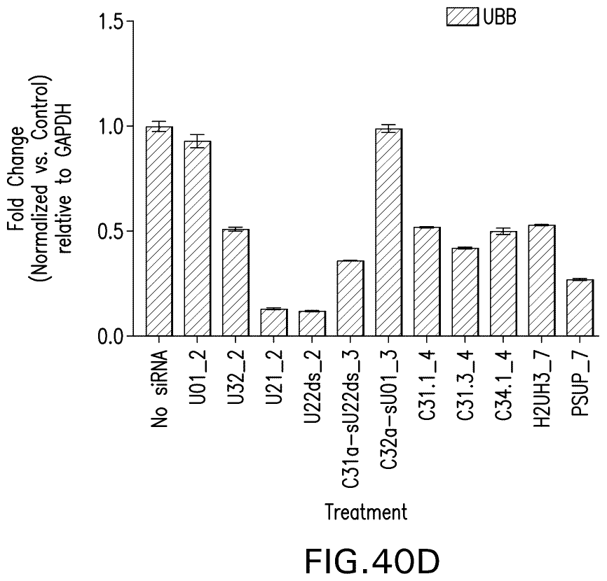

HCT-116 cells were transfected with various Aptamer-siRNA compositions with a transfection reagent ration of 6:1 for 48 hours and expression level of the target UBB was measured using qPCR. Compositions included previously disclosed controls as well as partial Aptamer-siRNA constructs shown in FIG. 40A and FIG. 40B. FIG. 40A (C31a/sU22ds) is an Epcam aptamer conjugated to the active U22 siRNA which FIG. 40B (C32a/sU01) is the same aptamer conjugated to control. C31.1 is the construct disclosed in FIG. 37A, C31.3 is the construct disclosed in FIG. 37E, C34.1 is the construct disclosed in FIG. 37C, H2UH3 is the construct disclosed in FIG. 36A, and PSUP is the construct disclosed in FIG. 38A. Results demonstrate that active aptamer-siRNA constructs are able to inhibit UBB expression over control (FIG. 40D)

### Example 25: In Vitro Viability Studies

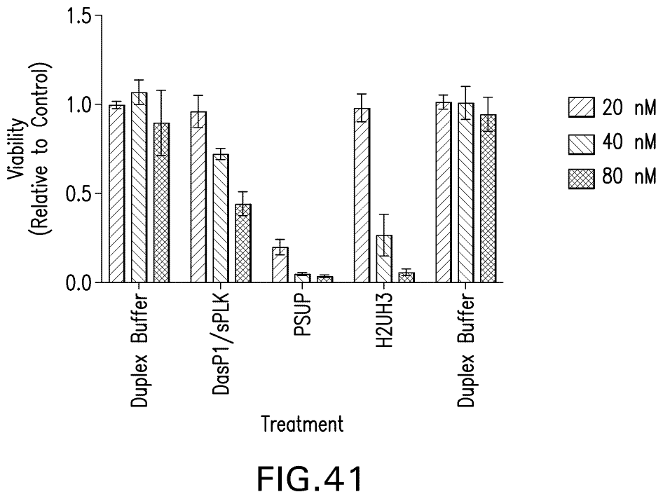

HCT116 cells were treated with previously described compositions as well as DasP1/sPLK, a PSMA aptamer-PLK1 siRNA construct. The cells treated with PSUP, PSMA aptamer-BIRC5 siRNA-UBB siRNA-PSMA aptamer, demonstrated the most significant toxicity at the lowest concentrations to colon cancer cells. H2UH3 (HER3 aptamer-U21 siRNA-HER2 aptamer) also demonstrated significant toxicity to cancer cells at a lower concentration than control (FIG. 41)

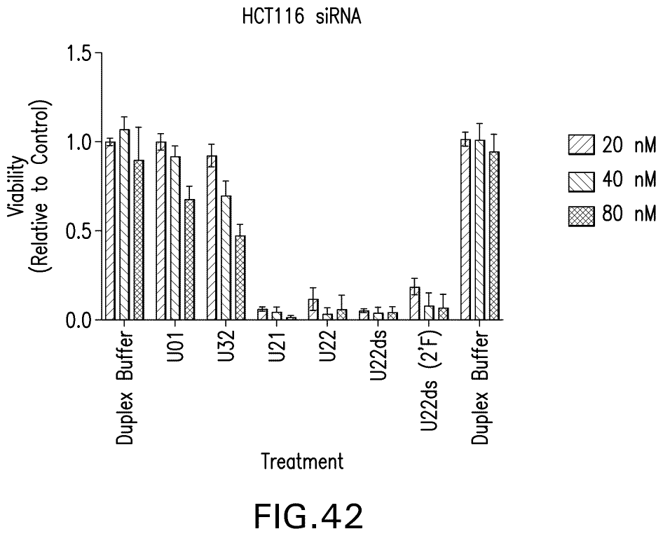

Additionally, HCT116 cells were transfected and treated for 72 hours with previously described variations of the multi-targeting UBB/UBC siRNA. Transfection reagent ratio was 6:1 and cells were treated with 20, 40, or 60 nM of RNA. Viability was measured using cell titer glow. The active siRNAs (U21, U22, U22ds, and U22ds (2′F) showed significant toxicity to the colon cancer cell compared to control (FIG. 42).

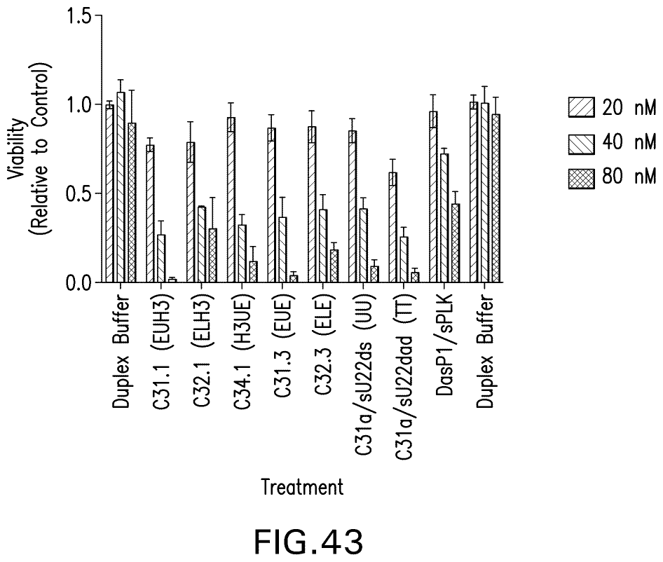

Additionally, HCT116 cells were transfected and treated for 72 hours with various aptamer-siRNA constructs, some previously described. C32.1 is the construct disclosed in FIG. 37B, C32.1 is the construct disclosed in FIG. 37F, C31a/sU22dad (TT) is the partial aptamer-siRNA construct disclosed in FIG. 40C. Transfection reagent ratio was 6:1 and cells were treated with 20, 40, or 60 nM of RNA. Viability was measured using cell titer glow. The constructs that included a dual targeting UBB/UBC siRNA demonstrated the most significant toxicity to the cancer cells compared to control at higher concentrations with 31.1 and 31.3 showing the most significant. (FIG. 43)

### Example 26: Various T Cell-Targeted Aptamers and Immune Checkpoint Inhibitors Useful in Embodiments of the Invention

### Anti-PD1 Aptamer Sequences:

(SEQ ID NO: 797):
5′-GGCUGGGGUGCGACAUUAUGUUCGUUAAGGAUCAAUCGCUC-3′
(SEQ ID NO: 798):
5′-UUAUGAUGCAAAAACGAACUGGAAUGGCCAUGCAGGUACA-3′
(SEQ ID NO: 799):
5′-GGUUGGGGUGCGACAUUAUGUUCGUUAAGGAUCAAUCGCUC-3′
(SEQ ID NO: 800):
5′-GAUUUGGAGAGCAUUAUGUUAGGUUAAGGAUCAAUCUUCUA-3′
(SEQ ID NO: 801):
5′-GGCUGGGGUGCGACAUUAUGUUCGUUAAGGAUCAAUCGCUU-3′

### 2′F Pyrimidine Modification:

(SEQ ID NO: 802):
5′-GGGGUAAACGAACAGGAUUUUUUUAACGAGCUAUAUUGUUUCCUGU
UGCCCGUCCGUCUAGUCGUGAAGAGAGCAAGGUUACU-3′ (10B-1)
(SEQ ID NO: 803):
5′-GGGGUAAACGAACAGGACGACGGGUCGAAGCUGAAUAGGUAACCAA
UCACGGCAUAACUAGUCGUGAAGAGAGCAAGGUUACU-3′ (10B-10)
(SEQ ID NO: 804):
5′-GGGGUAAACGAACAGGAUGAGGGAGCAAAAAGGGCGAAAAUGCAGU
AACUAAACGUUCUAGUCGUGAAGAGAGCAAGGUUACU-3′ (10B-14)
(SEQ ID NO: 805):
5′-GGGGUAAACGAACAGGAUUUUUUUAACGAGCUAUAUUAUUUCCUGU
UGCCCGUCCGUCUAGUCGUGAAGAGAGCAAGGUUACU-3′ (10B-68)
(SEQ ID NO: 806):
5′-GGGGUAAACGAACAGGAACCAUUAAAUCAUAAGGAGAAAGAUGAUG
UGCGCGACAUAACUAGUCGUGAAGAGAGCAAGGUUACU-3′ (10B-84)

2′F Cytidine with 2-Pyridyl the 5 Position Uridine:

(SEQ ID NO: 807):
5′-GGGAGACAAACAAAGAGCGACAAGGGCAGCGGGUUAUCACGUUGGG
AACGGGCCAUCAACUCUUCUCACCACCCAGAAGAAGCCAGAAG-3′
(SEQ ID NO: 808):
5′-GGGAGACAAACAAAGAGCGACAAGGGCAGUAGUGAGGGAUUCACCA
GAGUGAAUGCGCUCCUCGGAAAUCACCCAGAAGAAGCCAGAAG-3′
(SEQ ID NO: 809):
5′-GGGAGACAAACAAAGAGCGACAAGGGCAGAACGGGCAAUGUCCAAG
GUGAGGCAGUUUGUAUGGACACACACCCAGAAGAAGCCAGAAG-3′
2′F cytidine with Benzyl at the 5 position
uridine:
(SEQ ID NO: 810):
5′-GGGAGACAAACAAAGAGCGACAAGGGCAGUUGAGAUUGAGGAGUCA
GACCUGCGUCUCUAGUAACAAUGCACCCAGAAGAAGCCAGAAG-3′
(SEQ ID NO: 811):
5′-GGGAGACAAACAAAGAGCGACAAGGGCAGAGUGGACGGUCGGCUAG
AGCCGGGAGGAAUUCCUUGUGACCACCCAGAAGAAGCCAGAAG-3′
(SEQ ID NO: 812):
5′-GGGAGACAAACAAAGAGCGACAAGGGCAGUUUGACAAUGUACCUUU
AAUUACGGAUUGUACCUUGGGCGCACCCAGAAGAAGCCAGAAG-3′

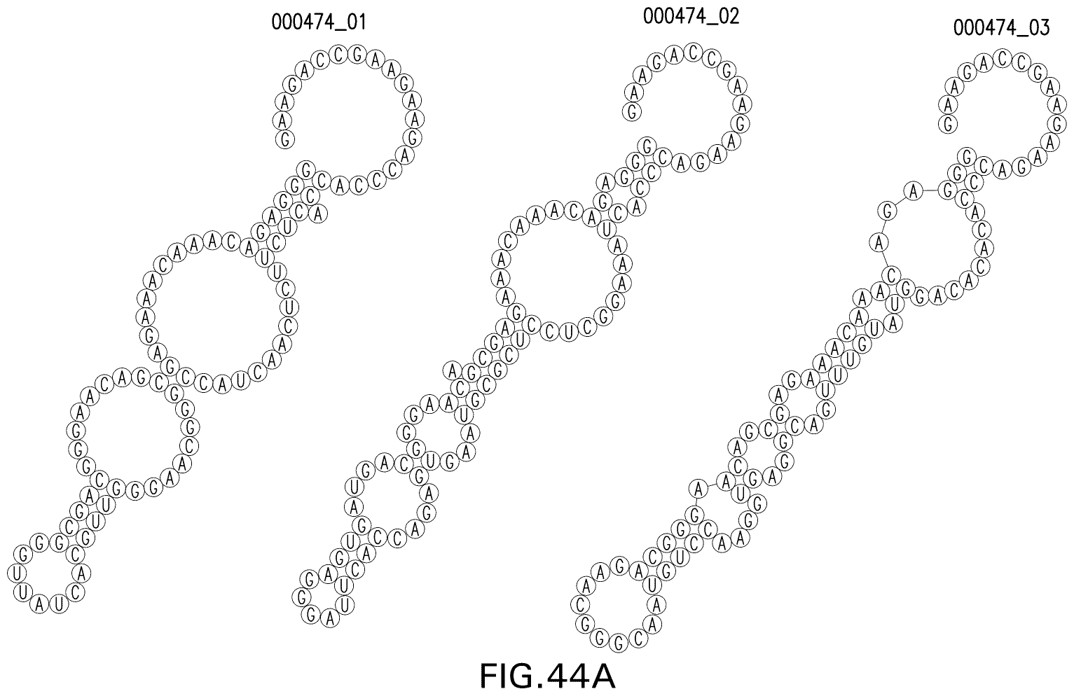

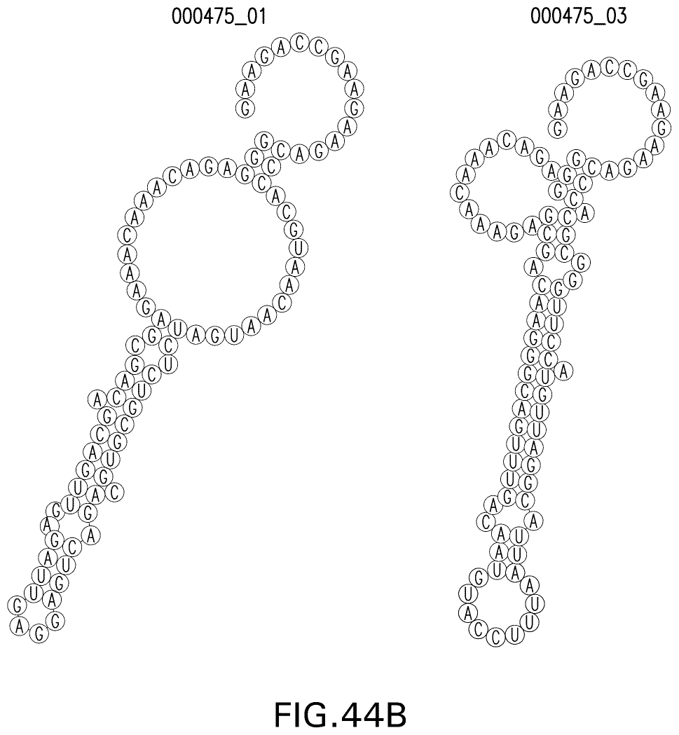

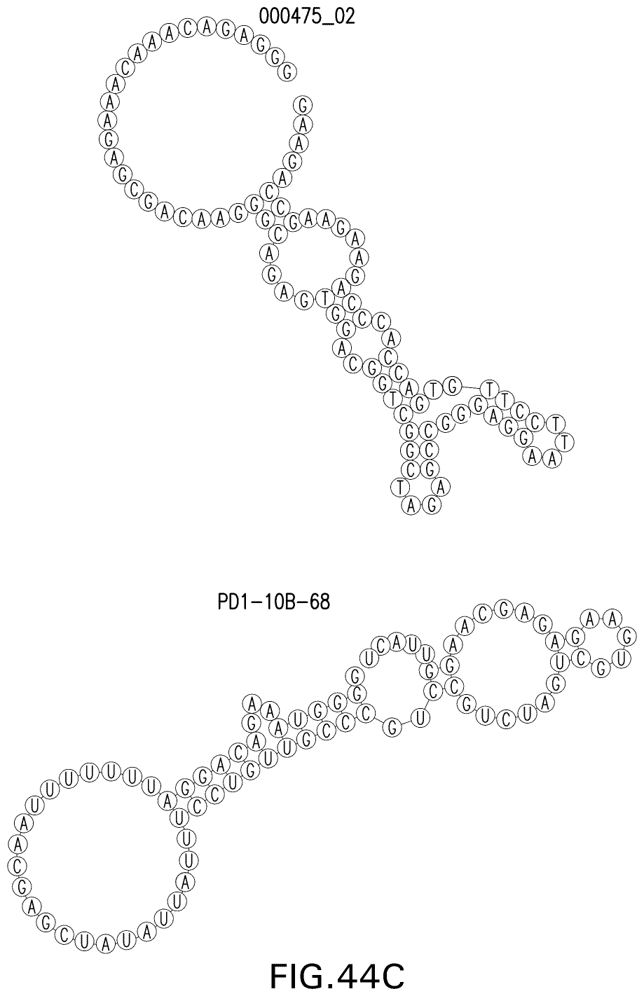

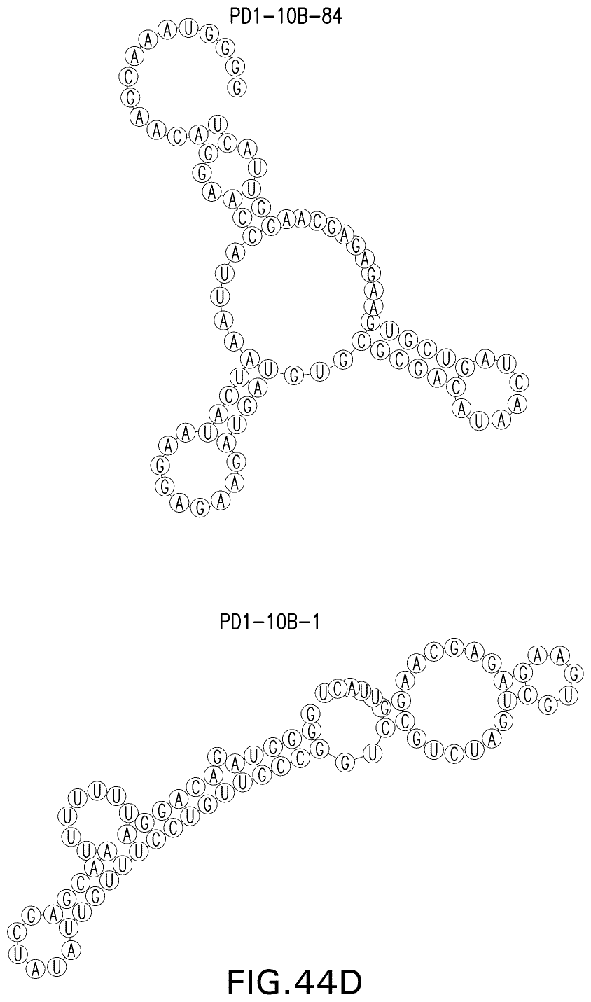

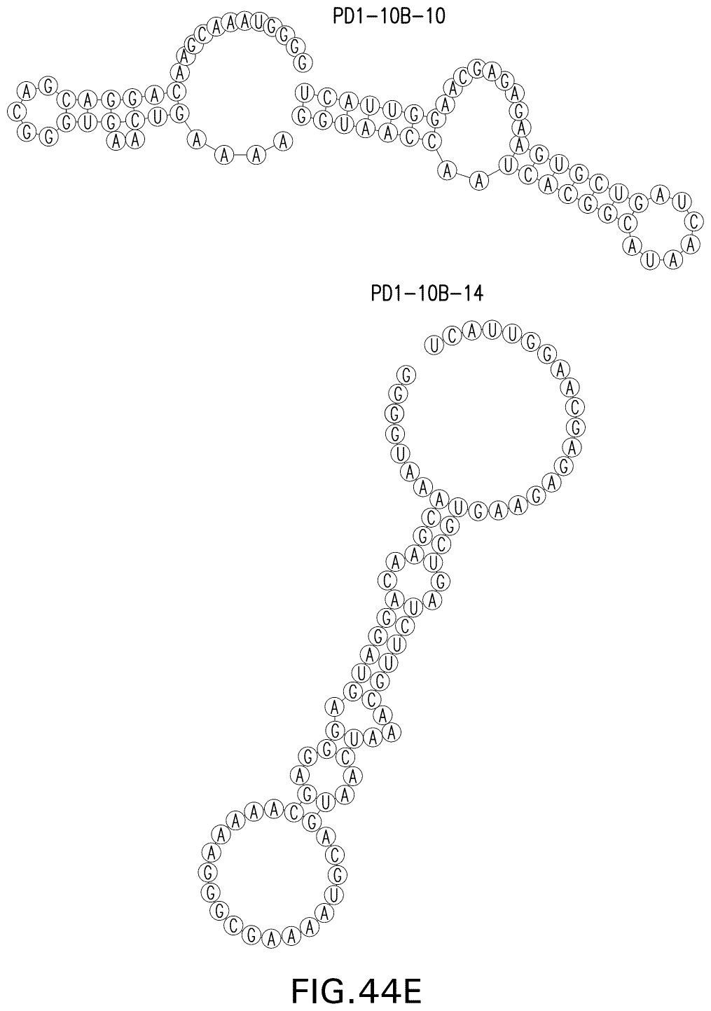

Binding structures of select aptamers are shown in FIG. 44.

### CTLA-4 Aptamer Sequence:

(SEQ ID NO: 813):
5′-GGGAGAGAGGAAGAGGGAUGGGCCGACGUGCCGCA-3′

2′F Cytidine with 2-Pyridyl the 5 Position Uridine:

(SEQ ID NO: 814):
5′-GGGAGACAAACAAAGAGCGACAAGGGCAGAAUUACAAUAGCUAUAG
UCCGGGCACCAUGCUUGUAAAUCCACCCAGAAGAAGCCAGAAG-3′
(SEQ ID NO: 815):
5′-GGGAGACAAACAAAGAGCGACAAGGGCAGGUAGGACGCUAGCAGAC
UAGAAUGUAUCUAUGCUUAGAUCCACCCAGAAGAAGCCAGAAG-3′
(SEQ ID NO: 816):
5′-GGGAGACAAACAAAGAGCGACAAGGGCAGGCUAGUAUUACAAUGUC
GUGGAAAAGCCGUGCGGGGUAUCCACCCAGAAGAAGCCAGAAG-3′

2′F Cytidine with Benzyl at the 5 Position Uridine:

(SEQ ID NO: 817):
5′-GGGAGACAAACAAAGAGCGACAAGGGCAGGGAGCCAUUCUUGAAAU
UGUCAGUUUGAUUGUGCUCAGGUCACCCAGAAGAAGCCAGAAG-3′
(SEQ ID NO: 818):
5′-GGGAGACAAACAAAGAGCGACAAGGGCAGAAAGUACAAUGGUUGAC
AUAUACCGUCGGUUUAUCCUAUGCACCCAGAAGAAGCCAGAAG-3′
(SEQ ID NO: 819):
5′-GGGAGACAAACAAAGAGCGACAAGGGCAGGUAGGCUAUCGCUGCUU
GAUCGUCUGAUCAGAGCCUAUACCACCCAGAAGAAGCCAGAAG-3′

### 2′F Pyrimidine Modification:

(SEQ ID NO: 820):
5′-GGGGUAAACGAACAGGAAACAGAUGGCCAACACAGGCGAAGCAUAG
ACUAGGAACGGCUAGUCGUGAAGAGAGCAAGGUUACU-3′ (CTLA4-
A10-6)
(SEQ ID NO: 821):
5′-GGGGUAAACGAACAGGACUUGAUGUGAAAAGGCGACGCGAUGAGAC
GAAGGGCUUCUAGUCGUGAAGAGAGCAAGGUUACU-3′ (CTLA4-
A10-38)
(SEQ ID NO: 822):
5′-GGGGUAAACGAACAGGAAGUAGACUAGACGGCGGCGAUAACCAGAU
AACGACAUUCUCUAGUCGUGAAGAGAGCAAGGUUACU-3′ (CTLA4-
A10-14)
(SEQ ID NO: 823):
5′-GGGGUAAACGAACAGGACCGAGUGAGACGGGUAGUGGACAAAUGAA
GUAGUGUGGUCCUAGUCGUGAAGAGAGCAAGGUUACU-3′ (CTLA4-
A10-2)
(SEQ ID NO: 824):
5′-GGGGUAAACGAACAGGACUUUUAAUUUCACGCCGCACGAUCCGGAA
AAACGACUUGACUAGUCGUGAAGAGAGCAAGGUUACU-3′ (CTLA4-
A10-13)

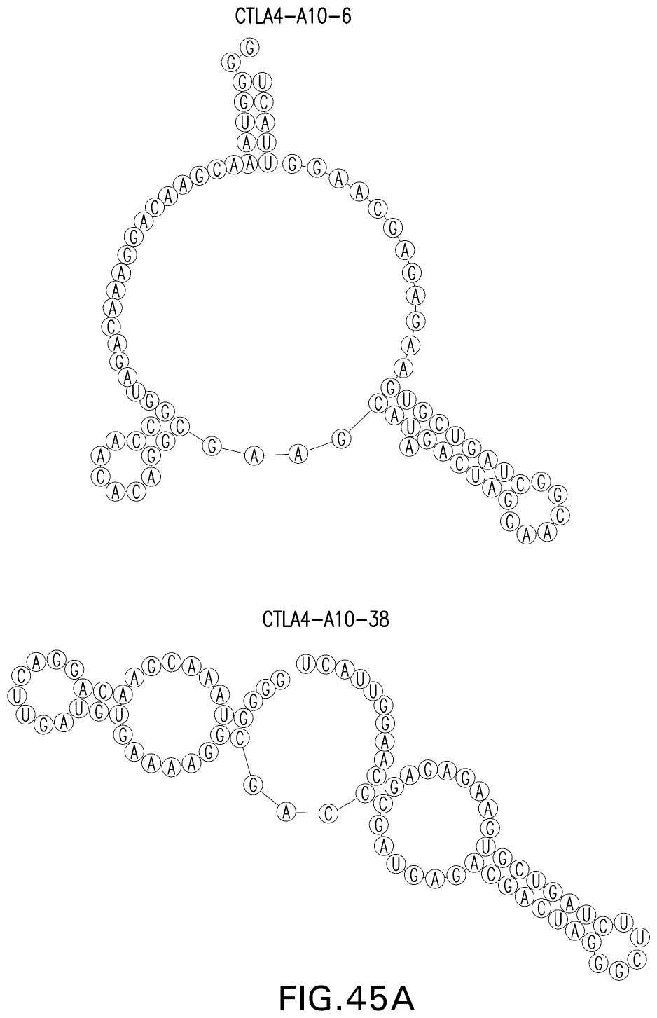

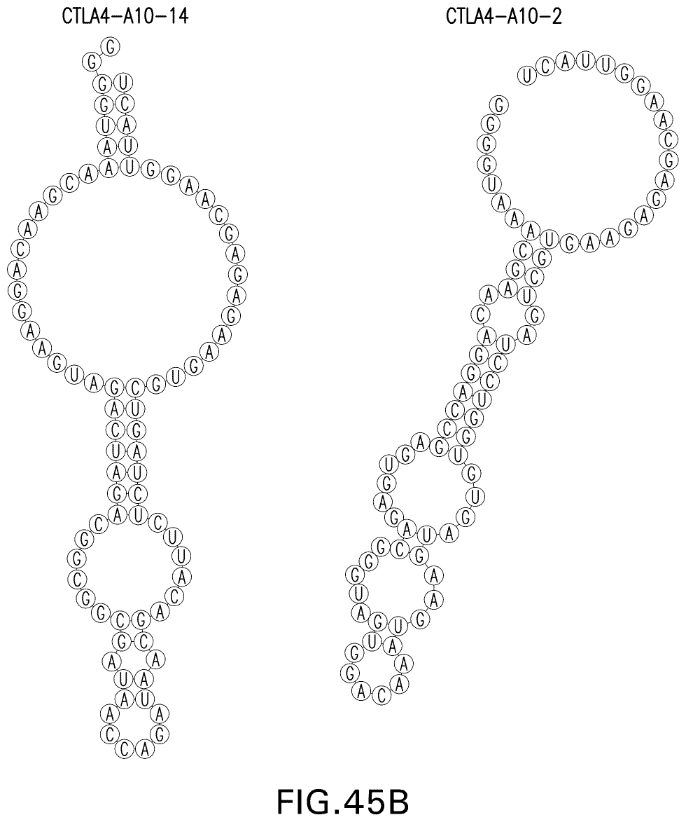

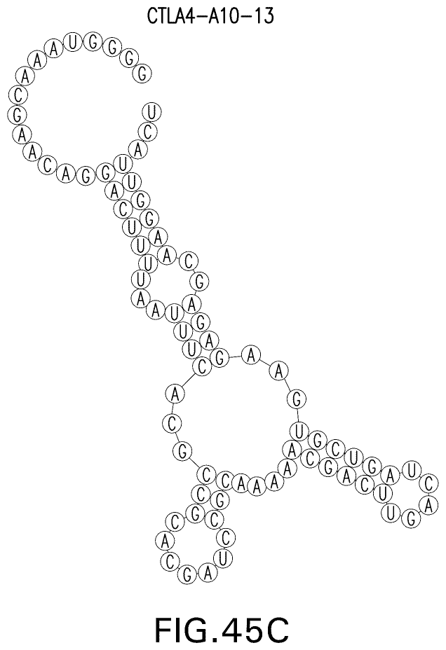

Binding structures of select aptamers are shown in FIG. 45.

### TIM3 Aptamer Sequences:

### 2′F Pyrimidine Modification:

(SEQ ID NO: 825):
5′-GGGGUAAACGAACAGGAAGGGAGUCGAUUUGAGUUGUAAUUUGACC
UAUGUUAUAAUCUAGUCGUGAAGAGAGCAAGGUUACU-3′ (TIM3-
A-4)
(SEQ ID NO: 826):
5′-GGGGUAAACGAACAGGAAUGGCUACAGUAUCGAUGCAGUUUUCGAA
UGAAGUAGAAACUAGUCGUGAAGAGAGCAAGGUUACU-3′ (TIM3-
A-8)
(SEQ ID NO: 827):
5′-GGGGUAAACGAACAGGACAGGACAGCAAGCAGUAGAAAACAAGCCA
CGAAGGGGACUCUAGUCGUGAAGAGAGCAAGGUUACU-3′ (TIM3-
A-25)
(SEQ ID NO: 828):
5′-GGGGUAAACGAACAGGAUUUUGGACUGUCUAGCCGAUGUACUUAAG
UUUAUCAUUUUCUAGUCGUGAAGAGAGCAAGGUUACU-3′ (TIM3-
A-43)
(SEQ ID NO: 829):
5′-GGGGUAAACGAACAGGAGCAGUCGCUGGCUUCAUUUUUUUUUUUUU
UUUGUGCUCAACUAGUCGUGAAGAGAGCAAGGUUACU-3′ (TIM3-
A-57)

### PSMA Aptamer Sequences:

2′F Cytidine with Benzyl at the 5 Position Uridine:

(SEQ ID NO: 830):
5′-AGGAGACACAUGUGACAAGAGGCUAUGAUCCUGAAUGCAUCCUUGGC
AAAC-3′

2′F Cytidine with 4-Pyridyl the 5 Position Uridine:

(SEQ ID NO: 831):
5′-AGGAGAAUCAUGAGUUAUCUGUGUAAGGAACCAAAGCCAUGCUUAU
CAAAC-3′

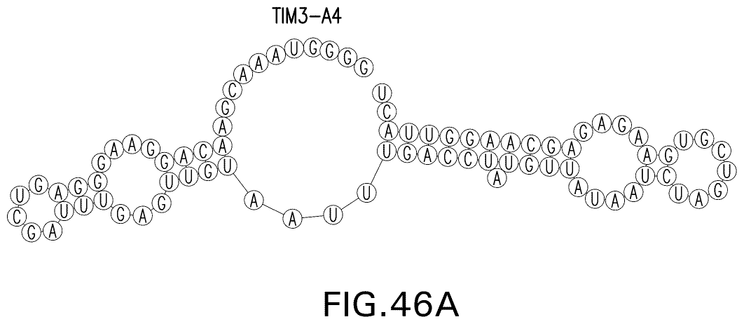

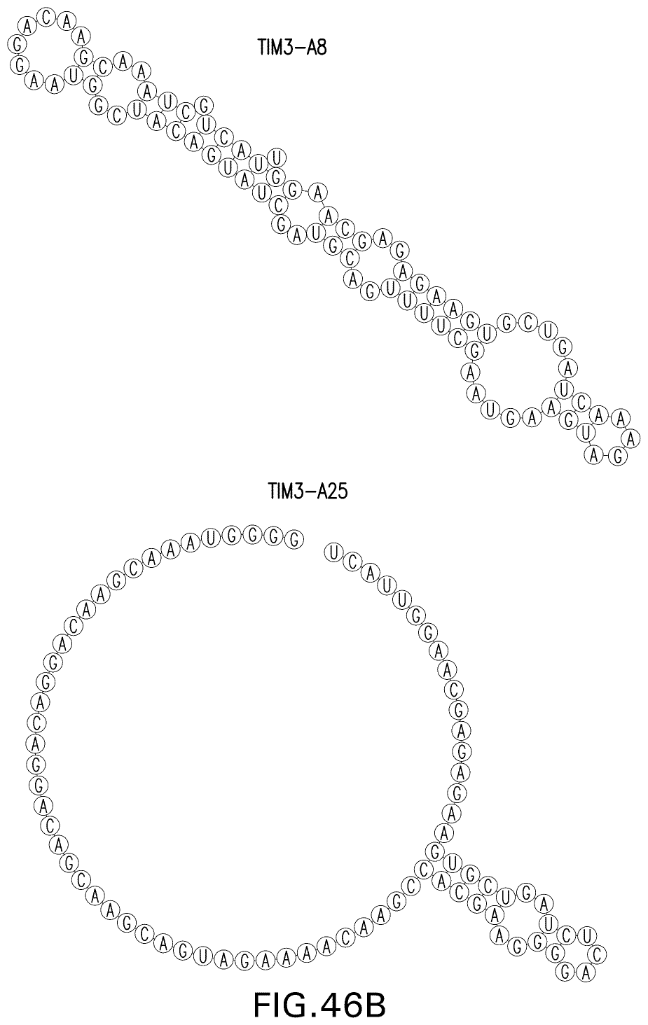

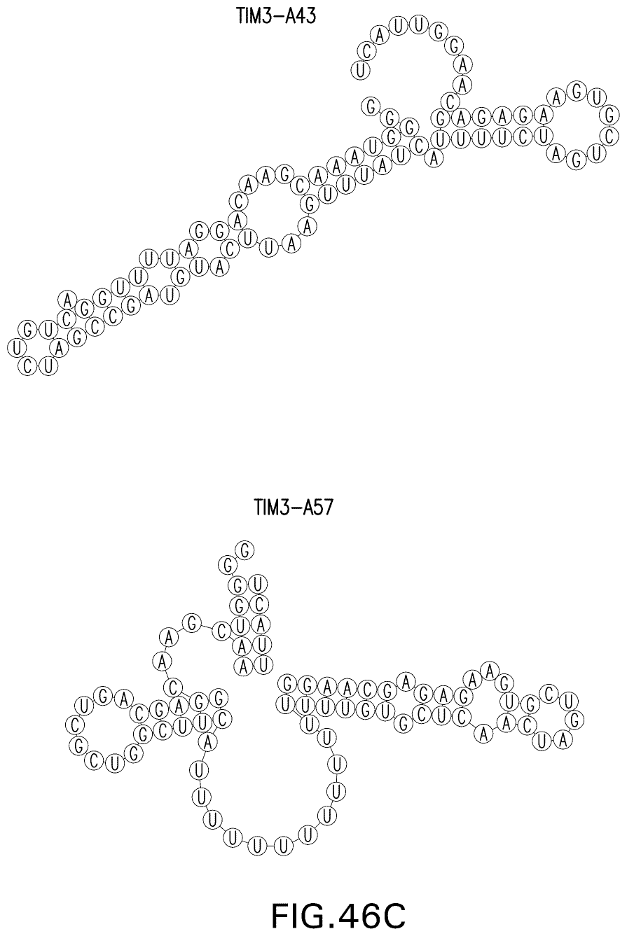

See FIG. 46 or example binding structures.

### Target Sequences of NR4A1:

(SEQ ID NO: 832):
5′-CTGATTAATATATTTAATATA-3′
(SEQ ID NO: 833):
5′-CTCCTTCCACATGTACATAAA-3′
(SEQ ID NO: 834):
5′-CAGCATTATGGTGTCCGCACA-3′
(SEQ ID NO: 835):
5′-CAGCACCTTCATGGACGGCTA-3′
(SEQ ID NO: 836):
5′-GCACCTTCATGGACGGCTA-3′ (hNR4A1.1E2)
(SEQ ID NO: 837):
5′-GCATATGGTGTCCGCACA-3′ (hNR4A1.2E2)
(SEQ ID NO: 838):
5′-TGAAGGAAGTGTCCGAAC-3′ (hNR4A1.3E2)
(SEQ ID NO: 839):
5′-CTGCAGAACCGCATCGCCA-3′ (hNR4A1.4E2)
(SEQ ID NO: 840):
5′-TGCTGTGTGTGGGGACAAC-3′
(SEQ ID NO: 841):
5′-GGGCTGCAAGGGCTTCTTC-3′
(SEQ ID NO: 842):
5′-GCGCACAGTGCAGAAAAAC-3′
(SEQ ID NO: 843):
5′-CAGTGGCTCTGACTACT-3′
(SEQ ID NO: 844):
5′-CCACTTCTCCACACCTTGA-3′
(SEQ ID NO: 845):
5′-GGCTTGAGCTGCAGAATG-3′
(SEQ ID NO: 846):
5′-CACAGCTTGCTTGTCGATGTC-3′
(SEQ ID NO: 847):
5′-GGTCCCTGCACAGCTTGCTTGTCGA-3′
(SEQ ID NO: 848):
5′-CCGGTTCTCTGGAGGTCATCCGCAA-3′
(SEQ ID NO: 849):
5′-CAGCATTATGGTGTCCGCACATGTG-3′

### Target Sequences of VHL:

(SEQ ID NO: 850):
5′-AATGTTGACGGACAGCCTATT-3′
(SEQ ID NO: 851):
5′-AAGAGTACGGCCCTGAAGAAG-3′
(SEQ ID NO: 852):
5′-AAGGAGGTTTGTATAAGTAAT-3′
(SEQ ID NO: 853):
5′-CAGGAGCGCATTGCACATCAA-3′
(SEQ ID NO: 854):
5′-CCCTATTAGATACACTTCTTA-3′
(SEQ ID NO: 855):
5′-TAAGGAGGTTTGTATAAGTAA-3′
(SEQ ID NO: 856):
5′-CCTAGTCAAGCCTGAGAATTA-3′
(SEQ ID NO: 857):
5′-CTGCCAGTGTATACTCTGA-3′
(SEQ ID NO: 858):
5′-ATACACTCGGTAGCTGTGG-3′

The PCR products are processed according to the methods previously stated.

### Lag-3 Aptamer Sequence:

(SEQ ID NO: 859):
5′-GGGAGAGAGAUAUAAGGGCCUCCUGAUACCCGCUGCUAUCUGGACC
GAUCCCAUUACCAAAUUCUCUCCC-3′

### 2′F Pyrimidine Modification:

(SEQ ID NO: 860):
5′-GGGGUAAACGAACAGGAAGACGGCGCAAUAAGACAGACUAGGACAC
GAUUAGAGGUACUAGUCGUGAAGAGAGCAAGGUUACU-3′ (LAG3-
A10-4)
(SEQ ID NO: 861):
5′-GGGGUAAACGAACAGGAUAAAAGAAAACAACUAGCGCGACGAGAGA
AUAAAAUGAAACUAGUCGUGAAGAGAGCAAGGUUACU (LAG3-A10-
71)
(SEQ ID NO: 862):
5′-GGGGUAAACGAACAGGAUAAUUGUUGGGGAAAUAAAUUGCUGGGAA
CGACUUAAAAGCUAGUCGUGAAGAGAGCAAGGUUACU-3′ (LAG3-
A10-79)
(SEQ ID NO: 863):
5′-GGGGUAAACGAACAGGAGUUAAUCAUGAGGUAGGUAACAAAAGGCA
ACGGCCAAUAACUAGUCGUGAAGAGAGCAAGGUUACU (LAG3-A10-
41)
(SEQ ID NO: 864):
5′-GGGGUAAACGAACAGGAUAACCAUGCAAAUAACAAGCAAACAGAGA
ACUCACGCCAGCUAGUCGUGAAGAGAGCAAGGUUACU-3′ (LAG3-
A10-7)

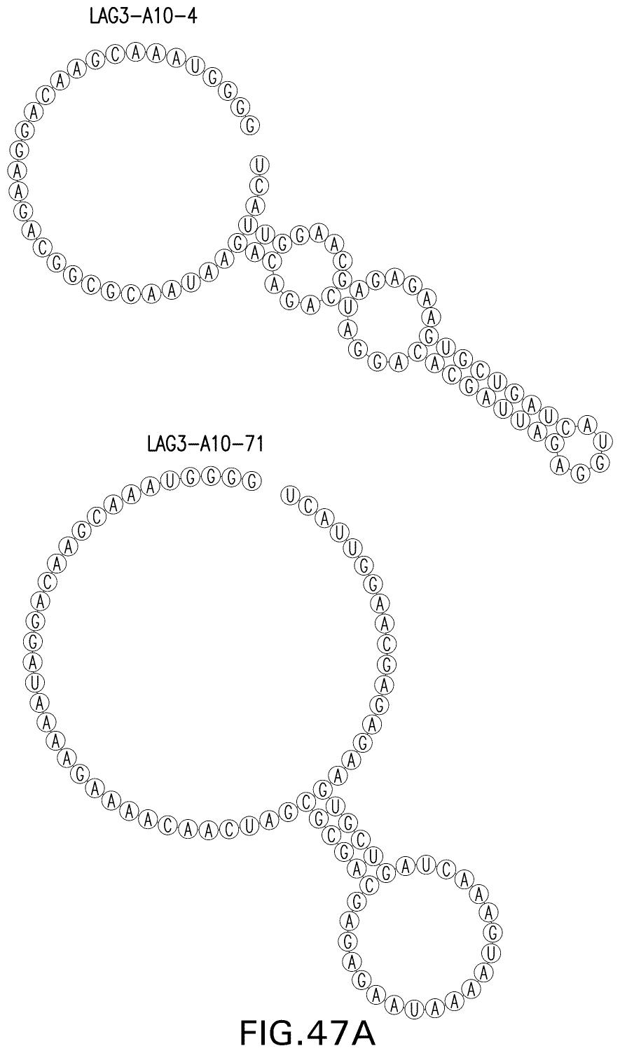

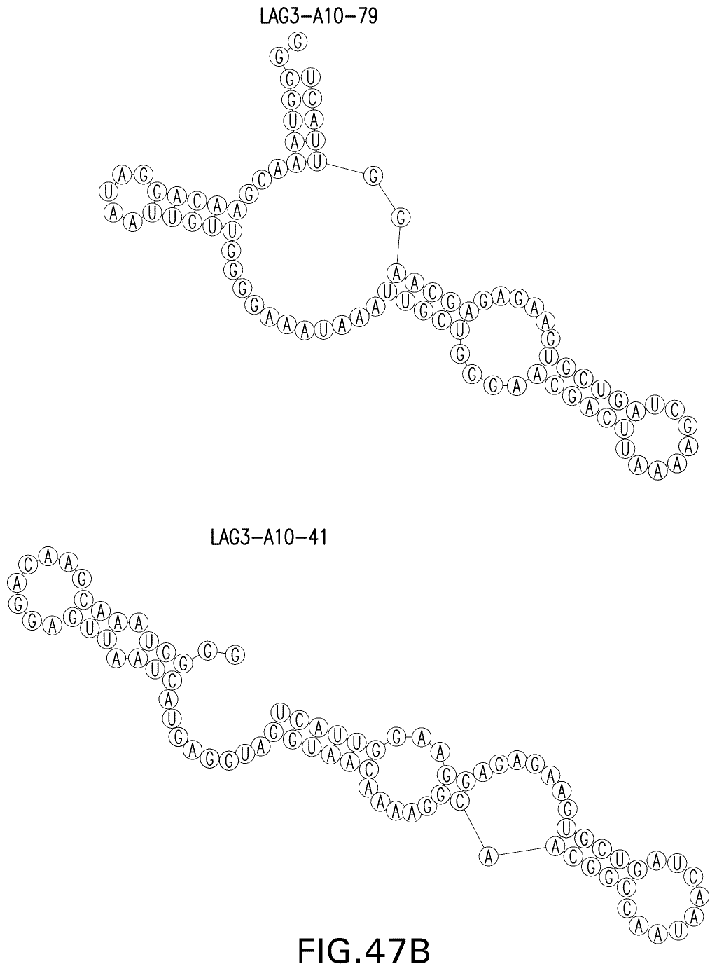

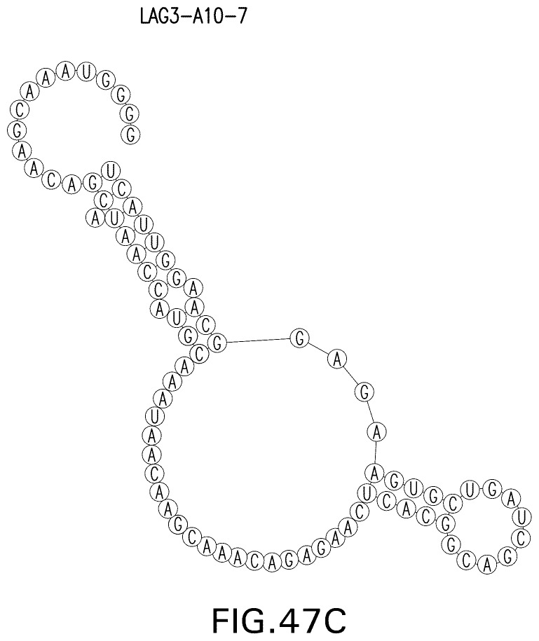

See FIG. 47 for example binding structures.

### Example 27: Building Bispecific Aptamer-siRNA: TROP-2-UBB/UBC-HER3

Two RNAs are generated by in vitro transcription, with PCR products as templates.
RNA1: TROP2 aptamer-UBB/UBC antisense RNA
RNA2: HER3 aptamer and UBB/UBC sense siRNA.

The PCR products are processed as previously described.

### Trop 2 Aptamer

(SEQ ID NO: 865):
5′-UAUACAUUCUUGGUUCAUAAAGGAUAAGGCCUAAGUCGGGU-3′

2′F Cytidine with Benzyl at the 5 Position Uridine:

(SEQ ID NO: 866):
5′-AGGAGUAUACAUUCUUGGUUCAUAAAGGAUAAGGCCUAAGUCGGGU
CAAAC-3′

2′F pyrimidine Modification:

(SEQ ID NO: 867):
5′-GGGAGACAAGAAUAAACGCUCAAGACACGGAUACAUAAUGCUGUCU
UGAUUUACAAACUGAGCUUCGACAGGAGGCUCACAACAGGC-3′ (S10)
(SEQ ID NO: 868):
5′-GGGAGACAAGAAUAAACGCUCAAUGAGCUUACAGCGGCCAUUGAUU
UACUAACGGACUGAGCAUUCGACAGGAGGCUCACAACAGGC-3′ (S09)

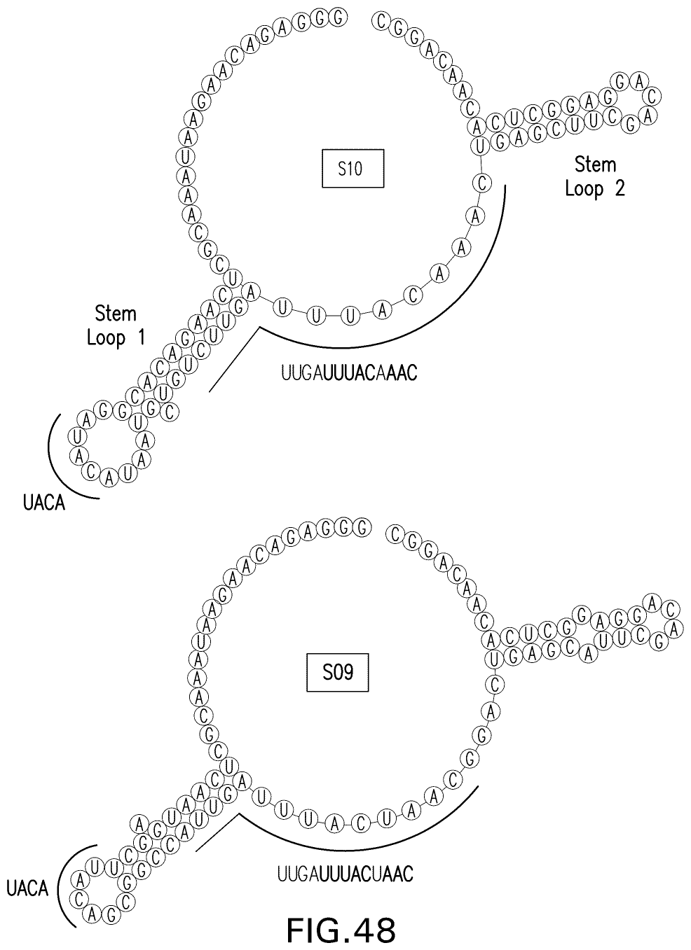

See FIG. 48 for example binding structures.

### HER3 Aptamer:

(SEQ ID NO: 869):
5′-GAAUUCCGCGUGUGCCAGCGAAAGUUGCGUAUGGGUCACAUCGCAG
GCACAUGUCAUCUGGGCGGUCCGUUCGGGAUCC-3′

2′F Cytidine with Benzyl at the 5 Position Uridine:

(SEQ ID NO: 870):
5′-AGGAGGGUUGCGUUGCAAGUAACAGAAAGGAAUUUGAAAAUUGUGG
CAAAC-3′

### 4-Pyridyl Modified: (

SEQ ID NO: 871):
5′-AGGAGGUUGGCAAUCCCGGAUUGAGGAAUCGCAUGACGCUAUUAAC
CAAAC-3′

### Example 28: Building Bispecific Aptamer-siRNA: CD73-UBB/UBC-TROP2

Two RNAs are generated by in vitro transcription, with PCR products as templates.
RNA1: CD73 aptamer-UBB/UBC antisense RNA
RNA2: TROP2 aptamer-UBB/UBC sense siRNA.

The PCR products are processed as previously described.

### CD73 Aptamer Sequence:

(SEQ ID NO: 872):
5′-UAGUAAAUGAGAGAUGAAAUCUGUAUGCGCCGCACUGAUUG-3′

2′F Cytidine with Benzyl at the 5 Position Uridine:

(SEQ ID NO: 873):
5′-AGGAGUAGUGCAGCAUUGACUGAAUGUCAUACGGCAUAAGCAUCUA
CAAAC-3′
(SEQ ID NO: 874):
5′-AGGAGGACAUCGGAAACGCUGAUCUUAAUAGUGAAUUAACAUGCGA
CAAAC-3′

2′F Cytidine with 4-Pyridyl at the 5 Position Uridine:

(SEQ ID NO: 875):
5′-AGGAGUAGUAAAUGAGAGAUGAAAUCUGUAUGCGCCGCACUGAUUG
CAAAC-3′

### Example 29: PSCA-MSI2-UBB-CD44

RNA1: PSCA aptamer-MSI2 antisense siRNA
RNA2: CD44 aptamer and UBB sense siRNA and MSI2 sense siRNA
RNA3: UBB anti-sense strand

PCR products are processed as previously discussed using sequences presented in this application.

### PSCA Aptamer Sequence:

2′F Cytidine with 2-Pyridyl the 5 Position Uridine:

(SEQ ID NO: 876):
5′-GGGAGACAAACAAAGAGCGACAAGGGCAGGGAUGACCGGUGUAUUA
AGGUCUAACUUAACUCGGUGAUACACCCAGAAGAAGCCAGAAG-3′
(SEQ ID NO: 877):
5′-GGGAGACAAACAAAGAGCGACAAGGGCAGCUCAAAAAGGGUAGUGU
GUGGUAUAGUCUAAUCGUACCCACACCCAGAAGAAGCCAGAAG-3′
(SEQ ID NO: 878):
5′-GGGAGACAAACAAAGAGCGACAAGGGCAGUAGUGUGGUAUUGUGUA
AUAAUACCCUACUGAGGUCAAAACACCCAGAAGAAGCCAGAAG-3′
(SEQ ID NO: 879):
5′-GGGAGACAAACAAAGAGCGACAAGGGCAGCGGGUUGUCAAGAUGGG
AACGGGCCCGGAUCUUUAGCGCACACCCAGAAGAAGCCAGAAG-3′
(SEQ ID NO: 880):
5′-GGGAGACAAACAAAGAGCGACAAGGGCAGUGGGCUGUGCGCGCGAU
GAGAUCACGUUAGCGUAAUUGUGCACCCAGAAGAAGCCAGAAG-3′
(SEQ ID NO: 881):
5′-GGGAGACAAACAAAGAGCGACAAGGGCAGACAGGUCCAUCAGGCAG
AACCGAGGGAGAGUGCGCGUCGUCACCCAGAAGAAGCCAGAAG-3′
2′F cytidine with Benzyl at the 5 position
uridine:
(SEQ ID NO: 882):
5′-GGGAGACAAACAAAGAGCGACAAGGGCAGAAGUUUGGAUUUCAAGA
UGCUCAUCACGCUCAAACUUUCACACCCAGAAGAAGCCAGAAG-3′
(SEQ ID NO: 883):
5′-GGGAGACAAACAAAGAGCGACAAGGGCAGGACGCUGCGAAAAGUGC
GAAGUUUGCAUCCUGGCCUAGUUCACCCAGAAGAAGCCAGAAG-3′
(SEQ ID NO: 884):
5′-GGGAGACAAACAAAGAGCGACAAGGGCAGUUCUCUCCACAAAGUUU
AGAUUUCAAGCGUGAGCAGGGAUCACCCAGAAGAAGCCAGAAG-3′
(SEQ ID NO: 885):
5′-GGGAGACAAACAAAGAGCGACAAGGGCAGGUGUUGAGAUGAAGGAG
UUCUAGCCCUUCGAAUGGUGUGACACCCAGAAGAAGCCAGAAG-3′
(SEQ ID NO: 886):
5′-GGGAGACAAACAAAGAGCGACAAGGGCAGAGUUGGGUAGGUUGUGA
CAGGAAUGUGAUUGGUAAGAUACCACCCAGAAGAAGCCAGAAG-3′
(SEQ ID NO: 887):
5′-GGGAGACAAACAAAGAGCGACAAGGGCAGUACUUGAGUCAUUGUAU
AGAUCUAAUUCGCGCAGAAUUGACACCCAGAAGAAGCCAGAAG-3′
(SEQ ID NO: 888):
5′-GGGAGACAAACAAAGAGCGACAAGGGCAGCCCUUGUCGCUCUUUGU
UUGUCUCCCUAUAGUGAGUCGUAUUACACCCAGAAGAAGCCAGAAG-3′

### Example 30: CD44-PIKFYVE-MAP2K1-CD133

RNA1: CD44 aptamer-PIKFYVE antisense siRNA
RNA2: CD133 aptamer and MAP2K1 sense siRNA and PIKFYVE sense siRNA
RNA3: MAP2K1 anti-sense strand

PCR products are processed as previously discussed using sequences presented in this application.

### Example 31: PSMA-UBB/UBC-PSMA

RNA1: PSMA aptamer-UBB/UBC antisense RNA
RNA2: PSMA aptamer and UBB/UBC sense siRNA.

### CD44 Aptamer Sequence:

2′F Cytidine with 2-Pyridyl the 5 Position Uridine:

(SEQ ID NO: 889):
5′-GGGAGACAAACAAAGAGCGACAAGGGCAGGUAGGACGCUAGCAGAC
UAGAAUGAAUCUAUGCUUAGAUCCACCCAGAAGAAGCCAGAAG-3′

2′F Cytidine with Benzyl at the 5 Position Uridine:

(SEQ ID NO: 890):
5′-GGGAGACAAACAAAGAGCGACAAGGGCAGCGGUUAAAAUAUAGUUC
UAAGUUAGUCUGGUGAAUCCACUCACCCAGAAGAAGCCAGAAG-3′
(SEQ ID NO: 891):
5′-GGGAGACAAACAAAGAGCGACAAGGGCAGUCUGGGGGAUGGCUGUG
AGGUCCCAAUGUAUCGCAUCUCACACCCAGAAGAAGCCAGAAG-3′
(SEQ ID NO: 892):
5′-GGGAGACAAACAAAGAGCGACAAGGGCAGUUAAAAUAUAGUUCUAA
GUUAGUCUGGUGAAUCCACUCACCCAGAAGAAGCCAGAAG-3′

### Example 32: Reverse Chimera Linkers

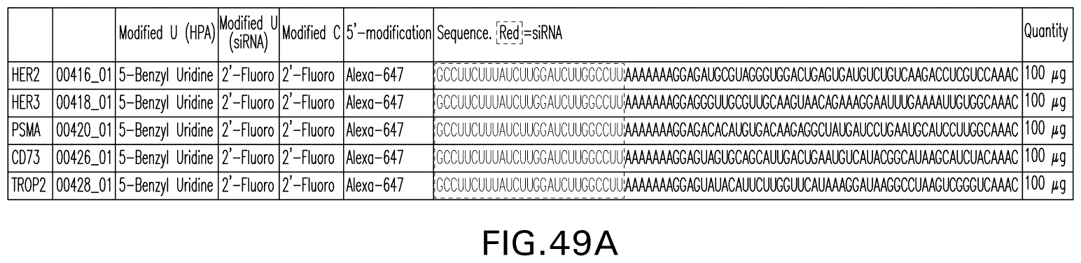

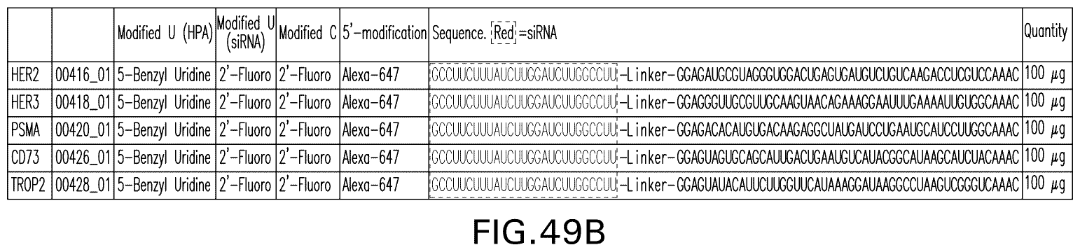

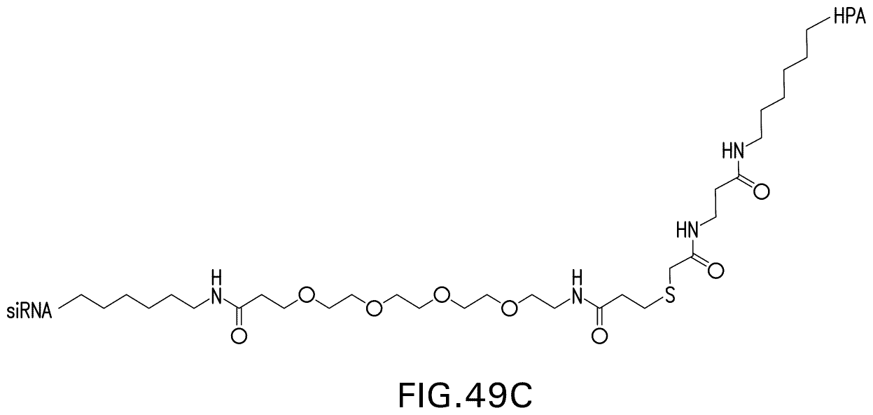

Standard linkage is 3′ end of an aptamer linked to 5′ of an siRNA. Here we provide an example of the 3′end of the siRNA linked to the 5′end of the aptamer. Linked via poly-adenosine linkage. siRNA is the guide strand (FIG. 49A). Provided is another example of a reverse chimera structure using an alternative linker shown in (FIG. 49C). However, alternative linkers as previously described can be used in place here.

### Example 33: In Vivo Inhibition of UBB and UBC mRNA by the UBB-UBC Dual Targeting siRNA

Male NSG mice are injected subcutaneously (HCT116) or intrasplenically (mHCT116) with human HCT116 CRC tumor cells to disseminate LM, whereas experimental controls receive saline. Huot et al. demonstrated elevated ubiquitin expression in this model (Huot et al., Dis Models & Mech, 13:1754-8403 (2020)).

Mice will be treated with the dual UBB-UBC targeting siRNAs conjugated to EPCAM aptamer, Epcam-scrambled siRNA, or vehicle by intraperitoneal injection of 0.1 ml of the indicated solution. Mice will be treated with a dose of dual targeting siRNA sufficient to inhibit expression of UBB and UBC by at least 10%, at least 15%, at least 20%, at least 25%, at least 30%, at least 40%, at least 50%, at least 60%, at least 70%, at least 80% or at least 90% or more, for at least 5, more preferably 7, 10, 14, or 18 days. Alternatively, mice will be dosed multiple times in order to inhibit expression of UBB and UBC by at least 10%, at least 15%, at least 20%, at least 25%, at least 30%, at least 40%, at least 50%, at least 60%, at least 70%, at least 80% or at least 90% or more, for at least 5, more preferably 7, 10, 14, or 18 days. All the mice are sacrificed on day 18, and tumors are collected for quantitation.

### Example 34: In Vivo Impact of UBB and UBC mRNA Inhibition on Tumor Size

To assess the impact of a compound comprising dual targeting siRNA conjugated to EPCAM aptamer on tumor growth in vivo, subcutaneous HCT-116 xenografts will be established in athymic nu/nu male mice. The compound will be injected intraperitoneally to tumor-bearing mice every other day for 1 week and every day for the following two weeks. Control mice will be injected intraperitoneally with equivalent volume of PBS or Epcam-scrambled siRNA. All the mice are sacrificed on day 21, and tumors are collected for quantitation.

In certain embodiments, the invention provides pharmaceutical compositions containing a dual targeting siRNA agent, as described herein, and a pharmaceutically acceptable carrier.

The pharmaceutical compositions featured herein are administered in dosages sufficient to inhibit expression of the target genes. In general, a suitable dose of siRNA will be in the range of 0.01 to 200.0 milligrams per kilogram body weight of the recipient per day, generally in the range of 1 to 50 mg per kilogram body weight per day.

The pharmaceutical composition may be administered once daily, or the siRNA may be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A siRNA construct that is processed by cellular machinery to produce one or more siRNA molecules wherein each molecule specifically inhibits expression of a target gene having a sequence from SEQ ID NO: 1 to SEQ ID NO: 594 in Table 1.

2. The construct according to claim 1 wherein the siRNA molecules bind more than one site in each different gene.

3. The construct according to claim 1 further comprising a single siRNA targeting both the UBB and UBC genes.

4. An aptamer-siRNA chimera comprising an aptamer that specifically binds at least one target protein and an siRNA construct that is processed by cellular machinery to produce one or more siRNAs according to claim 1.

5. An aptamer-siRNA chimera comprising:
a. a first end and second end comprising an aptamer that specifically binds at least one target protein; and
b. a siRNA construct between the first and second ends, wherein
i. the siRNA construct is processed by cellular RNAi machinery to produce one or more siRNAs wherein the siRNA molecule is selected from SEQ ID NO: 1 to SEQ ID NO: 594 from Table 1.

6. The chimera according to claim 5 wherein the siRNA construct is processed by cellular RNAi machinery to produce two siRNAs that specifically inhibit three or more different genes.

7. A composition for treating cancer comprising an aptamer-siRNA chimera comprising:
(a) first and second ends, wherein the first and second ends comprise an aptamer that specifically binds at least one target protein that is found on the surface of cancer cells; and
(b) an siRNA construct between the first and second ends, wherein the siRNA construct is processed by cellular RNAi machinery to produce one or more siRNA molecule selected from SEQ ID NO: 1 to SEQ ID NO: 594 from Table 1.

8. The composition according to claim 7 wherein the siRNA construct comprises an siRNA molecule that targets UBB and UBC.

9. The composition according to claim 7 wherein the siRNA construct comprises an siRNA molecule that targets NR4A1, NR4A2 and NR4A3.

10. The composition according to claim 7 wherein the siRNA construct comprises an siRNA molecule that targets ADORA2A and ADORA2B.

11. The composition according to claim 7 wherein the siRNA construct comprises an siRNA molecule that targets MAP2K1 and MAP2K2.

12. The composition according to claim 7 wherein the siRNA construct comprises an siRNA molecule that targets MAPK3 and MAPK1

13. The composition according to claim 7 wherein the siRNA construct comprises an siRNA molecule that targets MAPK11 and MAPK14.

14. The composition according to claim 7 wherein the siRNA construct comprises an siRNA molecule that targets MDM2 and MDM4.

15. The composition according to claim 7 wherein the siRNA construct comprises an siRNA molecule that targets PFKFB3 and PFKFB4.

16. The composition according to claim 7 wherein the siRNA construct comprises an siRNA molecule that targets TOX and TOX2.

17. The composition according to claim 7 wherein the siRNA construct comprises an siRNA molecule that targets BCL2, STAT3, MYC, SYK, CCNE2, CCND1, CCND2, BIRC5, EGFR, UBB, UBC, NR4A1, NR4A2, NR4A3, ADORA2a, ADORA2b, ADORA1, MAP2K1, MAP2K2, MAPK3 (ERK1), MAPK1 (ERK2), HIF1, HIF2, PFKFB3, PFKFB4, PLK1, PLK4, CDK11A, CDK11B, CDK4, CDK6, PARP1, PARP2, MAPK11, MAPK14, MDM2, MDM4, TOX, TOX2, PIKFYVE, MTOR, GRB7, IDO1, c-MYC, CBLB, RICTOR, MSI1, MSI2, AKT1, BATF, ME2, ME3, PTPN2, VHL, or YY1.

18. The composition according to claim 17 further comprising unpaired linkers comprising two to six adenines between each aptamer and siRNA and between each siRNA.

19. The composition according to claim 17, wherein the target protein comprises ERBB2, ERBB3, FOLH1, CD44, EPCAM, FOLH1, PSCA, PDCD1, TACSTD2, NT5E, PDCD1, CTLA4, LAG3, DHX9, AKT1, BATF, ME2, ME3, PTPN2, VHL, or HAVCR2.

20. The composition according to claim 19, wherein the siRNA construct inhibits expression of human and mouse genes.

21. The aptamer-siRNA chimera according to claim 4 comprising a single siRNA targeting both the UBB and UBC genes.

22. The aptamer-siRNA chimera according to claim 21 comprising:
(a) a first aptamer that specifically binds EPCAM; and
(b) a second aptamer that specifically binds EPCAM.

23. The aptamer-siRNA chimera according to claim 21 comprising:
1. a first aptamer that specifically binds PSMA; and
2. a second aptamer that specifically binds PSMA.

24. The aptamer-siRNA chimera according to claim 23 further comprising a siRNA molecule that targets BIRC5.

25. The aptamer-siRNA chimera according to claim 21 comprising:
(a) a first aptamer that specifically binds EPCAM; and
(b) a second aptamer that specifically binds HER3.

26. The aptamer-siRNA chimera according to claim 21 comprising:
(a) a first aptamer that specifically binds HER2; and
(b) a second aptamer that specifically binds HER3.

27. The aptamer-siRNA chimera according to claim 21 comprising:
(a) a first aptamer that specifically binds CD73; and
(b) a second aptamer that specifically binds TROP2.

28. The aptamer-siRNA chimera according to claim 21 comprising:
(a) a first aptamer that specifically binds TROP2; and
(b) a second aptamer that specifically binds HER3.

29. The aptamer-siRNA chimera according to claim 21 comprising:
(c) a first aptamer that specifically binds DHX9; and
(d) a second aptamer that specifically binds DHX9.

30. The aptamer-siRNA chimera according to claim 21 comprising an aptamer that specifically binds EPCAM.

31. The aptamer-siRNA chimera according to claim 4 comprising:
1. a first siRNA targeting the PIKFYVE gene;
2. a second siRNA targeting the MAP2K1 gene;
3. a first aptamer that specifically binds CD44; and
4. Currently Amended second aptamer that specifically binds CD133.

32. The aptamer-siRNA chimera according to claim 4 comprising:
1. a first siRNA targeting the MS12 gene;
2. a second siRNA targeting the UBB gene;
3. a first aptamer that specifically binds CD44; and
4. a second aptamer that specifically binds PSCA.

33. The aptamer-siRNA chimera according to claim 21 comprising:
a) a second siRNA targeting the MSI2 gene;
b) a first aptamer that specifically binds CD44; and
c) a second aptamer that specifically binds PSCA.

34. The aptamer-siRNA chimera according to claim 30 wherein the siRNA is U22ds (SEQ ID No. 645).
